SEQUENCE LISTING (268 sequences)

SEQ ID NO: 1-2 - skipped (2 entries)

SEQ ID NO: 3 - DNA, 10 bp, synthetic construct
  misc_feature 1..10: note = antisense oligonucleotide
  source 1..10: mol_type = other DNA
acacacacac

SEQ ID NO: 4 - DNA, 11 bp, synthetic construct
  misc_feature 1..11: note = antisense oligonucleotide
  source 1..11: mol_type = other DNA
acacacacaca

SEQ ID NO: 5 - DNA, 12 bp, synthetic construct
  misc_feature 1..12: note = antisense oligonucleotide
  source 1..12: mol_type = other DNA
acacacacacac

SEQ ID NO: 6 - DNA, 13 bp, synthetic construct
  misc_feature 1..13: note = antisense oligonucleotide
  source 1..13: mol_type = other DNA
acacacacacaca

SEQ ID NO: 7 - DNA, 16 bp, synthetic construct
  misc_feature 1..16: note = antisense oligonucleotide
  source 1..16: mol_type = other DNA
acacacacacacacac

SEQ ID NO: 8 - DNA, 18 bp, synthetic construct
  misc_feature 1..18: note = antisense oligonucleotide
  source 1..18: mol_type = other DNA
acacacacacacacacac

SEQ ID NO: 9-11 - skipped (3 entries)

SEQ ID NO: 12 - DNA, 10 bp, synthetic construct
  misc_feature 1..10: note = antisense oligonucleotide
  source 1..10: mol_type = other DNA
cacacacaca

SEQ ID NO: 13 - DNA, 11 bp, synthetic construct
  misc_feature 1..11: note = antisense oligonucleotide
  source 1..11: mol_type = other DNA
cacacacacac

SEQ ID NO: 14 - DNA, 12 bp, synthetic construct
  misc_feature 1..12: note = antisense oligonucleotide
  source 1..12: mol_type = other DNA
cacacacacaca

SEQ ID NO: 15 - DNA, 13 bp, synthetic construct
  misc_feature 1..13: note = antisense oligonucleotide
  source 1..13: mol_type = other DNA
cacacacacacac

SEQ ID NO: 16 - DNA, 16 bp, synthetic construct
  misc_feature 1..16: note = antisense oligonucleotide
  source 1..16: mol_type = other DNA
cacacacacacacaca

SEQ ID NO: 17 - skipped

SEQ ID NO: 18 - DNA, 18 bp, synthetic construct
  misc_feature 1..18: note = antisense oligonucleotide
  source 1..18: mol_type = other DNA
cacacacacacacacaca

SEQ ID NO: 19 - DNA, 17 bp, synthetic construct
  misc_feature 1..17: note = antisense oligonucleotide
  source 1..17: mol_type = other DNA
tccacactgaacaaacc

SEQ ID NO: 20 - DNA, 23 bp, synthetic construct
  misc_feature 1..23: note = antisense oligonucleotide
  source 1..23: mol_type = other DNA
cacacacacacacacacacacac

SEQ ID NO: 21 - DNA, 24 bp, synthetic construct
  misc_feature 1..24: note = antisense oligonucleotide
  source 1..24: mol_type = other DNA
acacacacacacacacacacacac

SEQ ID NO: 22 - DNA, 24 bp, synthetic construct
  misc_feature 1..24: note = antisense oligonucleotide
  source 1..24: mol_type = other DNA
cacacacacacaacacacacacac

SEQ ID NO: 23 - DNA, 25 bp, synthetic construct
  misc_feature 1..25: note = antisense oligonucleotide
  source 1..25: mol_type = other DNA
cacacacacacacacacacacacac

SEQ ID NO: 24 - DNA, 26 bp, synthetic construct
  misc_feature 1..26: note = antisense oligonucleotide
  source 1..26: mol_type = other DNA
acacacacacacacacacacacacac

SEQ ID NO: 25 - DNA, 27 bp, synthetic construct
  misc_feature 1..27: note = antisense oligonucleotide
  source 1..27: mol_type = other DNA
cacacacacacacacacacacacacac

SEQ ID NO: 26 - DNA, 28 bp, synthetic construct
  misc_feature 1..28: note = antisense oligonucleotide
  source 1..28: mol_type = other DNA
acacacacacacacacacacacacacac

SEQ ID NO: 27 - DNA, 28 bp, synthetic construct
  misc_feature 1..28: note = antisense oligonucleotide
  source 1..28: mol_type = other DNA
cacacacacacacacacacacacacaca

SEQ ID NO: 28 - DNA, 30 bp, synthetic construct
  misc_feature 1..30: note = antisense oligonucleotide
  source 1..30: mol_type = other DNA
acacacacacacacacacacacacacacac

SEQ ID NO: 29 - DNA, 31 bp, synthetic construct
  misc_feature 1..31: note = antisense oligonucleotide
  source 1..31: mol_type = other DNA
cacacacacacacacacacacacacacacac

SEQ ID NO: 30 - DNA, 34 bp, synthetic construct
  misc_feature 1..34: note = antisense oligonucleotide
  source 1..34: mol_type = other DNA
acacacacacacacacaccacacacacacacaca

SEQ ID NO: 31 - DNA, 34 bp, synthetic construct
  misc_feature 1..34: note = antisense oligonucleotide
  source 1..34: mol_type = other DNA
cacacacacacacacacacacacacacacacaca

SEQ ID NO: 32 - DNA, 36 bp, synthetic construct
  misc_feature 1..36: note = antisense oligonucleotide
  source 1..36: mol_type = other DNA
cacacacacacacacacaacacacacacacacacac

SEQ ID NO: 33 - DNA, 36 bp, synthetic construct
  misc_feature 1..36: note = antisense oligonucleotide
  source 1..36: mol_type = other DNA
acacacacacacacacaccacacacacacacacaca

SEQ ID NO: 34 - DNA, 32 bp, synthetic construct
  misc_feature 1..32: note = antisense oligonucleotide
  source 1..32: mol_type = other DNA
acacacacacacacacacacacacacacacac

SEQ ID NO: 35 - RNA, 12 nt, synthetic construct
  misc_feature 1..12: note = human RNA TDP-43 binding site
  source 1..12: mol_type = other RNA
tgtgtgtgtgtg

SEQ ID NO: 36 - DNA, 16 bp, synthetic construct
  misc_feature 1..16: note = LNA/DNA mixmer which targets and upregulates the expression of STMN2
  source 1..16: mol_type = other DNA
cacacacgcacacatg

SEQ ID NO: 37 - RNA, 10 nt, synthetic construct
  misc_feature 1..10: note = sequence complementary to antisense oligonucleotide
  source 1..10: mol_type = other RNA
tgtgtgtgtg

SEQ ID NO: 38 - RNA, 11 nt, synthetic construct
  misc_feature 1..11: note = sequence complementary to antisense oligonucleotide
  source 1..11: mol_type = other RNA
tgtgtgtgtgt

SEQ ID NO: 39 - RNA, 13 nt, synthetic construct
  misc_feature 1..13: note = sequence complementary to antisense oligonucleotide
  source 1..13: mol_type = other RNA
tgtgtgtgtgtgt

SEQ ID NO: 40 - RNA, 10 nt, synthetic construct
  misc_feature 1..10: note = sequence complementary to antisense oligonucleotide
  source 1..10: mol_type = other RNA
gtgtgtgtgt

SEQ ID NO: 41 - RNA, 11 nt, synthetic construct
  misc_feature 1..11: note = sequence complementary to antisense oligonucleotide
  source 1..11: mol_type = other RNA
gtgtgtgtgtg

SEQ ID NO: 42 - RNA, 12 nt, synthetic construct
  misc_feature 1..12: note = sequence complementary to antisense oligonucleotide
  source 1..12: mol_type = other RNA
gtgtgtgtgtgt

SEQ ID NO: 43 - RNA, 13 nt, synthetic construct
  misc_feature 1..13: note = sequence complementary to antisense oligonucleotide
  source 1..13: mol_type = other RNA
gtgtgtgtgtgtg

SEQ ID NO: 44 - DNA, 21 bp, synthetic construct
  misc_feature 1..21: note = primer
  source 1..21: mol_type = other DNA
cagctcatcctcagtcatgtc

SEQ ID NO: 45 - DNA, 21 bp, synthetic construct
  misc_feature 1..21: note = primer
  source 1..21: mol_type = other DNA
gatggtgtgactgcaaacttc

SEQ ID NO: 46 - RNA, 14 nt, synthetic construct
  misc_feature 1..14: note = sequence complementary to antisense oligonucleotide
  source 1..14: mol_type = other RNA
tgtgtgtgtgtgtg

SEQ ID NO: 47 - RNA, 14 nt, synthetic construct
  misc_feature 1..14: note = sequence complementary to antisense oligonucleotide
  source 1..14: mol_type = other RNA
gtgtgtgtgtgtgt

SEQ ID NO: 48 - RNA, 18 nt, synthetic construct
  misc_feature 1..18: note = sequence complementary to antisense oligonucleotide
  source 1..18: mol_type = other RNA
tgtgtgtgtgtgtgtgtg

SEQ ID NO: 49 - RNA, 18 nt, synthetic construct
  misc_feature 1..18: note = sequence complementary to antisense oligonucleotide
  source 1..18: mol_type = other RNA
gtgtgtgtgtgtgtgtgt

SEQ ID NO: 50 - DNA, 23 bp, synthetic construct
  misc_feature 1..23: note = antisense oligonucleotide
  modified_base 12: mod_base = i
  source 1..23: mol_type = other DNA
cacacacccacncacacacgcac

SEQ ID NO: 51 - DNA, 23 bp, synthetic construct
  misc_feature 1..23: note = antisense oligonucleotide
  modified_base 6: mod_base = i
  source 1..23: mol_type = other DNA
cacacncacacacacacacacac

SEQ ID NO: 52 - DNA, 23 bp, synthetic construct
  misc_feature 1..23: note = antisense oligonucleotide
  modified_base 12: mod_base = i
  source 1..23: mol_type = other DNA
cacacacacacncacacacacac

SEQ ID NO: 53 - DNA, 23 bp, synthetic construct
  misc_feature 1..23: note = antisense oligonucleotide
  modified_base 6: mod_base = i
  source 1..23: mol_type = other DNA
cacacncacacacacacacactc

SEQ ID NO: 54 - DNA, 23 bp, synthetic construct
  misc_feature 1..23: note = antisense oligonucleotide
  source 1..23: mol_type = other DNA
cacatacacacccacacacacac

SEQ ID NO: 55 - DNA, 23 bp, synthetic construct
  misc_feature 1..23: note = antisense oligonucleotide
  source 1..23: mol_type = other DNA
cacacacacacacacacgcacac

SEQ ID NO: 56 - DNA, 23 bp, synthetic construct
  misc_feature 1..23: note = antisense oligonucleotide
  modified_base 4: mod_base = i
  modified_base 8: mod_base = i
  modified_base 16: mod_base = i
  modified_base 20: mod_base = i
  source 1..23: mol_type = other DNA
cacncacncacacacncacncac

SEQ ID NO: 57 - DNA, 23 bp, synthetic construct
  misc_feature 1..23: note = antisense oligonucleotide
  source 1..23: mol_type = other DNA
cacacacacacacactctcacac

SEQ ID NO: 58 - DNA, 23 bp, synthetic construct
  misc_feature 1..23: note = antisense oligonucleotide
  source 1..23: mol_type = other DNA
ctcacacacacacacacacacac

SEQ ID NO: 59 - DNA, 23 bp, synthetic construct
  misc_feature 1..23: note = antisense oligonucleotide
  source 1..23: mol_type = other DNA
cacacacacacaaacacacacac

SEQ ID NO: 60 - DNA, 23 bp, synthetic construct
  misc_feature 1..23: note = antisense oligonucleotide
  source 1..23: mol_type = other DNA
cacacgcacgcacacacacacac

SEQ ID NO: 61 - DNA, 23 bp, synthetic construct
  misc_feature 1..23: note = antisense oligonucleotide
  source 1..23: mol_type = other DNA
cacgcacacacccacacactcac

SEQ ID NO: 62 - DNA, 23 bp, synthetic construct
  misc_feature 1..23: note = antisense oligonucleotide
  source 1..23: mol_type = other DNA
cacacacacacactctctcacac

SEQ ID NO: 63 - DNA, 23 bp, synthetic construct
  misc_feature 1..23: note = antisense oligonucleotide
  modified_base 14: mod_base = i
  modified_base 16: mod_base = i
  modified_base 18: mod_base = i
  modified_base 20: mod_base = i
  source 1..23: mol_type = other DNA
cacacacacacacncncncncac

SEQ ID NO: 64 - DNA, 23 bp, synthetic construct
  misc_feature 1..23: note = antisense oligonucleotide
  source 1..23: mol_type = other DNA
cactcacacactcacacacacac

SEQ ID NO: 65 - DNA, 23 bp, synthetic construct
  misc_feature 1..23: note = antisense oligonucleotide
  source 1..23: mol_type = other DNA
cacgcacacacgcacacacacac

SEQ ID NO: 66 - DNA, 23 bp, synthetic construct
  misc_feature 1..23: note = antisense oligonucleotide
  source 1..23: mol_type = other DNA
cacccacacacccacacacacac

SEQ ID NO: 67 - DNA, 23 bp, synthetic construct
  misc_feature 1..23: note = antisense oligonucleotide
  source 1..23: mol_type = other DNA
cacacgcacatacacacacccac

SEQ ID NO: 68 - DNA, 23 bp, synthetic construct
  misc_feature 1..23: note = antisense oligonucleotide
  source 1..23: mol_type = other DNA
ttcacacacacacacacacacac

SEQ ID NO: 69 - DNA, 23 bp, synthetic construct
  misc_feature 1..23: note = antisense oligonucleotide
  source 1..23: mol_type = other DNA
cacacacacacacacacacacaa

SEQ ID NO: 70 - DNA, 29 bp, synthetic construct
  misc_feature 1..29: note = antisense oligonucleotide
  modified_base 21: mod_base = i
  source 1..29: mol_type = other DNA
cacacacacacacacacacanacacacac

SEQ ID NO: 71 - DNA, 29 bp, synthetic construct
  misc_feature 1..29: note = antisense oligonucleotide
  modified_base 5: mod_base = i
  source 1..29: mol_type = other DNA
cacanacacacacacacacacacacacac

SEQ ID NO: 72 - DNA, 29 bp, synthetic construct
  misc_feature 1..29: note = antisense oligonucleotide
  source 1..29: mol_type = other DNA
catacacacacacacacacatacacacac

SEQ ID NO: 73 - DNA, 29 bp, synthetic construct
  misc_feature 1..29: note = antisense oligonucleotide
  source 1..29: mol_type = other DNA
caaacacacacacacacacacacacacac

SEQ ID NO: 74 - DNA, 29 bp, synthetic construct
  misc_feature 1..29: note = antisense oligonucleotide
  source 1..29: mol_type = other DNA
cacacacactcacacacacacacacacac

SEQ ID NO: 75 - DNA, 29 bp, synthetic construct
  misc_feature 1..29: note = antisense oligonucleotide
  source 1..29: mol_type = other DNA
cacagacacacacacacacacacacacac

SEQ ID NO: 76 - DNA, 29 bp, synthetic construct
  misc_feature 1..29: note = antisense oligonucleotide
  source 1..29: mol_type = other DNA
cacaaacacacacacacacacacacacac

SEQ ID NO: 77 - DNA, 29 bp, synthetic construct
  misc_feature 1..29: note = antisense oligonucleotide
  source 1..29: mol_type = other DNA
cacatacacacacacacacacacacacac

SEQ ID NO: 78 - DNA, 29 bp, synthetic construct
  misc_feature 1..29: note = antisense oligonucleotide
  source 1..29: mol_type = other DNA
catacgcacatacacgcacacacaaacac

SEQ ID NO: 79 - DNA, 29 bp, synthetic construct
  misc_feature 1..29: note = antisense oligonucleotide
  source 1..29: mol_type = other DNA
cacatatacacatacacacacacacacac

SEQ ID NO: 80 - DNA, 29 bp, synthetic construct
  misc_feature 1..29: note = antisense oligonucleotide
  source 1..29: mol_type = other DNA
cacacacacacacacacactctctctctc

SEQ ID NO: 81 - DNA, 29 bp, synthetic construct
  misc_feature 1..29: note = antisense oligonucleotide
  source 1..29: mol_type = other DNA
cacgcacacacacacacacacacacacac

SEQ ID NO: 82 - DNA, 29 bp, synthetic construct
  misc_feature 1..29: note = antisense oligonucleotide
  modified_base 17: mod_base = i
  modified_base 19: mod_base = i
  modified_base 21: mod_base = i
  source 1..29: mol_type = other DNA
cacacacacacacacantntntcacacac

SEQ ID NO: 83 - DNA, 29 bp, synthetic construct
  misc_feature 1..29: note = antisense oligonucleotide
  source 1..29: mol_type = other DNA
cgcacacacacacacacacacacacacac

SEQ ID NO: 84 - DNA, 29 bp, synthetic construct
  misc_feature 1..29: note = antisense oligonucleotide
  source 1..29: mol_type = other DNA
cacacacacacacacagacagacagacac

SEQ ID NO: 85 - DNA, 29 bp, synthetic construct
  misc_feature 1..29: note = antisense oligonucleotide
  source 1..29: mol_type = other DNA
cacacacacaaaaaaacacacacacacac

SEQ ID NO: 86 - DNA, 29 bp, synthetic construct
  misc_feature 1..29: note = antisense oligonucleotide
  source 1..29: mol_type = other DNA
cacacacacacccccccacacacacacac

SEQ ID NO: 87 - DNA, 25 bp, synthetic construct
  misc_feature 1..25: note = antisense oligonucleotide
  source 1..25: mol_type = other DNA
cacacacactctctcacacacacac

SEQ ID NO: 88 - DNA, 25 bp, synthetic construct
  misc_feature 1..25: note = antisense oligonucleotide
  source 1..25: mol_type = other DNA
cacaaacacacacacacacacacac

SEQ ID NO: 89 - DNA, 25 bp, synthetic construct
  misc_feature 1..25: note = antisense oligonucleotide
  source 1..25: mol_type = other DNA
cactcacacacacacacacacacac

SEQ ID NO: 90 - DNA, 25 bp, synthetic construct
  misc_feature 1..25: note = antisense oligonucleotide
  source 1..25: mol_type = other DNA
cacacacacaaacacacacacacac

SEQ ID NO: 91 - DNA, 25 bp, synthetic construct
  misc_feature 1..25: note = antisense oligonucleotide
  source 1..25: mol_type = other DNA
cacccacacacacacacacacacac

SEQ ID NO: 92 - DNA, 25 bp, synthetic construct
  misc_feature 1..25: note = antisense oligonucleotide
  source 1..25: mol_type = other DNA
ctctctcacacacacacacacacac

SEQ ID NO: 93 - DNA, 25 bp, synthetic construct
  misc_feature 1..25: note = antisense oligonucleotide
  source 1..25: mol_type = other DNA
caaacacacacacacacacacacac

SEQ ID NO: 94 - DNA, 25 bp, synthetic construct
  misc_feature 1..25: note = antisense oligonucleotide
  source 1..25: mol_type = other DNA
cacacacacccacacacacacacac

SEQ ID NO: 95 - DNA, 25 bp, synthetic construct
  misc_feature 1..25: note = antisense oligonucleotide
  source 1..25: mol_type = other DNA
cacacacacacacacccacacacac

SEQ ID NO: 96 - DNA, 25 bp, synthetic construct
  misc_feature 1..25: note = antisense oligonucleotide
  source 1..25: mol_type = other DNA
cacacacacacacacgcacacacac

SEQ ID NO: 97 - DNA, 25 bp, synthetic construct
  misc_feature 1..25: note = antisense oligonucleotide
  source 1..25: mol_type = other DNA
cacacacacacacactcacacacac

SEQ ID NO: 98 - DNA, 25 bp, synthetic construct
  misc_feature 1..25: note = antisense oligonucleotide
  source 1..25: mol_type = other DNA
cacacacacatacacacacacacac

SEQ ID NO: 99 - DNA, 25 bp, synthetic construct
  misc_feature 1..25: note = antisense oligonucleotide
  source 1..25: mol_type = other DNA
cacacacacagacacacacacacac

SEQ ID NO: 100 - DNA, 25 bp, synthetic construct
  misc_feature 1..25: note = antisense oligonucleotide
  source 1..25: mol_type = other DNA
tttacacacacacacacacacacac

SEQ ID NO: 101 - DNA, 25 bp, synthetic construct
  misc_feature 1..25: note = antisense oligonucleotide
  source 1..25: mol_type = other DNA
cacgcacacgcacacacacacacac

SEQ ID NO: 102 - DNA, 25 bp, synthetic construct
  misc_feature 1..25: note = antisense oligonucleotide
  source 1..25: mol_type = other DNA
cacgcacacacgcacacacacgcac

SEQ ID NO: 103 - DNA, 25 bp, synthetic construct
  misc_feature 1..25: note = antisense oligonucleotide
  source 1..25: mol_type = other DNA
cacacgcacacacacacacacacac

SEQ ID NO: 104 - DNA, 23 bp, synthetic construct
  misc_feature 1..23: note = Probe
  source 1..23: mol_type = other DNA
cagcgccccacaaacacttttct

SEQ ID NO: 105 - DNA, 15 bp, synthetic construct
  misc_feature 1..15: note = primer
  source 1..15: mol_type = other DNA
ctgctcagcgtctgc

SEQ ID NO: 106 - DNA, 15 bp, synthetic construct
  misc_feature 1..15: note = primer
  source 1..15: mol_type = other DNA
gttgcgaggttccgg

SEQ ID NO: 107 - DNA, 26 bp, synthetic construct
  misc_feature 1..26: note = probe
  source 1..26: mol_type = other DNA
caatggcctacaaggaaaaaatgaag

SEQ ID NO: 108 - DNA, 24 bp, synthetic construct
  misc_feature 1..24: note = primer
  source 1..24: mol_type = other DNA
ctaatggttaaacgtgaggacagc

SEQ ID NO: 109 - DNA, 20 bp, synthetic construct
  misc_feature 1..20: note = primer
  source 1..20: mol_type = other DNA
atctggtcctccaaagtggg

SEQ ID NO: 110 - DNA, 34 bp, synthetic construct
  misc_feature 1..34: note = probe
  source 1..34: mol_type = other DNA
ctggataaggaaactgagaagaaaagaagaacag

SEQ ID NO: 111 - DNA, 17 bp, synthetic construct
  misc_feature 1..17: note = primer
  source 1..17: mol_type = other DNA
cgagccctcggagtttg

SEQ ID NO: 112 - DNA, 20 bp, synthetic construct
  misc_feature 1..20: note = primer
  source 1..20: mol_type = other DNA
tccttccaagaaatggtggc

SEQ ID NO: 113 - DNA, 22 bp, synthetic construct
  misc_feature 1..22: note = probe
  source 1..22: mol_type = other DNA
gaatatgatgctgctgctgatg

SEQ ID NO: 114 - DNA, 20 bp, synthetic construct
  misc_feature 1..20: note = primer
  source 1..20: mol_type = other DNA
ctgacagtgccaataccacc

SEQ ID NO: 115 - DNA, 19 bp, synthetic construct
  misc_feature 1..19: note = primer
  source 1..19: mol_type = other DNA
gctgctccgtggtcttaat

SEQ ID NO: 116 - DNA, 26 bp, synthetic construct
  misc_feature 1..26: note = probe
  source 1..26: mol_type = other DNA
atgaagacgctaaagcccggaagcag

SEQ ID NO: 117 - DNA, 20 bp, synthetic construct
  misc_feature 1..20: note = primer
  source 1..20: mol_type = other DNA
gatcaaaggcgaggagaagg

SEQ ID NO: 118 - DNA, 19 bp, synthetic construct
  misc_feature 1..19: note = primer
  source 1..19: mol_type = other DNA
tggcatctgggatcttcac

SEQ ID NO: 119 - DNA, 21 bp, synthetic construct
  misc_feature 1..21: note = probe
  source 1..21: mol_type = other DNA
catgagaacctgttccacttc

SEQ ID NO: 120 - DNA, 333 bp, Homo sapiens
  source 1..333: mol_type = other DNA
agtcttctctctcgctctctccgctgctgtagccggaccctttgccttcgccactgctcagcgtctgcacatccctacaatggctaaaacagcaatgggactcggcagaagaccttcgagagaaaggtagaaaataagaatttggctctctgtgtgagcatgtgtgcgtgtgtgcgagagagagagacagacagcctgcctaagaagaaatgaatgtgaatgcggcttgtggcacagttgacaaggatgataaatcaataatgcaagcttactatcatttatgaatagcaatactgaagaaattaaaacaaaagattgctgtctcaatatatc

SEQ ID NO: 121 - DNA, 2755 bp, Homo sapiens
  source 1..2755: mol_type = other DNA
gaaattaatccttttggtagtcatattaggtagaatcatgatgcacattctaataagtcacactgtactcatgtccctttagcctggaaaccatctccctgaaactggaactcagctttttcgtctgtatctgtaatgctttcatgatgcctggaaaatagaagagacttaataaatacatgattgatgagtgagtgagcgagtgaatgaatgaagagaaaagagcagaaacgaaaccaaaactgtcaagaaaaaaaaatcaaagatcaattcttgtcatacttacttcctacttcttttagccatattctaatctaaaggatcatcgattttaatttaaatatgtacttcttattctgttgtcgtatgatgatattttaaaaactaagaatgtaaaatcaaagtaaatcagaagtgtattatgtggagaatgtgggaatgattctgaaagtcataccgtcttttggaggaagcattttggaggtttctaatgttatattttgactgggtcattattcgctgtgcatttggctggtttgttagatggtttgggggtagctagtaaatagcctttttttaaaactaggattttttttacaaaatatattggatttcttatattgctttcctactctttcgtgaaaaaataaaacagatcttttcttcttttttcttctgacattgtttttcagtttttgctccttttcttcaagattcaagtttatgcttttctttcattttgccacctatttgtattaaatgactcatttgtttcccttaaacttataagtaatagttatgtaattttatttattaagctcattatgtgatttttgtgtagacattcagtccttctactcagggaacacaaaatataagaaaacgattcagatgcttgccatgaagtattacgtgtgtagagacttggacatattatcaggaaaagacctctaattctccatattttcaatgttttcctacttatttggaagaataaacagatatgaatctaataacacacagatatacacatccaatgaaaattaccgatacttttttttataaatggtagcttatttttatgaaaaatttatgtgtgctcggagagcctgataatatggaaataaaatactgtgctctgaagggtttttcttgaagacaaaccttttgtgtacctgggtgaatcagcatgctcatttggagcatatataatgagaagcatcttctcagtgctttacaggaaagaattttaactattttcctaagtaatgatgttaattatcccagtaatctctaaatacattccctttgaagcagaagactgaaattttgtagccccaaattttattattccacttccagtttttagaattgaaacctaagacaccaagtttatcattggtgggacaatgtgagaaatagaacccaagcttttataaagatgctatgtattttgcatatcatttcgacacagaggttgcttctaatatcaattaaatccacaagaagtacccagcaattctacctgcccttcatctttactacataaccataaccttttcttgttactgaaagtcttgaatatttttagaggtagaaagcatgtagttgaaaatgtattcatatgaaaactaaggttccttccctgctaacttgaccagataatgaccatttcaggctctcagggcactggtaaattaattcacttgctaattttatacattgtgtcaggacggtgttcctgagttttaaatatacaaagatggacgatgcatggttttgctcttgaagagcccacagtcagaaaagggtaatagttgtatgaacaaataaatgataattttgaaatatttttctatgagcacatagaacacagtgctcagccacctctacttgtgggtattcaatgaaggctcgctaaagagtgatagtttacctggattttgaacaatagccaagaattttttctatacgaagagcgttatgggagagagtccagctagaggaaattgttttgtgaagaagtcatagcatggttggtttgtatggcaaaaggtaacacttcacttggcctagggttgtggaatatggaaatacaataataattaaatgtaaattatgaaaggccttctctgccaggcagtatattgcagtagtcaaaagcattggctctgactggataaagaaaaatgtggcacatatataccatggaatactatgcagccataaaaaaggatgagttcatgtcctttgcagggacatggatgaagttggaagccatcattctcagcaaactatcacaagaacagaaaaccaaacaccacatgttctcactcataagtgggagttgaacaatgagaacacatggaatagggaggggaacatcacacaccagggcctctcggggggttggggggctaggggagggataacattaggagaaatactcaatgtagatgatgggttgatgggtgcagcaaaccaccatggcatgtgtatacttacgtaacaaaactgcacattctgcacatgtaccccagaacttaaagtataataaaaaaagatatatgtatgtgtgtgtgtgtgtgtgtgtgtgtgtgtgtgtgtgtgtgaaataaaaaacaaaacaaaagcattggctctgtacttggactgcctgagttggaatctctcacttctgccagtgtttcctgcctgcccttgggtaaaacttactctctctaagatacaatttcttctctgtaaaaattgggaa

SEQ ID NO: 122 - DNA, 352 bp, Homo sapiens
  source 1..352: mol_type = other DNA
cttttgtcctgtggtattctgtaaaggtgatccagggtgatgttcctacagcaactttgactttcgattagtagagtcttttattggattgtttaacttttaggagagcagtcctggttcatagtgtacactctccattccagttgttcatccaggagacagacacttcaaaaatgtaaagatgactgacgaaggtggtatgtagtgtttgtctctgaaaaaaaaaaagatagtatttctctcacaagcagaaaacatatatggacgtgtgtgtgtctgtgtgtgtgtgtgtgtgtgtgtctgtgtgtgtgtgtttattgtaggaacacaggctaacagaaacagcattaag

SEQ ID NO: 123 - DNA, 404 bp, Homo sapiens
  source 1..404: mol_type = other DNA
acactctccaccaaagaacagccagaaaagatggcaggcagtattggttaaaatctggacactactgtgttttatagtcttcttacagatctatctgatgtcaaattttattaaccttttccactgtaattttttctcaaggttgaaataactggatcccagttacattttacatggaatgtaagtgtgtgtgtatttgcacatgtgtgtacacacagtatgtgtgtgttcatatatatatacataatatatgcagggtatgtgtgtatttattggtaagcaactctattcaagaactttctaatcttttgtatgtagagtgtgtgtgtgtgtgtgtgtgtatatatacacactaaagactagaaagttcttaaatagagaaagttgtttatcaacaaatacgt

SEQ ID NO: 124 - protein, 16 aa, Homo sapiens
MAKTAMGLGRRPSRER

SEQ ID NO: 125 - DNA, 10231 bp, Homo sapiens
  source 1..10231: mol_type = other DNA
gagtcttctgacagtgccaataccaccatagaggatgaagacgctaaaggtacctgcacttgagtccttgcccccccagcggccttggcattgctgggttgctctttgaggtgggtgggacttgggcagggtcaactctcctgcgacgcctagtttatgcatgtgttgaggggctcagggaccctgtagctgtaatcctgctccaagcctgggtgtcaggcctgcccagagcggagaagcatggcagagatgaccgacagctgggcagtctcggtcaccgcatccaagtgaggaagccacggctttgcatggaggcaggttctccacaccaggaccctcacggggaaacaggcccatgggtagaatttgttccaagatgctgtccttgtcttaaagctccttaagcttgcgtttctgtccagcatgcacttgccaagtggccgggcagctgggtgagtgtttccgtgtttgcctttgcttagccaggagtgtcctgctgcggtgggtttctgcaccacagattccagggccccctcccttgctcacccaggccaatgtcttgtgtgttccccaagaggcccccagggcaccaggcactggggcatgctccatggattctgccgcctccagaccacccacatggggcctcctgaccctcatcgctcacacggtcacctaataagccttatgctgttctcagggctaccctggtgcccaaaaagggtcagccactctgccagtttaggggagaaaacttctcacctgtccaaagcatagccttgctcctgcccggcctacccagctatgacactgtccctgagcagagatgagcacaggactttgggccctggatgccggagagtgggtgtttgtgtgattcccctgcagtctggaacaggccccaaaggcaacagcatgaaggctgtccagaggttctccatcaccctcagccgagtggggtgctgagcagtgagggaggggacctgggaggggggcccagcctggatcctgcaggggagaagagaagacagccagaagccagcagctgtggctcagatctgagcccgagcagcctctcgaggtggaggcagacaccccccaccccaccccgtgcagaaagaagccttgccagcctgccctgaggctggtacagagtccaggcaggctcagtggccatcatgcccctacgatgactgtcactccctctccgtgcgcctggcctctgctggctctggccaggggtggtcacagcactagggtggcagggtggcctctgactctgcgccagcctgcactggcctgtgctgccctggcctctgctggctctggctctggcaccggtcccgtgttggctccttcagccttcacatacctgctgcggccaccacaggcccaggacccccacaggtggccaccccacctccaccccaggagccccaggtatccagctgtcaccccctccctccctcctggcctccccctgtccttctccagttgccttcttttcctgcgggcgcaccacccacctgcctgcctcacctgttccgcctcagcccccagggtccccgacatcctgagctcagtgaggaggggctcgggagccccagaagccgaggggcccctgccctgcccatctccggctccctttagccccctgccagccccatgtaagtagcctgggtcctgctgctgtgggggtcatgttggagggctggcaaccccctagaggggccactccagagccgagggcaggctgagcgtggaccctggctccagcctcatcaccccacaatccctcactggggctttccagggtggccccagcccatcgagccccacctctttgtgaggagggccctggaccactttcctgctcaaggccactgggcaggatgggaggccctggaggctcgggcctcaattccagtcttcagggtcggtgcaggcctcactccacctcagcttgcgggcgggggggctccctgctattgaggcaggctctgattcagggcctgatcccagggcccaaggggtctagaacacgggacccctcccactggcctcctccgccttgccgccgcctcgtgtgtctgtctgcctcatgttcacgtctcatctgttccaccccagcccccaggatctctgacatcctgaactctgtgagaaggggttcaggaaccccagaagccgagggccccctctcagcggggcccccgccctgcctgtctccggctctcctaggccccctgtcctccccgtgtaagtagtggcccccaggcctgccgcctctgctgccggacagctccctgcgaatggccggcgctcagcagcttcccacctgcatgcacggcccagctaccctgccccggcgccgcagcctggagtcctgccctggcggggcttcctgtgggctcccatgctaaccagcagggcagctcctggcttctccctaaggggcccagacccctccacggctcctgctcccactgccactccccgctcgctgtccagccccaggcccctctccaaaatgtctgtcccagccctgggcagccctggcccctccgaggccccccatgcccctaggccctctctgctgatcactgtcccagccccacagacttcacacccacccaggggccctgcccatggtgcccaggagctgcactcagggccaccctggttcctgatgtggccccaacccctgagcaccctccctcagtctaggaggctgaggaaggtgccaaaactggaaccccgaccagggtctctggagctcaccaacaaggggatagtacggagaatcataagcctggcctctgctgacctgggctgtcctcatggggccaggccaggcctcctctgtaacgcccgtgactccctcctctccctgtaaccccgtccagcgttcctcaagggccacttacctgacagcttcttgctggccagcagcctctccctggagggtgccctctgcccccagcagcttcagcccacgccacccgacagccagagcatctgcccttcactcctgcagcctcctctccacgcaccacgctgtccgcagcagcaccctctgtccccctgtctccctccgtccccccatatccccctcggtcagcctacaacctctccacgtccccctaagtccacgctctatccctacatccccctctgtcccccaaattcccctctttccctcatttccattttcctccccaaactctgctctgcccctcacattctccctctgtcccccacaccctcctctgtcccccagactctccctctgtcccccacaccctcctctgtcccccatatacccctctgtcccccacacccaccttggtcccttcacgcccttttctgtcccccacaccccctctgttccctacactctccctctgtcctccagaccctcctctgtcccccacactccctctgtcccccacaccccctgtcccccacactctccctctgccccccagaccctcctctgtcccctacactccctctgtcccccatatccccctctgtcccccacaccctcctctgtcctccaccccctgccccccatacccccttctgtcccccacacttcctctgtcttccacaccccctcctgtcccccacaccccctctgtcccccagactctccctctgtcccccacactccgtctgtcccccacacctcctgtcttccacacccccttctgtcccccacaccccctctgtcccccatactctcctctgtcccccacctccccctctgttccccacaccgccttctgtcccccacaccccctctgtcttccacttcccctctgtcccccacatccccctctgtcccctgcaccctcctctgtcccctgcaccctcctctgtcccctgcacctctctctgtcccccacatccccctctgtcctccacactccctctgtcccccacatccaccttggtcccctcacgcacccccatcccccatgaccccttctgtcccccacaccccctctgtcttccacacccccctctgtcccccacacccaccttggtcccctcatgccccccatcccctacacccccactttgtccccccacatgcccctctgtcccccacgttcccttctgtctcccacgtctcctccatttcccgtttccctctctgtcccccaagctcccctccatcccccacatccccttctttcccctatatcccctctgtcggcccaggtccaccatcttccccccacacccccccattctcccttcctcccctctgtccccttgtgccccatcccccacatctgcctctgtgcccctcaatctctggcttggctgtctgcccatggtttctctcctgcgtgccccccgtgcctgccttgtgttcacgtctcgtctgttccgccccagcccccaggatctctgacatcctgaactctgtgaggaggggctcagggaccccagaagccgagggcccctcgccagtggggcccccgccctgcccatctccgactatccctggccccctgcccaccccatgtaagtagcaccttgagtggccgtggcagcggctgcccggaggggctcggggcgtgcgagcctggcagtggtgctctgggaagggccattcttgcggaggagggcggggcacaggatccctctgctgggtcccagggaattgctttgaagcacatgaaggtgccactgggtctcagaaaatggaggttatggttatgaagtgtgtatgacatatgtgtataggaagagcgtccgaaagagcaggtttgttgccgaccccagcattcgcaaccctgaggtccacagctttctcctgatgggaggggaatgggtggcaaagggtctgcgcgtgtggcaagggctagcacgccaggagctgctggcttgggtcaaggtggacctgctgggccgggacagaaaagtgtcagtcccggcctgagacgctctagcattagagctgtccaagtccagacagcagggagcaggtggggatcgggaggcgcggatctggggggcagctggggccaggctgaaacagagcgggcgggacaggaagcacaggctgggcagcctccccggccagggaggagccaggctgggccacctcccggtctgtctgccgactacccgcagtatcacttacagggatggatgacatcccagggctgctgccacccccacctgtggggagacaccagactgggggtggtgtggagatactcttagagaagaggctgctgggccacgggctcggcatggcagggcagtggctaggtaagtacttgagggacaggtggggtctgcttgccaccgtcccctctgcaggctgggcctgggggctgctgcaggcggccagggcagaagggtgtggggagagtgaacccacaggagcagcggctcgaggagggggatgcaggctgcaggctcaaaggggcactggatccaccctgggtgcccgagagagcagggggcagcccctggaggggtactcacccccagagcttctgtggtcggctgaggacccccagcaggggttgactgaggggatcagaggcaagcagctgaggggagaggccaggttcttgatgctgatagggtcggggtgcctgggcgaccagaactcaaggagggaggcatggggaggggccgccgtgcagctggggtgggtgcaccgcagagcctctgggagtggtcagaacccccgacacctgccacttctacagcagctcatctgattttaaggggcttgctgcccttgcagaagtggaggggtgtgcccaaaggagcctgcctggaaggtcaccccatcaggttggcatgaccccagcccaggactgcagcctgccctcaaggtctgtgcagtatctggggtgagtcctctgaggacagggcccagggtgggtgtggagtggccagctcggggctcggtgtccaggctcaccttcaggggccacagcacagacctgcccttccagagtcttccctgagcttggctggggaggagggggctgcaggaaggagctgtgagcagggcaggatggagattcgtgtggccctcctgggaggggctgggcagggctgggaaaggggtgggtgagatgttccggaactcagggaaaggaagagtctgggtactgccctgggggcacctgggcccaggtggcaggtggccagctttctgcctcctttccacctcctttctccagaaggcacccaccagctgtgtaaatagggcaggtgcccacggcccgcctcaggccccgtctcctccccacccacgctctctaatcgcggattatacacaatccagcctgatccctgggcagctgccctccctcccgcagccacctctggctctgagagatgggcttggggccagcctggggtcccaggagtccaggccaggatgagaacctgctctgaccccacctggacgcattaggcctgcctggacctgttgcctcaccccaagagagccacaggcaatgcaaaggctcctgttcatgtcagggcacctggaaggcctgacttgcagaggctcttggctcgtgcagacccctccaagcccaggccctgcccaccacctcccctttgtctctggaactgccaggacagcttgtcctcagccagcaggtttcccgacccgggcacctcttcatgttgggcccccctcctttccctccatcagggatcatgcccttcttcaggggcctggatatcaaggacacaaaagctcccatgtgctatgtggggaggcagagtgggggctgggttgagctggggtctgggcagcgccattccgcagggcaggggcagcctaggcttcccatctgtggaatgggtgggtgggtctcacaacggacctgcttcccgtacttcagcacggttaccactcttgattggaactctgaccatgcatctcctcttctgtttacttcacgctttctcttcccatcaactcccattttaattacaatttgtttaaaagcactgcatattacttcattaaacagaagattagtttcacttaccattagtgtaaggtgactatagaaccaaagcagactggaaaccaaatgacataatgtcattctcttctccattccagctgcctgctgctgtgcgcctgagaacccctgtggagtgggaggggcagctgtctctgtacattagaaagggaggttaactaagtgacaggaggtgtttgggacatgtggacaccagacttctctcttgatgcaaggagggcagagccaggcagcctagtgggggctggcttgggggctgctggaaggactggctacaggtggaagagaggtcagacctgaagcttggggccacctccaggaaaggacaggtgaaagtggaggcatgaggcaggggagaggcaggtgccaggcagagggtggagaggaggcaggaacatagcagctggggcgggggcgggcctcaagtgtcatatgctactttcctggggcccaggggcaaggacaggaacagccacagcatgtgttgggacagagccctgtgccttcctagagctgggcaggtggaatggggcaggaatgggactcgtggtggctgcagcaggaactggaggggaaggggcttctggatcctgcagcctaccttcctagaggccagctttccggggtccaccaggtgggtgggaactgggcttgtgtagcaagactgccctgaggaccatccatgacatggtctagatgaaagttaggaaagaaagggagacaagctggcagcagaagtacagctgggtcaggagcaagggcctttccagatagggacaacccaagagtgcacatgtgcccacgccacacaacacaggcacacacgacacgtgcacgctcataggcactgcacacacacatgcacaggtgctcatgcatatgtatgagcttcatctacacacattcacatgccgtcctgcttatgtgcatgtttccatacatgcacatgaatgcacaatcacgtgtacacacatgcatgtgatcacatacatgaacatgtgtgcaccccactcctcaggtgccatcgggctcctcctgctgtcactgtgcagcaggggacatgaggccccagagcagacaggtgcagcacaggcgttcccaggcagtgccccacacacatgcatgagcacacccgggcatgtggcgcctcctttgtggactcagtcacctgccaggtgggctccctggtggtgtgagctcccagaggtctggcgagagagataaaggcaaccccaccaccaggcgtgctgagaattccctcttctggctgggcacagtggctcatacctgtaatcccagcactttgggaggccgaggtgggcagatcacttgaggttaggagtttgagaccagcctggccaatatggtgaaacctcatctccactaaaaatatacacacacaaaaattagctgggtgtggtggtgtgcacctgtagttccagctactcgggaggctgaggcaggagaatcgcttgaacctgggagtcagagactgcagtgagccgagatcatgtcactgcactccagcccgggtgacagagtgagactccatctaaaaaaaaaaaagaattccctcctctgggaatttagaccacagacaggttgcatgtatgtggccgttggaggcagcactcacagcaaagagtggaaacgtcaccacagggcctgccttctggtgaaaatggtgtcctgcagggcgggcagctgtttgagggcaggtgtcccaggtgcggcctgcagcagcctgagggtcacagagcgcagtgctgggagtgcagagacttcccccacagggagagttcccaggaacctgcttccggtgcacttctgggggtttgagttttttccacggacgaattactttgagaaaccactgttactcgtgtgtataggtgagcgtgcgtgtgcatgtgtgttctgtgtgtgagtgtgcatgtatgtgcgtgcctgcgtatatatcctcgcagatacggctagggacctcactcaggacagtagttctgcctgaggagagtgaatgcggcaagattgaggagaacacaggcatcttcaaactacatgtgcggtgctttatttctttaaaaatgcgtctaaagcaaataggaaaatgttaagatttgaatccgtagagtgtgggttctattattctctccacatcttccatacgtttaaaatcttttgcaatgaaaataagctgtagttaaagcagcaatgcaggctgccagtgagcgccccggaggccagtgaggaccagcatggctgggtggcctgttggaatccaaggggggcgggcaggagctgcaggcaggcgcccgggagtagcccgggcatgggggtgcggggcaacagggatgtctgcaggggtagcatgtgggccccggactgcaagcaggtggagccagccggatgcggctcctatgagaaaagcggggaacaagagaccacgctcgttcttcctgctgcggggacagccctggtcatcgctccggggaaccctgcagcctgcgccgcacgtggccgccccctgctgcttcctcctccccggcctccgggtggccttgctgacggctccttctctgaggcaggtctctgccttctcgcctggtgcctgcactcagtagccccctcaccagagctgctgggtgaaggaagcactaagaacccaaggctcgggaggagagtggggccgggaagctgcagggaagcgcagggccaggcctggtgggcccaggggctggctcacgggagggcaggagggagactgtggcggacagcacgtggggccaggaggtgacctccaagtggattgtgggtgggttttttgtcctctttctgcattttccaggcattttgtaatgtggatagaatatttctgttcttcaaaaatactttagttaagaaaaataagatggaagctgttgcacttgaaaatgaggaagccactggtgatgcagggggggcggcggagaggacctcttctgcaaatagcggcaggaacacggcatggatgcagctcgcgctcccccaggccctcccctgggctgtgtggaggggtccggggggaatgggccagcgcccagtggtcacctggccatgtctccccacagcccggaagcaggagatcattaagaccacggagcagctcatcgaggccgtcaacaacggtgactttgaggcctacgcgt

SEQ ID NO: 126 - DNA, 291 bp, Homo sapiens
  source 1..291: mol_type = other DNA
ctcagaattcaagatggataagccgccttcagtctcagccaggctgtgctctgggaaacctgagggaactggctccagggtcagctgagccaagctgctcatgtgacccctctcctcccaggctgctcacttgctccagccccggatgatgctgtgtgttttgctatttgtgaaccttggtccccaacagatgacacaaatgcgtattgctgtgcttgctctgtgtgcgtgtgtgtgtgcacgcgtgcgtgaatcattgccaaggaattgacacatcacacaaggtaatac

SEQ ID NO: 127 - DNA, 175 bp, Homo sapiens
  source 1..175: mol_type = other DNA
cccaggctgctcacttgctccagccccggatgatgctgtgtgttttgctatttgtgaaccttggtccccaacagatgacacaaatgcgtattgctgtgcttgctctgtgtgcgtgtgtgtgtgcacgcgtgcgtgaatcattgccaaggaattgacacatcacacaaggtaatac

SEQ ID NO: 128 - DNA, 144 bp, Homo sapiens
  source 1..144: mol_type = other DNA
cttccagctgcctgggtttcctggaaagaactcttatccccaggaactagtttgttgaataaatgctggtgaatgaatgaatgattgaacagatgaatgagtgatgagtagataaaaggatggatggagagatgggtgagtaca

SEQ ID NO: 129 - DNA, 192 bp, Homo sapiens
  source 1..192: mol_type = other DNA
tccagccctaaccactcaggattgggccgtttgtgtctgggtatgtctcttccagctgcctgggtttcctggaaagaactcttatccccaggaactagtttgttgaataaatgctggtgaatgaatgaatgattgaacagatgaatgagtgatgagtagataaaaggatggatggagagatgggtgagtaca

SEQ ID NO: 130 - DNA, 10 bp, synthetic construct
  source 1..10: mol_type = other DNA
  modified_base 1..3: mod_base = OTHER; note = locked nucleic acid (LNA)
  modified_base 8..10: mod_base = OTHER; note = locked nucleic acid (LNA)
  misc_feature 1..10: note = linked by phosphorothioate internucleoside linkage
  modified_base 2: mod_base = m5c
  modified_base 6: mod_base = m5c
  modified_base 10: mod_base = m5c
acacacacac

SEQ ID NO: 131 - DNA, 11 bp, synthetic construct
  source 1..11: mol_type = other DNA
  misc_feature 1..11: note = linked by phosphorothioate internucleoside linkage
  modified_base 1..2: mod_base = OTHER; note = locked nucleic acid (LNA)
  modified_base 4..6: mod_base = OTHER; note = locked nucleic acid (LNA)
  modified_base 9..11: mod_base = OTHER; note = locked nucleic acid (LNA)
  modified_base 2: mod_base = m5c
  modified_base 6: mod_base = m5c
  modified_base 10: mod_base = m5c
acacacacaca

SEQ ID NO: 132 - DNA, 12 bp, synthetic construct
  source 1..12: mol_type = other DNA
  misc_feature 1..12: note = linked by phosphorothioate internucleoside linkage
  modified_base 1: mod_base = OTHER; note = locked nucleic acid (LNA)
  modified_base 3: mod_base = OTHER; note = locked nucleic acid (LNA)
  modified_base 6..8: mod_base = OTHER; note = locked nucleic acid (LNA)
  modified_base 10..12: mod_base = OTHER; note = locked nucleic acid (LNA)
  modified_base 6: mod_base = m5c
  modified_base 8: mod_base = m5c
  modified_base 10: mod_base = m5c
  modified_base 12: mod_base = m5c
acacacacacac

SEQ ID NO: 133 - DNA, 13 bp, synthetic construct
  source 1..13: mol_type = other DNA
  misc_feature 1..13: note = linked by phosphorothioate internucleoside linkage
  modified_base 1: mod_base = OTHER; note = locked nucleic acid (LNA)
  modified_base 3: mod_base = OTHER; note = locked nucleic acid (LNA)
  modified_base 5..6: mod_base = OTHER; note = locked nucleic acid (LNA)
  modified_base 8: mod_base = OTHER; note = locked nucleic acid (LNA)
  modified_base 10: mod_base = OTHER; note = locked nucleic acid (LNA)
  modified_base 12..13: mod_base = OTHER; note = locked nucleic acid (LNA)
  modified_base 6: mod_base = m5c
  modified_base 8: mod_base = m5c
  modified_base 12: mod_base = m5c
acacacacacaca

SEQ ID NO: 134 - DNA, 13 bp, synthetic construct
  source 1..13: mol_type = other DNA
  misc_feature 1..13: note = linked by phosphorothioate internucleoside linkage
  modified_base 1: mod_base = OTHER; note = locked nucleic acid (LNA)
  modified_base 5..6: mod_base = OTHER; note = locked nucleic acid (LNA)
  modified_base 10: mod_base = OTHER; note = locked nucleic acid (LNA)
  modified_base 12..13: mod_base = OTHER; note = locked nucleic acid (LNA)
  modified_base 6: mod_base = m5c
  modified_base 10: mod_base = m5c
  modified_base 12: mod_base = m5c
acacacacacaca

SEQ ID NO: 135 - DNA, 13 bp, synthetic construct
  source 1..13: mol_type = other DNA
  misc_feature 1..13: note = linked by phosphorothioate internucleoside linkage
  modified_base 1: mod_base = OTHER; note = locked nucleic acid (LNA)
  modified_base 3: mod_base = OTHER; note = locked nucleic acid (LNA)
  modified_base 5: mod_base = OTHER; note = locked nucleic acid (LNA)
  modified_base 8: mod_base = OTHER; note = locked nucleic acid (LNA)
  modified_base 12..13: mod_base = OTHER; note = locked nucleic acid (LNA)
  modified_base 8: mod_base = m5c
  modified_base 12: mod_base = m5c
acacacacacaca

SEQ ID NO: 136 - DNA, 13 bp, synthetic construct
  source 1..13: mol_type = other DNA
  misc_feature 1..13: note = linked by phosphorothioate internucleoside linkage
  modified_base 1: mod_base = OTHER; note = locked nucleic acid (LNA)
  modified_base 5..6: mod_base = OTHER; note = locked nucleic acid (LNA)
  modified_base 8: mod_base = OTHER; note = locked nucleic acid (LNA)
  modified_base 12..13: mod_base = OTHER; note = locked nucleic acid (LNA)
  modified_base 6: mod_base = m5c
  modified_base 8: mod_base = m5c
  modified_base 12: mod_base = m5c
acacacacacaca

SEQ ID NO: 137 - DNA, 16 bp, synthetic construct
  source 1..16: mol_type = other DNA
  misc_feature 1..16: note = linked by phosphorothioate internucleoside linkage
  modified_base 1: mod_base = OTHER; note = locked nucleic acid (LNA)
  modified_base 3: mod_base = OTHER; note = locked nucleic acid (LNA)
  modified_base 5: mod_base = OTHER; note = locked nucleic acid (LNA)
  modified_base 7: mod_base = OTHER; note = locked nucleic acid (LNA)
  modified_base 9: mod_base = OTHER; note = locked nucleic acid (LNA)
  modified_base 11: mod_base = OTHER; note = locked nucleic acid (LNA)
  modified_base 13: mod_base = OTHER; note = locked nucleic acid (LNA)
  modified_base 15..16: mod_base = OTHER; note = locked nucleic acid (LNA)
  modified_base 16: mod_base = m5c
acacacacacacacac

SEQ ID NO: 138 - DNA, 16 bp, synthetic construct
  source 1..16: mol_type = other DNA
  misc_feature 1..16: note = linked by phosphorothioate internucleoside linkage
  modified_base 1: mod_base = OTHER; note = locked nucleic acid (LNA)
  modified_base 3: mod_base = OTHER; note = locked nucleic acid (LNA)
  modified_base 7: mod_base = OTHER; note = locked nucleic acid (LNA)
  modified_base 11: mod_base = OTHER; note = locked nucleic acid (LNA)
  modified_base 13: mod_base = OTHER; note = locked nucleic acid (LNA)
  modified_base 15..16: mod_base = OTHER; note = locked nucleic acid (LNA)
  modified_base 16: mod_base = m5c
acacacacacacacac

SEQ ID NO: 139 - DNA, 16 bp, synthetic construct
  source 1..16: mol_type = other DNA
  misc_feature 1..16: note = linked by phosphorothioate internucleoside linkage
  modified_base 1..2: mod_base = OTHER; note = locked nucleic acid (LNA)
  modified_base 5: mod_base = OTHER; note = locked nucleic acid (LNA)
  modified_base 9: mod_base = OTHER; note = locked nucleic acid (LNA)
  modified_base 13: mod_base = OTHER; note = locked nucleic acid (LNA)
  modified_base 15..16: mod_base = OTHER; note = locked nucleic acid (LNA)
  modified_base 2: mod_base = m5c
  modified_base 16: mod_base = m5c
acacacacacacacac

SEQ ID NO: 140 - DNA, 18 bp, synthetic construct
  source 1..18: mol_type = other DNA
  misc_feature 1..18: note = linked by phosphorothioate internucleoside linkage
  modified_base 1: mod_base = OTHER; note = locked nucleic acid (LNA)
  modified_base 3: mod_base = OTHER; note = locked nucleic acid (LNA)
  modified_base 5: mod_base = OTHER; note = locked nucleic acid (LNA)
  modified_base 7: mod_base = OTHER; note = locked nucleic acid (LNA)
  modified_base 9: mod_base = OTHER; note = locked nucleic acid (LNA)
  modified_base 11: mod_base = OTHER; note = locked nucleic acid (LNA)
  modified_base 13: mod_base = OTHER; note = locked nucleic acid (LNA)
  modified_base 15: mod_base = OTHER; note = locked nucleic acid (LNA)
  modified_base 17..18: mod_base = OTHER; note = locked nucleic acid (LNA)
  modified_base 18: mod_base = m5c
acacacacacacacacac

SEQ ID NO: 141 - DNA, 18 bp, synthetic construct
  source 1..18: mol_type = other DNA
  misc_feature 1..18: note = linked by phosphorothioate internucleoside linkage
  modified_base 1: mod_base = OTHER; note = locked nucleic acid (LNA)
  modified_base 3: mod_base = OTHER; note = locked nucleic acid (LNA)
  modified_base 7: mod_base = OTHER; note = locked nucleic acid (LNA)
  modified_base 11: mod_base = OTHER; note = locked nucleic acid (LNA)
  modified_base 15: mod_base = OTHER; note = locked nucleic acid (LNA)
  modified_base 17..18: mod_base = OTHER; note = locked nucleic acid (LNA)
  modified_base 18: mod_base = m5c
acacacacacacacacac

SEQ ID NO: 142 - DNA, 18 bp, synthetic construct
  source 1..18: mol_type = other DNA
  misc_feature 1..18: note = linked by phosphorothioate internucleoside linkage
  modified_base 1..3: mod_base = OTHER; note = locked nucleic acid (LNA)
  modified_base 7: mod_base = OTHER; note = locked nucleic acid (LNA)
  modified_base 9: mod_base = OTHER; note = locked nucleic acid (LNA)
  modified_base 13: mod_base = OTHER; note = locked nucleic acid (LNA)
  modified_base 15: mod_base = OTHER; note = locked nucleic acid (LNA)
  modified_base 18: mod_base = OTHER; note = locked nucleic acid (LNA)
  modified_base 2: mod_base = m5c
  modified_base 18: mod_base = m5c
acacacacacacacacac

SEQ ID NO: 143 - DNA, 10 bp, synthetic construct
  source 1..10: mol_type = other DNA
  misc_feature 1..10: note = linked by phosphorothioate internucleoside linkage
  modified_base 1..3: mod_base = OTHER; note = locked nucleic acid (LNA)
  modified_base 5..7: mod_base = OTHER; note = locked nucleic acid (LNA)
  modified_base 9..10: mod_base = OTHER; note = locked nucleic acid (LNA)
  modified_base 1: mod_base = m5c
  modified_base 3: mod_base = m5c
  modified_base 5: mod_base = m5c
  modified_base 7: mod_base = m5c
  modified_base 9: mod_base = m5c
cacacacaca

SEQ ID NO: 144 - DNA, 11 bp, synthetic construct
  source 1..11: mol_type = other DNA
  misc_feature 1..11: note = linked by phosphorothioate internucleoside linkage
  modified_base 1..2: mod_base = OTHER; note = locked nucleic acid (LNA)
  modified_base 4..6: mod_base = OTHER; note = locked nucleic acid (LNA)
  modified_base 9..11: mod_base = OTHER; note = locked nucleic acid (LNA)
  modified_base 1: mod_base = m5c
  modified_base 5: mod_base = m5c
  modified_base 9: mod_base = m5c
  modified_base 11: mod_base = m5c
cacacacacac

SEQ ID NO: 145 - DNA, 12 bp, synthetic construct
  source 1..12: mol_type = other DNA
  misc_feature 1..12: note = linked by phosphorothioate internucleoside linkage
  modified_base 1..2: mod_base = OTHER; note = locked nucleic acid (LNA)
  modified_base 4: mod_base = OTHER; note = locked nucleic acid (LNA)
  modified_base 6..8: mod_base = OTHER; note = locked nucleic acid (LNA)
  modified_base 9: mod_base = OTHER; note = locked nucleic acid (LNA)
  modified_base 11..12: mod_base = OTHER; note = locked nucleic acid (LNA)
  modified_base 1: mod_base = m5c
  modified_base 7: mod_base = m5c
  modified_base 9: mod_base = m5c
  modified_base 11: mod_base = m5c
cacacacacaca

SEQ ID NO: 146 - DNA, 12 bp, synthetic construct
  source 1..12: mol_type = other DNA
  misc_feature 1..12: note = linked by phosphorothioate internucleoside linkage
  modified_base 1..2: mod_base = OTHER; note = locked nucleic acid (LNA)
  modified_base 4: mod_base = OTHER; note = locked nucleic acid (LNA)
  modified_base 7: mod_base = OTHER; note = locked nucleic acid (LNA)
  modified_base 11..12: mod_base = OTHER; note = locked nucleic acid (LNA)
  modified_base 1: mod_base = m5c
  modified_base 7: mod_base = m5c
  modified_base 11: mod_base = m5c
cacacacacaca

SEQ ID NO: 147 - DNA, 12 bp, synthetic construct
  source 1..12: mol_type = other DNA
  misc_feature 1..12: note = linked by phosphorothioate internucleoside linkage
  modified_base 1..2: mod_base = OTHER; note = locked nucleic acid (LNA)
  modified_base 6..7: mod_base = OTHER; note = locked nucleic acid (LNA)
  modified_base 9: mod_base = OTHER; note = locked nucleic acid (LNA)
  modified_base 12: mod_base = OTHER; note = locked nucleic acid (LNA)
  modified_base 1: mod_base = m5c
  modified_base 7: mod_base = m5c
  modified_base 9: mod_base = m5c
cacacacacaca

SEQ ID NO: 148 - DNA, 12 bp, synthetic construct
  source 1..12: mol_type = other DNA
  misc_feature 1..12: note = linked by phosphorothioate internucleoside linkage
  modified_base 1: mod_base = OTHER; note = locked nucleic acid (LNA)
  modified_base 4: mod_base = OTHER; note = locked nucleic acid (LNA)
  modified_base 6..7: mod_base = OTHER; note = locked nucleic acid (LNA)
  modified_base 11..12: mod_base = OTHER; note = locked nucleic acid (LNA)
  modified_base 1: mod_base = m5c
  modified_base 7: mod_base = m5c
  modified_base 11: mod_base = m5c
cacacacacaca

SEQ ID NO: 149 - DNA, 13 bp, synthetic construct
  source 1..13: mol_type = other DNA
  misc_feature 1..13: note = linked by phosphorothioate internucleoside linkage
  modified_base 1: mod_base = OTHER; note = locked nucleic acid (LNA)
  modified_base 3: mod_base = OTHER; note = locked nucleic acid (LNA)
  modified_base 5: mod_base = OTHER; note = locked nucleic acid (LNA)
  modified_base 7..8: mod_base = OTHER; note = locked nucleic acid (LNA)
  modified_base 10: mod_base = OTHER; note = locked nucleic acid (LNA)
  modified_base 12..13: mod_base = OTHER; note = locked nucleic acid (LNA)
  modified_base 1: mod_base = m5c
  modified_base 3: mod_base = m5c
  modified_base 5: mod_base = m5c
  modified_base 7: mod_base = m5c
  modified_base 13: mod_base = m5c
cacacacacacac

SEQ ID NO: 150 - DNA, 13 bp, synthetic construct
  source 1..13: mol_type = other DNA
  misc_feature 1..13: note = linked by phosphorothioate internucleoside linkage
  modified_base 1: mod_base = OTHER; note = locked nucleic acid (LNA)
  modified_base 5: mod_base = OTHER; note = locked nucleic acid (LNA)
  modified_base 8: mod_base = OTHER; note = locked nucleic acid (LNA)
  modified_base 10: mod_base = OTHER; note = locked nucleic acid (LNA)
  modified_base 12..13: mod_base = OTHER; note = locked nucleic acid (LNA)
  modified_base 1: mod_base = m5c
  modified_base 5: mod_base = m5c
  modified_base 13: mod_base = m5c
cacacacacacac

SEQ ID NO: 151 - DNA, 13 bp, synthetic construct
  source 1..13: mol_type = other DNA
  misc_feature 1..13: note = linked by phosphorothioate internucleoside linkage
  modified_base 1: mod_base = OTHER; note = locked nucleic acid (LNA)
  modified_base 3: mod_base = OTHER; note = locked nucleic acid (LNA)
  modified_base 7..8: mod_base = OTHER; note = locked nucleic acid (LNA)
  modified_base 12..13: mod_base = OTHER; note = locked nucleic acid (LNA)
  modified_base 1: mod_base = m5c
  modified_base 3: mod_base = m5c
  modified_base 7: mod_base = m5c
  modified_base 13: mod_base = m5c
cacacacacacac

SEQ ID NO: 152 - DNA, 13 bp, synthetic construct
  source 1..13: mol_type = other DNA
  misc_feature 1..13: note = linked by phosphorothioate internucleoside linkage
  modified_base 1: mod_base = OTHER; note = locked nucleic acid (LNA)
  modified_base 5: mod_base = OTHER; note = locked nucleic acid (LNA)
  modified_base 8: mod_base = OTHER; note = locked nucleic acid (LNA)
  modified_base 10: mod_base = OTHER; note = locked nucleic acid (LNA)
  modified_base 13: mod_base = OTHER; note = locked nucleic acid (LNA)
  modified_base 1: mod_base = m5c
  modified_base 5: mod_base = m5c
  modified_base 13: mod_base = m5c
cacacacacacac

SEQ ID NO: 153 - DNA, 13 bp, synthetic construct
  source 1..13: mol_type = other DNA
  misc_feature 1..13: note = linked by phosphorothioate internucleoside linkage
  modified_base 1: mod_base = OTHER; note = locked nucleic acid (LNA)
  modified_base 5: mod_base = OTHER; note = locked nucleic acid (LNA)
  modified_base 8: mod_base = OTHER; note = locked nucleic acid (LNA)
  modified_base 12..13: mod_base = OTHER; note = locked nucleic acid (LNA)
  modified_base 1: mod_base = m5c
  modified_base 5: mod_base = m5c
  modified_base 13: mod_base = m5c
cacacacacacac

SEQ ID NO: 154 - DNA, 16 bp, synthetic construct
  source 1..16: mol_type = other DNA
  misc_feature 1..16: note = linked by phosphorothioate internucleoside linkage
  modified_base 1: mod_base = OTHER; note = locked nucleic acid (LNA)
  modified_base 4: mod_base = OTHER; note = locked nucleic acid (LNA)
  modified_base 6: mod_base = OTHER; note = locked nucleic acid (LNA)
  modified_base 8: mod_base = OTHER; note = locked nucleic acid (LNA)
  modified_base 10: mod_base = OTHER; note = locked nucleic acid (LNA)
  modified_base 12: mod_base = OTHER; note = locked nucleic acid (LNA)
  modified_base 15..16: mod_base = OTHER; note = locked nucleic acid (LNA)
  modified_base 1: mod_base = m5c
  modified_base 15: mod_base = m5c
cacacacacacacaca

SEQ ID NO: 155 - DNA, 16 bp, synthetic construct
  source 1..16: mol_type = other DNA
  misc_feature 1..16: note = linked by phosphorothioate internucleoside linkage
  modified_base 1: mod_base = OTHER; note = locked nucleic acid (LNA)
  modified_base 4: mod_base = OTHER; note = locked nucleic acid (LNA)
  modified_base 8: mod_base = OTHER; note = locked nucleic acid (LNA)
  modified_base 10: mod_base = OTHER; note = locked nucleic acid (LNA)
  modified_base 12: mod_base = OTHER; note = locked nucleic acid (LNA)
  modified_base 15..16: mod_base = OTHER; note = locked nucleic acid (LNA)
  modified_base 1: mod_base = m5c
  modified_base 15: mod_base = m5c
cacacacacacacaca

SEQ ID NO: 156 - DNA, 16 bp, synthetic construct
  source 1..16: mol_type = other DNA
  misc_feature 1..16: note = linked by phosphorothioate internucleoside linkage
  modified_base 1: mod_base = OTHER; note = locked nucleic acid (LNA)
  modified_base 4: mod_base = OTHER; note = locked nucleic acid (LNA)
  modified_base 8: mod_base = OTHER; note = locked nucleic acid (LNA)
  modified_base 12: mod_base = OTHER; note = locked nucleic acid (LNA)
  modified_base 15..16: mod_base = OTHER; note = locked nucleic acid (LNA)
  modified_base 1: mod_base = m5c
  modified_base 15: mod_base = m5c
cacacacacacacaca

SEQ ID NO: 157 - DNA, 18 bp, synthetic construct
  source 1..18: mol_type = other DNA
  misc_feature 1..18: note = linked by phosphorothioate internucleoside linkage
  modified_base 1: mod_base = OTHER; note = locked nucleic acid (LNA)
  modified_base 4: mod_base = OTHER; note = locked nucleic acid (LNA)
  modified_base 6: mod_base = OTHER; note = locked nucleic acid (LNA)
  modified_base 8: mod_base = OTHER; note = locked nucleic acid (LNA)
  modified_base 10: mod_base = OTHER; note = locked nucleic acid (LNA)
  modified_base 12: mod_base = OTHER; note = locked nucleic acid (LNA)
  modified_base 14: mod_base = OTHER; note = locked nucleic acid (LNA)
  modified_base 17..18: mod_base = OTHER; note = locked nucleic acid (LNA)
  modified_base 1: mod_base = m5c
  modified_base 17: mod_base = m5c
cacacacacacacacaca

SEQ ID NO: 158 - DNA, 18 bp, synthetic construct
  source 1..18: mol_type = other DNA
  misc_feature 1..18: note = linked by phosphorothioate internucleoside linkage
  modified_base 1: mod_base = OTHER; note = locked nucleic acid (LNA)
  modified_base 4: mod_base = OTHER; note = locked nucleic acid (LNA)
  modified_base 8: mod_base = OTHER; note = locked nucleic acid (LNA)
  modified_base 12..13: mod_base = OTHER; note = locked nucleic acid (LNA)
  modified_base 17..18: mod_base = OTHER; note = locked nucleic acid (LNA)
  modified_base 1: mod_base = m5c
  modified_base 13: mod_base = m5c
  modified_base 17: mod_base = m5c
cacacacacacacacaca

SEQ ID NO: 159 - DNA, 18 bp, synthetic construct
  source 1..18: mol_type = other DNA
  misc_feature 1..18: note = linked by phosphorothioate internucleoside linkage
  modified_base 1: mod_base = OTHER; note = locked nucleic acid (LNA)
  modified_base 3: mod_base = OTHER; note = locked nucleic acid (LNA)
  modified_base 6: mod_base = OTHER; note = locked nucleic acid (LNA)
  modified_base 8: mod_base = OTHER; note = locked nucleic acid (LNA)
  modified_base 10: mod_base = OTHER; note = locked nucleic acid (LNA)
  modified_base 14: mod_base = OTHER; note = locked nucleic acid (LNA)
  modified_base 17..18: mod_base = OTHER; note = locked nucleic acid (LNA)
  modified_base 1: mod_base = m5c
  modified_base 3: mod_base = m5c
  modified_base 17: mod_base = m5c
cacacacacacacacaca

SEQ ID NO: 160 - DNA, 18 bp, synthetic construct
  source 1..18: mol_type = other DNA
  misc_feature 1..18: note = linked by phosphorothioate internucleoside linkage
  modified_base 1: mod_base = OTHER; note = locked nucleic acid (LNA)
  modified_base 4: mod_base = OTHER; note = locked nucleic acid (LNA)
  modified_base 6: mod_base = OTHER; note = locked nucleic acid (LNA)
  modified_base 10..11: mod_base = OTHER; note = locked nucleic acid (LNA)
  modified_base 14: mod_base = OTHER; note = locked nucleic acid (LNA)
  modified_base 17..18: mod_base = OTHER; note = locked nucleic acid (LNA)
  modified_base 1: mod_base = m5c
  modified_base 11: mod_base = m5c
  modified_base 17: mod_base = m5c
cacacacacacacacaca

SEQ ID NO: 161 - DNA, 17 bp, synthetic construct
  source 1..17: mol_type = other DNA
  misc_feature 1..17: note = linked by phosphorothioate internucleoside linkage
  modified_base 1..2: mod_base = OTHER; note = locked nucleic acid (LNA)
  modified_base 14..17: mod_base = OTHER; note = locked nucleic acid (LNA)
  modified_base 2: mod_base = m5c
  modified_base 16..17: mod_base = m5c
tccacactgaacaaacc

SEQ ID NO: 162 - DNA, 23 bp, synthetic construct
  source 1..23: mol_type = other DNA
  misc_feature 1..23: note = linked by phosphorothioate internucleoside linkage
  modified_base 1..2: mod_base = OTHER; note = locked nucleic acid (LNA)
  modified_base 5: mod_base = OTHER; note = locked nucleic acid (LNA)
  modified_base 7: mod_base = OTHER; note = locked nucleic acid (LNA)
  modified_base 9: mod_base = OTHER; note = locked nucleic acid (LNA)
  modified_base 11: mod_base = OTHER; note = locked nucleic acid (LNA)
  modified_base 13: mod_base = OTHER; note = locked nucleic acid (LNA)
  modified_base 15: mod_base = OTHER; note = locked nucleic acid (LNA)
  modified_base 17: mod_base = OTHER; note = locked nucleic acid (LNA)
  modified_base 19: mod_base = OTHER; note = locked nucleic acid (LNA)
  modified_base 22..23: mod_base = OTHER; note = locked nucleic acid (LNA)
  modified_base 1: mod_base = m5c
  modified_base 5: mod_base = m5c
  modified_base 7: mod_base = m5c
  modified_base 9: mod_base = m5c
  modified_base 11: mod_base = m5c
  modified_base 13: mod_base = m5c
  modified_base 15: mod_base = m5c
  modified_base 17: mod_base = m5c
  modified_base 19: mod_base = m5c
  modified_base 23: mod_base = m5c
cacacacacacacacacacacac

SEQ ID NO: 163 - DNA, 24 bp, synthetic construct
  source 1..24: mol_type = other DNA
  misc_feature 1..24: note = linked by phosphorothioate internucleoside linkage
  modified_base 1..2: mod_base = OTHER; note = locked nucleic acid (LNA)
  modified_base 4: mod_base = OTHER; note = locked nucleic acid (LNA)
  modified_base 6: mod_base = OTHER; note = locked nucleic acid (LNA)
  modified_base 8: mod_base = OTHER; note = locked nucleic acid (LNA)
  modified_base 10: mod_base = OTHER; note = locked nucleic acid (LNA)
  modified_base 12: mod_base = OTHER; note = locked nucleic acid (LNA)
  modified_base 14: mod_base = OTHER; note = locked nucleic acid (LNA)
  modified_base 16: mod_base = OTHER; note = locked nucleic acid (LNA)
  modified_base 18: mod_base = OTHER; note = locked nucleic acid (LNA)
  modified_base 20: mod_base = OTHER; note = locked nucleic acid (LNA)
  modified_base 22: mod_base = OTHER; note = locked nucleic acid (LNA)
  modified_base 24: mod_base = OTHER; note = locked nucleic acid (LNA)
  modified_base 2: mod_base = m5c
  modified_base 4: mod_base = m5c
  modified_base 6: mod_base = m5c
  modified_base 8: mod_base = m5c
  modified_base 10: mod_base = m5c
  modified_base 12: mod_base = m5c
  modified_base 14: mod_base = m5c
  modified_base 16: mod_base = m5c
acacacacacacacacacacacac

SEQ ID NO: 164 - DNA, 24 bp, synthetic construct
  source 1..24: mol_type = other DNA
  misc_feature 1..24: note = linked by phosphorothioate internucleoside linkage
  modified_base 1..2: mod_base = OTHER; note = locked nucleic acid (LNA)
  modified_base 4: mod_base = OTHER; note = locked nucleic acid (LNA)
  modified_base 6: mod_base = OTHER; note = locked nucleic acid (LNA)
  modified_base 8: mod_base = OTHER; note = locked nucleic acid (LNA)
  modified_base 11..14: mod_base = OTHER; note = locked nucleic acid (LNA)
  modified_base 17: mod_base = OTHER; note = locked nucleic acid (LNA)
  modified_base 19: mod_base = OTHER; note = locked nucleic acid (LNA)
  modified_base 21: mod_base = OTHER; note = locked nucleic acid (LNA)
  modified_base 23..24: mod_base = OTHER; note = locked nucleic acid (LNA)
  modified_base 1: mod_base = m5c
  modified_base 11: mod_base = m5c
  modified_base 14: mod_base = m5c
  modified_base 24: mod_base = m5c
cacacacacacaacacacacacac

SEQ ID NO: 165 - DNA, 24 bp, synthetic construct
  source 1..24: mol_type = other DNA
  misc_feature 1..24: note = linked by phosphorothioate internucleoside linkage
  modified_base 1..24: mod_base = OTHER; note = locked nucleic acid (LNA)
  modified_base 1: mod_base = m5c
  modified_base 3: mod_base = m5c
  modified_base 5: mod_base = m5c
  modified_base 7: mod_base = m5c
  modified_base 9: mod_base = m5c
  modified_base 11: mod_base = m5c
  modified_base 14: mod_base = m5c
  modified_base 16: mod_base = m5c
  modified_base 18: mod_base = m5c
  modified_base 20: mod_base = m5c
  modified_base 22: mod_base = m5c
  modified_base 24: mod_base = m5c
cacacacacacaacacacacacac

SEQ ID NO: 166 - DNA, 25 bp, synthetic construct
  source 1..25: mol_type = other DNA
  misc_feature 1..25: note = linked by phosphorothioate internucleoside linkage
  modified_base 1..2: mod_base = OTHER; note = locked nucleic acid (LNA)
  modified_base 5..6: mod_base = OTHER; note = locked nucleic acid (LNA)
  modified_base 9..10: mod_base = OTHER; note = locked nucleic acid (LNA)
  modified_base 13..14: mod_base = OTHER; note = locked nucleic acid (LNA)
  modified_base 17..18: mod_base = OTHER; note = locked nucleic acid (LNA)
  modified_base 21..22: mod_base = OTHER; note = locked nucleic acid (LNA)
  modified_base 25: mod_base = OTHER; note = locked nucleic acid (LNA)
  modified_base 1: mod_base = m5c
  modified_base 5: mod_base = m5c
  modified_base 9: mod_base = m5c
  modified_base 13: mod_base = m5c
  modified_base 17: mod_base = m5c
  modified_base 21: mod_base = m5c
  modified_base 25: mod_base = m5c
cacacacacacacacacacacacac

SEQ ID NO: 167 - DNA, 25 bp, synthetic construct
  source 1..25: mol_type = other DNA
  misc_feature 1..25: note = linked by phosphorothioate internucleoside linkage
  modified_base 1..2: mod_base = OTHER; note = locked nucleic acid (LNA)
  modified_base 6..7: mod_base = OTHER; note = locked nucleic acid (LNA)
  modified_base 10..11: mod_base = OTHER; note = locked nucleic acid (LNA)
  modified_base 15..16: mod_base = OTHER; note = locked nucleic acid (LNA)
  modified_base 20: mod_base = OTHER; note = locked nucleic acid (LNA)
  modified_base 24..25: mod_base = OTHER; note = locked nucleic acid (LNA)
  modified_base 1: mod_base = m5c
  modified_base 7: mod_base = m5c
  modified_base 11: mod_base = m5c
  modified_base 15: mod_base = m5c
  modified_base 25: mod_base = m5c
cacacacacacacacacacacacac

SEQ ID NO: 168 - DNA, 25 bp, synthetic construct
  source 1..25: mol_type = other DNA
  misc_feature 1..25: note = linked by phosphorothioate internucleoside linkage
  modified_base 1..2: mod_base = OTHER; note = locked nucleic acid (LNA)
  modified_base 5: mod_base = OTHER; note = locked nucleic acid (LNA)
  modified_base 7: mod_base = OTHER; note = locked nucleic acid (LNA)
  modified_base 9: mod_base = OTHER; note = locked nucleic acid (LNA)
  modified_base 11: mod_base = OTHER; note = locked nucleic acid (LNA)
  modified_base 13: mod_base = OTHER; note = locked nucleic acid (LNA)
  modified_base 15: mod_base = OTHER; note = locked nucleic acid (LNA)
  modified_base 17: mod_base = OTHER; note = locked nucleic acid (LNA)
  modified_base 19: mod_base = OTHER; note = locked nucleic acid (LNA)
  modified_base 21: mod_base = OTHER; note = locked nucleic acid (LNA)
  modified_base 24..25: mod_base = OTHER; note = locked nucleic acid (LNA)
  modified_base 1: mod_base = m5c
  modified_base 5: mod_base = m5c
  modified_base 7: mod_base = m5c
  modified_base 9: mod_base = m5c
  modified_base 11: mod_base = m5c
  modified_base 13: mod_base = m5c
  modified_base 15: mod_base = m5c
  modified_base 17: mod_base = m5c
  modified_base 19: mod_base = m5c
  modified_base 21: mod_base = m5c
  modified_base 25: mod_base = m5c
cacacacacacacacacacacacac

SEQ ID NO: 169 - DNA, 25 bp, synthetic construct
  source 1..25: mol_type = other DNA
  misc_feature 1..25: note = linked by phosphorothioate internucleoside linkage
  modified_base 1..2: mod_base = OTHER; note = locked nucleic acid (LNA)
  modified_base 4: mod_base = OTHER; note = locked nucleic acid (LNA)
  modified_base 6: mod_base = OTHER; note = locked nucleic acid (LNA)
  modified_base 8: mod_base = OTHER; note = locked nucleic acid (LNA)
  modified_base 10: mod_base = OTHER; note = locked nucleic acid (LNA)
  modified_base 12: mod_base = OTHER; note = locked nucleic acid (LNA)
  modified_base 14: mod_base = OTHER; note = locked nucleic acid (LNA)
  modified_base 16: mod_base = OTHER; note = locked nucleic acid (LNA)
  modified_base 18: mod_base = OTHER; note = locked nucleic acid (LNA)
  modified_base 20: mod_base = OTHER; note = locked nucleic acid (LNA)
  modified_base 22: mod_base = OTHER; note = locked nucleic acid (LNA)
  modified_base 24..25: mod_base = OTHER; note = locked nucleic acid (LNA)
  modified_base 1: mod_base = m5c
  modified_base 25: mod_base = m5c
cacacacacacacacacacacacac

SEQ ID NO: 170 - DNA, 26 bp, synthetic construct
  source 1..26: mol_type = other DNA
  misc_feature 1..26: note = linked by phosphorothioate internucleoside linkage
  modified_base 1..2: mod_base = OTHER; note = locked nucleic acid (LNA)
  modified_base 4: mod_base = OTHER; note = locked nucleic acid (LNA)
  modified_base 6: mod_base = OTHER; note = locked nucleic acid (LNA)
  modified_base 8: mod_base = OTHER; note = locked nucleic acid (LNA)
  modified_base 10: mod_base = OTHER; note = locked nucleic acid (LNA)
  modified_base 12: mod_base = OTHER; note = locked nucleic acid (LNA)
  modified_base 14: mod_base = OTHER; note = locked nucleic acid (LNA)
  modified_base 16: mod_base = OTHER; note = locked nucleic acid (LNA)
  modified_base 18: mod_base = OTHER; note = locked nucleic acid (LNA)
  modified_base 20: mod_base = OTHER; note = locked nucleic acid (LNA)
  modified_base 22: mod_base = OTHER; note = locked nucleic acid (LNA)
  modified_base 24: mod_base = OTHER; note = locked nucleic acid (LNA)
  modified_base 26: mod_base = OTHER; note = locked nucleic acid (LNA)
  modified_base 2: mod_base = m5c
  modified_base 4: mod_base = m5c
  modified_base 6: mod_base = m5c
  modified_base 8: mod_base = m5c
  modified_base 10: mod_base = m5c
  modified_base 12: mod_base = m5c
  modified_base 14: mod_base = m5c
  modified_base 16: mod_base = m5c
  modified_base 18: mod_base = m5c
  modified_base 20: mod_base = m5c
  modified_base 22: mod_base = m5c
  modified_base 26: mod_base = m5c
  modified_base 24: mod_base = m5c
acacacacacacacacacacacacac

SEQ ID NO: 171 - DNA, 27 bp, synthetic construct
  source 1..27: mol_type = other DNA
  misc_feature 1..27: note = linked by phosphorothioate internucleoside linkage
  modified_base 1..2: mod_base = OTHER; note = locked nucleic acid (LNA)
  modified_base 5..6: mod_base = OTHER; note = locked nucleic acid (LNA)
  modified_base 9..10: mod_base = OTHER; note = locked nucleic acid (LNA)
  modified_base 13..14: mod_base = OTHER; note = locked nucleic acid (LNA)
  modified_base 17..18: mod_base = OTHER; note = locked nucleic acid (LNA)
  modified_base 21..22: mod_base = OTHER; note = locked nucleic acid (LNA)
  modified_base 25..27: mod_base = OTHER; note = locked nucleic acid (LNA)
  modified_base 1: mod_base = m5c
  modified_base 5: mod_base = m5c
  modified_base 9: mod_base = m5c
  modified_base 13: mod_base = m5c
  modified_base 17: mod_base = m5c
  modified_base 21: mod_base = m5c
  modified_base 25: mod_base = m5c
  modified_base 27: mod_base = m5c
cacacacacacacacacacacacacac

SEQ ID NO: 172 - DNA, 27 bp, synthetic construct
  source 1..27: mol_type = other DNA
  misc_feature 1..27: note = linked by phosphorothioate internucleoside linkage
  modified_base 1..2: mod_base = OTHER; note = locked nucleic acid (LNA)
  modified_base 6..7: mod_base = OTHER; note = locked nucleic acid (LNA)
  modified_base 10..11: mod_base = OTHER; note = locked nucleic acid (LNA)
  modified_base 15..16: mod_base = OTHER; note = locked nucleic acid (LNA)
  modified_base 19: mod_base = OTHER; note = locked nucleic acid (LNA)
  modified_base 22: mod_base = OTHER; note = locked nucleic acid (LNA)
  modified_base 26..27: mod_base = OTHER; note = locked nucleic acid (LNA)
  modified_base 1: mod_base = m5c
  modified_base 7: mod_base = m5c
  modified_base 11: mod_base = m5c
  modified_base 15: mod_base = m5c
  modified_base 19: mod_base = m5c
  modified_base 27: mod_base = m5c
cacacacacacacacacacacacacac

SEQ ID NO: 173 - DNA, 27 bp, synthetic construct
  source 1..27: mol_type = other DNA
  misc_feature 1..27: note = linked by phosphorothioate internucleoside linkage
  modified_base 1..2: mod_base = OTHER; note = locked nucleic acid (LNA)
  modified_base 5: mod_base = OTHER; note = locked nucleic acid (LNA)
  modified_base 7: mod_base = OTHER; note = locked nucleic acid (LNA)
  modified_base 9: mod_base = OTHER; note = locked nucleic acid (LNA)
  modified_base 11: mod_base = OTHER; note = locked nucleic acid (LNA)
  modified_base 13: mod_base = OTHER; note = locked nucleic acid (LNA)
  modified_base 15: mod_base = OTHER; note = locked nucleic acid (LNA)
  modified_base 17: mod_base = OTHER; note = locked nucleic acid (LNA)
  modified_base 19: mod_base = OTHER; note = locked nucleic acid (LNA)
  modified_base 21: mod_base = OTHER; note = locked nucleic acid (LNA)
  modified_base 23: mod_base = OTHER; note = locked nucleic acid (LNA)
  modified_base 26..27: mod_base = OTHER; note = locked nucleic acid (LNA)
  modified_base 1: mod_base = m5c
  modified_base 5: mod_base = m5c
  modified_base 7: mod_base = m5c
  modified_base 9: mod_base = m5c
  modified_base 11: mod_base = m5c
  modified_base 13: mod_base = m5c
  modified_base 15: mod_base = m5c
  modified_base 17: mod_base = m5c
  modified_base 19: mod_base = m5c
  modified_base 21: mod_base = m5c
  modified_base 23: mod_base = m5c
  modified_base 27: mod_base = m5c
cacacacacacacacacacacacacac

SEQ ID NO: 174 - DNA, 27 bp, synthetic construct
  source 1..27: mol_type = other DNA
  misc_feature 1..27: note = linked by phosphorothioate internucleoside linkage
  modified_base 1..2: mod_base = OTHER; note = locked nucleic acid (LNA)
  modified_base 4: mod_base = OTHER; note = locked nucleic acid (LNA)
  modified_base 6: mod_base = OTHER; note = locked nucleic acid (LNA)
  modified_base 8: mod_base = OTHER; note = locked nucleic acid (LNA)
  modified_base 10: mod_base = OTHER; note = locked nucleic acid (LNA)
  modified_base 12: mod_base = OTHER; note = locked nucleic acid (LNA)
  modified_base 14: mod_base = OTHER; note = locked nucleic acid (LNA)
  modified_base 16: mod_base = OTHER; note = locked nucleic acid (LNA)
  modified_base 18: mod_base = OTHER; note = locked nucleic acid (LNA)
  modified_base 20: mod_base = OTHER; note = locked nucleic acid (LNA)
  modified_base 22: mod_base = OTHER; note = locked nucleic acid (LNA)
  modified_base 24: mod_base = OTHER; note = locked nucleic acid (LNA)
  modified_base 26..27: mod_base = OTHER; note = locked nucleic acid (LNA)
  modified_base 1: mod_base = m5c
  modified_base 27: mod_base = m5c
cacacacacacacacacacacacacac

SEQ ID NO: 175 - DNA, 28 bp, synthetic construct
  source 1..28: mol_type = other DNA
  misc_feature 1..28: note = linked by phosphorothioate internucleoside linkage
  modified_base 1..2: mod_base = OTHER; note = locked nucleic acid (LNA)
  modified_base 4: mod_base = OTHER; note = locked nucleic acid (LNA)
  modified_base 6: mod_base = OTHER; note = locked nucleic acid (LNA)
  modified_base 8: mod_base = OTHER; note = locked nucleic acid (LNA)
  modified_base 10: mod_base = OTHER; note = locked nucleic acid (LNA)
  modified_base 12: mod_base = OTHER; note = locked nucleic acid (LNA)
  modified_base 14: mod_base = OTHER; note = locked nucleic acid (LNA)
  modified_base 16: mod_base = OTHER; note = locked nucleic acid (LNA)
  modified_base 18: mod_base = OTHER; note = locked nucleic acid (LNA)
  modified_base 20: mod_base = OTHER; note = locked nucleic acid (LNA)
  modified_base 22: mod_base = OTHER; note = locked nucleic acid (LNA)
  modified_base 24: mod_base = OTHER; note = locked nucleic acid (LNA)
  modified_base 26: mod_base = OTHER; note = locked nucleic acid (LNA)
  modified_base 28: mod_base = OTHER; note = locked nucleic acid (LNA)
  modified_base 2: mod_base = m5c
  modified_base 4: mod_base = m5c
  modified_base 6: mod_base = m5c
  modified_base 8: mod_base = m5c
  modified_base 10: mod_base = m5c
  modified_base 12: mod_base = m5c
  modified_base 14: mod_base = m5c
  modified_base 16: mod_base = m5c
  modified_base 18: mod_base = m5c
  modified_base 20: mod_base = m5c
  modified_base 22: mod_base = m5c
  modified_base 24: mod_base = m5c
  modified_base 26: mod_base = m5c
  modified_base 28: mod_base = m5c
acacacacacacacacacacacacacac

SEQ ID NO: 176 - DNA, 29 bp, synthetic construct
  source 1..29: mol_type = other DNA
  misc_feature 1..29: note = linked by phosphorothioate internucleoside linkage
  modified_base 1..2: mod_base = OTHER; note = locked nucleic acid (LNA)
  modified_base 4: mod_base = OTHER; note = locked nucleic acid (LNA)
  modified_base 6: mod_base = OTHER; note = locked nucleic acid (LNA)
  modified_base 8: mod_base = OTHER; note = locked nucleic acid (LNA)
  modified_base 10: mod_base = OTHER; note = locked nucleic acid (LNA)
  modified_base 12: mod_base = OTHER; note = locked nucleic acid (LNA)
  modified_base 14: mod_base = OTHER; note = locked nucleic acid (LNA)
  modified_base 16: mod_base = OTHER; note = locked nucleic acid (LNA)
  modified_base 18: mod_base = OTHER; note = locked nucleic acid (LNA)
  modified_base 20: mod_base = OTHER; note = locked nucleic acid (LNA)
  modified_base 22: mod_base = OTHER; note = locked nucleic acid (LNA)
  modified_base 24: mod_base = OTHER; note = locked nucleic acid (LNA)
  modified_base 26: mod_base = OTHER; note = locked nucleic acid (LNA)
  modified_base 28..29: mod_base = OTHER; note = locked nucleic acid (LNA)
  modified_base 1: mod_base = m5c
  modified_base 29: mod_base = m5c
cacacacacacacacacacacacacacac

SEQ ID NO: 177 - DNA, 29 bp, synthetic construct
  source 1..29: mol_type = other DNA
  misc_feature 1..29: note = linked by phosphorothioate internucleoside linkage
  modified_base 1..2: mod_base = OTHER; note = locked nucleic acid (LNA)
  modified_base 5..6: mod_base = OTHER; note = locked nucleic acid (LNA)
  modified_base 9..10: mod_base = OTHER; note = locked nucleic acid (LNA)
  modified_base 13..14: mod_base = OTHER; note = locked nucleic acid (LNA)
  modified_base 17..18: mod_base = OTHER; note = locked nucleic acid (LNA)
  modified_base 21..22: mod_base = OTHER; note = locked nucleic acid (LNA)
  modified_base 25..26: mod_base = OTHER; note = locked nucleic acid (LNA)
  modified_base 29: mod_base = OTHER; note = locked nucleic acid (LNA)
  modified_base 1: mod_base = m5c
  modified_base 5: mod_base = m5c
  modified_base 9: mod_base = m5c
  modified_base 13: mod_base = m5c
  modified_base 17: mod_base = m5c
  modified_base 21: mod_base = m5c
  modified_base 25: mod_base = m5c
  modified_base 29: mod_base = m5c
cacacacacacacacacacacacacacac

SEQ ID NO: 178 - DNA, 29 bp, synthetic construct
  source 1..29: mol_type = other DNA
  misc_feature 1..29: note = linked by phosphorothioate internucleoside linkage
  modified_base 1..2: mod_base = OTHER; note = locked nucleic acid (LNA)
  modified_base 5: mod_base = OTHER; note = locked nucleic acid (LNA)
  modified_base 7: mod_base = OTHER; note = locked nucleic acid (LNA)
  modified_base 9: mod_base = OTHER; note = locked nucleic acid (LNA)
  modified_base 11: mod_base = OTHER; note = locked nucleic acid (LNA)
  modified_base 13: mod_base = OTHER; note = locked nucleic acid (LNA)
  modified_base 15: mod_base = OTHER; note = locked nucleic acid (LNA)
  modified_base 17: mod_base = OTHER; note = locked nucleic acid (LNA)
  modified_base 19: mod_base = OTHER; note = locked nucleic acid (LNA)
  modified_base 21: mod_base = OTHER; note = locked nucleic acid (LNA)
  modified_base 23: mod_base = OTHER; note = locked nucleic acid (LNA)
  modified_base 25: mod_base = OTHER; note = locked nucleic acid (LNA)
  modified_base 29: mod_base = OTHER; note = locked nucleic acid (LNA)
  modified_base 1: mod_base = m5c
  modified_base 7: mod_base = m5c
  modified_base 11: mod_base = m5c
  modified_base 15: mod_base = m5c
  modified_base 19: mod_base = m5c
  modified_base 29: mod_base = m5c
  modified_base 5: mod_base = m5c
  modified_base 9: mod_base = m5c
  modified_base 13: mod_base = m5c
  modified_base 17: mod_base = m5c
  modified_base 21: mod_base = m5c
  modified_base 23: mod_base = m5c
  modified_base 25: mod_base = m5c
cacacacacacacacacacacacacacac

SEQ ID NO: 179 - DNA, 29 bp, synthetic construct
  source 1..29: mol_type = other DNA
  misc_feature 1..29: note = linked by phosphorothioate internucleoside linkage
  modified_base 1..2: mod_base = OTHER; note = locked nucleic acid (LNA)
  modified_base 6..7: mod_base = OTHER; note = locked nucleic acid (LNA)
  modified_base 10..11: mod_base = OTHER; note = locked nucleic acid (LNA)
  modified_base 15..16: mod_base = OTHER; note = locked nucleic acid (LNA)
  modified_base 19..20: mod_base = OTHER; note = locked nucleic acid (LNA)
  modified_base 24: mod_base = OTHER; note = locked nucleic acid (LNA)
  modified_base 28..29: mod_base = OTHER; note = locked nucleic acid (LNA)
  modified_base 1: mod_base = m5c
  modified_base 7: mod_base = m5c
  modified_base 11: mod_base = m5c
  modified_base 15: mod_base = m5c
  modified_base 19: mod_base = m5c
  modified_base 29: mod_base = m5c
cacacacacacacacacacacacacacac

SEQ ID NO: 180 - DNA, 30 bp, synthetic construct; identical to SEQ ID NO: 28
  source 1..30: mol_type = other DNA
  misc_feature 1..30: note = linked by phosphorothioate internucleoside linkage
  modified_base 1..2: mod_base = OTHER; note = locked nucleic acid (LNA)
  modified_base 4: mod_base = OTHER; note = locked nucleic acid (LNA)
  modified_base 6: mod_base = OTHER; note = locked nucleic acid (LNA)
  modified_base 8: mod_base = OTHER; note = locked nucleic acid (LNA)
  modified_base 10: mod_base = OTHER; note = locked nucleic acid (LNA)
  modified_base 12: mod_base = OTHER; note = locked nucleic acid (LNA)
  modified_base 14: mod_base = OTHER; note = locked nucleic acid (LNA)
  modified_base 16: mod_base = OTHER; note = locked nucleic acid (LNA)
  modified_base 18: mod_base = OTHER; note = locked nucleic acid (LNA)
  modified_base 20: mod_base = OTHER; note = locked nucleic acid (LNA)
  modified_base 22: mod_base = OTHER; note = locked nucleic acid (LNA)
  modified_base 24: mod_base = OTHER; note = locked nucleic acid (LNA)
  modified_base 26: mod_base = OTHER; note = locked nucleic acid (LNA)
  modified_base 28: mod_base = OTHER; note = locked nucleic acid (LNA)
  modified_base 30: mod_base = OTHER; note = locked nucleic acid (LNA)
  modified_base 2: mod_base = m5c
  modified_base 4: mod_base = m5c
  modified_base 6: mod_base = m5c
  modified_base 8: mod_base = m5c
  modified_base 10: mod_base = m5c
  modified_base 12: mod_base = m5c
  modified_base 14: mod_base = m5c
  modified_base 16: mod_base = m5c
  modified_base 18: mod_base = m5c
  modified_base 20: mod_base = m5c
  modified_base 22: mod_base = m5c
  modified_base 24: mod_base = m5c
  modified_base 26: mod_base = m5c
  modified_base 28: mod_base = m5c
  modified_base 30: mod_base = m5c

SEQ ID NO: 181 - DNA, 31 bp, synthetic construct; identical to SEQ ID NO: 29
  source 1..31: mol_type = other DNA
  misc_feature 1..31: note = linked by phosphorothioate internucleoside linkage
  modified_base 1..2: mod_base = OTHER; note = locked nucleic acid (LNA)
  modified_base 4: mod_base = OTHER; note = locked nucleic acid (LNA)
  modified_base 6: mod_base = OTHER; note = locked nucleic acid (LNA)
  modified_base 8: mod_base = OTHER; note = locked nucleic acid (LNA)
  modified_base 10: mod_base = OTHER; note = locked nucleic acid (LNA)
  modified_base 12: mod_base = OTHER; note = locked nucleic acid (LNA)
  modified_base 14: mod_base = OTHER; note = locked nucleic acid (LNA)
  modified_base 16: mod_base = OTHER; note = locked nucleic acid (LNA)
  modified_base 18: mod_base = OTHER; note = locked nucleic acid (LNA)
  modified_base 20: mod_base = OTHER; note = locked nucleic acid (LNA)
  modified_base 22: mod_base = OTHER; note = locked nucleic acid (LNA)
  modified_base 24: mod_base = OTHER; note = locked nucleic acid (LNA)
  modified_base 26: mod_base = OTHER; note = locked nucleic acid (LNA)
  modified_base 28: mod_base = OTHER; note = locked nucleic acid (LNA)
  modified_base 30..31: mod_base = OTHER; note = locked nucleic acid (LNA)
  modified_base 1: mod_base = m5c
  modified_base 31: mod_base = m5c

SEQ ID NO: 182 - DNA, 31 bp, synthetic construct; identical to SEQ ID NO: 29
  source 1..31: mol_type = other DNA
  misc_feature 1..31: note = linked by phosphorothioate internucleoside linkage
  modified_base 1..2: mod_base = OTHER; note = locked nucleic acid (LNA)
  modified_base 5..6: mod_base = OTHER; note = locked nucleic acid (LNA)
  modified_base 9..10: mod_base = OTHER; note = locked nucleic acid (LNA)
  modified_base 13..14: mod_base = OTHER; note = locked nucleic acid (LNA)
  modified_base 17..18: mod_base = OTHER; note = locked nucleic acid (LNA)
  modified_base 21..22: mod_base = OTHER; note = locked nucleic acid (LNA)
  modified_base 25..26: mod_base = OTHER; note = locked nucleic acid (LNA)
  modified_base 29..31: mod_base = OTHER; note = locked nucleic acid (LNA)
  modified_base 1: mod_base = m5c
  modified_base 5: mod_base = m5c
  modified_base 9: mod_base = m5c
  modified_base 13: mod_base = m5c
  modified_base 17: mod_base = m5c
  modified_base 21: mod_base = m5c
  modified_base 25: mod_base = m5c
  modified_base 29: mod_base = m5c
  modified_base 31: mod_base = m5c

SEQ ID NO: 183 - DNA, 31 bp, synthetic construct; identical to SEQ ID NO: 29
  source 1..31: mol_type = other DNA
  misc_feature 1..31: note = linked by phosphorothioate internucleoside linkage
  modified_base 1..2: mod_base = OTHER; note = locked nucleic acid (LNA)
  modified_base 5: mod_base = OTHER; note = locked nucleic acid (LNA)
  modified_base 7: mod_base = OTHER; note = locked nucleic acid (LNA)
  modified_base 9: mod_base = OTHER; note = locked nucleic acid (LNA)
  modified_base 11: mod_base = OTHER; note = locked nucleic acid (LNA)
  modified_base 13: mod_base = OTHER; note = locked nucleic acid (LNA)
  modified_base 15: mod_base = OTHER; note = locked nucleic acid (LNA)
  modified_base 17: mod_base = OTHER; note = locked nucleic acid (LNA)
  modified_base 19: mod_base = OTHER; note = locked nucleic acid (LNA)
  modified_base 21: mod_base = OTHER; note = locked nucleic acid (LNA)
  modified_base 23: mod_base = OTHER; note = locked nucleic acid (LNA)
  modified_base 25: mod_base = OTHER; note = locked nucleic acid (LNA)
  modified_base 27: mod_base = OTHER; note = locked nucleic acid (LNA)
  modified_base 30..31: mod_base = OTHER; note = locked nucleic acid (LNA)
  modified_base 1: mod_base = m5c
  modified_base 5: mod_base = m5c
  modified_base 7: mod_base = m5c
  modified_base 9: mod_base = m5c
  modified_base 11: mod_base = m5c
  modified_base 13: mod_base = m5c
  modified_base 15: mod_base = m5c
  modified_base 17: mod_base = m5c
  modified_base 19: mod_base = m5c
  modified_base 21: mod_base = m5c
  modified_base 23: mod_base = m5c
  modified_base 25: mod_base = m5c
  modified_base 27: mod_base = m5c
  modified_base 31: mod_base = m5c

SEQ ID NO: 184 - DNA, 31 bp, synthetic construct; identical to SEQ ID NO: 29
  source 1..31: mol_type = other DNA
  misc_feature 1..31: note = linked by phosphorothioate internucleoside linkage
  modified_base 1..2: mod_base = OTHER; note = locked nucleic acid (LNA)
  modified_base 6..7: mod_base = OTHER; note = locked nucleic acid (LNA)
  modified_base 10..11: mod_base = OTHER; note = locked nucleic acid (LNA)
  modified_base 15..16: mod_base = OTHER; note = locked nucleic acid (LNA)
  modified_base 19..20: mod_base = OTHER; note = locked nucleic acid (LNA)
  modified_base 24..26: mod_base = OTHER; note = locked nucleic acid (LNA)
  modified_base 30..31: mod_base = OTHER; note = locked nucleic acid (LNA)
  modified_base 1: mod_base = m5c
  modified_base 7: mod_base = m5c
  modified_base 11: mod_base = m5c
  modified_base 15: mod_base = m5c
  modified_base 19: mod_base = m5c
  modified_base 25: mod_base = m5c
  modified_base 31: mod_base = m5c

SEQ ID NO: 185 - DNA, 34 bp, synthetic construct; identical to SEQ ID NO: 30
  source 1..34: mol_type = other DNA
  misc_feature 1..34: note = linked by phosphorothioate internucleoside linkage
  modified_base 1..3: mod_base = OTHER; note = locked nucleic acid (LNA)
  modified_base 7: mod_base = OTHER; note = locked nucleic acid (LNA)
  modified_base 9: mod_base = OTHER; note = locked nucleic acid (LNA)
  modified_base 13: mod_base = OTHER; note = locked nucleic acid (LNA)
  modified_base 15: mod_base = OTHER; note = locked nucleic acid (LNA)
  modified_base 18..19: mod_base = OTHER; note = locked nucleic acid (LNA)
  modified_base 22: mod_base = OTHER; note = locked nucleic acid (LNA)
  modified_base 24: mod_base = OTHER; note = locked nucleic acid (LNA)
  modified_base 26: mod_base = OTHER; note = locked nucleic acid (LNA)
  modified_base 28: mod_base = OTHER; note = locked nucleic acid (LNA)
  modified_base 30: mod_base = OTHER; note = locked nucleic acid (LNA)
  modified_base 33..34: mod_base = OTHER; note = locked nucleic acid (LNA)
  modified_base 2: mod_base = m5c
  modified_base 18..19: mod_base = m5c
  modified_base 33: mod_base = m5c

SEQ ID NO: 186 - DNA, 34 bp, synthetic construct; identical to SEQ ID NO: 31
  source 1..34: mol_type = other DNA
  misc_feature 1..34: note = linked by phosphorothioate internucleoside linkage
  modified_base 1: mod_base = OTHER; note = locked nucleic acid (LNA)
  modified_base 4: mod_base = OTHER; note = locked nucleic acid (LNA)
  modified_base 6: mod_base = OTHER; note = locked nucleic acid (LNA)
  modified_base 8: mod_base = OTHER; note = locked nucleic acid (LNA)
  modified_base 12: mod_base = OTHER; note = locked nucleic acid (LNA)
  modified_base 15..17: mod_base = OTHER; note = locked nucleic acid (LNA)
  modified_base 20: mod_base = OTHER; note = locked nucleic acid (LNA)
  modified_base 22: mod_base = OTHER; note = locked nucleic acid (LNA)
  modified_base 24: mod_base = OTHER; note = locked nucleic acid (LNA)
  modified_base 26: mod_base = OTHER; note = locked nucleic acid (LNA)
  modified_base 28: mod_base = OTHER; note = locked nucleic acid (LNA)
  modified_base 30: mod_base = OTHER; note = locked nucleic acid (LNA)
  modified_base 33..34: mod_base = OTHER; note = locked nucleic acid (LNA)
  modified_base 1: mod_base = m5c
  modified_base 15: mod_base = m5c
  modified_base 17: mod_base = m5c
  modified_base 33: mod_base = m5c

SEQ ID NO: 187 - DNA, 34 bp, synthetic construct; identical to SEQ ID NO: 31
  source 1..34: mol_type = other DNA
  misc_feature 1..34: note = linked by phosphorothioate internucleoside linkage
  modified_base 1: mod_base = OTHER; note = locked nucleic acid (LNA)
  modified_base 4: mod_base = OTHER; note = locked nucleic acid (LNA)
  modified_base 6: mod_base = OTHER; note = locked nucleic acid (LNA)
  modified_base 8: mod_base = OTHER; note = locked nucleic acid (LNA)
  modified_base 10: mod_base = OTHER; note = locked nucleic acid (LNA)
  modified_base 12: mod_base = OTHER; note = locked nucleic acid (LNA)
  modified_base 14: mod_base = OTHER; note = locked nucleic acid (LNA)
  modified_base 17..19: mod_base = OTHER; note = locked nucleic acid (LNA)
  modified_base 20: mod_base = OTHER; note = locked nucleic acid (LNA)
  modified_base 22: mod_base = OTHER; note = locked nucleic acid (LNA)
  modified_base 24: mod_base = OTHER; note = locked nucleic acid (LNA)
  modified_base 26: mod_base = OTHER; note = locked nucleic acid (LNA)
  modified_base 28: mod_base = OTHER; note = locked nucleic acid (LNA)
  modified_base 28: mod_base = OTHER; note = locked nucleic acid (LNA)
  modified_base 30: mod_base = OTHER; note = locked nucleic acid (LNA)
  modified_base 33..34: mod_base = OTHER; note = locked nucleic acid (LNA)
  modified_base 1: mod_base = m5c
  modified_base 17: mod_base = m5c
  modified_base 19: mod_base = m5c
  modified_base 33: mod_base = m5c

SEQ ID NO: 188 - DNA, 36 bp, synthetic construct; identical to SEQ ID NO: 32
  source 1..36: mol_type = other DNA
  misc_feature 1..36: note = linked by phosphorothioate internucleoside linkage
  modified_base 1: mod_base = OTHER; note = locked nucleic acid (LNA)
  modified_base 4: mod_base = OTHER; note = locked nucleic acid (LNA)
  modified_base 6: mod_base = OTHER; note = locked nucleic acid (LNA)
  modified_base 8: mod_base = OTHER; note = locked nucleic acid (LNA)
  modified_base 10: mod_base = OTHER; note = locked nucleic acid (LNA)
  modified_base 12: mod_base = OTHER; note = locked nucleic acid (LNA)
  modified_base 14: mod_base = OTHER; note = locked nucleic acid (LNA)
  modified_base 17..21: mod_base = OTHER; note = locked nucleic acid (LNA)
  modified_base 25: mod_base = OTHER; note = locked nucleic acid (LNA)
  modified_base 27: mod_base = OTHER; note = locked nucleic acid (LNA)
  modified_base 31: mod_base = OTHER; note = locked nucleic acid (LNA)
  modified_base 33: mod_base = OTHER; note = locked nucleic acid (LNA)
  modified_base 36: mod_base = OTHER; note = locked nucleic acid (LNA)
  modified_base 1: mod_base = m5c
  modified_base 17: mod_base = m5c
  modified_base 20: mod_base = m5c
  modified_base 36: mod_base = m5c

SEQ ID NO: 189 - DNA, 36 bp, synthetic construct; identical to SEQ ID NO: 33
  source 1..36: mol_type = other DNA
  misc_feature 1..36: note = linked by phosphorothioate internucleoside linkage
  modified_base 1..3: mod_base = OTHER; note = locked nucleic acid (LNA)
  modified_base 7: mod_base = OTHER; note = locked nucleic acid (LNA)
  modified_base 9: mod_base = OTHER; note = locked nucleic acid (LNA)
  modified_base 13: mod_base = OTHER; note = locked nucleic acid (LNA)
  modified_base 15: mod_base = OTHER; note = locked nucleic acid (LNA)
  modified_base 18..19: mod_base = OTHER; note = locked nucleic acid (LNA)
  modified_base 22: mod_base = OTHER; note = locked nucleic acid (LNA)
  modified_base 24: mod_base = OTHER; note = locked nucleic acid (LNA)
  modified_base 26: mod_base = OTHER; note = locked nucleic acid (LNA)
  modified_base 28: mod_base = OTHER; note = locked nucleic acid (LNA)
  modified_base 30: mod_base = OTHER; note = locked nucleic acid (LNA)
  modified_base 32: mod_base = OTHER; note = locked nucleic acid (LNA)
  modified_base 35..36: mod_base = OTHER; note = locked nucleic acid (LNA)
  modified_base 2: mod_base = m5c
  modified_base 18..19: mod_base = m5c
  modified_base 35: mod_base = m5c

SEQ ID NO: 190 - DNA, 24 bp, synthetic construct
  source 1..24: mol_type = other DNA
  misc_feature 1..24: note = linked by phosphorothioate internucleoside linkage
  modified_base 1..24: mod_base = OTHER; note = 2-O-(2-methoxyethyl) nucleoside
  modified_base 1: mod_base = m5c
  modified_base 3: mod_base = m5c
  modified_base 5: mod_base = m5c
  modified_base 7: mod_base = m5c
  modified_base 9: mod_base = m5c
  modified_base 11: mod_base = m5c
  modified_base 14: mod_base = m5c
  modified_base 16: mod_base = m5c
  modified_base 18: mod_base = m5c
  modified_base 20: mod_base = m5c
  modified_base 22: mod_base = m5c
  modified_base 24: mod_base = m5c
cacacacacacaacacacacacac

SEQ ID NO: 191 - DNA, 25 bp, synthetic construct
  source 1..25: mol_type = other DNA
  misc_feature 1..25: note = linked by phosphorothioate internucleoside linkage
  modified_base 1..25: mod_base = OTHER; note = 2-O-(2-methoxyethyl) nucleoside
  modified_base 1: mod_base = m5c
  modified_base 3: mod_base = m5c
  modified_base 5: mod_base = m5c
  modified_base 7: mod_base = m5c
  modified_base 9: mod_base = m5c
  modified_base 11: mod_base = m5c
  modified_base 13: mod_base = m5c
  modified_base 15: mod_base = m5c
  modified_base 17: mod_base = m5c
  modified_base 19: mod_base = m5c
  modified_base 21: mod_base = m5c
  modified_base 23: mod_base = m5c
  modified_base 25: mod_base = m5c
cacacacacacacacacacacacac

SEQ ID NO: 192 - DNA, 27 bp, synthetic construct
  source 1..27: mol_type = other DNA
  misc_feature 1..27: note = linked by phosphorothioate internucleoside linkage
  modified_base 1..27: mod_base = OTHER; note = 2-O-(2-methoxyethyl) nucleoside
  modified_base 1: mod_base = m5c
  modified_base 3: mod_base = m5c
  modified_base 5: mod_base = m5c
  modified_base 7: mod_base = m5c
  modified_base 9: mod_base = m5c
  modified_base 11: mod_base = m5c
  modified_base 13: mod_base = m5c
  modified_base 15: mod_base = m5c
  modified_base 17: mod_base = m5c
  modified_base 19: mod_base = m5c
  modified_base 21: mod_base = m5c
  modified_base 21: mod_base = m5c
  modified_base 23: mod_base = m5c
  modified_base 25: mod_base = m5c
  modified_base 27: mod_base = m5c
cacacacacacacacacacacacacac

SEQ ID NO: 193 - DNA, 29 bp, synthetic construct
  source 1..29: mol_type = other DNA
  misc_feature 1..29: note = linked by phosphorothioate internucleoside linkage
  modified_base 1..29: mod_base = OTHER; note = 2-O-(2-methoxyethyl) nucleoside
  modified_base 1: mod_base = m5c
  modified_base 3: mod_base = m5c
  modified_base 5: mod_base = m5c
  modified_base 7: mod_base = m5c
  modified_base 9: mod_base = m5c
  modified_base 11: mod_base = m5c
  modified_base 13: mod_base = m5c
  modified_base 15: mod_base = m5c
  modified_base 17: mod_base = m5c
  modified_base 19: mod_base = m5c
  modified_base 21: mod_base = m5c
  modified_base 23: mod_base = m5c
  modified_base 25: mod_base = m5c
  modified_base 27: mod_base = m5c
  modified_base 29: mod_base = m5c
cacacacacacacacacacacacacacac

SEQ ID NO: 194 - DNA, 31 bp, synthetic construct; identical to SEQ ID NO: 29
  source 1..31: mol_type = other DNA
  misc_feature 1..31: note = linked by phosphorothioate internucleoside linkage
  modified_base 1..31: mod_base = OTHER; note = 2-O-(2-methoxyethyl) nucleoside
  modified_base 1: mod_base = m5c
  modified_base 3: mod_base = m5c
  modified_base 5: mod_base = m5c
  modified_base 7: mod_base = m5c
  modified_base 9: mod_base = m5c
  modified_base 11: mod_base = m5c
  modified_base 13: mod_base = m5c
  modified_base 15: mod_base = m5c
  modified_base 17: mod_base = m5c
  modified_base 19: mod_base = m5c
  modified_base 21: mod_base = m5c
  modified_base 23: mod_base = m5c
  modified_base 25: mod_base = m5c
  modified_base 27: mod_base = m5c
  modified_base 29: mod_base = m5c
  modified_base 31: mod_base = m5c

SEQ ID NO: 195 - DNA, 25 bp, synthetic construct
  source 1..25: mol_type = other DNA
  misc_feature 1..25: note = linked by phosphorothioate internucleoside linkage
  modified_base 1..2: mod_base = OTHER; note = 2-O-(2-methoxyethyl) nucleoside
  modified_base 4: mod_base = OTHER; note = 2-O-(2-methoxyethyl) nucleoside
  modified_base 6: mod_base = OTHER; note = 2-O-(2-methoxyethyl) nucleoside
  modified_base 8: mod_base = OTHER; note = 2-O-(2-methoxyethyl) nucleoside
  modified_base 10: mod_base = OTHER; note = 2-O-(2-methoxyethyl) nucleoside
  modified_base 12: mod_base = OTHER; note = 2-O-(2-methoxyethyl) nucleoside
  modified_base 14: mod_base = OTHER; note = 2-O-(2-methoxyethyl) nucleoside
  modified_base 16: mod_base = OTHER; note = 2-O-(2-methoxyethyl) nucleoside
  modified_base 18: mod_base = OTHER; note = 2-O-(2-methoxyethyl) nucleoside
  modified_base 20: mod_base = OTHER; note = 2-O-(2-methoxyethyl) nucleoside
  modified_base 22: mod_base = OTHER; note = 2-O-(2-methoxyethyl) nucleoside
  modified_base 24..25: mod_base = OTHER; note = 2-O-(2-methoxyethyl) nucleoside
  modified_base 1: mod_base = m5c
  modified_base 25: mod_base = m5c
cacacacacacacacacacacacac

SEQ ID NO: 196 - DNA, 27 bp, synthetic construct
  source 1..27: mol_type = other DNA
  misc_feature 1..27: note = linked by phosphorothioate internucleoside linkage
  modified_base 1..2: mod_base = OTHER; note = 2-O-(2-methoxyethyl) nucleoside
  modified_base 4: mod_base = OTHER; note = 2-O-(2-methoxyethyl) nucleoside
  modified_base 6: mod_base = OTHER; note = 2-O-(2-methoxyethyl) nucleoside
  modified_base 8: mod_base = OTHER; note = 2-O-(2-methoxyethyl) nucleoside
  modified_base 10: mod_base = OTHER; note = 2-O-(2-methoxyethyl) nucleoside
  modified_base 12: mod_base = OTHER; note = 2-O-(2-methoxyethyl) nucleoside
  modified_base 14: mod_base = OTHER; note = 2-O-(2-methoxyethyl) nucleoside
  modified_base 16: mod_base = OTHER; note = 2-O-(2-methoxyethyl) nucleoside
  modified_base 18: mod_base = OTHER; note = 2-O-(2-methoxyethyl) nucleoside
  modified_base 20: mod_base = OTHER; note = 2-O-(2-methoxyethyl) nucleoside
  modified_base 22: mod_base = OTHER; note = 2-O-(2-methoxyethyl) nucleoside
  modified_base 24: mod_base = OTHER; note = 2-O-(2-methoxyethyl) nucleoside
  modified_base 26..27: mod_base = OTHER; note = 2-O-(2-methoxyethyl) nucleoside
  modified_base 1: mod_base = m5c
  modified_base 27: mod_base = m5c
cacacacacacacacacacacacacac

SEQ ID NO: 197 - DNA, 25 bp, synthetic construct
  source 1..25: mol_type = other DNA
  misc_feature 1..25: note = linked by phosphorothioate internucleoside linkage
  modified_base 1..2: mod_base = OTHER; note = 2-O-(2-methoxyethyl) nucleoside
  modified_base 5: mod_base = OTHER; note = 2-O-(2-methoxyethyl) nucleoside
  modified_base 7: mod_base = OTHER; note = 2-O-(2-methoxyethyl) nucleoside
  modified_base 9: mod_base = OTHER; note = 2-O-(2-methoxyethyl) nucleoside
  modified_base 11: mod_base = OTHER; note = 2-O-(2-methoxyethyl) nucleoside
  modified_base 13: mod_base = OTHER; note = 2-O-(2-methoxyethyl) nucleoside
  modified_base 15: mod_base = OTHER; note = 2-O-(2-methoxyethyl) nucleoside
  modified_base 17: mod_base = OTHER; note = 2-O-(2-methoxyethyl) nucleoside
  modified_base 19: mod_base = OTHER; note = 2-O-(2-methoxyethyl) nucleoside
  modified_base 21: mod_base = OTHER; note = 2-O-(2-methoxyethyl) nucleoside
  modified_base 24..25: mod_base = OTHER; note = 2-O-(2-methoxyethyl) nucleoside
  modified_base 1: mod_base = m5c
  modified_base 5: mod_base = m5c
  modified_base 7: mod_base = m5c
  modified_base 9: mod_base = m5c
  modified_base 11: mod_base = m5c
  modified_base 13: mod_base = m5c
  modified_base 17: mod_base = m5c
  modified_base 19: mod_base = m5c
  modified_base 21: mod_base = m5c
  modified_base 25: mod_base = m5c
cacacacacacacacacacacacac

SEQ ID NO: 198 - DNA, 27 bp, synthetic construct
  source 1..27: mol_type = other DNA
  misc_feature 1..27: note = linked by phosphorothioate internucleoside linkage
  modified_base 1..2: mod_base = OTHER; note = 2-O-(2-methoxyethyl) nucleoside
  modified_base 5: mod_base = OTHER; note = 2-O-(2-methoxyethyl) nucleoside
  modified_base 7: mod_base = OTHER; note = 2-O-(2-methoxyethyl) nucleoside
  modified_base 9: mod_base = OTHER; note = 2-O-(2-methoxyethyl) nucleoside
  modified_base 11: mod_base = OTHER; note = 2-O-(2-methoxyethyl) nucleoside
  modified_base 13: mod_base = OTHER; note = 2-O-(2-methoxyethyl) nucleoside
  modified_base 15: mod_base = OTHER; note = 2-O-(2-methoxyethyl) nucleoside
  modified_base 17: mod_base = OTHER; note = 2-O-(2-methoxyethyl) nucleoside
  modified_base 19: mod_base = OTHER; note = 2-O-(2-methoxyethyl) nucleoside
  modified_base 21: mod_base = OTHER; note = 2-O-(2-methoxyethyl) nucleoside
  modified_base 23: mod_base = OTHER; note = 2-O-(2-methoxyethyl) nucleoside
  modified_base 26..27: mod_base = OTHER; note = 2-O-(2-methoxyethyl) nucleoside
  modified_base 1: mod_base = m5c
  modified_base 3: mod_base = m5c
  modified_base 5: mod_base = m5c
  modified_base 7: mod_base = m5c
  modified_base 9: mod_base = m5c
  modified_base 11: mod_base = m5c
  modified_base 13: mod_base = m5c
  modified_base 15: mod_base = m5c
  modified_base 17: mod_base = m5c
  modified_base 19: mod_base = m5c
  modified_base 21: mod_base = m5c
  modified_base 23: mod_base = m5c
  modified_base 27: mod_base = m5c
cacacacacacacacacacacacacac

SEQ ID NO: 199 - DNA, 29 bp, synthetic construct
  source 1..29: mol_type = other DNA
  misc_feature 1..29: note = linked by phosphorothioate internucleoside linkage
  modified_base 1..2: mod_base = OTHER; note = 2-O-(2-methoxyethyl) nucleoside
  modified_base 4: mod_base = OTHER; note = 2-O-(2-methoxyethyl) nucleoside
  modified_base 6: mod_base = OTHER; note = 2-O-(2-methoxyethyl) nucleoside
  modified_base 8: mod_base = OTHER; note = 2-O-(2-methoxyethyl) nucleoside
  modified_base 10: mod_base = OTHER; note = 2-O-(2-methoxyethyl) nucleoside
  modified_base 12: mod_base = OTHER; note = 2-O-(2-methoxyethyl) nucleoside
  modified_base 14: mod_base = OTHER; note = 2-O-(2-methoxyethyl) nucleoside
  modified_base 16: mod_base = OTHER; note = 2-O-(2-methoxyethyl) nucleoside
  modified_base 18: mod_base = OTHER; note = 2-O-(2-methoxyethyl) nucleoside
  modified_base 20: mod_base = OTHER; note = 2-O-(2-methoxyethyl) nucleoside
  modified_base 22: mod_base = OTHER; note = 2-O-(2-methoxyethyl) nucleoside
  modified_base 24: mod_base = OTHER; note = 2-O-(2-methoxyethyl) nucleoside
  modified_base 26: mod_base = OTHER; note = 2-O-(2-methoxyethyl) nucleoside
  modified_base 28..29: mod_base = OTHER; note = 2-O-(2-methoxyethyl) nucleoside
  modified_base 1: mod_base = m5c
  modified_base 29: mod_base = m5c
cacacacacacacacacacacacacacac

SEQ ID NO: 200 - DNA, 29 bp, synthetic construct
  source 1..29: mol_type = other DNA
  misc_feature 1..29: note = linked by phosphorothioate internucleoside linkage
  modified_base 1..2: mod_base = OTHER; note = 2-O-(2-methoxyethyl) nucleoside
  modified_base 5: mod_base = OTHER; note = 2-O-(2-methoxyethyl) nucleoside
  modified_base 7: mod_base = OTHER; note = 2-O-(2-methoxyethyl) nucleoside
  modified_base 9: mod_base = OTHER; note = 2-O-(2-methoxyethyl) nucleoside
  modified_base 11: mod_base = OTHER; note = 2-O-(2-methoxyethyl) nucleoside
  modified_base 13: mod_base = OTHER; note = 2-O-(2-methoxyethyl) nucleoside
  modified_base 15: mod_base = OTHER; note = 2-O-(2-methoxyethyl) nucleoside
  modified_base 17: mod_base = OTHER; note = 2-O-(2-methoxyethyl) nucleoside
  modified_base 19: mod_base = OTHER; note = 2-O-(2-methoxyethyl) nucleoside
  modified_base 21: mod_base = OTHER; note = 2-O-(2-methoxyethyl) nucleoside
  modified_base 23: mod_base = OTHER; note = 2-O-(2-methoxyethyl) nucleoside
  modified_base 25: mod_base = OTHER; note = 2-O-(2-methoxyethyl) nucleoside
  modified_base 28..29: mod_base = OTHER; note = 2-O-(2-methoxyethyl) nucleoside
  modified_base 1: mod_base = m5c
  modified_base 5: mod_base = m5c
  modified_base 7: mod_base = m5c
  modified_base 9: mod_base = m5c
  modified_base 11: mod_base = m5c
  modified_base 13: mod_base = m5c
  modified_base 15: mod_base = m5c
  modified_base 17: mod_base = m5c
  modified_base 19: mod_base = m5c
  modified_base 21: mod_base = m5c
  modified_base 23: mod_base = m5c
  modified_base 25: mod_base = m5c
  modified_base 29: mod_base = m5c
cacacacacacacacacacacacacacac

SEQ ID NO: 201 - DNA, 31 bp, synthetic construct; identical to SEQ ID NO: 29
  source 1..31: mol_type = other DNA
  misc_feature 1..31: note = linked by phosphorothioate internucleoside linkage
  modified_base 1..2: mod_base = OTHER; note = 2-O-(2-methoxyethyl) nucleoside
  modified_base 4: mod_base = OTHER; note = 2-O-(2-methoxyethyl) nucleoside
  modified_base 6: mod_base = OTHER; note = 2-O-(2-methoxyethyl) nucleoside
  modified_base 8: mod_base = OTHER; note = 2-O-(2-methoxyethyl) nucleoside
  modified_base 10: mod_base = OTHER; note = 2-O-(2-methoxyethyl) nucleoside
  modified_base 12: mod_base = OTHER; note = 2-O-(2-methoxyethyl) nucleoside
  modified_base 14: mod_base = OTHER; note = 2-O-(2-methoxyethyl) nucleoside
  modified_base 16: mod_base = OTHER; note = 2-O-(2-methoxyethyl) nucleoside
  modified_base 18: mod_base = OTHER; note = 2-O-(2-methoxyethyl) nucleoside
  modified_base 20: mod_base = OTHER; note = 2-O-(2-methoxyethyl) nucleoside
  modified_base 22: mod_base = OTHER; note = 2-O-(2-methoxyethyl) nucleoside
  modified_base 24: mod_base = OTHER; note = 2-O-(2-methoxyethyl) nucleoside
  modified_base 26: mod_base = OTHER; note = 2-O-(2-methoxyethyl) nucleoside
  modified_base 28: mod_base = OTHER; note = 2-O-(2-methoxyethyl) nucleoside
  modified_base 30..31: mod_base = OTHER; note = 2-O-(2-methoxyethyl) nucleoside
  modified_base 1: mod_base = m5c
  modified_base 31: mod_base = m5c

SEQ ID NO: 202 - DNA, 31 bp, synthetic construct; identical to SEQ ID NO: 29
  source 1..31: mol_type = other DNA
  misc_feature 1..31: note = linked by phosphorothioate internucleoside linkage
  modified_base 1..2: mod_base = OTHER; note = 2-O-(2-methoxyethyl) nucleoside
  modified_base 5: mod_base = OTHER; note = 2-O-(2-methoxyethyl) nucleoside
  modified_base 7: mod_base = OTHER; note = 2-O-(2-methoxyethyl) nucleoside
  modified_base 9: mod_base = OTHER; note = 2-O-(2-methoxyethyl) nucleoside
  modified_base 11: mod_base = OTHER; note = 2-O-(2-methoxyethyl) nucleoside
  modified_base 13: mod_base = OTHER; note = 2-O-(2-methoxyethyl) nucleoside
  modified_base 15: mod_base = OTHER; note = 2-O-(2-methoxyethyl) nucleoside
  modified_base 17: mod_base = OTHER; note = 2-O-(2-methoxyethyl) nucleoside
  modified_base 19: mod_base = OTHER; note = 2-O-(2-methoxyethyl) nucleoside
  modified_base 21: mod_base = OTHER; note = 2-O-(2-methoxyethyl) nucleoside
  modified_base 23: mod_base = OTHER; note = 2-O-(2-methoxyethyl) nucleoside
  modified_base 25: mod_base = OTHER; note = 2-O-(2-methoxyethyl) nucleoside
  modified_base 27: mod_base = OTHER; note = 2-O-(2-methoxyethyl) nucleoside
  modified_base 30..31: mod_base = OTHER; note = 2-O-(2-methoxyethyl) nucleoside
  modified_base 1: mod_base = m5c
  modified_base 5: mod_base = m5c
  modified_base 7: mod_base = m5c
  modified_base 9: mod_base = m5c
  modified_base 11: mod_base = m5c
  modified_base 13: mod_base = m5c
  modified_base 15: mod_base = m5c
  modified_base 17: mod_base = m5c
  modified_base 19: mod_base = m5c
  modified_base 21: mod_base = m5c
  modified_base 23: mod_base = m5c
  modified_base 25: mod_base = m5c
  modified_base 27: mod_base = m5c
  modified_base 31: mod_base = m5c

SEQ ID NO: 203 - DNA, 25 bp, synthetic construct
  source 1..25: mol_type = other DNA
  misc_feature 1..25: note = linked by phosphorothioate internucleoside linkage
  modified_base 1..25: mod_base = OTHER; note = 2-O-Methyl nucleoside
cacacacacacacacacacacacac

SEQ ID NO: 204 - DNA, 27 bp, synthetic construct
  source 1..27: mol_type = other DNA
  misc_feature 1..27: note = linked by phosphorothioate internucleoside linkage
  modified_base 1..27: mod_base = OTHER; note = 2-O-Methyl nucleoside
cacacacacacacacacacacacacac

SEQ ID NO: 205 - DNA, 29 bp, synthetic construct
  source 1..29: mol_type = other DNA
  misc_feature 1..29: note = linked by phosphorothioate internucleoside linkage
  modified_base 1..29: mod_base = OTHER; note = 2-O-Methyl nucleoside
cacacacacacacacacacacacacacac

SEQ ID NO: 206 - DNA, 31 bp, synthetic construct; identical to SEQ ID NO: 29
  source 1..31: mol_type = other DNA
  misc_feature 1..31: note = linked by phosphorothioate internucleoside linkage
  modified_base 1..31: mod_base = OTHER; note = 2-O-Methyl nucleoside

SEQ ID NO: 207 - DNA, 25 bp, synthetic construct
  source 1..25: mol_type = other DNA
  misc_feature 1..25: note = linked by phosphorothioate internucleoside linkage
  modified_base 1..2: mod_base = OTHER; note = 2-O-Methyl nucleoside
  modified_base 5: mod_base = OTHER; note = 2-O-Methyl nucleoside
  modified_base 7: mod_base = OTHER; note = 2-O-Methyl nucleoside
  modified_base 9: mod_base = OTHER; note = 2-O-Methyl nucleoside
  modified_base 11: mod_base = OTHER; note = 2-O-Methyl nucleoside
  modified_base 13: mod_base = OTHER; note = 2-O-Methyl nucleoside
  modified_base 15: mod_base = OTHER; note = 2-O-Methyl nucleoside
  modified_base 17: mod_base = OTHER; note = 2-O-Methyl nucleoside
  modified_base 19: mod_base = OTHER; note = 2-O-Methyl nucleoside
  modified_base 21: mod_base = OTHER; note = 2-O-Methyl nucleoside
  modified_base 24..25: mod_base = OTHER; note = 2-O-Methyl nucleoside
cacacacacacacacacacacacac

SEQ ID NO: 208 - DNA, 27 bp, synthetic construct
  source 1..27: mol_type = other DNA
  misc_feature 1..27: note = linked by phosphorothioate internucleoside linkage
  modified_base 1..2: mod_base = OTHER; note = 2-O-Methyl nucleoside
  modified_base 5: mod_base = OTHER; note = 2-O-Methyl nucleoside
  modified_base 7: mod_base = OTHER; note = 2-O-Methyl nucleoside
  modified_base 9: mod_base = OTHER; note = 2-O-Methyl nucleoside
  modified_base 11: mod_base = OTHER; note = 2-O-Methyl nucleoside
  modified_base 13: mod_base = OTHER; note = 2-O-Methyl nucleoside
  modified_base 15: mod_base = OTHER; note = 2-O-Methyl nucleoside
  modified_base 17: mod_base = OTHER; note = 2-O-Methyl nucleoside
  modified_base 19: mod_base = OTHER; note = 2-O-Methyl nucleoside
  modified_base 21: mod_base = OTHER; note = 2-O-Methyl nucleoside
  modified_base 23: mod_base = OTHER; note = 2-O-Methyl nucleoside
  modified_base 26..27: mod_base = OTHER; note = 2-O-Methyl nucleoside
cacacacacacacacacacacacacac

SEQ ID NO: 209 - DNA, 29 bp, synthetic construct
  source 1..29: mol_type = other DNA
  misc_feature 1..29: note = linked by phosphorothioate internucleoside linkage
  modified_base 1..2: mod_base = OTHER; note = 2-O-Methyl nucleoside
  modified_base 5: mod_base = OTHER; note = 2-O-Methyl nucleoside
  modified_base 7: mod_base = OTHER; note = 2-O-Methyl nucleoside
  modified_base 9: mod_base = OTHER; note = 2-O-Methyl nucleoside
  modified_base 11: mod_base = OTHER; note = 2-O-Methyl nucleoside
  modified_base 13: mod_base = OTHER; note = 2-O-Methyl nucleoside
  modified_base 15: mod_base = OTHER; note = 2-O-Methyl nucleoside
  modified_base 17: mod_base = OTHER; note = 2-O-Methyl nucleoside
  modified_base 19: mod_base = OTHER; note = 2-O-Methyl nucleoside
  modified_base 21: mod_base = OTHER; note = 2-O-Methyl nucleoside
  modified_base 23: mod_base = OTHER; note = 2-O-Methyl nucleoside
  modified_base 25: mod_base = OTHER; note = 2-O-Methyl nucleoside
  modified_base 28..29: mod_base = OTHER; note = 2-O-Methyl nucleoside
cacacacacacacacacacacacacacac

SEQ ID NO: 210 - DNA, 31 bp, synthetic construct; identical to SEQ ID NO: 29
  source 1..31: mol_type = other DNA
  misc_feature 1..31: note = linked by phosphorothioate internucleoside linkage
  modified_base 1..2: mod_base = OTHER; note = 2-O-Methyl nucleoside
  modified_base 5: mod_base = OTHER; note = 2-O-Methyl nucleoside
  modified_base 7: mod_base = OTHER; note = 2-O-Methyl nucleoside
  modified_base 9: mod_base = OTHER; note = 2-O-Methyl nucleoside
  modified_base 11: mod_base = OTHER; note = 2-O-Methyl nucleoside
  modified_base 13: mod_base = OTHER; note = 2-O-Methyl nucleoside
  modified_base 15: mod_base = OTHER; note = 2-O-Methyl nucleoside
  modified_base 17: mod_base = OTHER; note = 2-O-Methyl nucleoside
  modified_base 19: mod_base = OTHER; note = 2-O-Methyl nucleoside
  modified_base 21: mod_base = OTHER; note = 2-O-Methyl nucleoside
  modified_base 23: mod_base = OTHER; note = 2-O-Methyl nucleoside
  modified_base 25: mod_base = OTHER; note = 2-O-Methyl nucleoside
  modified_base 27: mod_base = OTHER; note = 2-O-Methyl nucleoside
  modified_base 30..31: mod_base = OTHER; note = 2-O-Methyl nucleoside

SEQ ID NO: 211 - DNA, 32 bp, synthetic construct; identical to SEQ ID NO: 34
  source 1..32: mol_type = other DNA
  misc_feature 1..32: note = linked by phosphorothioate internucleoside linkage
  modified_base 1..2: mod_base = OTHER; note = locked nucleic acid (LNA)
  modified_base 4: mod_base = OTHER; note = locked nucleic acid (LNA)
  modified_base 6: mod_base = OTHER; note = locked nucleic acid (LNA)
  modified_base 8: mod_base = OTHER; note = locked nucleic acid (LNA)
  modified_base 10: mod_base = OTHER; note = locked nucleic acid (LNA)
  modified_base 14: mod_base = OTHER; note = locked nucleic acid (LNA)
  modified_base 16: mod_base = OTHER; note = locked nucleic acid (LNA)
  modified_base 18: mod_base = OTHER; note = locked nucleic acid (LNA)
  modified_base 20: mod_base = OTHER; note = locked nucleic acid (LNA)
  modified_base 22: mod_base = OTHER; note = locked nucleic acid (LNA)
  modified_base 24: mod_base = OTHER; note = locked nucleic acid (LNA)
  modified_base 26: mod_base = OTHER; note = locked nucleic acid (LNA)
  modified_base 28: mod_base = OTHER; note = locked nucleic acid (LNA)
  modified_base 30: mod_base = OTHER; note = locked nucleic acid (LNA)
  modified_base 32: mod_base = OTHER; note = locked nucleic acid (LNA)
  modified_base 2: mod_base = m5c
  modified_base 4: mod_base = m5c
  modified_base 6: mod_base = m5c
  modified_base 8: mod_base = m5c
  modified_base 10: mod_base = m5c
  modified_base 12: mod_base = m5c
  modified_base 14: mod_base = m5c
  modified_base 16: mod_base = m5c
  modified_base 18: mod_base = m5c
  modified_base 20: mod_base = m5c
  modified_base 22: mod_base = m5c
  modified_base 24: mod_base = m5c
  modified_base 26: mod_base = m5c
  modified_base 28: mod_base = m5c
  modified_base 32: mod_base = m5c

SEQ ID NO: 212 - DNA, 23 bp, synthetic construct
  source 1..23: mol_type = other DNA
  misc_feature 1..23: note = linked by phosphorothioate internucleoside linkage
  modified_base 1..2: mod_base = OTHER; note = locked nucleic acid (LNA)
  modified_base 5: mod_base = OTHER; note = locked nucleic acid (LNA)
  modified_base 7: mod_base = OTHER; note = locked nucleic acid (LNA)
  modified_base 9: mod_base = OTHER; note = locked nucleic acid (LNA)
  modified_base 11: mod_base = OTHER; note = locked nucleic acid (LNA)
  modified_base 13: mod_base = OTHER; note = locked nucleic acid (LNA)
  modified_base 15: mod_base = OTHER; note = locked nucleic acid (LNA)
  modified_base 17: mod_base = OTHER; note = locked nucleic acid (LNA)
  modified_base 19: mod_base = OTHER; note = locked nucleic acid (LNA)
  modified_base 22..23: mod_base = OTHER; note = locked nucleic acid (LNA)
  modified_base 4: mod_base = OTHER; note = 2-O-(2-methoxyethyl) nucleoside
  modified_base 6: mod_base = OTHER; note = 2-O-(2-methoxyethyl) nucleoside
  modified_base 1: mod_base = m5c
  modified_base 5: mod_base = m5c
  modified_base 7: mod_base = m5c
  modified_base 9: mod_base = m5c
  modified_base 11: mod_base = m5c
  modified_base 13: mod_base = m5c
  modified_base 15: mod_base = m5c
  modified_base 17: mod_base = m5c
  modified_base 19: mod_base = m5c
  modified_base 23: mod_base = m5c
cacacacacacacacacacacac

SEQ ID NO: 213 - DNA, 23 bp, synthetic construct
  source 1..23: mol_type = other DNA
  modified_base 12: mod_base = i
  misc_feature 1..23: note = linked by phosphorothioate internucleoside linkage
  modified_base 1..2: mod_base = OTHER; note = locked nucleic acid (LNA)
  modified_base 5: mod_base = OTHER; note = locked nucleic acid (LNA)
  modified_base 7: mod_base = OTHER; note = locked nucleic acid (LNA)
  modified_base 9: mod_base = OTHER; note = locked nucleic acid (LNA)
  modified_base 11: mod_base = OTHER; note = locked nucleic acid (LNA)
  modified_base 13: mod_base = OTHER; note = locked nucleic acid (LNA)
  modified_base 15: mod_base = OTHER; note = locked nucleic acid (LNA)
  modified_base 17: mod_base = OTHER; note = locked nucleic acid (LNA)
  modified_base 19: mod_base = OTHER; note = locked nucleic acid (LNA)
  modified_base 22..23: mod_base = OTHER; note = locked nucleic acid (LNA)
  modified_base 1: mod_base = m5c
  modified_base 5: mod_base = m5c
  modified_base 7: mod_base = m5c
  modified_base 9: mod_base = m5c
  modified_base 11: mod_base = m5c
  modified_base 13: mod_base = m5c
  modified_base 15: mod_base = m5c
  modified_base 17: mod_base = m5c
  modified_base 19: mod_base = m5c
  modified_base 23: mod_base = m5c
cacacacccacncacacacgcac

SEQ ID NO: 214 - DNA, 23 bp, synthetic construct
  source 1..23: mol_type = other DNA
  misc_feature 1..23: note = linked by phosphorothioate internucleoside linkage
  modified_base 6: mod_base = i
  modified_base 1..2: mod_base = OTHER; note = locked nucleic acid (LNA)
  modified_base 5: mod_base = OTHER; note = locked nucleic acid (LNA)
  modified_base 7: mod_base = OTHER; note = locked nucleic acid (LNA)
  modified_base 9: mod_base = OTHER; note = locked nucleic acid (LNA)
  modified_base 11: mod_base = OTHER; note = locked nucleic acid (LNA)
  modified_base 13: mod_base = OTHER; note = locked nucleic acid (LNA)
  modified_base 15: mod_base = OTHER; note = locked nucleic acid (LNA)
  modified_base 17: mod_base = OTHER; note = locked nucleic acid (LNA)
  modified_base 19: mod_base = OTHER; note = locked nucleic acid (LNA)
  modified_base 22..23: mod_base = OTHER; note = locked nucleic acid (LNA)
  modified_base 1: mod_base = m5c
  modified_base 5: mod_base = m5c
  modified_base 7: mod_base = m5c
  modified_base 9: mod_base = m5c
  modified_base 11: mod_base = m5c
  modified_base 13: mod_base = m5c
  modified_base 15: mod_base = m5c
  modified_base 17: mod_base = m5c
  modified_base 19: mod_base = m5c
  modified_base 23: mod_base = m5c
cacacncacacacacacacacac

SEQ ID NO: 215 - DNA, 23 bp, synthetic construct
  source 1..23: mol_type = other DNA
  misc_feature 1..23: note = linked by phosphorothioate internucleoside linkage
  modified_base 12: mod_base = i
  modified_base 1..2: mod_base = OTHER; note = locked nucleic acid (LNA)
  modified_base 5: mod_base = OTHER; note = locked nucleic acid (LNA)
  modified_base 7: mod_base = OTHER; note = locked nucleic acid (LNA)
  modified_base 9: mod_base = OTHER; note = locked nucleic acid (LNA)
  modified_base 11: mod_base = OTHER; note = locked nucleic acid (LNA)
  modified_base 13: mod_base = OTHER; note = locked nucleic acid (LNA)
  modified_base 15: mod_base = OTHER; note = locked nucleic acid (LNA)
  modified_base 17: mod_base = OTHER; note = locked nucleic acid (LNA)
  modified_base 19: mod_base = OTHER; note = locked nucleic acid (LNA)
  modified_base 22..23: mod_base = OTHER; note = locked nucleic acid (LNA)
  modified_base 1: mod_base = m5c
  modified_base 5: mod_base = m5c
  modified_base 7: mod_base = m5c
  modified_base 9: mod_base = m5c
  modified_base 11: mod_base = m5c
  modified_base 13: mod_base = m5c
  modified_base 15: mod_base = m5c
  modified_base 17: mod_base = m5c
  modified_base 19: mod_base = m5c
  modified_base 23: mod_base = m5c
cacacacacacncacacacacac

SEQ ID NO: 216 - DNA, 23 bp, synthetic construct
  source 1..23: mol_type = other DNA
  misc_feature 1..23: note = linked by phosphorothioate internucleoside linkage
  modified_base 6: mod_base = i
  modified_base 1..2: mod_base = OTHER; note = locked nucleic acid (LNA)
  modified_base 5: mod_base = OTHER; note = locked nucleic acid (LNA)
  modified_base 7: mod_base = OTHER; note = locked nucleic acid (LNA)
  modified_base 9: mod_base = OTHER; note = locked nucleic acid (LNA)
  modified_base 11: mod_base = OTHER; note = locked nucleic acid (LNA)
  modified_base 13: mod_base = OTHER; note = locked nucleic acid (LNA)
  modified_base 15: mod_base = OTHER; note = locked nucleic acid (LNA)
  modified_base 17: mod_base = OTHER; note = locked nucleic acid (LNA)
  modified_base 19: mod_base = OTHER; note = locked nucleic acid (LNA)
  modified_base 22..23: mod_base = OTHER; note = locked nucleic acid (LNA)
  modified_base 1: mod_base = m5c
  modified_base 5: mod_base = m5c
  modified_base 7: mod_base = m5c
  modified_base 9: mod_base = m5c
  modified_base 11: mod_base = m5c
  modified_base 13: mod_base = m5c
  modified_base 15: mod_base = m5c
  modified_base 17: mod_base = m5c
  modified_base 19: mod_base = m5c
  modified_base 23: mod_base = m5c
cacacncacacacacacacactc

SEQ ID NO: 217 - DNA, 23 bp, synthetic construct
  source 1..23: mol_type = other DNA
  misc_feature 1..23: note = linked by phosphorothioate internucleoside linkage
  modified_base 1..2: mod_base = OTHER; note = locked nucleic acid (LNA)
  modified_base 5..7: mod_base = OTHER; note = locked nucleic acid (LNA)
  modified_base 9: mod_base = OTHER; note = locked nucleic acid (LNA)
  modified_base 11: mod_base = OTHER; note = locked nucleic acid (LNA)
  modified_base 13: mod_base = OTHER; note = locked nucleic acid (LNA)
  modified_base 15: mod_base = OTHER; note = locked nucleic acid (LNA)
  modified_base 17: mod_base = OTHER; note = locked nucleic acid (LNA)
  modified_base 19: mod_base = OTHER; note = locked nucleic acid (LNA)
  modified_base 22..23: mod_base = OTHER; note = locked nucleic acid (LNA)
  modified_base 1: mod_base = m5c
  modified_base 7: mod_base = m5c
  modified_base 9: mod_base = m5c
  modified_base 11: mod_base = m5c
  modified_base 13: mod_base = m5c
  modified_base 15: mod_base = m5c
  modified_base 17: mod_base = m5c
  modified_base 19: mod_base = m5c
  modified_base 23: mod_base = m5c
cacatacacacccacacacacac

SEQ ID NO: 218 - DNA, 23 bp, synthetic construct
  source 1..23: mol_type = other DNA
  misc_feature 1..23: note = linked by phosphorothioate internucleoside linkage
  modified_base 1..2: mod_base = OTHER; note = locked nucleic acid (LNA)
  modified_base 5: mod_base = OTHER; note = locked nucleic acid (LNA)
  modified_base 7: mod_base = OTHER; note = locked nucleic acid (LNA)
  modified_base 9: mod_base = OTHER; note = locked nucleic acid (LNA)
  modified_base 11: mod_base = OTHER; note = locked nucleic acid (LNA)
  modified_base 13: mod_base = OTHER; note = locked nucleic acid (LNA)
  modified_base 15: mod_base = OTHER; note = locked nucleic acid (LNA)
  modified_base 17: mod_base = OTHER; note = locked nucleic acid (LNA)
  modified_base 19: mod_base = OTHER; note = locked nucleic acid (LNA)
  modified_base 22..23: mod_base = OTHER; note = locked nucleic acid (LNA)
  modified_base 1: mod_base = m5c
  modified_base 5: mod_base = m5c
  modified_base 7: mod_base = m5c
  modified_base 9: mod_base = m5c
  modified_base 11: mod_base = m5c
  modified_base 13: mod_base = m5c
  modified_base 15: mod_base = m5c
  modified_base 17: mod_base = m5c
  modified_base 19: mod_base = m5c
  modified_base 23: mod_base = m5c
cacacacacacacacacgcacac

SEQ ID NO: 219 - DNA, 23 bp, synthetic construct
  source 1..23: mol_type = other DNA
  misc_feature 1..23: note = linked by phosphorothioate internucleoside linkage
  modified_base 4: mod_base = i
  modified_base 8: mod_base = i
  modified_base 16: mod_base = i
  modified_base 20: mod_base = i
  modified_base 1..2: mod_base = OTHER; note = locked nucleic acid (LNA)
  modified_base 5: mod_base = OTHER; note = locked nucleic acid (LNA)
  modified_base 7: mod_base = OTHER; note = locked nucleic acid (LNA)
  modified_base 9: mod_base = OTHER; note = locked nucleic acid (LNA)
  modified_base 11: mod_base = OTHER; note = locked nucleic acid (LNA)
  modified_base 13: mod_base = OTHER; note = locked nucleic acid (LNA)
  modified_base 15: mod_base = OTHER; note = locked nucleic acid (LNA)
  modified_base 17: mod_base = OTHER; note = locked nucleic acid (LNA)
  modified_base 19: mod_base = OTHER; note = locked nucleic acid (LNA)
  modified_base 22..23: mod_base = OTHER; note = locked nucleic acid (LNA)
  modified_base 1: mod_base = m5c
  modified_base 5: mod_base = m5c
  modified_base 7: mod_base = m5c
  modified_base 9: mod_base = m5c
  modified_base 11: mod_base = m5c
  modified_base 13: mod_base = m5c
  modified_base 15: mod_base = m5c
  modified_base 17: mod_base = m5c
  modified_base 19: mod_base = m5c
  modified_base 23: mod_base = m5c
cacncacncacacacncacncac

SEQ ID NO: 220 - DNA, 23 bp, synthetic construct
  source 1..23: mol_type = other DNA
  misc_feature 1..23: note = linked by phosphorothioate internucleoside linkage
  modified_base 1..2: mod_base = OTHER; note = locked nucleic acid (LNA)
  modified_base 5: mod_base = OTHER; note = locked nucleic acid (LNA)
  modified_base 7: mod_base = OTHER; note = locked nucleic acid (LNA)
  modified_base 9: mod_base = OTHER; note = locked nucleic acid (LNA)
  modified_base 11: mod_base = OTHER; note = locked nucleic acid (LNA)
  modified_base 13: mod_base = OTHER; note = locked nucleic acid (LNA)
  modified_base 15: mod_base = OTHER; note = locked nucleic acid (LNA)
  modified_base 17: mod_base = OTHER; note = locked nucleic acid (LNA)
  modified_base 19: mod_base = OTHER; note = locked nucleic acid (LNA)
  modified_base 23: mod_base = OTHER; note = locked nucleic acid (LNA)
  modified_base 1: mod_base = m5c
  modified_base 5: mod_base = m5c
  modified_base 7: mod_base = m5c
  modified_base 9: mod_base = m5c
  modified_base 11: mod_base = m5c
  modified_base 13: mod_base = m5c
  modified_base 15: mod_base = m5c
  modified_base 17: mod_base = m5c
  modified_base 19: mod_base = m5c
  modified_base 23: mod_base = m5c
cacacacacacacactctcacac

SEQ ID NO: 221 - DNA, 23 bp, synthetic construct
  source 1..23: mol_type = other DNA
  misc_feature 1..23: note = linked by phosphorothioate internucleoside linkage
  modified_base 1..2: mod_base = OTHER; note = locked nucleic acid (LNA)
  modified_base 5: mod_base = OTHER; note = locked nucleic acid (LNA)
  modified_base 7: mod_base = OTHER; note = locked nucleic acid (LNA)
  modified_base 9: mod_base = OTHER; note = locked nucleic acid (LNA)
  modified_base 11: mod_base = OTHER; note = locked nucleic acid (LNA)
  modified_base 13: mod_base = OTHER; note = locked nucleic acid (LNA)
  modified_base 15: mod_base = OTHER; note = locked nucleic acid (LNA)
  modified_base 17: mod_base = OTHER; note = locked nucleic acid (LNA)
  modified_base 19: mod_base = OTHER; note = locked nucleic acid (LNA)
  modified_base 22..23: mod_base = OTHER; note = locked nucleic acid (LNA)
  modified_base 1: mod_base = m5c
  modified_base 5: mod_base = m5c
  modified_base 7: mod_base = m5c
  modified_base 9: mod_base = m5c
  modified_base 11: mod_base = m5c
  modified_base 13: mod_base = m5c
  modified_base 15: mod_base = m5c
  modified_base 17: mod_base = m5c
  modified_base 19: mod_base = m5c
  modified_base 23: mod_base = m5c
ctcacacacacacacacacacac

SEQ ID NO: 222 - DNA, 23 bp, synthetic construct
  source 1..23: mol_type = other DNA
  misc_feature 1..23: note = linked by phosphorothioate internucleoside linkage
  modified_base 1..2: mod_base = OTHER; note = locked nucleic acid (LNA)
  modified_base 5: mod_base = OTHER; note = locked nucleic acid (LNA)
  modified_base 7: mod_base = OTHER; note = locked nucleic acid (LNA)
  modified_base 9: mod_base = OTHER; note = locked nucleic acid (LNA)
  modified_base 11: mod_base = OTHER; note = locked nucleic acid (LNA)
  modified_base 13: mod_base = OTHER; note = locked nucleic acid (LNA)
  modified_base 15: mod_base = OTHER; note = locked nucleic acid (LNA)
  modified_base 17: mod_base = OTHER; note = locked nucleic acid (LNA)
  modified_base 19: mod_base = OTHER; note = locked nucleic acid (LNA)
  modified_base 22..23: mod_base = OTHER; note = locked nucleic acid (LNA)
  modified_base 1: mod_base = m5c
  modified_base 5: mod_base = m5c
  modified_base 7: mod_base = m5c
  modified_base 9: mod_base = m5c
  modified_base 11: mod_base = m5c
  modified_base 15: mod_base = m5c
  modified_base 17: mod_base = m5c
  modified_base 19: mod_base = m5c
  modified_base 23: mod_base = m5c
cacacacacacaaacacacacac

SEQ ID NO: 223 - DNA, 23 bp, synthetic construct
  source 1..23: mol_type = other DNA
  misc_feature 1..23: note = linked by phosphorothioate internucleoside linkage
  modified_base 1..2: mod_base = OTHER; note = locked nucleic acid (LNA)
  modified_base 5: mod_base = OTHER; note = locked nucleic acid (LNA)
  modified_base 7: mod_base = OTHER; note = locked nucleic acid (LNA)
  modified_base 9: mod_base = OTHER; note = locked nucleic acid (LNA)
  modified_base 11: mod_base = OTHER; note = locked nucleic acid (LNA)
  modified_base 13: mod_base = OTHER; note = locked nucleic acid (LNA)
  modified_base 15: mod_base = OTHER; note = locked nucleic acid (LNA)
  modified_base 17: mod_base = OTHER; note = locked nucleic acid (LNA)
  modified_base 19: mod_base = OTHER; note = locked nucleic acid (LNA)
  modified_base 22..23: mod_base = OTHER; note = locked nucleic acid (LNA)
  modified_base 1: mod_base = m5c
  modified_base 5: mod_base = m5c
  modified_base 7: mod_base = m5c
  modified_base 9: mod_base = m5c
  modified_base 11: mod_base = m5c
  modified_base 13: mod_base = m5c
  modified_base 15: mod_base = m5c
  modified_base 17: mod_base = m5c
  modified_base 19: mod_base = m5c
  modified_base 23: mod_base = m5c
cacacgcacgcacacacacacac

SEQ ID NO: 224 - DNA, 23 bp, synthetic construct
  source 1..23: mol_type = other DNA
  misc_feature 1..23: note = linked by phosphorothioate internucleoside linkage
  modified_base 1..2: mod_base = OTHER; note = locked nucleic acid (LNA)
  modified_base 5: mod_base = OTHER; note = locked nucleic acid (LNA)
  modified_base 7: mod_base = OTHER; note = locked nucleic acid (LNA)
  modified_base 9: mod_base = OTHER; note = locked nucleic acid (LNA)
  modified_base 11: mod_base = OTHER; note = locked nucleic acid (LNA)
  modified_base 13: mod_base = OTHER; note = locked nucleic acid (LNA)
  modified_base 15: mod_base = OTHER; note = locked nucleic acid (LNA)
  modified_base 17: mod_base = OTHER; note = locked nucleic acid (LNA)
  modified_base 19: mod_base = OTHER; note = locked nucleic acid (LNA)
  modified_base 22..23: mod_base = OTHER; note = locked nucleic acid (LNA)
  modified_base 1: mod_base = m5c
  modified_base 5: mod_base = m5c
  modified_base 7: mod_base = m5c
  modified_base 9: mod_base = m5c
  modified_base 11: mod_base = m5c
  modified_base 13: mod_base = m5c
  modified_base 15: mod_base = m5c
  modified_base 17: mod_base = m5c
  modified_base 19: mod_base = m5c
  modified_base 23: mod_base = m5c
cacgcacacacccacacactcac

SEQ ID NO: 225 - DNA, 23 bp, synthetic construct
  source 1..23: mol_type = other DNA
  misc_feature 1..23: note = linked by phosphorothioate internucleoside linkage
  modified_base 1..2: mod_base = OTHER; note = locked nucleic acid (LNA)
  modified_base 5: mod_base = OTHER; note = locked nucleic acid (LNA)
  modified_base 7: mod_base = OTHER; note = locked nucleic acid (LNA)
  modified_base 9: mod_base = OTHER; note = locked nucleic acid (LNA)
  modified_base 11: mod_base = OTHER; note = locked nucleic acid (LNA)
  modified_base 13: mod_base = OTHER; note = locked nucleic acid (LNA)
  modified_base 15: mod_base = OTHER; note = locked nucleic acid (LNA)
  modified_base 17: mod_base = OTHER; note = locked nucleic acid (LNA)
  modified_base 19: mod_base = OTHER; note = locked nucleic acid (LNA)
  modified_base 20..23: mod_base = OTHER; note = locked nucleic acid (LNA)
  modified_base 1: mod_base = m5c
  modified_base 5: mod_base = m5c
  modified_base 7: mod_base = m5c
  modified_base 9: mod_base = m5c
  modified_base 11: mod_base = m5c
  modified_base 13: mod_base = m5c
  modified_base 15: mod_base = m5c
  modified_base 17: mod_base = m5c
  modified_base 19: mod_base = m5c
  modified_base 23: mod_base = m5c
cacacacacacactctctcacac

SEQ ID NO: 226 - DNA, 23 bp, synthetic construct
  source 1..23: mol_type = other DNA
  misc_feature 1..23: note = linked by phosphorothioate internucleoside linkage
  modified_base 1..2: mod_base = OTHER; note = locked nucleic acid (LNA)
  modified_base 5: mod_base = OTHER; note = locked nucleic acid (LNA)
  modified_base 7: mod_base = OTHER; note = locked nucleic acid (LNA)
  modified_base 9: mod_base = OTHER; note = locked nucleic acid (LNA)
  modified_base 11: mod_base = OTHER; note = locked nucleic acid (LNA)
  modified_base 13: mod_base = OTHER; note = locked nucleic acid (LNA)
  modified_base 15: mod_base = OTHER; note = locked nucleic acid (LNA)
  modified_base 17: mod_base = OTHER; note = locked nucleic acid (LNA)
  modified_base 19: mod_base = OTHER; note = locked nucleic acid (LNA)
  modified_base 22..23: mod_base = OTHER; note = locked nucleic acid (LNA)
  modified_base 1: mod_base = m5c
  modified_base 5: mod_base = m5c
  modified_base 7: mod_base = m5c
  modified_base 9: mod_base = m5c
  modified_base 11: mod_base = m5c
  modified_base 13: mod_base = m5c
  modified_base 15: mod_base = m5c
  modified_base 17: mod_base = m5c
  modified_base 19: mod_base = m5c
  modified_base 23: mod_base = m5c
  modified_base 14: mod_base = i
  modified_base 16: mod_base = i
  modified_base 18: mod_base = i
  modified_base 20: mod_base = i
cacacacacacacncncncncac

SEQ ID NO: 227 - DNA, 23 bp, synthetic construct
  source 1..23: mol_type = other DNA
  misc_feature 1..23: note = linked by phosphorothioate internucleoside linkage
  modified_base 1..2: mod_base = OTHER; note = locked nucleic acid (LNA)
  modified_base 5: mod_base = OTHER; note = locked nucleic acid (LNA)
  modified_base 7: mod_base = OTHER; note = locked nucleic acid (LNA)
  modified_base 9: mod_base = OTHER; note = locked nucleic acid (LNA)
  modified_base 11: mod_base = OTHER; note = locked nucleic acid (LNA)
  modified_base 13: mod_base = OTHER; note = locked nucleic acid (LNA)
  modified_base 15: mod_base = OTHER; note = locked nucleic acid (LNA)
  modified_base 17: mod_base = OTHER; note = locked nucleic acid (LNA)
  modified_base 19: mod_base = OTHER; note = locked nucleic acid (LNA)
  modified_base 22..23: mod_base = OTHER; note = locked nucleic acid (LNA)
  modified_base 1: mod_base = m5c
  modified_base 5: mod_base = m5c
  modified_base 7: mod_base = m5c
  modified_base 9: mod_base = m5c
  modified_base 11: mod_base = m5c
  modified_base 13: mod_base = m5c
  modified_base 15: mod_base = m5c
  modified_base 17: mod_base = m5c
  modified_base 19: mod_base = m5c
  modified_base 23: mod_base = m5c
cactcacacactcacacacacac

SEQ ID NO: 228 - DNA, 23 bp, synthetic construct
  source 1..23: mol_type = other DNA
  misc_feature 1..23: note = linked by phosphorothioate internucleoside linkage
  modified_base 1..2: mod_base = OTHER; note = locked nucleic acid (LNA)
  modified_base 5: mod_base = OTHER; note = locked nucleic acid (LNA)
  modified_base 7: mod_base = OTHER; note = locked nucleic acid (LNA)
  modified_base 9: mod_base = OTHER; note = locked nucleic acid (LNA)
  modified_base 11: mod_base = OTHER; note = locked nucleic acid (LNA)
  modified_base 13: mod_base = OTHER; note = locked nucleic acid (LNA)
  modified_base 15: mod_base = OTHER; note = locked nucleic acid (LNA)
  modified_base 17: mod_base = OTHER; note = locked nucleic acid (LNA)
  modified_base 19: mod_base = OTHER; note = locked nucleic acid (LNA)
  modified_base 22..23: mod_base = OTHER; note = locked nucleic acid (LNA)
  modified_base 1: mod_base = m5c
  modified_base 5: mod_base = m5c
  modified_base 7: mod_base = m5c
  modified_base 9: mod_base = m5c
  modified_base 11: mod_base = m5c
  modified_base 13: mod_base = m5c
  modified_base 15: mod_base = m5c
  modified_base 17: mod_base = m5c
  modified_base 19: mod_base = m5c
  modified_base 23: mod_base = m5c
cacgcacacacgcacacacacac

SEQ ID NO: 229 - DNA, 23 bp, synthetic construct
  source 1..23: mol_type = other DNA
  misc_feature 1..23: note = linked by phosphorothioate internucleoside linkage
  modified_base 1..2: mod_base = OTHER; note = locked nucleic acid (LNA)
  modified_base 5: mod_base = OTHER; note = locked nucleic acid (LNA)
  modified_base 7: mod_base = OTHER; note = locked nucleic acid (LNA)
  modified_base 9: mod_base = OTHER; note = locked nucleic acid (LNA)
  modified_base 11: mod_base = OTHER; note = locked nucleic acid (LNA)
  modified_base 13: mod_base = OTHER; note = locked nucleic acid (LNA)
  modified_base 15: mod_base = OTHER; note = locked nucleic acid (LNA)
  modified_base 17: mod_base = OTHER; note = locked nucleic acid (LNA)
  modified_base 19: mod_base = OTHER; note = locked nucleic acid (LNA)
  modified_base 22..23: mod_base = OTHER; note = locked nucleic acid (LNA)
  modified_base 1: mod_base = m5c
  modified_base 5: mod_base = m5c
  modified_base 7: mod_base = m5c
  modified_base 9: mod_base = m5c
  modified_base 11: mod_base = m5c
  modified_base 13: mod_base = m5c
  modified_base 15: mod_base = m5c
  modified_base 17: mod_base = m5c
  modified_base 19: mod_base = m5c
  modified_base 23: mod_base = m5c
cacccacacacccacacacacac

SEQ ID NO: 230 - DNA, 23 bp, synthetic construct
  source 1..23: mol_type = other DNA
  misc_feature 1..23: note = linked by phosphorothioate internucleoside linkage
  modified_base 1..2: mod_base = OTHER; note = locked nucleic acid (LNA)
  modified_base 5: mod_base = OTHER; note = locked nucleic acid (LNA)
  modified_base 7: mod_base = OTHER; note = locked nucleic acid (LNA)
  modified_base 9: mod_base = OTHER; note = locked nucleic acid (LNA)
  modified_base 11: mod_base = OTHER; note = locked nucleic acid (LNA)
  modified_base 13: mod_base = OTHER; note = locked nucleic acid (LNA)
  modified_base 15: mod_base = OTHER; note = locked nucleic acid (LNA)
  modified_base 17: mod_base = OTHER; note = locked nucleic acid (LNA)
  modified_base 19: mod_base = OTHER; note = locked nucleic acid (LNA)
  modified_base 22..23: mod_base = OTHER; note = locked nucleic acid (LNA)
  modified_base 1: mod_base = m5c
  modified_base 5: mod_base = m5c
  modified_base 7: mod_base = m5c
  modified_base 9: mod_base = m5c
  modified_base 13: mod_base = m5c
  modified_base 15: mod_base = m5c
  modified_base 17: mod_base = m5c
  modified_base 19: mod_base = m5c
  modified_base 23: mod_base = m5c
cacacgcacatacacacacccac

SEQ ID NO: 231 - DNA, 23 bp, synthetic construct
  source 1..23: mol_type = other DNA
  misc_feature 1..23: note = linked by phosphorothioate internucleoside linkage
  modified_base 1..2: mod_base = OTHER; note = locked nucleic acid (LNA)
  modified_base 5: mod_base = OTHER; note = locked nucleic acid (LNA)
  modified_base 7: mod_base = OTHER; note = locked nucleic acid (LNA)
  modified_base 9: mod_base = OTHER; note = locked nucleic acid (LNA)
  modified_base 11: mod_base = OTHER; note = locked nucleic acid (LNA)
  modified_base 13: mod_base = OTHER; note = locked nucleic acid (LNA)
  modified_base 15: mod_base = OTHER; note = locked nucleic acid (LNA)
  modified_base 17: mod_base = OTHER; note = locked nucleic acid (LNA)
  modified_base 19: mod_base = OTHER; note = locked nucleic acid (LNA)
  modified_base 22..23: mod_base = OTHER; note = locked nucleic acid (LNA)
  modified_base 5: mod_base = m5c
  modified_base 7: mod_base = m5c
  modified_base 9: mod_base = m5c
  modified_base 11: mod_base = m5c
  modified_base 13: mod_base = m5c
  modified_base 15: mod_base = m5c
  modified_base 17: mod_base = m5c
  modified_base 19: mod_base = m5c
  modified_base 23: mod_base = m5c
ttcacacacacacacacacacac

SEQ ID NO: 232 - DNA, 23 bp, synthetic construct
  source 1..23: mol_type = other DNA
  misc_feature 1..23: note = linked by phosphorothioate internucleoside linkage
  modified_base 1..2: mod_base = OTHER; note = locked nucleic acid (LNA)
  modified_base 5: mod_base = OTHER; note = locked nucleic acid (LNA)
  modified_base 7: mod_base = OTHER; note = locked nucleic acid (LNA)
  modified_base 9: mod_base = OTHER; note = locked nucleic acid (LNA)
  modified_base 11: mod_base = OTHER; note = locked nucleic acid (LNA)
  modified_base 13: mod_base = OTHER; note = locked nucleic acid (LNA)
  modified_base 15: mod_base = OTHER; note = locked nucleic acid (LNA)
  modified_base 17: mod_base = OTHER; note = locked nucleic acid (LNA)
  modified_base 19: mod_base = OTHER; note = locked nucleic acid (LNA)
  modified_base 22..23: mod_base = OTHER; note = locked nucleic acid (LNA)
  modified_base 1: mod_base = m5c
  modified_base 5: mod_base = m5c
  modified_base 7: mod_base = m5c
  modified_base 9: mod_base = m5c
  modified_base 11: mod_base = m5c
  modified_base 13: mod_base = m5c
  modified_base 15: mod_base = m5c
  modified_base 17: mod_base = m5c
  modified_base 19: mod_base = m5c
cacacacacacacacacacacaa

SEQ ID NO: 233 - DNA, 29 bp, synthetic construct
  source 1..29: mol_type = other DNA
  misc_feature 1..29: note = linked by phosphorothioate internucleoside linkage
  modified_base 1..2: mod_base = OTHER; note = locked nucleic acid (LNA)
  modified_base 4: mod_base = OTHER; note = locked nucleic acid (LNA)
  modified_base 6: mod_base = OTHER; note = locked nucleic acid (LNA)
  modified_base 8: mod_base = OTHER; note = locked nucleic acid (LNA)
  modified_base 10: mod_base = OTHER; note = locked nucleic acid (LNA)
  modified_base 12: mod_base = OTHER; note = locked nucleic acid (LNA)
  modified_base 14: mod_base = OTHER; note = locked nucleic acid (LNA)
  modified_base 16: mod_base = OTHER; note = locked nucleic acid (LNA)
  modified_base 18: mod_base = OTHER; note = locked nucleic acid (LNA)
  modified_base 22: mod_base = OTHER; note = locked nucleic acid (LNA)
  modified_base 1: mod_base = m5c
  modified_base 29: mod_base = m5c
  modified_base 24: mod_base = OTHER; note = locked nucleic acid (LNA)
  modified_base 26: mod_base = OTHER; note = locked nucleic acid (LNA)
  modified_base 28..29: mod_base = OTHER; note = locked nucleic acid (LNA)
  modified_base 20: mod_base = OTHER; note = locked nucleic acid (LNA)
  modified_base 21: mod_base = i
cacacacacacacacacacanacacacac

SEQ ID NO: 234 - DNA, 29 bp, synthetic construct
  source 1..29: mol_type = other DNA
  misc_feature 1..29: note = linked by phosphorothioate internucleoside linkage
  modified_base 1..2: mod_base = OTHER; note = locked nucleic acid (LNA)
  modified_base 4: mod_base = OTHER; note = locked nucleic acid (LNA)
  modified_base 6: mod_base = OTHER; note = locked nucleic acid (LNA)
  modified_base 8: mod_base = OTHER; note = locked nucleic acid (LNA)
  modified_base 10: mod_base = OTHER; note = locked nucleic acid (LNA)
  modified_base 12: mod_base = OTHER; note = locked nucleic acid (LNA)
  modified_base 14: mod_base = OTHER; note = locked nucleic acid (LNA)
  modified_base 16: mod_base = OTHER; note = locked nucleic acid (LNA)
  modified_base 18: mod_base = OTHER; note = locked nucleic acid (LNA)
  modified_base 22: mod_base = OTHER; note = locked nucleic acid (LNA)
  modified_base 1: mod_base = m5c
  modified_base 29: mod_base = m5c
  modified_base 24: mod_base = OTHER; note = locked nucleic acid (LNA)
  modified_base 26: mod_base = OTHER; note = locked nucleic acid (LNA)
  modified_base 28..29: mod_base = OTHER; note = locked nucleic acid (LNA)
  modified_base 20: mod_base = OTHER; note = locked nucleic acid (LNA)
  modified_base 5: mod_base = i
cacanacacacacacacacacacacacac

SEQ ID NO: 235 - DNA, 29 bp, synthetic construct
  source 1..29: mol_type = other DNA
  misc_feature 1..29: note = linked by phosphorothioate internucleoside linkage
  modified_base 1..2: mod_base = OTHER; note = locked nucleic acid (LNA)
  modified_base 4: mod_base = OTHER; note = locked nucleic acid (LNA)
  modified_base 6: mod_base = OTHER; note = locked nucleic acid (LNA)
  modified_base 8: mod_base = OTHER; note = locked nucleic acid (LNA)
  modified_base 10: mod_base = OTHER; note = locked nucleic acid (LNA)
  modified_base 12: mod_base = OTHER; note = locked nucleic acid (LNA)
  modified_base 14: mod_base = OTHER; note = locked nucleic acid (LNA)
  modified_base 16: mod_base = OTHER; note = locked nucleic acid (LNA)
  modified_base 18: mod_base = OTHER; note = locked nucleic acid (LNA)
  modified_base 22: mod_base = OTHER; note = locked nucleic acid (LNA)
  modified_base 1: mod_base = m5c
  modified_base 29: mod_base = m5c
  modified_base 24: mod_base = OTHER; note = locked nucleic acid (LNA)
  modified_base 26: mod_base = OTHER; note = locked nucleic acid (LNA)
  modified_base 28..29: mod_base = OTHER; note = locked nucleic acid (LNA)
  modified_base 20: mod_base = OTHER; note = locked nucleic acid (LNA)
catacacacacacacacacatacacacac

SEQ ID NO: 236 - DNA, 29 bp, synthetic construct
  source 1..29: mol_type = other DNA
  misc_feature 1..29: note = linked by phosphorothioate internucleoside linkage
  modified_base 1..2: mod_base = OTHER; note = locked nucleic acid (LNA)
  modified_base 4: mod_base = OTHER; note = locked nucleic acid (LNA)
  modified_base 6: mod_base = OTHER; note = locked nucleic acid (LNA)
  modified_base 8: mod_base = OTHER; note = locked nucleic acid (LNA)
  modified_base 10: mod_base = OTHER; note = locked nucleic acid (LNA)
  modified_base 12: mod_base = OTHER; note = locked nucleic acid (LNA)
  modified_base 14: mod_base = OTHER; note = locked nucleic acid (LNA)
  modified_base 16: mod_base = OTHER; note = locked nucleic acid (LNA)
  modified_base 18: mod_base = OTHER; note = locked nucleic acid (LNA)
  modified_base 22: mod_base = OTHER; note = locked nucleic acid (LNA)
  modified_base 1: mod_base = m5c
  modified_base 29: mod_base = m5c
  modified_base 24: mod_base = OTHER; note = locked nucleic acid (LNA)
  modified_base 26: mod_base = OTHER; note = locked nucleic acid (LNA)
  modified_base 28..29: mod_base = OTHER; note = locked nucleic acid (LNA)
  modified_base 20: mod_base = OTHER; note = locked nucleic acid (LNA)
caaacacacacacacacacacacacacac

SEQ ID NO: 237 - DNA, 29 bp, synthetic construct
  source 1..29: mol_type = other DNA
  misc_feature 1..29: note = linked by phosphorothioate internucleoside linkage
  modified_base 1..2: mod_base = OTHER; note = locked nucleic acid (LNA)
  modified_base 4: mod_base = OTHER; note = locked nucleic acid (LNA)
  modified_base 6: mod_base = OTHER; note = locked nucleic acid (LNA)
  modified_base 8..10: mod_base = OTHER; note = locked nucleic acid (LNA)
  modified_base 12: mod_base = OTHER; note = locked nucleic acid (LNA)
  modified_base 14: mod_base = OTHER; note = locked nucleic acid (LNA)
  modified_base 16: mod_base = OTHER; note = locked nucleic acid (LNA)
  modified_base 18: mod_base = OTHER; note = locked nucleic acid (LNA)
  modified_base 22: mod_base = OTHER; note = locked nucleic acid (LNA)
  modified_base 1: mod_base = m5c
  modified_base 29: mod_base = m5c
  modified_base 24: mod_base = OTHER; note = locked nucleic acid (LNA)
  modified_base 26: mod_base = OTHER; note = locked nucleic acid (LNA)
  modified_base 28..29: mod_base = OTHER; note = locked nucleic acid (LNA)
  modified_base 20: mod_base = OTHER; note = locked nucleic acid (LNA)
  modified_base 9: mod_base = m5c
cacacacactcacacacacacacacacac

SEQ ID NO: 238 - DNA, 29 bp, synthetic construct
  source 1..29: mol_type = other DNA
  misc_feature 1..29: note = linked by phosphorothioate internucleoside linkage
  modified_base 1..2: mod_base = OTHER; note = locked nucleic acid (LNA)
  modified_base 4: mod_base = OTHER; note = locked nucleic acid (LNA)
  modified_base 6: mod_base = OTHER; note = locked nucleic acid (LNA)
  modified_base 8: mod_base = OTHER; note = locked nucleic acid (LNA)
  modified_base 10: mod_base = OTHER; note = locked nucleic acid (LNA)
  modified_base 12: mod_base = OTHER; note = locked nucleic acid (LNA)
  modified_base 14: mod_base = OTHER; note = locked nucleic acid (LNA)
  modified_base 16: mod_base = OTHER; note = locked nucleic acid (LNA)
  modified_base 18: mod_base = OTHER; note = locked nucleic acid (LNA)
  modified_base 22: mod_base = OTHER; note = locked nucleic acid (LNA)
  modified_base 1: mod_base = m5c
  modified_base 29: mod_base = m5c
  modified_base 24: mod_base = OTHER; note = locked nucleic acid (LNA)
  modified_base 26: mod_base = OTHER; note = locked nucleic acid (LNA)
  modified_base 28..29: mod_base = OTHER; note = locked nucleic acid (LNA)
  modified_base 20: mod_base = OTHER; note = locked nucleic acid (LNA)
cacagacacacacacacacacacacacac

SEQ ID NO: 239 - DNA, 29 bp, synthetic construct
  source 1..29: mol_type = other DNA
  misc_feature 1..29: note = linked by phosphorothioate internucleoside linkage
  modified_base 1..2: mod_base = OTHER; note = locked nucleic acid (LNA)
  modified_base 4: mod_base = OTHER; note = locked nucleic acid (LNA)
  modified_base 6: mod_base = OTHER; note = locked nucleic acid (LNA)
  modified_base 8: mod_base = OTHER; note = locked nucleic acid (LNA)
  modified_base 10: mod_base = OTHER; note = locked nucleic acid (LNA)
  modified_base 12: mod_base = OTHER; note = locked nucleic acid (LNA)
  modified_base 14: mod_base = OTHER; note = locked nucleic acid (LNA)
  modified_base 16: mod_base = OTHER; note = locked nucleic acid (LNA)
  modified_base 18: mod_base = OTHER; note = locked nucleic acid (LNA)
  modified_base 22: mod_base = OTHER; note = locked nucleic acid (LNA)
  modified_base 1: mod_base = m5c
  modified_base 29: mod_base = m5c
  modified_base 24: mod_base = OTHER; note = locked nucleic acid (LNA)
  modified_base 26: mod_base = OTHER; note = locked nucleic acid (LNA)
  modified_base 28..29: mod_base = OTHER; note = locked nucleic acid (LNA)
  modified_base 20: mod_base = OTHER; note = locked nucleic acid (LNA)
  modified_base 5: mod_base = i
cacanacacacacacacacacacacacac

SEQ ID NO: 240 - DNA, 29 bp, synthetic construct
  source 1..29: mol_type = other DNA
  misc_feature 1..29: note = linked by phosphorothioate internucleoside linkage
  modified_base 1..2: mod_base = OTHER; note = locked nucleic acid (LNA)
  modified_base 4: mod_base = OTHER; note = locked nucleic acid (LNA)
  modified_base 6: mod_base = OTHER; note = locked nucleic acid (LNA)
  modified_base 8: mod_base = OTHER; note = locked nucleic acid (LNA)
  modified_base 10: mod_base = OTHER; note = locked nucleic acid (LNA)
  modified_base 12: mod_base = OTHER; note = locked nucleic acid (LNA)
  modified_base 14: mod_base = OTHER; note = locked nucleic acid (LNA)
  modified_base 16: mod_base = OTHER; note = locked nucleic acid (LNA)
  modified_base 18: mod_base = OTHER; note = locked nucleic acid (LNA)
  modified_base 22: mod_base = OTHER; note = locked nucleic acid (LNA)
  modified_base 1: mod_base = m5c
  modified_base 29: mod_base = m5c
  modified_base 24: mod_base = OTHER; note = locked nucleic acid (LNA)
  modified_base 26: mod_base = OTHER; note = locked nucleic acid (LNA)
  modified_base 28..29: mod_base = OTHER; note = locked nucleic acid (LNA)
  modified_base 20: mod_base = OTHER; note = locked nucleic acid (LNA)
cacaaacacacacacacacacacacacac

SEQ ID NO: 241 - DNA, 29 bp, synthetic construct
  source 1..29: mol_type = other DNA
  misc_feature 1..29: note = linked by phosphorothioate internucleoside linkage
  modified_base 1..2: mod_base = OTHER; note = locked nucleic acid (LNA)
  modified_base 4: mod_base = OTHER; note = locked nucleic acid (LNA)
  modified_base 6: mod_base = OTHER; note = locked nucleic acid (LNA)
  modified_base 8: mod_base = OTHER; note = locked nucleic acid (LNA)
  modified_base 10: mod_base = OTHER; note = locked nucleic acid (LNA)
  modified_base 12: mod_base = OTHER; note = locked nucleic acid (LNA)
  modified_base 14: mod_base = OTHER; note = locked nucleic acid (LNA)
  modified_base 16: mod_base = OTHER; note = locked nucleic acid (LNA)
  modified_base 18: mod_base = OTHER; note = locked nucleic acid (LNA)
  modified_base 22: mod_base = OTHER; note = locked nucleic acid (LNA)
  modified_base 1: mod_base = m5c
  modified_base 29: mod_base = m5c
  modified_base 24: mod_base = OTHER; note = locked nucleic acid (LNA)
  modified_base 26: mod_base = OTHER; note = locked nucleic acid (LNA)
  modified_base 28..29: mod_base = OTHER; note = locked nucleic acid (LNA)
  modified_base 20: mod_base = OTHER; note = locked nucleic acid (LNA)
cacatacacacacacacacacacacacac

SEQ ID NO: 242 - DNA, 29 bp, synthetic construct
  source 1..29: mol_type = other DNA
  misc_feature 1..29: note = linked by phosphorothioate internucleoside linkage
  modified_base 1..2: mod_base = OTHER; note = locked nucleic acid (LNA)
  modified_base 4: mod_base = OTHER; note = locked nucleic acid (LNA)
  modified_base 6: mod_base = OTHER; note = locked nucleic acid (LNA)
  modified_base 8: mod_base = OTHER; note = locked nucleic acid (LNA)
  modified_base 10: mod_base = OTHER; note = locked nucleic acid (LNA)
  modified_base 12: mod_base = OTHER; note = locked nucleic acid (LNA)
  modified_base 14: mod_base = OTHER; note = locked nucleic acid (LNA)
  modified_base 16: mod_base = OTHER; note = locked nucleic acid (LNA)
  modified_base 18: mod_base = OTHER; note = locked nucleic acid (LNA)
  modified_base 22: mod_base = OTHER; note = locked nucleic acid (LNA)
  modified_base 1: mod_base = m5c
  modified_base 29: mod_base = m5c
  modified_base 24: mod_base = OTHER; note = locked nucleic acid (LNA)
  modified_base 26: mod_base = OTHER; note = locked nucleic acid (LNA)
  modified_base 28..29: mod_base = OTHER; note = locked nucleic acid (LNA)
  modified_base 20: mod_base = OTHER; note = locked nucleic acid (LNA)
catacgcacatacacgcacacacaaacac

SEQ ID NO: 243 - DNA, 29 bp, synthetic construct
  source 1..29: mol_type = other DNA
  misc_feature 1..29: note = linked by phosphorothioate internucleoside linkage
  modified_base 1..2: mod_base = OTHER; note = locked nucleic acid (LNA)
  modified_base 4: mod_base = OTHER; note = locked nucleic acid (LNA)
  modified_base 6: mod_base = OTHER; note = locked nucleic acid (LNA)
  modified_base 8: mod_base = OTHER; note = locked nucleic acid (LNA)
  modified_base 10: mod_base = OTHER; note = locked nucleic acid (LNA)
  modified_base 12: mod_base = OTHER; note = locked nucleic acid (LNA)
  modified_base 14: mod_base = OTHER; note = locked nucleic acid (LNA)
  modified_base 16: mod_base = OTHER; note = locked nucleic acid (LNA)
  modified_base 18: mod_base = OTHER; note = locked nucleic acid (LNA)
  modified_base 22: mod_base = OTHER; note = locked nucleic acid (LNA)
  modified_base 1: mod_base = m5c
  modified_base 29: mod_base = m5c
  modified_base 24: mod_base = OTHER; note = locked nucleic acid (LNA)
  modified_base 26: mod_base = OTHER; note = locked nucleic acid (LNA)
  modified_base 28..29: mod_base = OTHER; note = locked nucleic acid (LNA)
  modified_base 20: mod_base = OTHER; note = locked nucleic acid (LNA)
cacatatacacatacacacacacacacac

SEQ ID NO: 244 - DNA, 29 bp, synthetic construct
  source 1..29: mol_type = other DNA
  misc_feature 1..29: note = linked by phosphorothioate internucleoside linkage
  modified_base 1..2: mod_base = OTHER; note = locked nucleic acid (LNA)
  modified_base 4: mod_base = OTHER; note = locked nucleic acid (LNA)
  modified_base 6: mod_base = OTHER; note = locked nucleic acid (LNA)
  modified_base 8: mod_base = OTHER; note = locked nucleic acid (LNA)
  modified_base 10: mod_base = OTHER; note = locked nucleic acid (LNA)
  modified_base 12: mod_base = OTHER; note = locked nucleic acid (LNA)
  modified_base 14: mod_base = OTHER; note = locked nucleic acid (LNA)
  modified_base 16: mod_base = OTHER; note = locked nucleic acid (LNA)
  modified_base 18: mod_base = OTHER; note = locked nucleic acid (LNA)
  modified_base 22: mod_base = OTHER; note = locked nucleic acid (LNA)
  modified_base 1: mod_base = m5c
  modified_base 29: mod_base = m5c
  modified_base 24: mod_base = OTHER; note = locked nucleic acid (LNA)
  modified_base 26: mod_base = OTHER; note = locked nucleic acid (LNA)
  modified_base 28..29: mod_base = OTHER; note = locked nucleic acid (LNA)
  modified_base 20: mod_base = OTHER; note = locked nucleic acid (LNA)
cacacacacacacacacactctctctctc

SEQ ID NO: 245 - DNA, 29 bp, synthetic construct
  source 1..29: mol_type = other DNA
  misc_feature 1..29: note = linked by phosphorothioate internucleoside linkage
  modified_base 1..2: mod_base = OTHER; note = locked nucleic acid (LNA)
  modified_base 4: mod_base = OTHER; note = locked nucleic acid (LNA)
  modified_base 6: mod_base = OTHER; note = locked nucleic acid (LNA)
  modified_base 8: mod_base = OTHER; note = locked nucleic acid (LNA)
  modified_base 10: mod_base = OTHER; note = locked nucleic acid (LNA)
  modified_base 12: mod_base = OTHER; note = locked nucleic acid (LNA)
  modified_base 14: mod_base = OTHER; note = locked nucleic acid (LNA)
  modified_base 16: mod_base = OTHER; note = locked nucleic acid (LNA)
  modified_base 18: mod_base = OTHER; note = locked nucleic acid (LNA)
  modified_base 22: mod_base = OTHER; note = locked nucleic acid (LNA)
  modified_base 1: mod_base = m5c
  modified_base 29: mod_base = m5c
  modified_base 24: mod_base = OTHER; note = locked nucleic acid (LNA)
  modified_base 26: mod_base = OTHER; note = locked nucleic acid (LNA)
  modified_base 28..29: mod_base = OTHER; note = locked nucleic acid (LNA)
  modified_base 20: mod_base = OTHER; note = locked nucleic acid (LNA)
cacgcacacacacacacacacacacacac

SEQ ID NO: 246 - DNA, 29 bp, synthetic construct
  source 1..29: mol_type = other DNA
  misc_feature 1..29: note = linked by phosphorothioate internucleoside linkage
  modified_base 1..2: mod_base = OTHER; note = locked nucleic acid (LNA)
  modified_base 4: mod_base = OTHER; note = locked nucleic acid (LNA)
  modified_base 6: mod_base = OTHER; note = locked nucleic acid (LNA)
  modified_base 8: mod_base = OTHER; note = locked nucleic acid (LNA)
  modified_base 10: mod_base = OTHER; note = locked nucleic acid (LNA)
  modified_base 12: mod_base = OTHER; note = locked nucleic acid (LNA)
  modified_base 14: mod_base = OTHER; note = locked nucleic acid (LNA)
  modified_base 16: mod_base = OTHER; note = locked nucleic acid (LNA)
  modified_base 18: mod_base = OTHER; note = locked nucleic acid (LNA)
  modified_base 22: mod_base = OTHER; note = locked nucleic acid (LNA)
  modified_base 1: mod_base = m5c
  modified_base 29: mod_base = m5c
  modified_base 24: mod_base = OTHER; note = locked nucleic acid (LNA)
  modified_base 26: mod_base = OTHER; note = locked nucleic acid (LNA)
  modified_base 28..29: mod_base = OTHER; note = locked nucleic acid (LNA)
  modified_base 20: mod_base = OTHER; note = locked nucleic acid (LNA)
  modified_base 17: mod_base = i
  modified_base 19: mod_base = i
  modified_base 21: mod_base = i
cacacacacacacacantntntcacacac

SEQ ID NO: 247 - DNA, 29 bp, synthetic construct
  source 1..29: mol_type = other DNA
  misc_feature 1..29: note = linked by phosphorothioate internucleoside linkage
  modified_base 1..2: mod_base = OTHER; note = locked nucleic acid (LNA)
  modified_base 4: mod_base = OTHER; note = locked nucleic acid (LNA)
  modified_base 6: mod_base = OTHER; note = locked nucleic acid (LNA)
  modified_base 8: mod_base = OTHER; note = locked nucleic acid (LNA)
  modified_base 10: mod_base = OTHER; note = locked nucleic acid (LNA)
  modified_base 12: mod_base = OTHER; note = locked nucleic acid (LNA)
  modified_base 14: mod_base = OTHER; note = locked nucleic acid (LNA)
  modified_base 16: mod_base = OTHER; note = locked nucleic acid (LNA)
  modified_base 18: mod_base = OTHER; note = locked nucleic acid (LNA)
  modified_base 22: mod_base = OTHER; note = locked nucleic acid (LNA)
  modified_base 1: mod_base = m5c
  modified_base 29: mod_base = m5c
  modified_base 24: mod_base = OTHER; note = locked nucleic acid (LNA)
  modified_base 26: mod_base = OTHER; note = locked nucleic acid (LNA)
  modified_base 28..29: mod_base = OTHER; note = locked nucleic acid (LNA)
  modified_base 20: mod_base = OTHER; note = locked nucleic acid (LNA)
cgcacacacacacacacacacacacacac

SEQ ID NO: 248 - DNA, 29 bp, synthetic construct
  source 1..29: mol_type = other DNA
  misc_feature 1..29: note = linked by phosphorothioate internucleoside linkage
  modified_base 1..2: mod_base = OTHER; note = locked nucleic acid (LNA)
  modified_base 4: mod_base = OTHER; note = locked nucleic acid (LNA)
  modified_base 6: mod_base = OTHER; note = locked nucleic acid (LNA)
  modified_base 8: mod_base = OTHER; note = locked nucleic acid (LNA)
  modified_base 10: mod_base = OTHER; note = locked nucleic acid (LNA)
  modified_base 12: mod_base = OTHER; note = locked nucleic acid (LNA)
  modified_base 14: mod_base = OTHER; note = locked nucleic acid (LNA)
  modified_base 16: mod_base = OTHER; note = locked nucleic acid (LNA)
  modified_base 18: mod_base = OTHER; note = locked nucleic acid (LNA)
  modified_base 22: mod_base = OTHER; note = locked nucleic acid (LNA)
  modified_base 1: mod_base = m5c
  modified_base 29: mod_base = m5c
  modified_base 24: mod_base = OTHER; note = locked nucleic acid (LNA)
  modified_base 26: mod_base = OTHER; note = locked nucleic acid (LNA)
  modified_base 28..29: mod_base = OTHER; note = locked nucleic acid (LNA)
  modified_base 20: mod_base = OTHER; note = locked nucleic acid (LNA)
cacacacacacacacagacagacagacac

SEQ ID NO: 249 - DNA, 29 bp, synthetic construct
  source 1..29: mol_type = other DNA
  misc_feature 1..29: note = linked by phosphorothioate internucleoside linkage
  modified_base 1..2: mod_base = OTHER; note = locked nucleic acid (LNA)
  modified_base 4: mod_base = OTHER; note = locked nucleic acid (LNA)
  modified_base 6: mod_base = OTHER; note = locked nucleic acid (LNA)
  modified_base 8: mod_base = OTHER; note = locked nucleic acid (LNA)
  modified_base 10: mod_base = OTHER; note = locked nucleic acid (LNA)
  modified_base 12: mod_base = OTHER; note = locked nucleic acid (LNA)
  modified_base 14: mod_base = OTHER; note = locked nucleic acid (LNA)
  modified_base 16: mod_base = OTHER; note = locked nucleic acid (LNA)
  modified_base 18: mod_base = OTHER; note = locked nucleic acid (LNA)
  modified_base 22: mod_base = OTHER; note = locked nucleic acid (LNA)
  modified_base 1: mod_base = m5c
  modified_base 29: mod_base = m5c
  modified_base 24: mod_base = OTHER; note = locked nucleic acid (LNA)
  modified_base 26: mod_base = OTHER; note = locked nucleic acid (LNA)
  modified_base 28..29: mod_base = OTHER; note = locked nucleic acid (LNA)
  modified_base 20: mod_base = OTHER; note = locked nucleic acid (LNA)
cacacacacaaaaaaacacacacacacac

SEQ ID NO: 250 - DNA, 29 bp, synthetic construct
  source 1..29: mol_type = other DNA
  misc_feature 1..29: note = linked by phosphorothioate internucleoside linkage
  modified_base 1..2: mod_base = OTHER; note = locked nucleic acid (LNA)
  modified_base 4: mod_base = OTHER; note = locked nucleic acid (LNA)
  modified_base 6: mod_base = OTHER; note = locked nucleic acid (LNA)
  modified_base 8: mod_base = OTHER; note = locked nucleic acid (LNA)
  modified_base 10: mod_base = OTHER; note = locked nucleic acid (LNA)
  modified_base 12: mod_base = OTHER; note = locked nucleic acid (LNA)
  modified_base 14: mod_base = OTHER; note = locked nucleic acid (LNA)
  modified_base 16: mod_base = OTHER; note = locked nucleic acid (LNA)
  modified_base 18: mod_base = OTHER; note = locked nucleic acid (LNA)
  modified_base 22: mod_base = OTHER; note = locked nucleic acid (LNA)
  modified_base 1: mod_base = m5c
  modified_base 29: mod_base = m5c
  modified_base 24: mod_base = OTHER; note = locked nucleic acid (LNA)
  modified_base 26: mod_base = OTHER; note = locked nucleic acid (LNA)
  modified_base 28..29: mod_base = OTHER; note = locked nucleic acid (LNA)
  modified_base 20: mod_base = OTHER; note = locked nucleic acid (LNA)
  modified_base 12: mod_base = m5c
  modified_base 14: mod_base = m5c
  modified_base 16: mod_base = m5c
cacacacacacccccccacacacacacac

SEQ ID NO: 251 - DNA, 25 bp, synthetic construct
  source 1..25: mol_type = other DNA
  misc_feature 1..25: note = linked by phosphorothioate internucleoside linkage
  modified_base 1: mod_base = m5c
  modified_base 25: mod_base = m5c
  modified_base 1..25: mod_base = OTHER; note = 2-O-(2-methoxyethyl) nucleoside
  modified_base 3: mod_base = m5c
  modified_base 5: mod_base = m5c
  modified_base 7: mod_base = m5c
  modified_base 9: mod_base = m5c
  modified_base 11: mod_base = m5c
  modified_base 13: mod_base = m5c
  modified_base 15: mod_base = m5c
  modified_base 17: mod_base = m5c
  modified_base 19: mod_base = m5c
  modified_base 21: mod_base = m5c
  modified_base 23: mod_base = m5c
cacacacactctctcacacacacac

SEQ ID NO: 252 - DNA, 25 bp, synthetic construct
  source 1..25: mol_type = other DNA
  misc_feature 1..25: note = linked by phosphorothioate internucleoside linkage
  modified_base 1: mod_base = m5c
  modified_base 25: mod_base = m5c
  modified_base 1..25: mod_base = OTHER; note = 2-O-(2-methoxyethyl) nucleoside
  modified_base 3: mod_base = m5c
  modified_base 7: mod_base = m5c
  modified_base 9: mod_base = m5c
  modified_base 11: mod_base = m5c
  modified_base 13: mod_base = m5c
  modified_base 15: mod_base = m5c
  modified_base 17: mod_base = m5c
  modified_base 19: mod_base = m5c
  modified_base 21: mod_base = m5c
  modified_base 23: mod_base = m5c
cacaaacacacacacacacacacac

SEQ ID NO: 253 - DNA, 25 bp, synthetic construct
  source 1..25: mol_type = other DNA
  misc_feature 1..25: note = linked by phosphorothioate internucleoside linkage
  modified_base 1..2: mod_base = OTHER; note = 2-O-(2-methoxyethyl) nucleoside
  modified_base 4: mod_base = OTHER; note = 2-O-(2-methoxyethyl) nucleoside
  modified_base 6: mod_base = OTHER; note = 2-O-(2-methoxyethyl) nucleoside
  modified_base 8: mod_base = OTHER; note = 2-O-(2-methoxyethyl) nucleoside
  modified_base 10: mod_base = OTHER; note = 2-O-(2-methoxyethyl) nucleoside
  modified_base 12: mod_base = OTHER; note = 2-O-(2-methoxyethyl) nucleoside
  modified_base 14: mod_base = OTHER; note = 2-O-(2-methoxyethyl) nucleoside
  modified_base 16: mod_base = OTHER; note = 2-O-(2-methoxyethyl) nucleoside
  modified_base 18: mod_base = OTHER; note = 2-O-(2-methoxyethyl) nucleoside
  modified_base 20: mod_base = OTHER; note = 2-O-(2-methoxyethyl) nucleoside
  modified_base 22: mod_base = OTHER; note = 2-O-(2-methoxyethyl) nucleoside
  modified_base 24..25: mod_base = OTHER; note = 2-O-(2-methoxyethyl) nucleoside
  modified_base 3: mod_base = OTHER; note = locked nucleic acid (LNA)
  modified_base 5: mod_base = OTHER; note = locked nucleic acid (LNA)
  modified_base 7: mod_base = OTHER; note = locked nucleic acid (LNA)
  modified_base 9: mod_base = OTHER; note = locked nucleic acid (LNA)
  modified_base 11: mod_base = OTHER; note = locked nucleic acid (LNA)
  modified_base 13: mod_base = OTHER; note = locked nucleic acid (LNA)
  modified_base 15: mod_base = OTHER; note = locked nucleic acid (LNA)
  modified_base 17: mod_base = OTHER; note = locked nucleic acid (LNA)
  modified_base 19: mod_base = OTHER; note = locked nucleic acid (LNA)
  modified_base 21: mod_base = OTHER; note = locked nucleic acid (LNA)
  modified_base 23: mod_base = OTHER; note = locked nucleic acid (LNA)
  modified_base 1: mod_base = m5c
  modified_base 3: mod_base = m5c
  modified_base 5: mod_base = m5c
  modified_base 7: mod_base = m5c
  modified_base 9: mod_base = m5c
  modified_base 11: mod_base = m5c
  modified_base 13: mod_base = m5c
  modified_base 15: mod_base = m5c
  modified_base 17: mod_base = m5c
  modified_base 19: mod_base = m5c
  modified_base 21: mod_base = m5c
  modified_base 23: mod_base = m5c
  modified_base 25: mod_base = m5c
cacacacacacacacacacacacac

SEQ ID NO: 254 - DNA, 25 bp, synthetic construct
  source 1..25: mol_type = other DNA
  misc_feature 1..25: note = linked by phosphorothioate internucleoside linkage
  modified_base 1: mod_base = m5c
  modified_base 25: mod_base = m5c
  modified_base 1..25: mod_base = OTHER; note = 2-O-(2-methoxyethyl) nucleoside
  modified_base 3: mod_base = m5c
  modified_base 5: mod_base = m5c
  modified_base 7: mod_base = m5c
  modified_base 9: mod_base = m5c
  modified_base 11: mod_base = m5c
  modified_base 13: mod_base = m5c
  modified_base 15: mod_base = m5c
  modified_base 17: mod_base = m5c
  modified_base 19: mod_base = m5c
  modified_base 21: mod_base = m5c
  modified_base 23: mod_base = m5c
cactcacacacacacacacacacac

SEQ ID NO: 255 - DNA, 25 bp, synthetic construct
  source 1..25: mol_type = other DNA
  misc_feature 1..25: note = linked by phosphorothioate internucleoside linkage
  modified_base 1: mod_base = m5c
  modified_base 25: mod_base = m5c
  modified_base 1..25: mod_base = OTHER; note = 2-O-(2-methoxyethyl) nucleoside
  modified_base 3: mod_base = m5c
  modified_base 5: mod_base = m5c
  modified_base 7: mod_base = m5c
  modified_base 9: mod_base = m5c
  modified_base 13: mod_base = m5c
  modified_base 15: mod_base = m5c
  modified_base 17: mod_base = m5c
  modified_base 19: mod_base = m5c
  modified_base 21: mod_base = m5c
  modified_base 23: mod_base = m5c
cacacacacaaacacacacacacac

SEQ ID NO: 256 - DNA, 25 bp, synthetic construct
  source 1..25: mol_type = other DNA
  misc_feature 1..25: note = linked by phosphorothioate internucleoside linkage
  modified_base 1: mod_base = m5c
  modified_base 25: mod_base = m5c
  modified_base 1..25: mod_base = OTHER; note = 2-O-(2-methoxyethyl) nucleoside
  modified_base 3..5: mod_base = m5c
  modified_base 7: mod_base = m5c
  modified_base 9: mod_base = m5c
  modified_base 11: mod_base = m5c
  modified_base 13: mod_base = m5c
  modified_base 15: mod_base = m5c
  modified_base 17: mod_base = m5c
  modified_base 19: mod_base = m5c
  modified_base 21: mod_base = m5c
  modified_base 23: mod_base = m5c
cacccacacacacacacacacacac

SEQ ID NO: 257 - DNA, 25 bp, synthetic construct
  source 1..25: mol_type = other DNA
  misc_feature 1..25: note = linked by phosphorothioate internucleoside linkage
  modified_base 1: mod_base = m5c
  modified_base 25: mod_base = m5c
  modified_base 1..25: mod_base = OTHER; note = 2-O-(2-methoxyethyl) nucleoside
  modified_base 3: mod_base = m5c
  modified_base 5: mod_base = m5c
  modified_base 7: mod_base = m5c
  modified_base 9: mod_base = m5c
  modified_base 11: mod_base = m5c
  modified_base 13: mod_base = m5c
  modified_base 15: mod_base = m5c
  modified_base 17: mod_base = m5c
  modified_base 19: mod_base = m5c
  modified_base 21: mod_base = m5c
  modified_base 23: mod_base = m5c
ctctctcacacacacacacacacac

SEQ ID NO: 258 - DNA, 25 bp, synthetic construct
  source 1..25: mol_type = other DNA
  misc_feature 1..25: note = linked by phosphorothioate internucleoside linkage
  modified_base 1: mod_base = m5c
  modified_base 25: mod_base = m5c
  modified_base 1..25: mod_base = OTHER; note = 2-O-(2-methoxyethyl) nucleoside
  modified_base 5: mod_base = m5c
  modified_base 7: mod_base = m5c
  modified_base 9: mod_base = m5c
  modified_base 11: mod_base = m5c
  modified_base 13: mod_base = m5c
  modified_base 15: mod_base = m5c
  modified_base 17: mod_base = m5c
  modified_base 19: mod_base = m5c
  modified_base 21: mod_base = m5c
  modified_base 23: mod_base = m5c
caaacacacacacacacacacacac

SEQ ID NO: 259 - DNA, 25 bp, synthetic construct
  source 1..25: mol_type = other DNA
  misc_feature 1..25: note = linked by phosphorothioate internucleoside linkage
  modified_base 1: mod_base = m5c
  modified_base 25: mod_base = m5c
  modified_base 1..25: mod_base = OTHER; note = 2-O-(2-methoxyethyl) nucleoside
  modified_base 3: mod_base = m5c
  modified_base 5: mod_base = m5c
  modified_base 7: mod_base = m5c
  modified_base 9..11: mod_base = m5c
  modified_base 13: mod_base = m5c
  modified_base 15: mod_base = m5c
  modified_base 17: mod_base = m5c
  modified_base 19: mod_base = m5c
  modified_base 21: mod_base = m5c
  modified_base 23: mod_base = m5c
cacacacacccacacacacacacac

SEQ ID NO: 260 - DNA, 25 bp, synthetic construct
  source 1..25: mol_type = other DNA
  misc_feature 1..25: note = linked by phosphorothioate internucleoside linkage
  modified_base 1: mod_base = m5c
  modified_base 25: mod_base = m5c
  modified_base 1..25: mod_base = OTHER; note = 2-O-(2-methoxyethyl) nucleoside
  modified_base 3: mod_base = m5c
  modified_base 5: mod_base = m5c
  modified_base 7: mod_base = m5c
  modified_base 9: mod_base = m5c
  modified_base 11: mod_base = m5c
  modified_base 13: mod_base = m5c
  modified_base 15..17: mod_base = m5c
  modified_base 19: mod_base = m5c
  modified_base 21: mod_base = m5c
  modified_base 23: mod_base = m5c
cacacacacacacacccacacacac

SEQ ID NO: 261 - DNA, 25 bp, synthetic construct
  source 1..25: mol_type = other DNA
  misc_feature 1..25: note = linked by phosphorothioate internucleoside linkage
  modified_base 1: mod_base = m5c
  modified_base 25: mod_base = m5c
  modified_base 1..25: mod_base = OTHER; note = 2-O-(2-methoxyethyl) nucleoside
  modified_base 3: mod_base = m5c
  modified_base 5: mod_base = m5c
  modified_base 7: mod_base = m5c
  modified_base 9: mod_base = m5c
  modified_base 11: mod_base = m5c
  modified_base 13: mod_base = m5c
  modified_base 15: mod_base = m5c
  modified_base 17: mod_base = m5c
  modified_base 19: mod_base = m5c
  modified_base 21: mod_base = m5c
  modified_base 23: mod_base = m5c
cacacacacacacacgcacacacac

SEQ ID NO: 262 - DNA, 25 bp, synthetic construct
  source 1..25: mol_type = other DNA
  misc_feature 1..25: note = linked by phosphorothioate internucleoside linkage
  modified_base 1: mod_base = m5c
  modified_base 25: mod_base = m5c
  modified_base 1..25: mod_base = OTHER; note = 2-O-(2-methoxyethyl) nucleoside
  modified_base 3: mod_base = m5c
  modified_base 5: mod_base = m5c
  modified_base 7: mod_base = m5c
  modified_base 9: mod_base = m5c
  modified_base 11: mod_base = m5c
  modified_base 13: mod_base = m5c
  modified_base 15: mod_base = m5c
  modified_base 17: mod_base = m5c
  modified_base 19: mod_base = m5c
  modified_base 21: mod_base = m5c
  modified_base 23: mod_base = m5c
cacacacacacacactcacacacac

SEQ ID NO: 263 - DNA, 25 bp, synthetic construct
  source 1..25: mol_type = other DNA
  misc_feature 1..25: note = linked by phosphorothioate internucleoside linkage
  modified_base 1: mod_base = m5c
  modified_base 25: mod_base = m5c
  modified_base 1..25: mod_base = OTHER; note = 2-O-(2-methoxyethyl) nucleoside
  modified_base 3: mod_base = m5c
  modified_base 5: mod_base = m5c
  modified_base 7: mod_base = m5c
  modified_base 9: mod_base = m5c
  modified_base 13: mod_base = m5c
  modified_base 15: mod_base = m5c
  modified_base 17: mod_base = m5c
  modified_base 19: mod_base = m5c
  modified_base 21: mod_base = m5c
  modified_base 23: mod_base = m5c
cacacacacatacacacacacacac

SEQ ID NO: 264 - DNA, 25 bp, synthetic construct
  source 1..25: mol_type = other DNA
  misc_feature 1..25: note = linked by phosphorothioate internucleoside linkage
  modified_base 1: mod_base = m5c
  modified_base 25: mod_base = m5c
  modified_base 1..25: mod_base = OTHER; note = 2-O-(2-methoxyethyl) nucleoside
  modified_base 3: mod_base = m5c
  modified_base 5: mod_base = m5c
  modified_base 7: mod_base = m5c
  modified_base 9: mod_base = m5c
  modified_base 13: mod_base = m5c
  modified_base 15: mod_base = m5c
  modified_base 17: mod_base = m5c
  modified_base 19: mod_base = m5c
  modified_base 21: mod_base = m5c
  modified_base 23: mod_base = m5c
cacacacacagacacacacacacac

SEQ ID NO: 265 - DNA, 25 bp, synthetic construct
  source 1..25: mol_type = other DNA
  misc_feature 1..25: note = linked by phosphorothioate internucleoside linkage
  modified_base 25: mod_base = m5c
  modified_base 1..25: mod_base = OTHER; note = 2-O-(2-methoxyethyl) nucleoside
  modified_base 5: mod_base = m5c
  modified_base 7: mod_base = m5c
  modified_base 9: mod_base = m5c
  modified_base 11: mod_base = m5c
  modified_base 13: mod_base = m5c
  modified_base 15: mod_base = m5c
  modified_base 17: mod_base = m5c
  modified_base 19: mod_base = m5c
  modified_base 21: mod_base = m5c
  modified_base 23: mod_base = m5c
tttacacacacacacacacacacac

SEQ ID NO: 266 - DNA, 25 bp, synthetic construct
  source 1..25: mol_type = other DNA
  misc_feature 1..25: note = linked by phosphorothioate internucleoside linkage
  modified_base 1: mod_base = m5c
  modified_base 25: mod_base = m5c
  modified_base 1..25: mod_base = OTHER; note = 2-O-(2-methoxyethyl) nucleoside
  modified_base 3: mod_base = m5c
  modified_base 5: mod_base = m5c
  modified_base 7: mod_base = m5c
  modified_base 9: mod_base = m5c
  modified_base 11: mod_base = m5c
  modified_base 13: mod_base = m5c
  modified_base 15: mod_base = m5c
  modified_base 17: mod_base = m5c
  modified_base 19: mod_base = m5c
  modified_base 21: mod_base = m5c
  modified_base 23: mod_base = m5c
cacgcacacgcacacacacacacac

SEQ ID NO: 267 - DNA, 25 bp, synthetic construct
  source 1..25: mol_type = other DNA
  misc_feature 1..25: note = linked by phosphorothioate internucleoside linkage
  modified_base 1: mod_base = m5c
  modified_base 25: mod_base = m5c
  modified_base 1..25: mod_base = OTHER; note = 2-O-(2-methoxyethyl) nucleoside
  modified_base 3: mod_base = m5c
  modified_base 5: mod_base = m5c
  modified_base 7: mod_base = m5c
  modified_base 9: mod_base = m5c
  modified_base 11: mod_base = m5c
  modified_base 13: mod_base = m5c
  modified_base 15: mod_base = m5c
  modified_base 17: mod_base = m5c
  modified_base 19: mod_base = m5c
  modified_base 21: mod_base = m5c
  modified_base 23: mod_base = m5c
cacgcacacacgcacacacacgcac

SEQ ID NO: 268 - DNA, 25 bp, synthetic construct
  source 1..25: mol_type = other DNA
  misc_feature 1..25: note = linked by phosphorothioate internucleoside linkage
  modified_base 1: mod_base = m5c
  modified_base 25: mod_base = m5c
  modified_base 1..25: mod_base = OTHER; note = 2-O-(2-methoxyethyl) nucleoside
  modified_base 3: mod_base = m5c
  modified_base 5: mod_base = m5c
  modified_base 7: mod_base = m5c
  modified_base 9: mod_base = m5c
  modified_base 11: mod_base = m5c
  modified_base 13: mod_base = m5c
  modified_base 15: mod_base = m5c
  modified_base 17: mod_base = m5c
  modified_base 19: mod_base = m5c
  modified_base 21: mod_base = m5c
  modified_base 23: mod_base = m5c
cacacgcacacacacacacacacac

(19) United States
(12) Patent Application Publication
(10) Pub. No.: US 2024/0327837 A1
(43) Pub. Date: Oct. 3, 2024

(54) OLIGONUCLEOTIDES TARGETING RNA BINDING PROTEIN SITES

(71) Applicant: Hoffmann-La Roche Inc., Little Falls, NJ (US)

(72) Inventors: Congwei WANG, Basel, Basel-City (CH); Christian WEILE, Farum (DK); Martin EBELING, Grenzach-Wyhlen (DE); Lars JOENSON, Viby Sjaelland (DK); Jonas VIKESAA, Fredensborg (DK); Ravi JAGASIA, Riehen, Basel-City (CH); Meiling LI, Rheinfelden, Aargau (CH)

(21) Appl. No.: 18/435,935

(22) Filed: Feb. 7, 2024

Related U.S. Application Data
(62) Division of application No. 17/383,709, filed on Jul. 23, 2021.

(30) Foreign Application Priority Data
Jul. 23, 2020 (EP) 20187354.4

(51) Int. Cl. C12N 15/113

(52) U.S. Cl. CPC C12N 15/113; C12N 2310/11; C12N 2310/3231

(57) ABSTRACT

The present invention relates to antisense oligonucleotides which are complementary to conserved TDP-43 binding sites on pre-mRNA transcripts, which are capable of restoring RNA binding protein function in the processing of multiple independent mRNAs in TDP-43 depleted cells.

### SEQUENCE LISTING

The instant application contains a Sequence Listing which has been submitted electronically in XML format and is hereby incorporated by reference in its entirety. Said XML copy, created on Feb. 7, 2024, is named 51551-008002-Sequence_Listing_2_7_24 and is 1,322,274 bytes in size.

The present invention relates to antisense oligonucleotides which are complementary, such as fully complementary, to RNA binding protein target sites on multiple RNAs, such as TDP-43 binding sites on multiple RNA transcripts, and are capable of restoring RNA binding protein functionality to the multiple RNA transcript, such as for use in conditions and medical indications where the RNA binding protein is functionally depleted.

### BACKGROUND

TAR DNA binding protein 43 (TDP-43) is a versatile RNA/DNA binding protein involved in RNA-related metabolism. Dysregulation of TDP-43 deposits act as inclusion bodies in the brain and spinal cord of patients with the motor neuron diseases: amyotrophic lateral sclerosis (ALS) and frontotemporal lobar degeneration (FTLD) (Prasad et al., Front. Mol. Neurosci., 2019).

TDP-43 is predominantly localized in the nucleus but also shuttles to the cytoplasm for some of its functions (Ayala et al., 2008). In disease, such as in ALS and FTLD, there is an increase in the cytoplasmic TDP-43 concentration leading to cytoplasmic inclusion formation (Neumann et al., 2006; Winton et al., 2008a). The cytoplasmic mis-localization can be associated with nuclear depletion, resulting in a reduction or loss of TDP-43 function. There are TDP-43 mutations which result in aberrant splicing of TDP-43 target RNAs, resulting in widespread splicing aberration (see for example Arnold et al., PNAS 2013 110 E736-745 and Yang et al., PNAS. U.S.A. 111, E1121-E1129).

Klim et al., reports that STMN2 loss upon reduced TDP-43 function is due to altered STMN2 splicing, and suggests restoring STMN2 as a therapeutic strategy for ALS.

TDP-43 depletion is indicated in a range of diseases, referred to as TDP-43 pathologies, and including for example diseases such as amyotrophic lateral sclerosis (ALS), frontotemporal lobar degeneration (FTLD), Progressive supranuclear palsy (PSP), Primary lateral sclerosis, Progressive muscular atrophy, Alzheimer's disease, Parkinson's disease, autism, Hippocampal sclerosis dementia, Down syndrome, Huntington's disease, polyglutamine diseases, such as spinocerebellar ataxia 3, myopathies and Chronic Traumatic Encephalopathy.

Tollervey et al., Nature Neuroscience 2010, 452-458 reports on the characterization of the RNA targets and position dependent splicing regulation of TDP-43 in healthy brain tissue and brain tissue from FTLD patients. Most TDP-43 binding sites mapped to introns, long non-coding RNAs (IncRNA) and intergenic transcripts, which were enriched for UG-rich motifs. The conserved RNP segments in TDP-43 are involved in binding to TAR DNA sequences and RNA sequences with UG-repeats (Ayala et al., J. Mol. Biol. 2005;348:575-588). TDP-43 depletion in cells, such as in TDP-pathologies is correlated to the loss of RNA binding of TDP-43 to TDP-43 RNA targets.

TDP-43 binding sites in human RNAs are available on line from the A database of RNA binding proteins and associated motifs—see attract.cnic.es/results/e9f29380-8921-406e-84a8-27ce9b9398b4. Certain characterized human RNA TDP-43 binding sites disclosed include the following RNA sequences: GUGAAUGA, GUUGUGC, UGUGUGUGUGUG (SEQ ID NO: 20), GAAUGG, UGUGUGUG, GAAUGA, UGUGUG, GUUGUUC, and GUUUUGC.

Melamed et al., reports on premature polyadenylation-mediated loss of STMN2 as a hallmark of TDP-43 neurodegeneration. WO2019/241648 discloses 2′O-methoxyethyl ASOs for increasing STMN2 expression.

The present inventors have identified antisense oligonucleotides which are complementary, such as fully complementary to TDP-43 nucleic acid binding sites and are capable of restoring the processing or regulation of TDP-43 RNA transcripts targets, e.g. the expression and splicing of RNA transcripts, that are dysregulated in cells showing TDP-43 loss of function, and thereby provides a novel approach to restore TDP-43 functionality in TDP-43 depleted cells (i.e. cells with a loss of TDP-43 function), as well as a novel therapeutic approach for the treatment of TDP-43 pathologies.

### OBJECTIVE OF THE INVENTION

The present invention relates to antisense oligonucleotides which are complementary to conserved TDP-43 binding sites on pre-mRNA transcripts, which are capable of restoring RNA binding protein function in the processing of multiple independent mRNAs in TDP-43 depleted cells.

The present invention provides oligonucleotides for restoring RNA binding protein functionality, such as TDP-43 functionality, or TDP-43 like functionality, in cells which have a reduced level of functioning TDP-43.

The present invention provides oligonucleotide which are capable of restoring the nuclear function of TDP-43 in the RNA processing or expression of one or more TDP-43 target RNAs, and thereby restore, at least partially, or enhance, the functional phenotype of the TDP-43 target RNA(s). Such oligonucleotide compounds are referred to herein as RNA binding protein mimics, such as TDP-43 mimics.

The present invention provides antisense oligonucleotides which are complementary to TDP-43 binding sites, and their use in therapy, such as for the treatment of TDP-43 pathologies.

The present invention further provides oligonucleotides which are complementary to TDP-43 binding sites on multiple RNA transcripts, i.e. RNA transcripts which are transcribed from distinct genetic loci. The multiple RNA transcripts may, for example, be independently selected from the group consisting of pre-mRNAs, mRNAs, and IncRNA.

### SUMMARY OF INVENTION

The invention provides an antisense oligonucleotide of 8 to 40 nucleotides in length, which comprises a contiguous nucleotide sequence of at least 8 nucleotides in length which is complementary to, such as fully complementarity, a sequence selected from the group consisting of (5′-3′) (UG)n, (GU)n, wherein n is 4-20, UGUGUGUG, UGUGUGUGU, UGUGUGUGUG (SEQ ID NO: 37), UGUGUGUGUGU (SEQ ID NO: 38), UGUGUGUGUGUG (SEQ ID NO: 35), UGUGUGUGUGUGU (SEQ ID NO: 39), GUGUGUGU, GUGUGUGUG, GUGUGUGUGU (SEQ ID NO: 40), GUGUGUGUGUG (SEQ ID NO: 41), GUGUGUGUGUGU (SEQ ID NO: 42), GUGUGUGUGUGUG (SEQ ID NO: 43), and GUGAAUGA, wherein the antisense oligonucleotide is capable of restoring the functional phenotype of one or more TDP-43 target RNA(s) in a cell which is TDP-43 depleted, such as a cell which is expressing aberrant TDP-43 protein; or a pharmaceutically acceptable salt thereof.

The contiguous nucleotide sequence may comprise one or more modified nucleosides.

As explained in the background section, functional TDP-43 is primarily a nuclear localized protein, which may exist in the cytoplasm. However, aggregation of TDP-43 in the cytoplasm, what are referred to as cytoplasmic inclusions (also referred to as aberrant TDP-43), is associated with non-functional TDP-43, and this is associated with a loss of nuclear TDP-43 functionality for example in the processing of numerous pre-mRNAs. Cells which express TDP-43 in cytoplasmic inclusions are therefore to be considered TDP-43 depleted.

The antisense oligonucleotide may be an isolated antisense oligonucleotide or a purified oligonucleotide. The antisense oligonucleotide of the invention is a manufactured (man-made) antisense oligonucleotide.

The functional phenotype may for example be RNA processing events which are modulated by or dependent upon functional TDP-43 (i.e. non-aberrant TDP-43, typically nuclear TDP-43), and/or whose fidelity is dependent upon functional TDP-43. The enhancement of TDP-43 functionality by use of the antisense oligonucleotides of the invention may therefore be evaluated by assessing the fidelity of RNA processing events which are modulated by or dependent upon functional TDP-43, as for example illustrated herein, with reference to the STMN2, ARHGAP32, SLC5A7, CERT1, CAMK2B, KALRN and UNC13A RNA processing.

The invention provides for an antisense oligonucleotide, of 8 to 40 nucleotides in length, which comprises a contiguous nucleotide sequence of at least 8 nucleotides in length with at least 75% complementarity, such as at least 90% complementarity or 100% complementarity, to a sequence selected from the group consisting of (5′-3′) (UG)n, (GU)n, wherein n is 4-20, UGUGUGUG, UGUGUGUGU, UGUGUGUGUG (SEQ ID NO: 37), UGUGUGUGUGU (SEQ ID NO: 38), UGUGUGUGUGUG (SEQ ID NO: 35), UGUGUGUGUGUGU (SEQ ID NO: 39), GUGUGUGU, GUGUGUGUG, GUGUGUGUGU (SEQ ID NO: 40), GUGUGUGUGUGU (SEQ ID NO: 42), GUGUGUGUGUGUG (SEQ ID NO: 43), and GUGAAUGA, or a pharmaceutically acceptable salt thereof, for use in medicine.

The contiguous nucleotide sequence may comprise one or more modified nucleosides.

The invention provides for an antisense oligonucleotide, of 8 to 40 nucleotides in length, which comprises a contiguous nucleotide sequence of at least 8 nucleotides in length with at least 75% complementarity, such as at least 90% complementarity or 100% complementarity to a sequence selected from the group consisting of (5′-3′) (UG)n, (GU)n, wherein n is 4-20, UGUGUGUG, UGUGUGUGU, UGUGUGUGUG (SEQ ID NO: 37), UGUGUGUGUGU (SEQ ID NO: 38), UGUGUGUGUGUG (SEQ ID NO: 35), UGUGUGUGUGUGU (SEQ ID NO: 39), GUGUGUGU, GUGUGUGUG, GUGUGUGUGU (SEQ ID NO: 40), GUGUGUGUGUGU (SEQ ID NO: 42), GUGUGUGUGUGUG (SEQ ID NO: 43), and GUGAAUGA, or a pharmaceutically acceptable salt thereof, for use in the treatment of a disease characterized by TDP-43 pathology.

The contiguous nucleotide sequence may comprise one or more modified nucleosides.

Advantageously, the antisense oligonucleotide or contiguous nucleotide sequence thereof comprises at least 12 or at least 13 contiguous nucleotides, which are complementary, such as fully complementary, to the sequence UGUGUGUGUGUG (SEQ ID NO: 35), or GUGUGUGUGUGU (SEQ ID NO: 42), or UGUGUGUGUGUGU (SEQ ID NO: 39), or GUGUGUGUGUGUG (SEQ ID NO: 43).

In some embodiments, the antisense oligonucleotide or contiguous nucleotide sequence comprises at least 14 contiguous nucleotides which are complementary, such as fully complementary, to the sequence UGUGUGUGUGUGUG (SEQ ID NO: 46), or GUGUGUGUGUGUGU (SEQ ID NO: 47).

In some embodiments, the antisense oligonucleotide or contiguous nucleotide sequence comprises at least 18 contiguous nucleotides which are complementary, such as fully complementary, to the sequence (UG)n or (GU)n, wherein n is an integer 6-20, such as 7-9.

In some embodiments, the antisense oligonucleotide or contiguous nucleotide sequence comprises at least 18 contiguous nucleotides which are complementary, such as fully complementary, to the sequence UGUGUGUGUGUGUGUGUG (SEQ ID NO: 48), or GUGUGUGUGUGUGUGUGU (SEQ ID NO: 49).

In some embodiments, the antisense oligonucleotide according to the invention, or the contiguous nucleotide sequence thereof comprises the sequence CACACACA or ACACACAC.

In some embodiments, the antisense oligonucleotide according to the invention, or the contiguous nucleotide sequence thereof comprises the sequence CACACACACACA (SEQ ID NO: 14) or ACACACACACAC (SEQ ID NO: 5).

In some embodiments, the antisense oligonucleotide according to the invention, or the contiguous nucleotide sequence thereof comprises the sequence CACACACACACAC (SEQ ID NO: 15) or ACACACACACACA (SEQ ID NO: 6).

In some embodiments, the antisense oligonucleotide according to the invention, or the contiguous nucleotide sequence thereof comprises the sequence CACACACACACACACA (SEQ ID NO: 16) or ACACACACACACACAC (SEQ ID NO: 7).

In some embodiments, the antisense oligonucleotide according to the invention, or the contiguous nucleotide sequence thereof comprises the sequence (SEQ ID NO: 20).

In some embodiments, the antisense oligonucleotide according to the invention, or the contiguous nucleotide sequence thereof comprises the sequence (SEQ ID NO: 21).

In some embodiments, the antisense oligonucleotide according to the invention, or the contiguous nucleotide sequence thereof comprises the sequence (SEQ ID NO: 22).

In some embodiments, the antisense oligonucleotide according to the invention, or the contiguous nucleotide sequence thereof comprises the sequence (SEQ ID NO: 23).

In some embodiments, the antisense oligonucleotide according to the invention, or the contiguous nucleotide sequence thereof comprises the sequence (SEQ ID NO: 24).

In some embodiments, the antisense oligonucleotide according to the invention, or the contiguous nucleotide sequence thereof comprises the sequence (SEQ ID NO: 25).

In some embodiments, the antisense oligonucleotide according to the invention, or the contiguous nucleotide sequence thereof comprises the sequence (SEQ ID NO: 26).

In some embodiments, the antisense oligonucleotide according to the invention, or the contiguous nucleotide sequence thereof comprises the sequence (SEQ ID NO: 27).

In some embodiments, the antisense oligonucleotide according to the invention, or the contiguous nucleotide sequence thereof comprises the sequence (SEQ ID NO: 28).

In some embodiments, the antisense oligonucleotide according to the invention, or the contiguous nucleotide sequence thereof comprises the sequence (SEQ ID NO: 29).

In some embodiments, the antisense oligonucleotide according to the invention, or the contiguous nucleotide sequence thereof comprises the sequence (SEQ ID NO: 30).

In some embodiments, the antisense oligonucleotide according to the invention, or the contiguous nucleotide sequence thereof comprises the sequence (SEQ ID NO: 31).

In some embodiments, the antisense oligonucleotide according to the invention, or the contiguous nucleotide sequence thereof comprises the sequence (SEQ ID NO: 32).

In some embodiments, the antisense oligonucleotide according to the invention, or the contiguous nucleotide sequence thereof comprises the sequence (SEQ ID NO: 33).

In some embodiments, the antisense oligonucleotide according to the invention, or the contiguous nucleotide sequence thereof comprises the sequence (SEQ ID NO: 34).

In some embodiments, the antisense oligonucleotide according to the invention, or the contiguous nucleotide sequence thereof comprises the sequence (SEQ ID NO: 50).

In some embodiments, the antisense oligonucleotide according to the invention, or the contiguous nucleotide sequence thereof comprises the sequence (SEQ ID NO: 51).

In some embodiments, the antisense oligonucleotide according to the invention, or the contiguous nucleotide sequence thereof comprises the sequence (SEQ ID NO: 52).

In some embodiments, the antisense oligonucleotide according to the invention, or the contiguous nucleotide sequence thereof comprises the sequence (SEQ ID NO: 53).

In some embodiments, the antisense oligonucleotide according to the invention, or the contiguous nucleotide sequence thereof comprises the sequence (SEQ ID NO: 54).

In some embodiments, the antisense oligonucleotide according to the invention, or the contiguous nucleotide sequence thereof comprises the sequence (SEQ ID NO: 55).

In some embodiments, the antisense oligonucleotide according to the invention, or the contiguous nucleotide sequence thereof comprises the sequence (SEQ ID NO: 56).

In some embodiments, the antisense oligonucleotide according to the invention, or the contiguous nucleotide sequence thereof comprises the sequence (SEQ ID NO: 57).

In some embodiments, the antisense oligonucleotide according to the invention, or the contiguous nucleotide sequence thereof comprises the sequence (SEQ ID NO: 58).

In some embodiments, the antisense oligonucleotide according to the invention, or the contiguous nucleotide sequence thereof comprises the sequence (SEQ ID NO: 59).

In some embodiments, the antisense oligonucleotide according to the invention, or the contiguous nucleotide sequence thereof comprises the sequence (SEQ ID NO: 60).

In some embodiments, the antisense oligonucleotide according to the invention, or the contiguous nucleotide sequence thereof comprises the sequence (SEQ ID NO: 61).

In some embodiments, the antisense oligonucleotide according to the invention, or the contiguous nucleotide sequence thereof comprises the sequence (SEQ ID NO: 62).

In some embodiments, the antisense oligonucleotide according to the invention, or the contiguous nucleotide sequence thereof comprises the sequence (SEQ ID NO: 63).

In some embodiments, the antisense oligonucleotide according to the invention, or the contiguous nucleotide sequence thereof comprises the sequence (SEQ ID NO: 64).

In some embodiments, the antisense oligonucleotide according to the invention, or the contiguous nucleotide sequence thereof comprises the sequence (SEQ ID NO: 65).

In some embodiments, the antisense oligonucleotide according to the invention, or the contiguous nucleotide sequence thereof comprises the sequence (SEQ ID NO: 66).

In some embodiments, the antisense oligonucleotide according to the invention, or the contiguous nucleotide sequence thereof comprises the sequence (SEQ ID NO: 67).

In some embodiments, the antisense oligonucleotide according to the invention, or the contiguous nucleotide sequence thereof comprises the sequence (SEQ ID NO: 68).

In some embodiments, the antisense oligonucleotide according to the invention, or the contiguous nucleotide sequence thereof comprises the sequence (SEQ ID NO: 69).

In some embodiments, the antisense oligonucleotide according to the invention, or the contiguous nucleotide sequence thereof comprises the sequence (SEQ ID NO: 70).

In some embodiments, the antisense oligonucleotide according to the invention, or the contiguous nucleotide sequence thereof comprises the sequence (SEQ ID NO: 71).

In some embodiments, the antisense oligonucleotide according to the invention, or the contiguous nucleotide sequence thereof comprises the sequence (SEQ ID NO: 72).

In some embodiments, the antisense oligonucleotide according to the invention, or the contiguous nucleotide sequence thereof comprises the sequence (SEQ ID NO: 73).

In some embodiments, the antisense oligonucleotide according to the invention, or the contiguous nucleotide sequence thereof comprises the sequence (SEQ ID NO: 74).

In some embodiments, the antisense oligonucleotide according to the invention, or the contiguous nucleotide sequence thereof comprises the sequence (SEQ ID NO: 75).

In some embodiments, the antisense oligonucleotide according to the invention, or the contiguous nucleotide sequence thereof comprises the sequence (SEQ ID NO: 76).

In some embodiments, the antisense oligonucleotide according to the invention, or the contiguous nucleotide sequence thereof comprises the sequence (SEQ ID NO: 77).

In some embodiments, the antisense oligonucleotide according to the invention, or the contiguous nucleotide sequence thereof comprises the sequence (SEQ ID NO: 78).

In some embodiments, the antisense oligonucleotide according to the invention, or the contiguous nucleotide sequence thereof comprises the sequence (SEQ ID NO: 79).

In some embodiments, the antisense oligonucleotide according to the invention, or the contiguous nucleotide sequence thereof comprises the sequence (SEQ ID NO: 80).

In some embodiments, the antisense oligonucleotide according to the invention, or the contiguous nucleotide sequence thereof comprises the sequence (SEQ ID NO: 81).

In some embodiments, the antisense oligonucleotide according to the invention, or the contiguous nucleotide sequence thereof comprises the sequence (SEQ ID NO: 82).

In some embodiments, the antisense oligonucleotide according to the invention, or the contiguous nucleotide sequence thereof comprises the sequence (SEQ ID NO: 83).

In some embodiments, the antisense oligonucleotide according to the invention, or the contiguous nucleotide sequence thereof comprises the sequence (SEQ ID NO: 84).

In some embodiments, the antisense oligonucleotide according to the invention, or the contiguous nucleotide sequence thereof comprises the sequence (SEQ ID NO: 85).

In some embodiments, the antisense oligonucleotide according to the invention, or the contiguous nucleotide sequence thereof comprises the sequence (SEQ ID NO: 86).

In some embodiments, the antisense oligonucleotide according to the invention, or the contiguous nucleotide sequence thereof comprises the sequence (SEQ ID NO: 87).

In some embodiments, the antisense oligonucleotide according to the invention, or the contiguous nucleotide sequence thereof comprises the sequence (SEQ ID NO: 88).

In some embodiments, the antisense oligonucleotide according to the invention, or the contiguous nucleotide sequence thereof comprises the sequence (SEQ ID NO: 89).

In some embodiments, the antisense oligonucleotide according to the invention, or the contiguous nucleotide sequence thereof comprises the sequence (SEQ ID NO: 90).

In some embodiments, the antisense oligonucleotide according to the invention, or the contiguous nucleotide sequence thereof comprises the sequence (SEQ ID NO: 91).

In some embodiments, the antisense oligonucleotide according to the invention, or the contiguous nucleotide sequence thereof comprises the sequence (SEQ ID NO: 92).

In some embodiments, the antisense oligonucleotide according to the invention, or the contiguous nucleotide sequence thereof comprises the sequence (SEQ ID NO: 93).

In some embodiments, the antisense oligonucleotide according to the invention, or the contiguous nucleotide sequence thereof comprises the sequence (SEQ ID NO: 94).

In some embodiments, the antisense oligonucleotide according to the invention, or the contiguous nucleotide sequence thereof comprises the sequence (SEQ ID NO: 95).

In some embodiments, the antisense oligonucleotide according to the invention, or the contiguous nucleotide sequence thereof comprises the sequence (SEQ ID NO: 96).

In some embodiments, the antisense oligonucleotide according to the invention, or the contiguous nucleotide sequence thereof comprises the sequence (SEQ ID NO: 97).

In some embodiments, the antisense oligonucleotide according to the invention, or the contiguous nucleotide sequence thereof comprises the sequence (SEQ ID NO: 98).

In some embodiments, the antisense oligonucleotide according to the invention, or the contiguous nucleotide sequence thereof comprises the sequence (SEQ ID NO: 99).

In some embodiments, the antisense oligonucleotide according to the invention, or the contiguous nucleotide sequence thereof comprises the sequence (SEQ ID NO: 100).

In some embodiments, the antisense oligonucleotide according to the invention, or the contiguous nucleotide sequence thereof comprises the sequence (SEQ ID NO: 101).

In some embodiments, the antisense oligonucleotide according to the invention, or the contiguous nucleotide sequence thereof comprises the sequence (SEQ ID NO: 102).

In some embodiments, the antisense oligonucleotide according to the invention, or the contiguous nucleotide sequence thereof comprises the sequence (SEQ ID NO: 103).

In some embodiments, the antisense oligonucleotide according to the invention, or the contiguous nucleotide sequence thereof comprises the sequence (CA)n or (AC)n, wherein n is an integer 6-20, such as 7-9.

In some embodiments, the antisense oligonucleotide according to the invention, or the contiguous nucleotide sequence thereof comprises a sequence selected from the group consisting of SEQ ID NO: 1-18 or a sequence selected from SEQ ID NOs 1-34 and SEQ ID NOs 50-103.

In some embodiments, the antisense oligonucleotide according to the invention, or the contiguous nucleotide sequence thereof, consists of a sequence selected from the group consisting of SEQ ID NO: 1-18 or a sequence selected from SEQ ID NOs 1-34 and SEQ ID NOs 50-103.

The invention provides an antisense oligonucleotide selected from the group consisting of compound ID No #1-18, or a pharmaceutically acceptable salt thereof. The oligonucleotide of the invention may therefore be an antisense oligonucleotide selected from the group consisting of compound ID No #1-18, or a pharmaceutically acceptable salt thereof.

In some embodiments, the antisense oligonucleotide according to the invention has a length of at least 12 or at least 13 nucleotides.

In some embodiments, the contiguous nucleotide sequence of the antisense oligonucleotide is at least 12 nucleotides in length.

In some embodiments the antisense oligonucleotide or contiguous nucleotide sequence thereof according to any aspect of the invention has a length of at least 12 contiguous nucleotides.

In some embodiments the antisense oligonucleotide or contiguous nucleotide sequence thereof according to any aspect of the invention has a length of at least 13 contiguous nucleotides.

In some embodiments the antisense oligonucleotide or contiguous nucleotide sequence thereof according to any aspect of the invention has a length of at least 14 contiguous nucleotides.

In some embodiments the antisense oligonucleotide or contiguous nucleotide sequence thereof according to any aspect of the invention has a length of at least 15 contiguous nucleotides.

In some embodiments the antisense oligonucleotide or contiguous nucleotide sequence thereof according to any aspect of the invention has a length of at least 16 contiguous nucleotides.

In some embodiments the antisense oligonucleotide or contiguous nucleotide sequence thereof according to any aspect of the invention has a length of at least 17 contiguous nucleotides.

In some embodiments the antisense oligonucleotide or contiguous nucleotide sequence thereof according to any aspect of the invention has a length of at least 18 contiguous nucleotides.

In some embodiments the antisense oligonucleotide or contiguous nucleotide sequence thereof according to any aspect of the invention has a length of at least 19 contiguous nucleotides.

In some embodiments the antisense oligonucleotide or contiguous nucleotide sequence thereof according to any aspect of the invention has a length of at least 20 contiguous nucleotides.

In some embodiments the antisense oligonucleotide or contiguous nucleotide sequence thereof according to any aspect of the invention has a length of at least 21 contiguous nucleotides.

In some embodiments the antisense oligonucleotide or contiguous nucleotide sequence thereof according to any aspect of the invention has a length of at least 22 contiguous nucleotides.

In some embodiments the antisense oligonucleotide or contiguous nucleotide sequence thereof according to any aspect of the invention has a length of at least 23 contiguous nucleotides.

In some embodiments the antisense oligonucleotide or contiguous nucleotide sequence thereof according to any aspect of the invention has a length of at least 24 contiguous nucleotides.

In some embodiments the antisense oligonucleotide or contiguous nucleotide sequence thereof according to any aspect of the invention has a length of at least 25 contiguous nucleotides.

In some embodiments the antisense oligonucleotide or contiguous nucleotide sequence thereof according to any aspect of the invention has a length of at least 26 contiguous nucleotides.

In some embodiments the antisense oligonucleotide or contiguous nucleotide sequence thereof according to any aspect of the invention has a length of at least 27 contiguous nucleotides.

In some embodiments the antisense oligonucleotide or contiguous nucleotide sequence thereof according to any aspect of the invention has a length of at least 28 contiguous nucleotides.

In some embodiments the antisense oligonucleotide or contiguous nucleotide sequence thereof according to any aspect of the invention has a length of at least 29 contiguous nucleotides.

In some embodiments the antisense oligonucleotide or contiguous nucleotide sequence thereof according to any aspect of the invention has a length of at least 30 contiguous nucleotides.

In some embodiments the antisense oligonucleotide or contiguous nucleotide sequence thereof according to any aspect of the invention has a length of at least 31 contiguous nucleotides.

In some embodiments the antisense oligonucleotide or contiguous nucleotide sequence thereof according to any aspect of the invention has a length of at least 32 contiguous nucleotides.

In some embodiments, the antisense oligonucleotide according to the invention has a Gibbs free energy of the antisense oligonucleotide to a complementary target RNA, of lower than about −10 ΔG, such as lower than about −15 ΔG, such as lower than about −17 ΔG. Advantageously, the contiguous nucleotide sequence of such antisense oligonucleotides is at least 12, such as at least 13 nucleotides in length.

Advantageously the antisense oligonucleotide of the invention may comprise one or more modified nucleosides.

Advantageously the antisense oligonucleotide of the invention may comprise LNA nucleosides. LNA nucleotides within the contiguous nucleotide sequence are further advantageous. In some embodiments, the antisense oligonucleotide of the invention may comprise LNA nucleosides and non-LNA nucleosides, such as DNA nucleosides. In some embodiments, the antisense oligonucleotide, or contiguous nucleotide sequence thereof, may comprise LNA and DNA nucleosides. In some embodiments, all of the nucleosides of the antisense oligonucleotide, or contiguous nucleotide sequence thereof, are independently selected from LNA and DNA nucleosides. Advantageously, the length of contiguous DNA nucleosides present within the antisense oligonucleotide, or contiguous nucleotide sequence thereof, is limited so as to prevent RNaseH recruitment which results in target RNA degradation. Suitably the antisense oligonucleotide or contiguous nucleotide sequence thereof does not comprise more than four contiguous DNA nucleosides, more advantageously does not comprise more than 3 contiguous DNA nucleosides.

When used, advantageously, the antisense oligonucleotide according to the invention is capable of modulating the splicing of two or more TDP-43 target pre-mRNAs (target RNAs). By way of example, the two or more TDP-43 target RNAs may be independently selected from the group consisting of STMN2 pre-mRNA, ARHGAP32 pre-mRNA, SLC5A7 pre-mRNA, CERT1 pre-mRNA, CAMK2B pre-mRNA, KALRN pre-mRNA and UNC13A pre-mRNA.

In some embodiments the antisense oligonucleotide according to the invention is capable of modulating the splicing of two or more TDP-43 target pre-mRNAs (target RNAs).

In some embodiments the antisense oligonucleotide according to the invention is capable of modulating the splicing of three or more TDP-43 target pre-mRNAs (target RNAs).

In some embodiments the antisense oligonucleotide according to the invention is capable of modulating the splicing of four or more TDP-43 target pre-mRNAs (target RNAs).

In some embodiments the antisense oligonucleotide according to the invention is capable of modulating the splicing of two, three, four, five, six, seven, eight, nine, ten or more TDP-43 target pre-mRNAs (target RNAs).

In some embodiments, the antisense oligonucleotide is capable of enhancing the expression of STMN2 (wild type), and is further capable of enhancing the fidelity of pre-mRNA splicing of at least one, such as two or more pre-mRNAs selected from the group consisting of ARHGAP32 pre-mRNA, SLC5A7 pre-mRNA, CERT1 pre-mRNA, CAMK2B pre-mRNA, KALRN pre-mRNA and UNC13A pre-mRNA, when administered to a TDP-43 depleted cell. In some embodiments, the two or more selected pre-mRNAs are selected from the groups consisting of STMN2 and ARHGAP32; STMN2 and SLC5A7; STMN2 and CERT1; ARHGAP32 and SLC5A7; ARHGAP32 and CERT1; and SLC5A7 and CERT1.

In some embodiments, the antisense oligonucleotide is capable of enhancing the fidelity of pre-mRNA splicing of two or more pre-mRNAs selected from the group consisting of STMN2 pre-mRNA, ARHGAP32 pre-mRNA, SLC5A7 pre-mRNA, CERT1 pre-mRNA, CAMK2B pre-mRNA, KALRN pre-mRNA and UNC13A pre-mRNA, when administered to a TDP-43 depleted cell. In some embodiments, the two or more selected pre-mRNAs are selected from the groups consisting of STMN2 and ARHGAP32; STMN2 and SLC5A7; STMN2 and CERT1; ARHGAP32 and SLC5A7; ARHGAP32 and CERT1; and SLC5A7 and CERT1.

In some embodiments, the antisense oligonucleotide is capable of enhancing the fidelity of pre-mRNA splicing of three or more pre-mRNAs selected from the group consisting of STMN2 pre-mRNA, ARHGAP32 pre-mRNA, SLC5A7 pre-mRNA, CERT1 pre-mRNA, CAMK2B pre-mRNA, KALRN pre-mRNA and UNC13A pre-mRNA when administered to a TDP-43 depleted cell.

In some embodiments, the two or more selected pre-mRNAs are selected from the groups consisting of STMN2 and ARHGAP32; STMN2 and SLC5A7; and STMN2 and CERT1 pre-mRNA.

In some embodiments, the antisense oligonucleotide is capable of enhancing the fidelity of pre-mRNA splicing of STMN2 pre-mRNA, ARHGAP32 pre-mRNA, SLC5A7 pre-mRNA, CERT1 pre-mRNA, CAMK2B pre-mRNA, KALRN pre-mRNA and UNC13A pre-mRNA when administered to a TDP-43 depleted cell.

In some embodiments, the antisense oligonucleotide is capable of increasing the expression of STMN2 when administered to a TDP-43 depleted cell which is expressing STMN2 pre-mRNA.

In some embodiments, the antisense oligonucleotide is capable of decreasing the proportion of STMN2 mature mRNAs which comprise a cryptic exon (ce1) between exon 1 and exon 2, as compared to the wildtype STMN2 mature mRNA with a contiguous exon1/exon2 junction, when administered to a TDP-43 depleted cell which is expressing STMN2 pre-mRNA.

In some embodiments, the antisense oligonucleotide is capable of decreasing the level of aberrantly spliced ARHGAP32 mature mRNA, when administered to a TDP-43 depleted cell which is expressing ARHGAP32 pre-mRNA.

In some embodiments, the antisense oligonucleotide is capable of decreasing the level of aberrant exon inclusion in SLC5A7 mRNA transcript, when administered to a TDP-43 depleted cell which is expressing SLC5A7 pre-mRNA.

In some embodiments, the antisense oligonucleotide is capable of decreasing the level of aberrant exon inclusion in CERT1 mRNA transcript, when administered to a TDP-43 depleted cell which is expressing CERT1 pre-mRNA.

In some embodiments, the antisense oligonucleotide is capable of decreasing the level of aberrant exon inclusion in CAMK2B mRNA transcript, when administered to a TDP-43 depleted cell which is expressing CAMK2B pre-mRNA.

In some embodiments, the antisense oligonucleotide is capable of decreasing the level of aberrant exon inclusion in KALRN mRNA transcript, when administered to a TDP-43 depleted cell which is expressing KALRN pre-mRNA.

In some embodiments, the antisense oligonucleotide is capable of decreasing the level of aberrant exon inclusion in UNC13A mRNA transcript, when administered to a TDP-43 depleted cell which is expressing UNC13A pre-mRNA.

In some embodiments, the antisense oligonucleotide is capable of correcting the aberrant splicing of two or more of STMN2, CERT1, SLC5A7, ARHGAP32, CAMK2B, KALRN and UNC13A pre-mRNA in a TDP-43 depleted cell.

In some embodiments, the antisense oligonucleotide does not comprise a region of more than 3, or more than 4, contiguous DNA nucleosides.

In some embodiments, the antisense oligonucleotide, is not capable of mediating RNAseH cleavage.

In some embodiments, the antisense oligonucleotide is a morpholino antisense oligonucleotide.

In some embodiments, the antisense oligonucleotide, or contiguous nucleotide sequence thereof, comprises one or more affinity enhancing nucleosides, such as 2′sugar modified nucleosides which enhance the binding affinity between the antisense oligonucleotide and a complementary RNA molecule, for example and advantageously, to provide a lower Gibbs free energy, such as a Gibbs free energy lower −10, such as lower than −15.

In some embodiments, the antisense oligonucleotide or contiguous nucleotide sequence thereof, comprises one or more modified nucleosides, such as one or more affinity enhancing 2′ sugar modified nucleoside, such as a 2′ sugar modified nucleoside independently selected from the group consisting of: 2′-O-alkyl-RNA; 2′-O-methyl RNA (2′-OMe); 2′-alkoxy-RNA; 2′-O-methoxyethyl-RNA (2′-MOE); 2′-amino-DNA; 2′-fluro-RNA; 2′-fluoro-DNA; arabino nucleic acid (ANA); 2′-fluoro-DNA; locked nucleic acid (LNA), or a combination thereof.

In some embodiments the antisense oligonucleotide, or contiguous nucleotide sequence thereof, comprises 2′-O-methoxyethyl-RNA (2′-MOE) nucleosides. In some embodiments, all the nucleosides in the antisense oligonucleotide, or contiguous nucleotide sequence thereof, are 2′-O-methoxyethyl-RNA (2′-MOE) nucleosides, optionally linked by phosphorothioate internucleoside linkages.

In some embodiments the antisense oligonucleotide, or contiguous nucleotide sequence thereof, comprises 2′-O-methy nucleosides. In some embodiments, all the nucleosides in the antisense oligonucleotide, or contiguous nucleotide sequence thereof, are 2′-O-methy nucleosides, optionally linked by phosphorothioate internucleoside linkages.

In some embodiments, one or more of the modified nucleosides within the antisense oligonucleotide, or contiguous nucleotide sequence thereof, is a locked nucleic acid nucleoside (LNA), such as an LNA nucleoside selected from the group consisting of constrained ethyl nucleoside (cEt), or β-D-oxy-LNA.

In some embodiments, the contiguous nucleotide sequence of the antisense oligonucleotide comprises of nucleosides LNA nucleosides and DNA nucleosides, optionally linked by phosphorothioate internucleoside linkages.

In some embodiments, the antisense oligonucleotide or contiguous nucleotide sequence thereof is a mixmer or a totalmer.

In some embodiments, the antisense oligonucleotide or contiguous nucleotide sequence thereof comprises a sequence of nucleobases selected from the group consisting of SEQ ID Nos 1-18, a sequence selected from SEQ ID NOs 1-34 and SEQ ID NOs 50-103, or at least 8 contiguous nucleotides thereof.

In some embodiments, the antisense oligonucleotide or contiguous nucleotide sequence thereof comprises a sequence of nucleobases selected from the group consisting of SEQ ID Nos 1-18, a sequence selected from SEQ ID NOs 1-34 and SEQ ID NOs 50-103 or at least 9 contiguous nucleotides thereof.

In some embodiments, the antisense oligonucleotide or contiguous nucleotide sequence thereof comprises a sequence of nucleobases selected from the group consisting of SEQ ID Nos 1-18, a sequence selected from SEQ ID NOs 1-34 and SEQ ID NOs 50-103 or at least 10 contiguous nucleotides thereof.

In some embodiments, the antisense oligonucleotide or contiguous nucleotide sequence thereof comprises a sequence of nucleobases selected from the group consisting of SEQ ID Nos 1-18, a sequence selected from SEQ ID NOs 1-34 and SEQ ID NOs 50-103 or at least 11 contiguous nucleotides thereof.

In some embodiments, the antisense oligonucleotide or contiguous nucleotide sequence thereof comprises a sequence of nucleobases selected from the group consisting of SEQ ID Nos 1-18, a sequence selected from SEQ ID NOs 1-34 and SEQ ID NOs 50-103 or at least 12 contiguous nucleotides thereof.

In some embodiments, the antisense oligonucleotide or contiguous nucleotide sequence thereof comprises a sequence of nucleobases selected from the group consisting of SEQ ID Nos 1-18, a sequence selected from SEQ ID NOs 1-34 and SEQ ID NOs 50-103 or at least 13 contiguous nucleotides thereof.

In some embodiments, the antisense oligonucleotide or contiguous nucleotide sequence thereof comprises a sequence of nucleobases selected from the group consisting of SEQ ID Nos 1-18, a sequence selected from SEQ ID NOs 1-34 and SEQ ID NOs 50-103 or at least 14 contiguous nucleotides thereof.

In some embodiments, the antisense oligonucleotide or contiguous nucleotide sequence thereof comprises a sequence of nucleobases selected from the group consisting of SEQ ID Nos 1-18, a sequence selected from SEQ ID NOs 1-34 and SEQ ID NOs 50-103 or at least 15 contiguous nucleotides thereof.

In some embodiments, the antisense oligonucleotide or contiguous nucleotide sequence thereof comprises a sequence of nucleobases selected from the group consisting of SEQ ID Nos 1-18, a sequence selected from SEQ ID NOs 1-34 and SEQ ID NOs 50-103 or at least 16 contiguous nucleotides thereof.

In some embodiments, the antisense oligonucleotide or contiguous nucleotide sequence thereof comprises a sequence of nucleobases selected from the group consisting of SEQ ID Nos 1-18, a sequence selected from SEQ ID NOs 1-34 and SEQ ID NOs 50-103 or at least 17 contiguous nucleotides thereof.

In some embodiments, wherein the cytosine bases present in the antisense oligonucleotide or contiguous nucleotide sequence thereof are independently selected from the group consisting of cytosine and 5-methyl cytosine.

In some embodiments, the cytosine bases present in the antisense oligonucleotide or contiguous nucleotide sequence thereof are 5-methyl cytosine.

In some embodiments, the LNA cytosine bases present in the antisense oligonucleotide or contiguous nucleotide sequence thereof are LNA 5-methyl cytosine.

In some embodiments, the LNA cytosine bases present in the antisense oligonucleotide or contiguous nucleotide sequence thereof are LNA 5-methyl cytosine, and DNA cytosine bases are cytosine.

Advantageously, one or more of the internucleoside linkages positioned between the nucleosides on the contiguous nucleotide sequence are modified. In some embodiments, at least about 75%, at least about 80%, at least about 85%, at least about 90%, at least about 95%, or about 100% of the internucleoside linkages positioned between the nucleosides on the contiguous nucleotide sequence are modified.

In some embodiments, one or more, or all of the modified internucleoside linkages are phosphorothioate linkage. In some embodiments, one or more, or all of the linkages within the contiguous nucleotide sequence, are phosphorothioate linkages.

In some embodiments, all of the internucleoside linkages present in the contiguous nucleotide sequence are phosphorothioate internucleoside linkages.

In some embodiments, all of the internucleoside linkages present in the antisense oligonucleotide are phosphorothioate internucleoside linkages.

In some embodiments, the length of the contiguous nucleotide sequence is 8-32 nucleotides.

In some embodiments, the length of the contiguous nucleotide sequence is 8-20 nucleotides.

In some embodiments, the length of the contiguous nucleotide sequence is 12-18 nucleotides.

In some embodiments, the length of the contiguous nucleotide sequence is 13-18 nucleotides.

In some embodiments, the length of the contiguous nucleotide sequence is 14-18 nucleotides.

In some embodiments, the length of the contiguous nucleotide sequence is 10-32 nucleotides.

In some embodiments, the length of the contiguous nucleotide sequence is 20-32 nucleotides.

In some embodiments, the length of the contiguous nucleotide sequence is 21-32 nucleotides.

In some embodiments, the length of the contiguous nucleotide sequence is 22-32 nucleotides.

In some embodiments, the length of the contiguous nucleotide sequence is 23-32 nucleotides.

In some embodiments, the length of the contiguous nucleotide sequence is 24-32 nucleotides.

In some embodiments, the length of the contiguous nucleotide sequence is 25-32 nucleotides.

In some embodiments, the length of the contiguous nucleotide sequence is 26-32 nucleotides.

In some embodiments, the length of the contiguous nucleotide sequence is 27-32 nucleotides.

In some embodiments, the antisense oligonucleotide consists of the contiguous nucleotide sequence.

In some embodiments, the antisense oligonucleotide comprises or consists of an oligonucleotide selected from the group consisting of 16,1, 8,3, 18,3, 18,4, 16,2, 18,2, 7,2, 15,2, 16,3, 15,3, 8,1, 8,2, 7,3, 6,4, 6,3, 18,1, 7,1, 14,2, 6,2, 14,4, 15,4, 10,1, and 15,5.

The invention provides for an antisense oligonucleotide selected from the group consisting of (compound ID #) 16,1, 8,3, 18,3, 18,4, 16,2, 18,2, 7,2, 15,2, 16,3, 15,3, 8,1, 8,2, 7,3, 6,4, 6,3, 18,1, 7,1, 14,2, 6,2, 14,4, 15,4, 10,1, and 15,5.

The oligonucleotide of the invention may comprise one or more conjugate groups, i.e. the oligonucleotide may be an antisense oligonucleotide conjugate.

The invention provides for a conjugate comprising the oligonucleotide according to the invention, and at least one conjugate moiety covalently attached to said oligonucleotide.

The invention provides for a salt, such as a pharmaceutically acceptable salt of the antisense oligonucleotide, or the conjugate, of the invention, such as a sodium salt or a potassium salt.

The invention provides for a pharmaceutical composition comprising the oligonucleotide of claim, or the conjugate of the invention, and a pharmaceutically acceptable diluent, solvent, carrier, salt and/or adjuvant.

The invention provides for a pharmaceutical composition comprising the oligonucleotide of the invention, or the conjugate of the invention, and a pharmaceutically acceptable diluent or solvent, and a cation. The cation may for example be a sodium cation or a potassium cation. The diluent/solvent maybe water.

The invention provides for a method, such as an in vivo or in vitro method, for enhancing TDP-43 functionality in a cell which is expressing aberrant or depleted levels of TDP-43, said method comprising administering an oligonucleotide of the invention or the conjugate according to the invention, or the salt or composition according to the invention, in an effective amount to said cell.

The invention provides for a method for treating or preventing a TDP-43 pathology in a subject comprising administering a therapeutically or prophylactically effective amount of an oligonucleotide of the invention or the conjugate according to the invention, or the salt or composition according to the invention, to a subject suffering from or susceptible to the TDP-43 pathology.

The invention provides for an oligonucleotide of the invention or the conjugate according to the invention, or the salt or composition according to the invention, for use as a medicament.

The invention provides for an oligonucleotide of the invention or the conjugate according to the invention, or the salt or composition according to the invention, for use in the treatment of a TDP-43 pathology.

The invention provides for the use of the oligonucleotide of the invention or the conjugate according to the invention, or the salt or composition according to the invention, for the preparation of a medicament for treatment or prevention of a TDP-43 pathology.

The invention provides for the use or method of the invention, wherein the TDP-43 pathology is a neurological disorder selected from the group consisting of amyotrophic lateral sclerosis (ALS), frontotemporal lobar degeneration (FTLD), Progressive supranuclear palsy (PSP), Primary lateral sclerosis, Progressive muscular atrophy, Alzheimer's disease, Parkinson's disease, Autism, Hippocampal sclerosis dementia, Down syndrome, Huntington's disease, polyglutamine diseases, such as spinocerebellar ataxia 3, myopathies and Chronic Traumatic Encephalopathy.

In some embodiments, the TDP-43 pathology is a neurological disorder selected from the group consisting of amyotrophic lateral sclerosis (ALS), frontotemporal lobar degeneration (FTLD).

The invention provides pharmaceutical compositions comprising the antisense oligonucleotide of the invention and a pharmaceutically acceptable diluents, carriers, salts and/or adjuvants.

The invention provides for a pharmaceutically acceptable salt of the antisense oligonucleotide of the invention. In some embodiments, the pharmaceutically acceptable salt is a sodium salt, a potassium salt or an ammonium salt.

The invention provides for a pharmaceutical solution of the oligonucleotide of the invention, wherein the pharmaceutical solution comprises the oligonucleotide of the invention and a pharmaceutically acceptable solvent, such as phosphate buffered saline.

The invention provides for the oligonucleotide of the invention in solid powdered form, such as in the form of a lyophilized powder.

Typically, the antisense oligonucleotide of the invention comprises a contiguous nucleotide sequence of at least 8 or at least 10 nucleotides in length, such as 10-32, 15-32, 20-32, 21-32, 22-32, 23-32, 24-32, 25-32, 26-32, 27-32 or 10-20 nucleotides in length, wherein the contiguous nucleotide sequence is at least 75% complementary, such as at least 90% complementary to or fully complementary to a TDP-43 RNA binding sequence. In some embodiments, all of the nucleosides of the antisense oligonucleotide form the contiguous nucleotide sequence.

In some embodiments, the antisense oligonucleotide of the invention is capable of modulating the splicing of the at least two human pre-mRNAs. For example, the splicing of human STMN2, CERT1, SLC5A7, ARHGAP32, CAMK2B, KALRN and UNC13A pre-mRNAs are dependent upon TDP-43 binding, as illustrated in the examples.

In a further aspect the invention provides methods for treating or preventing neurodegenerative disease such as amyotrophic lateral sclerosis (ALS), comprising administering a therapeutically or prophylactically effective amount of the oligonucleotide of the invention to a subject suffering from or susceptible to the disease.

In a further aspect the oligonucleotide or composition of the invention is used for the treatment or prevention of a neurodegenerative disease as neurodegenerative disorders characterized by TDP-43 pathology or mis-localization of TDP-43 from the nucleus, such as amyotrophic lateral sclerosis (ALS).

### BRIEF DESCRIPTION OF FIGURES

FIG. 1—Correlation between oligonucleotide length and the efficacy in correction of STMN2 mRNA processing in TDP-43 depleted cells, as measured by the ratio of aberrant STMN2 mRNA vs. WT STMN2 mRNA expression level. Of note the more effective compounds were at least 12 nucleotide in length.

FIG. 2—Correlation between oligonucleotide Gibbs Free energy (ΔG) and the efficacy in correction of STMN2 mRNA processing in TDP-43 depleted cells, as measured by the ratio of aberrant STMN2 mRNA vs. WT STMN2 mRNA expression level. Of note the more effective compounds had a Gibbs free energy of at least −10 ΔG.

FIG. 3—Lack of a correlation between oligonucleotide melting temperature (predicted Tm) and the efficacy in correction of STMN2 mRNA processing in TDP-43 depleted cells, as measured by the ratio of aberrant STMN2 mRNA vs. WT STMN2 mRNA expression level.

FIG. 4—TARDBP knock down in Glutaneurons upon treatment with compound A. Relative expression of TARDBP in untreated Glutaneurons, Glutaneurons treated with compound A, compound A+B and compound A+C.

FIGS. 5A and 5B—STMN2 wild type expression in Glutaneurons upon treatment with compound A. (FIG. 5A) Graphical user interface illustrating normalized reads mapped to the 5′ end of the STMN2 gene of untreated Glutaneurons, Glutaneurons treated with compound A, compound A+B and compound A+C. Grey rectangle highlights the aligned reads that usage of alternative splice acceptor site. (FIG. 5B) Relative expression of wild type STMN2 transcript in untreated Glutaneurons, Glutaneurons treated with compound A, compound A+B and compound A+C.

FIG. 6—Illustration of an example of the aberrant splicing event which results in the cryptic exon inclusion in STMN2 mRNA in TDP-43 depleted cells. The figure illustrates the nucleotide sequence of the STMN2 cryptic exon containing mRNA transcript (SEQ ID NO: 120) and the amino acid sequence (SEQ ID NO: 124) is the resulting truncated stathmin-2 protein (referred to herein as STMN2 protein). The shaded bold nucleotide sequence is the STMN2 exon 1 sequence and the underlined nucleotide sequence is the cryptic exon sequence which originates from intron 1, and the cryptic splice site is indicated by a [symbol] symbol. The ATG start codon, TAG stop codon and polyadenylation signal are highlighted.

FIGS. 7A and 7B—ARHGAP32 expression in Glutaneurons upon treatment with compound A, B and C. (FIG. 7A) Graphical user interface illustrating normalized reads aligned to hg38 (partial zoom to ARHGAP32) of untreated Glutaneurons, Glutaneurons treated with compound A, compound A+B and compound A+C. Grey rectangle illustrates the aligned reads showing usage of alternative splice acceptor site. (FIG. 7B) Relative expression of the ARHGAP32 isoform with inclusion of alternative last exon (position 200153 in ENSG00000134909) in untreated Glutaneurons, Glutaneurons treated with compound A, compound A+B and compound A+C.

FIG. 8—Nucleotide sequence of the novel last exon of the ARHGAP32 gene (SEQ ID NO: 121), the novel cryptic splice site is indicated by a \hat{ } symbol, with the non-bold test representing the cryptic exon which is enriched in the TDP-43 depleted cells. The inclusion of the cryptic exon results in the inclusion of two polyadenylation sites (shaded text) within the cryptic exon, resulting in the expression of a truncated version of the Rho GTPase-activating protein 32, protein encoded by ARHGAP32. The italicized texts is the GT rich RNA binding protein binding site.

FIGS. 9A and 9B—SLC5A7 expression in Glutaneurons upon treatment with compound A, B and C. (FIG. 9A) Graphical user interface illustrating normalized reads aligned to SLC5A7 of untreated Glutaneurons, Glutaneurons treated with compound A, compound A+B and compound A+C. Grey rectangle illustrates the aligned reads showing inclusion of the alternative exon. (FIG. 9B) Relative expression of the SLC5A7 isoform with inclusion of alternative exon in untreated Glutaneurons, Glutaneurons treated with compound A, compound A+B and compound A+C.

FIG. 10—Nucleotide sequence of the novel exon to be included from the SLC5A7 gene (SEQ ID NO: 122). The novel cryptic splice site is indicated by a [symbol] symbol, with the non-bold underlined text representing the cryptic exon which is enriched in the TDP-43 depleted cells. The italicized texts is the GT rich RNA binding protein binding site (TDP-43 binding site).

FIGS. 11A and 11B—CERT1 expression in Glutaneurons upon treatment with compound A, B and C. (FIG. 11A) Graphical user interface illustrating normalized reads aligned to CERT1 of untreated Glutaneurons, Glutaneurons treated with compound A, compound A+B and compound A+C. Grey rectangle illustrates the aligned reads showing inclusion of the alternative exon. (FIG. 11B) Relative expression of the CERT1 isoform with inclusion of alternative exon in untreated Glutaneurons, Glutaneurons treated with compound A, compound A+B and compound A+C.

FIG. 12—Nucleotide sequence of the novel in frame exon included from the CERT1 gene in TDP-43 depleted cells (SEQ ID NO: 123). The new exon of CERT1 is shown in the underlined text, and the TDP-43 binding sites is in italics.

FIG. 13—Venn diagram. Number of transcripts that shows alternative splicing with a p-value below 0.01 and which shows more than a two-fold change compared to the control. The control for compound A is untreated Glutaneurons, whereas the control for compound A+B, and A+C is the TDP-43 knock down (compound A). In the TDP-43 depleted cells (treated with compound A only), there were a total of 925 transcripts which were alternatively spliced due to TDP-43 depletion. When the TDP-43 depleted cells were subsequently treated with a STMN2 targeting oligonucleotide (compound B), there were 114 transcripts which were alternatively spliced as compared to the compound A treated cells. When the TDP-43 depleted cells were subsequently treated with a RNA protein binding site targeting oligonucleotide of the invention (compound C), there were 351 transcripts which were alternatively spliced as compared to the compound A treated cells. This indicates that the compounds of the invention are more effective in modulating global RNA splicing as compared to the STMN2 targeting oligonucleotides.

FIGS. 14A and 14B—FIG. 14A shows a graphical illustration showing mapping of normalized reads mapped to CAMK2B gene. Glutaneurons treated with compound A or untreated cells (PBS) as control, followed mRNA isolation and NGS. The position of the novel exons within the CAMK2B gene upon loss of TDP43 protein is illustrated with a horizontal grey rectangle (only exons surrounding the novel exon of CAMK2B is shown). The novel splice donor site is shown with a grey arrow. The CAMK2B pre-mRNA is transcribe from the minus strand. FIG. 14B shows the sequence (SEQ ID NO: 125) of the novel CAMK2B exon that is observed upon loss of TDP43 protein. In bold is the canonical exon observed in Glutaneurons. Underlined is the novel exon, and in italic is shown the first of many stop codons. \hat{ } shows the canonical splice sites and v shows the position of the novel splice acceptor site.

FIGS. 15A-15D—FIG. 15A shows Graphical illustration showing mapping of normalized reads mapped to KALRN gene. Glutaneurons treated with compound A or untreated cells (PBS) as control, followed mRNA isolation and NGS. The position of the novel exons within the KALRN gene upon loss of TDP43 protein is illustrated in the grey rectangle (only exons surrounding the novel exon of KALRN is shown). FIG. 15B shows a graphical illustration zooming in on the mapping of normalized reads mapped to KALRN gene containing the aberrantly spliced exons. Glutaneurons treated with compound A or untreated cells (PBS) as control, followed mRNA isolation and NGS. The position of the novel exons within the KALRN gene upon loss of TDP43 protein is illustrated in the horizontal grey rectangles. Arrows indicates the splice sites used upon loss of TDP43 protein. FIG. 15C shows the sequence (SEQ ID NO: 126) of the novel KALRN exon that is observed upon loss of TDP43 protein. In bold is the novel exon observed in Glutaneurons. In italic is shown the first of many stop codons within the coding sequence. [symbol] shows the position of the novel splice sites. FIG. 15D shows ng the sequence (SEQ ID NO: 127) of the novel KALRN exon that is observed upon loss of TDP43 protein. In bold is the novel exon observed in Glutaneurons. In italic is shown the first of many stop codons within the coding sequence. [symbol] shows the position of the novel splice sites.

FIGS. 16A-16C—FIG. 16A shows Illustration of the aberrant splicing event which results in the inclusion of either the cryptic exon of 128 nucleotides (A) (SEQ ID NO: 128), or 178 nucleotides (B) (SEQ ID NO: 129). In bold is the nucleotide sequence of the two cryptic exons of UNC13A. The splice sites are illustrated by [symbol] and the intronic sequence 5′ and 3′ to the exon is shown in regular capital letters. Shown is the reverse complement sequence as UNC13A gene is positioned don the minus strand of chromosome 19. FIG. 16B shows a graphical illustration showing mapping of normalized reads mapped to UNC13A gene. Glutaneurons treated with compound A or untreated cells (PBS) as control, followed mRNA isolation and NGS. The position of the novel exons within the UNC13A gene upon loss of TDP43 protein is illustrated in the grey rectangle (only exons surrounding the novel exon of UNC13A is shown). FIG. 16C shows Graphical illustration showing the novel exons within the UNC13A gene upon loss of TDP43 protein. Glutaneurons treated with compound A or untreated cells (PBS) as control, followed mRNA isolation and NGS. The arrows illustrate the novel splice sites.

### DEFINITIONS

### RNA Binding Protein Mimics and TDP-43 Mimics

TDP-43 is the TAR RNA/DNA binding protein which in humans is encoded on the human chromosome 1: 11,012,653-11,022,858 forward strand (Gene ENSG00000120948, Chr 1: 11,012,344-11,025,739, example of a typical TDP-43 transcript=ENST00000439080.6), and is widely involved in RNA splicing, stability and metabolism. In healthy cells the TDP-43 protein is located in the nucleus, however in several neurodegenerative diseases, dysfunction TDP-43 aggregates form in the cytoplasm (often associated with hyper-phosphorylated and ubiquitinated TDP-43).

TDP-43 is an example of a RNA binding protein which binds to GU repeats in numerous independent RNA transcripts. The interaction of RNA binding proteins, such as TDP-43 with the population of numerous RNA transcripts has a profound effect on the biology of the RNA transcripts, such as the splicing on pre-mRNA, RNA stability, RNA accumulation, and therefore provides a mechanism for effecting the expression of populations of independent RNAs in a cell. This is of particular relevance in the case of TDP-43 depletion, where the loss of RNA binding of functional TDP-43 is closely associated with neurodegeneration.

The present invention provides antisense oligonucleotides which are complementary to GU rich regions on multiple RNA transcripts, such as conserved TDP-43 binding sites on a population of pre-mRNA transcripts. As is illustrated in the examples, the administration of the oligonucleotides of the invention can restore the functional processing of multiple independent RNA transcripts, which are otherwise aberrantly processed in the depletion or absence of the RNA binding protein, for example TDP-43. The antisense oligonucleotides of the invention, otherwise referred to as compounds of the invention, may therefore be referred to as RNA binding protein mimics, or TDP-43 mimics, in that they restore the functionality of the RNA binding protein, such as TDP-43, in regulating the RNA biology of multiple RNA transcripts.

By way of example, the RNA binding protein functionality, such as TDP-43 functionality, which is restored or enhanced by the use of the compounds of the invention (e.g. in TDP-43 depleted cells), is the expression, processing, e.g. splicing events of pre-mRNA transcripts, resulting in a restoration of functional gene expression which is otherwise dysregulated in cells with reduced level of functional TDP-43 (referred to herein as TDP-43 depleted cells). This may result in an enhanced gene expression or an enhanced quality of gene expression.

Advantageously, the compounds of the invention are capable of mimicking the functional TDP-43, and restoring the nuclear function of TDP-43 in the expression of one or more TDP-43 target RNAs, and thereby restore the functional phenotype of the TDP-43 target RNA(s).

It will be understood that other RNA binding proteins may bind to the TDP-43 binding sites, and as such the TDP-43 mimics referred to herein are oligonucleotides, which are complementary to the TDP-43 binding sites of one or more RNA targets, such as multiple nucleic acid targets (i.e. RNA targets which are described from distinct genetic loci), and which are capable of restoring the expression of the normal (wildtype)

As reported in Arnold et al., PNAS 2013, some TDP-43 pathologies are associated with certain TDP-43 mutations, and these may not necessarily be associated with TDP-43 cytoplasmic depletion. In the context of the present invention the normal function of TDP-43 may be genetically disrupted, and this is therefore also considered a potential source of depletion or normal TDP-43, a phenotype which can be addressed using the TDP-43 mimics of the present invention.

### Examples of TDP-43 RNA Targets

As is illustrated in FIG. 13, the depletion of TDP-43 in neuronal cells results in a profound alteration in the RNA processing of a large population of RNA transcripts in the cell—In this example, 749 RNA transcripts illustrated alternative RNA processing as determined by RNA sequencing, after depletion of TDP-43.

The examples illustrate seven of these TDP-43 target RNAs: STMN2, ARHGAP32, SLC5A7, CERT1, CAMK2B, KALRN and UNC13A.

Arnold et al., PNAS 2013 110 E736-745 identifies widespread aberrations of pre-mRNA splicing of TDP-43 binding RNAs in TDP-43 depleted cells (TDP-43 ASO depleted mice), and illustrates the identification of indicative TDP-43 regulated splicing events using micro-array analysis. RNAs identified by Arnold et al., whose splicing is regulated by TDP-43 include Eif4h, Taf1b, Kcnip2, (TDP-43 mutation dependent), Sort1, Kcnd3, Ahi1, Atxn2, Ctnnd (dose dependent).

STMN2 (Klim et al., Nat Neurosci. 2019 February; 22 (2): 167-179)—TDP-43 depletion in neuronal cells (e.g. in ALS) results in a mis-splicing of the STMN2 transcript. STMN2 encodes a microtubule regulator, whose expression declines after TDP-43 knockdown and TDP-43 mis-localization as well as in patient-specific motor neurons and postmortem patient spinal cord.

Post-translational stabilization of STMN2 rescued neurite outgrowth and axon regeneration deficits induced by TDP-43 depletion. TDP-43 depletion results in the incorporation of a cryptic intron between exon1 and 2 of STMN2. WO2019/241648 discloses fully MOE modified phosphorothioate ASOs which are used to suppress the mis-splicing of STMN2.

The above-mentioned transcripts and the associated TDP-43 depletion splicing events may be used to assay for restoration of TDP-43 functionality using the compounds of the invention.

### TDP-43 Pathologies

A TDP-43 pathology is a disease which is associated with reduced or aberrant expression of TDP-43, often associated by an increase in cytoplasmic TDP-43, particularly hyper-phosphorylated and ubiquitinated TDP-43.

TDP-43 depletion is indicated in a range of diseases, referred to as TDP-43 pathologies, and include for example such as amyotrophic lateral sclerosis (ALS), frontotemporal lobar degeneration (FTLD), Progressive supranuclear palsy (PSP), Primary lateral sclerosis, Progressive muscular atrophy Alzheimer's disease, Parkinson's disease, Autism, Hippocampal sclerosis dementia, Down syndrome, Huntington's disease, polyglutamine diseases, such as spinocerebellar ataxia 3, myopathies and Chronic Traumatic Encephalopathy.

### Cells Which are Depleted in TDP-43

Cells which are depleted in TDP-43 refer to cells where the functional level of TDP-43 is reduced. It will be understood that in TDP-43 pathologies, aberrant TDP-43 expression results in accumulation of dysfunctional cytoplasmic TDP-43, and a reduction in the functional nuclear TDP-43 level—as such cells which are depleted in TDP-43 may be characterized by a reduction in the functional level of TDP-43, and may therefore be associated with an increase in the level of dysfunctional TDP-43. For in vitro assessment, TDP-43 depletion may be engineered for example by genetic engineering approaches (e.g. CRISPR/CAS9), or as illustrated in the examples, by use of an antisense oligonucleotide inhibitor of TDP-43 (illustrated by a gapmer oligonucleotide targeting the human TDP-43 transcript).

In some embodiments the cell which is depleted in TDP-43 is a neuronal cell.

### Sequences Complementary to TDP-43 Binding Sites

TDP-43 binding sites are characterized by poly GU motifs (see the A database of RNA binding attract.cnic.es/results/e9f29380-8921-406e-84a8-27ce9b9398b4 #) for example, and suitably for antisense oligonucleotide intervention may comprise a motif of (GU)n or (UG)n, where n is at least 3 or preferably at least 4. In some embodiments n is 4, 5, 6, 7, 8, 9 or 10.

In some embodiments the TDP-43 binding site may comprise a sequence selected from the group consisting of (UG)n, (GU)n, wherein n is 4-20, UGUGUGUG, UGUGUGUGU, UGUGUGUGUG, UGUGUGUGUGU, UGUGUGUGUGUG, UGUGUGUGUGUGU, GUGUGUGU, GUGUGUGUG, GUGUGUGUGU, GUGUGUGUGUG, GUGUGUGUGUGU (SEQ ID NO: 42), GUGUGUGUGUGUG, and GUGAAUGA.

In some embodiments, the TDP-43 binding site may comprise a sequence selected from the group consisting of GUGAAUGA, GUUGUGC, UGUGUGUGUGUG (SEQ ID NO: 35), GAAUGG, UGUGUGUG, GAAUGA, UGUGUG, GUUGUUC, and GUUUUGC. In some embodiments, the TDP-43 binding site may comprise the sequence UGUGUGUGUGUGUG (SEQ ID NO: 46).

In some embodiments the oligonucleotide of the present invention may comprise a sequence which is complementary to, such as fully complementary to one or more sequences selected from the group consisting of (UG)n, (GU)n, wherein n is 4-20, UGUGUGUG, UGUGUGUGU, UGUGUGUGUG, UGUGUGUGUGU, UGUGUGUGUGUG, UGUGUGUGUGUGU, GUGUGUGU, GUGUGUGUG, GUGUGUGUGU, GUGUGUGUGUG, GUGUGUGUGUGU, GUGUGUGUGUGUG, and GUGAAUGA.

The oligonucleotide of the present invention may comprise a sequence which is complementary to, such as fully complementary to, the TDP-43 binding site sequence, such as one or more sequences, selected from the group consisting of (GU)n, (UG)n, GUGAAUGA, GUUGUGC, GAAUGG, UGUGUGUG, GAAUGA, UGUGUG, UGUGUGUGUGUG (SEQ ID NO: 35), GUUGUUC, and GUUUUGC.

### Oligonucleotide

The term “oligonucleotide” as used herein is defined as it is generally understood by the skilled person as a molecule comprising two or more covalently linked nucleosides. Such covalently bound nucleosides may also be referred to as nucleic acid molecules or oligomers. Oligonucleotides are commonly made in the laboratory by solid-phase chemical synthesis followed by purification and isolation. When referring to a sequence of the oligonucleotide, reference is made to the sequence or order of nucleobase moieties, or modifications thereof, of the covalently linked nucleotides or nucleosides. The oligonucleotides of the invention are man-made, and are chemically synthesized, and are typically purified or isolated.

The oligonucleotides of the invention may comprise one or more modified nucleosides such as 2′ sugar modified nucleosides. The oligonucleotides of the invention may comprise one or more modified internucleoside linkages, such as one or more phosphorothioate internucleoside linkages.

### Antisense Oligonucleotides

The term “antisense oligonucleotide” as used herein is defined as an oligonucleotide capable of modulating expression of a target gene by hybridizing to a target nucleic acid, in particular to a contiguous sequence on a target nucleic acid. Antisense oligonucleotides are not essentially double stranded and are therefore not siRNAs or shRNAs. The antisense oligonucleotides of the present invention may be single stranded. It is understood that single stranded oligonucleotides of the present invention can form hairpins or intermolecular duplex structures (duplex between two molecules of the same oligonucleotide), as long as the degree of intra or inter self-complementarity is less than approximately 50% across of the full length of the oligonucleotide.

In some embodiments, the single stranded antisense oligonucleotides of the invention may not contain RNA nucleosides.

Advantageously, the antisense oligonucleotides of the invention comprise one or more modified nucleosides or nucleotides, such as 2′ sugar modified nucleosides. Furthermore, in some antisense oligonucleotides of the invention, it may be advantageous that the nucleosides which are not modified are DNA nucleosides.

### Contiguous Nucleotide Sequence

The term “contiguous nucleotide sequence” refers to the region of the oligonucleotide which is complementary to the target nucleic acid. The term is used interchangeably herein with the term “contiguous nucleobase sequence” and the term “oligonucleotide motif sequence”. In some embodiments all the nucleosides of the antisense oligonucleotide constitute the contiguous nucleotide sequence. The contiguous nucleotide sequence is the sequence of nucleotides in the oligonucleotide of the invention which are complementary to, and in some instances fully complementary to, the target nucleic acid or target sequence.

In some embodiments the antisense oligonucleotide comprises the contiguous nucleotide sequence, and may optionally comprise further nucleotide(s), for example a nucleotide linker region which may be used to attach a functional group (e.g. a conjugate group) to the contiguous nucleotide sequence. The nucleotide linker region may or may not be complementary to the target nucleic acid. It is understood that the contiguous nucleotide sequence of the oligonucleotide cannot be longer than the oligonucleotide as such and that the oligonucleotide cannot be shorter than the contiguous nucleotide sequence.

### Nucleotides and Nucleosides

Nucleotides and nucleosides are the building blocks of oligonucleotides and polynucleotides, and for the purposes of the present invention include both naturally occurring and non-naturally occurring nucleotides and nucleosides. In nature, nucleotides, such as DNA and RNA nucleotides comprise a ribose sugar moiety, a nucleobase moiety and one or more phosphate groups (which is absent in nucleosides). Nucleosides and nucleotides may also interchangeably be referred to as “units” or “monomers”.

### Modified Nucleoside

The term “modified nucleoside” or “nucleoside modification” as used herein refers to nucleosides modified as compared to the equivalent DNA or RNA nucleoside by the introduction of one or more modifications of the sugar moiety or the (nucleo) base moiety. Advantageously, one or more of the modified nucleosides of the antisense oligonucleotides of the invention comprise a modified sugar moiety. The term modified nucleoside may also be used herein interchangeably with the term “nucleoside analogue” or modified “units” or modified “monomers”. Nucleosides with an unmodified DNA or RNA sugar moiety are termed DNA or RNA nucleosides herein. Nucleosides with modifications in the base region of the DNA or RNA nucleoside are still generally termed DNA or RNA if they allow Watson Crick base pairing. Exemplary modified nucleosides which may be used in the compounds of the invention include LNA, 2′-O-MOE and morpholino nucleoside analogues.

### Modified Internucleoside Linkage

The term “modified internucleoside linkage” is defined as generally understood by the skilled person as linkages other than phosphodiester (PO) linkages, that covalently couple two nucleosides together. The oligonucleotides of the invention may therefore comprise one or more modified internucleoside linkages such as one or more phosphorothioate internucleoside linkage.

In some embodiments at least 50% of the internucleoside linkages in the oligonucleotide, or contiguous nucleotide sequence thereof, are phosphorothioate, such as at least 60%, such as at least 70%, such as at least 75%, such as at least 80% or such as at least 90% or more of the internucleoside linkages in the oligonucleotide, or contiguous nucleotide sequence thereof, are phosphorothioate. In some embodiments all of the internucleoside linkages of the oligonucleotide, or contiguous nucleotide sequence thereof, are phosphorothioate.

Advantageously, all the internucleoside linkages of the contiguous nucleotide sequence of the oligonucleotide are phosphorothioate, or all the internucleoside linkages of the oligonucleotide are phosphorothioate linkages.

### Nucleobase

The term nucleobase includes the purine (e.g. adenine and guanine) and pyrimidine (e.g. uracil, thymine and cytosine) moiety present in nucleosides and nucleotides which form hydrogen bonds in nucleic acid hybridization. In the context of the present invention the term nucleobase also encompasses modified nucleobases which may differ from naturally occurring nucleobases, but which are functional during nucleic acid hybridization. In this context “nucleobase” refers to both naturally occurring nucleobases such as adenine, guanine, cytosine, thymidine, uracil, xanthine and hypoxanthine, as well as non-naturally occurring variants. Such variants are for example described in Hirao et al (2012) Accounts of Chemical Research 45:2055 and Bergstrom (2009) Current Protocols in Nucleic Acid Chemistry Suppl. 37 1.4.1.

In some embodiments the nucleobase moiety is modified by changing the purine or pyrimidine into a modified purine or pyrimidine, such as substituted purine or substituted pyrimidine, such as a nucleobase selected from isocytosine, pseudoisocytosine, 5-methyl cytosine, 5-thiozolo-cytosine, 5-propynyl-cytosine, 5-propynyl-uracil, 5-bromouracil 5-thiazolo-uracil, 2-thio-uracil, 2′thio-thymine, inosine, diaminopurine, 6-aminopurine, 2-aminopurine, 2,6-diaminopurine and 2-chloro-6-aminopurine, 5′ nitroindole.

The nucleobase moieties may be indicated by the letter code for each corresponding nucleobase, e.g. A, T, G, C or U, wherein each letter may optionally include modified nucleobases of equivalent function. For example, in the exemplified oligonucleotides, the nucleobase moieties are selected from A, T, G, C, and 5-methyl cytosine. Optionally, for LNA gapmers, 5-methyl cytosine LNA nucleosides may be used.

### Modified Oligonucleotide

The term modified oligonucleotide describes an oligonucleotide comprising one or more sugar-modified nucleosides and/or modified internucleoside linkages. The term “chimeric oligonucleotide” is a term that has been used in the literature to describe oligonucleotides comprising sugar modified nucleosides and DNA nucleosides. In some embodiments, it may be advantageous for the antisense oligonucleotide of the invention to be a chimeric oligonucleotide.

### Complementarity

The term “complementarity” describes the capacity for Watson-Crick base-pairing of nucleosides/nucleotides. Watson-Crick base pairs are guanine (G)-cytosine (C) and adenine (A)-thymine (T)/uracil (U). It will be understood that oligonucleotides may comprise nucleosides with modified nucleobases, for example 5-methyl cytosine is often used in place of cytosine, and as such the term complementarity encompasses Watson Crick base-paring between non-modified and modified nucleobases (see for example Hirao et al (2012) Accounts of Chemical Research 45:2055 and Bergstrom (2009) Current Protocols in Nucleic Acid Chemistry Suppl. 37 1.4.1).

The term “% complementary” as used herein, refers to the proportion of nucleotides (in percent) of a contiguous nucleotide sequence in a nucleic acid molecule (e.g. oligonucleotide) which, across the contiguous nucleotide sequence, are complementary to a reference sequence (e.g. a target sequence or sequence motif). The percentage of complementarity is thus calculated by counting the number of aligned nucleobases that are complementary (from Watson Crick base pairs) between the two sequences (when aligned with the target sequence 5′-3′ and the oligonucleotide sequence from 3′-5′), dividing that number by the total number of nucleotides in the oligonucleotide and multiplying by 100. In such a comparison a nucleobase/nucleotide which does not align (form a base pair) is termed a mismatch. Insertions and deletions are not allowed in the calculation of % complementarity of a contiguous nucleotide sequence. It will be understood that in determining complementarity, chemical modifications of the nucleobases are disregarded as long as the functional capacity of the nucleobase to form Watson Crick base pairing is retained (e.g. 5′-methyl cytosine is considered identical to a cytosine for the purpose of calculating % identity).

Within the present invention the level of complementarity between the contiguous nucleotide sequence of the antisense oligonucleotide and the TDP-43 binding site, or target sequence, may be at least about 75%.

Within the present invention the level of complementarity between the contiguous nucleotide sequence of the antisense oligonucleotide and the target TDP-43 binding site, or target sequence, may be at least about 80%.

Within the present invention the level of complementarity between the contiguous nucleotide sequence of the antisense oligonucleotide and the TDP-43 binding site, or target sequence, may be at least about 85%.

Within the present invention the level of complementarity between the contiguous nucleotide sequence of the antisense oligonucleotide and the TDP-43 binding site, or target sequence, may be at least about 90%.

Within the present invention the level of complementarity between the contiguous nucleotide sequence of the antisense oligonucleotide and the TDP-43 binding site, or target sequence, may be at least about 95%.

In some embodiments the contiguous nucleotide sequence may be fully complementary to the TDP-43 binding site, or target sequence. The term “fully complementary”, refers to 100% complementarity.

The compounds of the invention are complementary to TDP-43 binding sites in TDP-43 target RNAs.

As illustrated in the examples, complete complementarity may not be required and in some embodiments, the oligonucleotide may comprise one, two, three, four, five, six, seven, eight or more mismatches to a TDP-43 target RNA TDP-43 RNA binding site to which it effectively binds. In this regard, oligonucleotides maybe designed which are sufficiently complementary to multiple but not identical TDP-43 binding sites in different TDP-43 Target RNAs. In some embodiments an universal base such as inosine may be used in complementary positions in the antisense oligonucleotide where these is not perfect identity of TDP-43 binding site sequence in the multiple TDP-43 RNA targets.

In some embodiments the contiguous nucleotide sequence may include one or more mismatches to a TDP-43 binding site, or target sequence.

In some embodiments the contiguous nucleotide sequence may include two or more mismatches to a TDP-43 binding site, or target sequence.

In some embodiments the contiguous nucleotide sequence may include three or more mismatches to a TDP-43 binding site, or target sequence.

In some embodiments the contiguous nucleotide sequence may include four or more mismatches to a TDP-43 binding site, or target sequence.

In some embodiments the contiguous nucleotide sequence may include five or more mismatches to a TDP-43 binding site, or target sequence.

In some embodiments the contiguous nucleotide sequence may include six or more mismatches to a TDP-43 binding site, or target sequence.

In some embodiments the contiguous nucleotide sequence may include seven or more mismatches to a TDP-43 binding site, or target sequence.

In some embodiments the contiguous nucleotide sequence may include eight or more mismatches to a TDP-43 binding site, or target sequence.

In some embodiments, oligonucleotides of the present invention which contain one more, such as two or more, three or more, four or more, five or more, six or more, seven or more, or eight or more mismatches, may hybridize to a target nucleic acid with estimated ΔG° values below −10 kcal for oligonucleotides that are 10-32 nucleotides in length.

In some embodiments, oligonucleotides of the present invention which contain one more, such as two or more, three or more, four or more, five or more, six or more, seven or more, or eight or more mismatches, may hybridize to a target nucleic acid with estimated ΔG° values below −12 kcal, −15 kcal, −17 kcal, −20 kcal, −30 kcal, −40 kcal, −50 kcal or −60 kcal for oligonucleotides that are 10-32 nucleotides in length.

Calculation of ΔG° values is discussed below.

### Identity

The term “Identity” as used herein, refers to the proportion of nucleotides (expressed in percent) of a contiguous nucleotide sequence in a nucleic acid molecule (e.g. oligonucleotide) which across the contiguous nucleotide sequence, are identical to a reference sequence (e.g. a sequence motif). The percentage of identity is thus calculated by counting the number of aligned nucleobases that are identical (a Match) between two sequences (in the contiguous nucleotide sequence of the compound of the invention and in the reference sequence), dividing that number by the total number of nucleotides in the oligonucleotide and multiplying by 100. Therefore, Percentage of Identity=(Matches×100)/Length of aligned region (e.g. the contiguous nucleotide sequence). Insertions and deletions are not allowed in the calculation the percentage of identity of a contiguous nucleotide sequence. It will be understood that in determining identity, chemical modifications of the nucleobases are disregarded as long as the functional capacity of the nucleobase to form Watson Crick base pairing is retained (e.g. 5-methyl cytosine is considered identical to a cytosine for the purpose of calculating % identity).

### Hybridization

The term “hybridizing” or “hybridizes” as used herein is to be understood as two nucleic acid strands (e.g. an oligonucleotide and a target nucleic acid) forming hydrogen bonds between base pairs on opposite strands thereby forming a duplex. The affinity of the binding between two nucleic acid strands is the strength of the hybridization. It is often described in terms of the melting temperature (Tₘ) defined as the temperature at which half of the oligonucleotides are duplexed with the target nucleic acid. At physiological conditions Tm is not strictly proportional to the affinity (Mergny and Lacroix, 2003, Oligonucleotides 13:515-537). The standard state Gibbs free energy ΔG° is a more accurate representation of binding affinity and is related to the dissociation constant (K_{d}) of the reaction by ΔG°=−RTIn (K_{d}), where R is the gas constant and T is the absolute temperature. Therefore, a very low ΔG° of the reaction between an oligonucleotide and the target nucleic acid reflects a strong hybridization between the oligonucleotide and target nucleic acid. ΔG° is the energy associated with a reaction where aqueous concentrations are 1M, the pH is 7, and the temperature is 37° C. The hybridization of oligonucleotides to a target nucleic acid is a spontaneous reaction and for spontaneous reactions ΔG° is less than zero. ΔG° can be measured experimentally, for example, by use of the isothermal titration calorimetry (ITC) method as described in Hansen et al., 1965, Chem. Comm. 36-38 and Holdgate et al., 2005, Drug Discov Today. The skilled person will know that commercial equipment is available for ΔG° measurements. ΔG° can also be estimated numerically by using the nearest neighbor model as described by SantaLucia, 1998, Proc Natl Acad Sci USA. 95:1460-1465 using appropriately derived thermodynamic parameters described by Sugimoto et al., 1995, Biochemistry 34:11211-11216 and McTigue et al., 2004, Biochemistry 43:5388-5405. In some embodiments, oligonucleotides of the present invention hybridize to a target nucleic acid with estimated ΔG° values below −10 kcal for oligonucleotides that are 10-30 nucleotides in length. In some embodiments the degree or strength of hybridization is measured by the standard state Gibbs free energy ΔG°. The oligonucleotides may hybridize to a target nucleic acid with estimated ΔG° values below the range of −10 kcal, such as below −15 kcal, such as below −20 kcal and such as below −25 kcal for oligonucleotides that are 8-30 nucleotides in length. In some embodiments the oligonucleotides hybridize to a target nucleic acid with an estimated ΔG° value of −10 to −60 kcal, such as −12 to −40, such as from −15 to −30 kcal or −16 to −27 kcal such as −18 to −25 kcal.

### Exemplary TDP-43 RNA Targets

In some embodiments, a TDP-43 target RNA is the mammalian protein known as stathmin 2, or SCG10, SCGN10, for example the human STMN2 as disclosed as Gene: ENSG00000104435 (ensemble.org), encoded on human Chromosome 8:79,610,814-79,666,175 forward strand (GRCh38: CM000670.2).

In some embodiments, a TDP-43 target RNA is the mammalian protein known as Ceramide transporter 1 (CERT1) for example the human CERT1 as disclosed as Gene: ENSG00000113163 (ensemble.org), encoded on human Chromosome 5:75,356,345-75,512,138 reverse strand (GRCh38: CM000667.2).

In some embodiments, a TDP-43 target RNA is the mammalian protein known as solute carrier family 5 member 7, for example the human SLC5A7 as disclosed as Gene: ENSG00000115665 (ensemble.org), encoded on human Chromosome 2:107,986,523-108,013,994 forward strand (GRCh38: CM000664.2).

In some embodiments, a TDP-43 target RNA is the mammalian protein known as Rho GTPase activating protein 32, for example the human ARHGAP32, as disclosed as Gene: ENSG00000134909 (ensemble.org), encoded on human Chromosome 11:128,965,060-129,279,324 reverse strand (GRCh38: CM000673.2).

In some embodiments, a TDP-43 target RNA is CAMK2B.

In some embodiments, a TDP-43 target RNA is KALRN.

In some embodiments, a TDP-43 target RNA is UNC13A.

### Target Cell

The term “target cell” as used herein refers to a cell which is expressing the targeted TDP-43 RNA targets whose expression is to be corrected by the administration of the compound of the invention. Suitably the target cell is further TDP-43 depleted. For experimental use, TDP-43 depletion may be engineered into the cell, e.g. via genetic engineering (e.g. CRISPR/CAS9) or via the use of ASO inhibitors of TDP-43.

In some embodiments the target cell may be in vivo or in vitro. In some embodiments the target cell is a mammalian cell such as a rodent cell, such as a mouse cell or a rat cell, or a primate cell such as a monkey cell or a human cell.

In some embodiments the target cell is a neuronal cell.

For in vitro evaluation, the target cell may be a glutamatergic neuron (also referred to herein as a glutaneuron cell), such as a human glutamatergic neuron, such as a human glutamatergic neuron which is TDP-43 depleted. Human glutamatergic neuron are available from Cellular Dynamics (iCell GlutaNeurons). For in vitro evaluation the target cell, such as the glutaneuron are in vitro. TDP-43 depletion of the target cell, for example for in vitro evaluation, may be achieved for example using antisense oligonucleotides or siRNA reagents, or may be engineered into the cells e.g. via CRISPR/Cas9 editing, or shRNA vector expression. As further illustrated in the examples, the target cell, for example for in vitro use, may be a human pluripotent stem cell-derived neuron, for example these are obtainable as iCell GlutaNeurons Kit, 01279 Cat. R1034 (Fujifilm Cellular Dynamics).

### Splice Modulation

Splice modulation can be used to correct cryptic splicing, modulate alternative splicing, restore the open reading frame, and induce protein knockdown.

Splice modulation may be assayed by RNA sequencing (RNAseq), which allows for a quantitative assessment of the different splice products of a pre-mRNA, or by digital droplet PCR using PCR assays designed to be specific for one or the other splice form. In some embodiments of the invention, the antisense oligonucleotide modulates the splicing of the STMN2 pre-mRNA, e.g. they reduce the level of mature STMN2 mRNA which comprises a RNA sequence positioned between the exon 1 and exon 2 (as illustrated in the examples), for example in the target cell or TDP-43 depleted cells. In some embodiments of the invention, the antisense oligonucleotide modulates the splicing of the STMN2 pre-mRNA, e.g. they enhance the level of mature correctly spliced STMN2 mRNA which does not comprise a RNA sequence positioned between the exon 1 and exon 2, referred to as WT STMN2 transcript, for example in the target cell.

In some embodiments of the invention, the antisense oligonucleotide of the invention modulates the splicing of CERT1 in the target cell, such as by reducing the inclusion of an aberrant exon in the mature CERT1 mRNA, as illustrated in the examples.

In some embodiments of the invention, the antisense oligonucleotide of the invention modulates the splicing of SLC5A7 in the target cell, such as by reducing the inclusion of an aberrant exon in the mature SLC5A7 mRNA, as illustrated in the examples.

In some embodiments of the invention, the antisense oligonucleotide of the invention modulates the splicing of ARHGAP32in the target cell, such as by reducing the inclusion of an aberrant exon in the mature ARHGAP32 mRNA, as illustrated in the examples.

### High Affinity Modified Nucleosides

A high affinity modified nucleoside is a modified nucleotide which, when incorporated into the oligonucleotide enhances the affinity of the oligonucleotide for its complementary target, for example as measured by the melting temperature (T^{m}). A high affinity modified nucleoside of the present invention preferably result in an increase in melting temperature between +0.5 to +12° C., more preferably between +1.5 to +10° C. and most preferably between+3 to +8° C. per modified nucleoside. Numerous high affinity modified nucleosides are known in the art and include for example, many 2′ substituted nucleosides as well as locked nucleic acids (LNA) (see e.g. Freier & Altmann; Nucl. Acid Res., 1997, 25, 4429-4443 and Uhlmann; Curr. Opinion in Drug Development, 2000, 3 (2), 293-213).

### Sugar Modifications

The oligomer of the invention may comprise one or more nucleosides which have a modified sugar moiety, i.e. a modification of the sugar moiety when compared to the ribose sugar moiety found in DNA and RNA.

Numerous nucleosides with modification of the ribose sugar moiety have been made, primarily with the aim of improving certain properties of oligonucleotides, such as affinity and/or nuclease resistance.

Such modifications include those where the ribose ring structure is modified, e.g. by replacement with a hexose ring (HNA), or a bicyclic ring, which typically have a biradicle bridge between the C2 and C4 carbons on the ribose ring (LNA), or an unlinked ribose ring which typically lacks a bond between the C2 and C3 carbons (e.g. UNA). Other sugar modified nucleosides include, for example, bicyclohexose nucleic acids (WO2011/017521) or tricyclic nucleic acids (WO2013/154798). Modified nucleosides also include nucleosides where the sugar moiety is replaced with a non-sugar moiety, for example in the case of peptide nucleic acids (PNA), or morpholino nucleic acids.

Sugar modifications also include modifications made via altering the substituent groups on the ribose ring to groups other than hydrogen, or the 2′-OH group naturally found in DNA and RNA nucleosides. Substituents may, for example be introduced at the 2′, 3′, 4′ or 5′ positions.

### 2′ Sugar Modified Nucleosides

A 2′ sugar modified nucleoside is a nucleoside which has a substituent other than H or —OH at the 2′ position (2′ substituted nucleoside) or comprises a 2′ linked biradicle capable of forming a bridge between the 2′ carbon and a second carbon in the ribose ring, such as LNA (2′-4′ biradicle bridged) nucleosides.

Indeed, much focus has been spent on developing 2′ sugar substituted nucleosides, and numerous 2′ substituted nucleosides have been found to have beneficial properties when incorporated into oligonucleotides. For example, the 2′ modified sugar may provide enhanced binding affinity and/or increased nuclease resistance to the oligonucleotide. Examples of 2′ substituted modified nucleosides are 2′-O-alkyl-RNA, 2′-O-methyl-RNA, 2′-alkoxy-RNA, 2′-O-methoxyethyl-RNA (MOE), 2′-amino-DNA, 2′-Fluoro-RNA, and 2′-F-ANA nucleoside. For further examples, please see e.g. Freier & Altmann; Nucl. Acid Res., 1997, 25, 4429-4443 and Uhlmann; Curr. Opinion in Drug Development, 2000, 3 (2), 293-213, and Deleavey and Damha, Chemistry and Biology 2012, 19, 937. Below are illustrations of some 2′ substituted modified nucleosides.

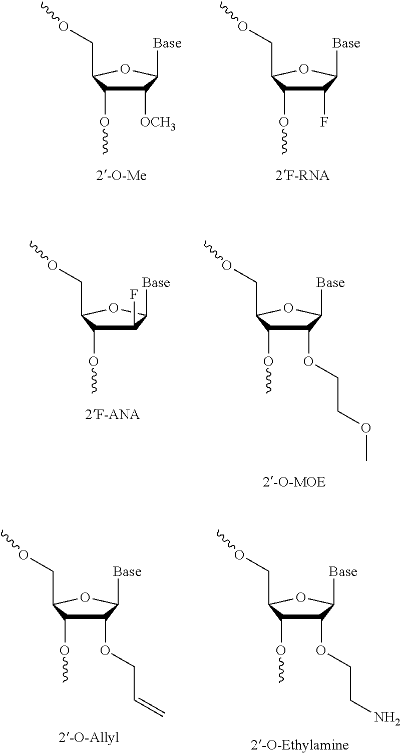

In relation to the present invention 2′ substituted sugar modified nucleosides does not include 2′ bridged nucleosides like LNA.

### Locked Nucleic Acid Nucleosides (LNA Nucleoside)

A “LNA nucleoside” is a 2′-modified nucleoside which comprises a biradical linking the C2′ and C4′ of the ribose sugar ring of said nucleoside (also referred to as a “2′-4′ bridge”), which restricts or locks the conformation of the ribose ring. These nucleosides are also termed bridged nucleic acid or bicyclic nucleic acid (BNA) in the literature. The locking of the conformation of the ribose is associated with an enhanced affinity of hybridization (duplex stabilization) when the LNA is incorporated into an oligonucleotide for a complementary RNA or DNA molecule. This can be routinely determined by measuring the melting temperature of the oligonucleotide/complement duplex.

Non limiting, exemplary LNA nucleosides are disclosed in WO 99/014226, WO 00/66604, WO 98/039352, WO 2004/046160, WO 00/047599, WO 2007/134181, WO 2010/077578, WO 2010/036698, WO 2007/090071, WO 2009/006478, WO 2011/156202, WO 2008/154401, WO 2009/067647, WO 2008/150729, Morita et al., Bioorganic & Med. Chem. Lett. 12, 73-76, Seth et al. J. Org. Chem. 2010, Vol 75 (5) pp. 1569-81, Mitsuoka et al., Nucleic Acids Research 2009, 37 (4), 1225-1238, and Wan and Seth, J. Medical Chemistry 2016, 59, 9645-9667.

Further non limiting, exemplary LNA nucleosides are disclosed in Scheme 1.

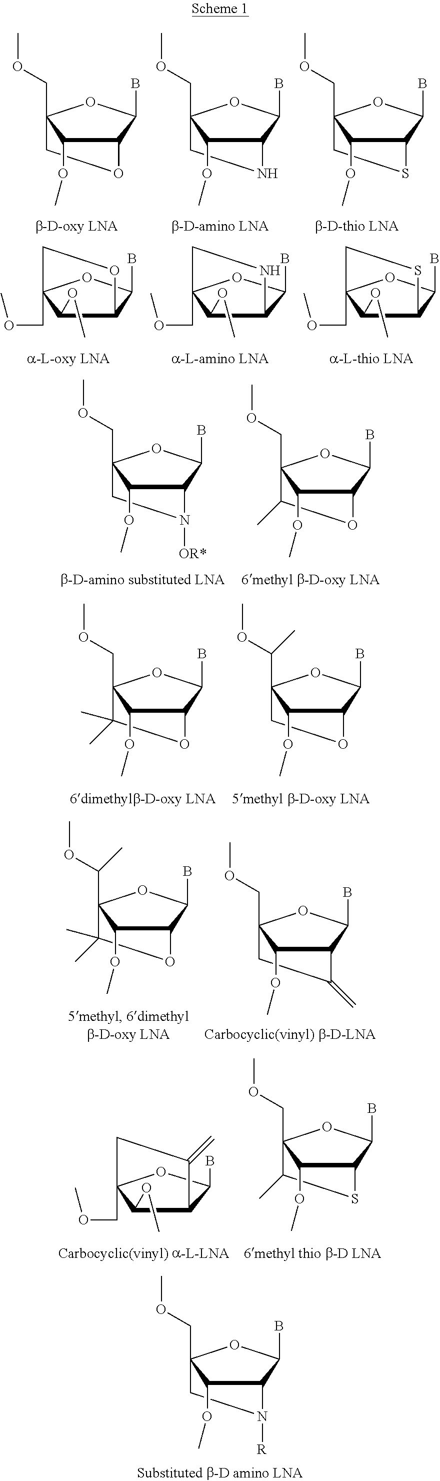

Particular LNA nucleosides are beta-D-oxy-LNA, 6′-methyl-beta-D-oxy LNA such as(S)-6′-methyl-beta-D-oxy-LNA (ScET) and ENA.

A particularly advantageous LNA is beta-D-oxy-LNA.

### Morpholino Oligonucleotides

In some embodiments, the oligonucleotide of the invention comprises or consists of morpholino nucleosides (i.e. is a Morpholino oligomer and as a phosphorodiamidate Morpholino oligomer (PMO)). Splice modulating morpholino oligonucleotides have been approved for clinical use—see for example eteplirsen, a 30 nt morpholino oligonucleotide targeting a frame shift mutation in DMD, used to treat Duchenne muscular dystrophy. Morpholino oligonucleotides have nucleobases attached to six membered morpholine rings rather ribose, such as methylenemorpholine rings linked through phosphorodiamidate groups, for example as illustrated by the following illustration of 4 consecutive morpholino nucleotides:

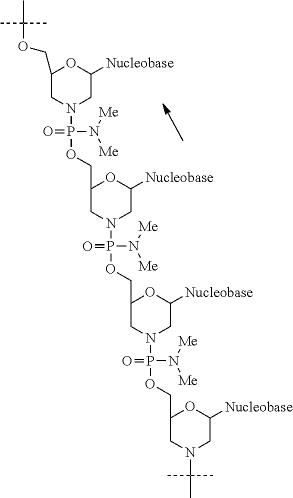

In some embodiments, morpholino oligonucleotides of the invention may be, for example 20-40 morpholino nucleotides in length, such as morpholino 25-35 nucleotides in length.

### RNase H Activity and Recruitment

The RNase H activity of an antisense oligonucleotide refers to its ability to recruit RNase H when in a duplex with a complementary RNA molecule. WO01/23613 provides in vitro methods for determining RNaseH activity, which may be used to determine the ability to recruit RNaseH. Typically an oligonucleotide is deemed capable of recruiting RNase H if it, when provided with a complementary target nucleic acid sequence, has an initial rate, as measured in pmol/l/min, of at least 5%, such as at least 10% or more than 20% of the of the initial rate determined when using a oligonucleotide having the same base sequence as the modified oligonucleotide being tested, but containing only DNA monomers with phosphorothioate linkages between all monomers in the oligonucleotide, and using the methodology provided by Example 91-95 of WO01/23613 (hereby incorporated by reference). For use in determining RHase H activity, recombinant RNase H1 is available from Lubio Science GmbH, Lucerne, Switzerland.

DNA oligonucleotides are known to effectively recruit RNaseH, as are gapmer oligonucleotides which comprise a region of DNA nucleosides (typically at least 5 or 6 contiguous DNA nucleosides), flanked 5′ and 3′ by regions comprising 2′ sugar modified nucleosides, typically high affinity 2′ sugar modified nucleosides, such as 2-O-MOE and/or LNA. For effective modulation of splicing, degradation of the pre-mRNA is not desirable, and as such it is preferable to avoid the RNaseH degradation of the target. Therefore, the oligonucleotides of the invention is preferably not gapmer oligonucleotide. RNaseH recruitment may be avoided by limiting the number of contiguous DNA nucleotides in the oligonucleotide—therefore for effective splice modulation mixmers and totalmers designs may therefore be used.

### Mixmers and Totalmers

For splice modulation it is often advantageous to use antisense oligonucleotides which do not recruit RNAseH. As RNaseH activity requires a contiguous sequence of DNA nucleotides, RNaseH activity of antisense oligonucleotide may be achieved by designing antisense oligonucleotides which do not comprise a region of more than 3 or more than 4 contiguous DNA nucleosides. This may be achieved by using antisense oligonucleotides or contiguous nucleoside regions thereof with a mixmer design, which comprise sugar modified nucleosides, such as 2′ sugar modified nucleosides, and short regions of DNA nucleosides, such as 1, 2 or 3 DNA nucleosides. Mixmers are exemplified herein by every second design, wherein the nucleosides alternative between 1 LNA and 1 DNA nucleoside, e.g. LDLDLDLDLDLDLDLL, with 5′ and 3′ terminal LNA nucleosides, and every third design, such as LDDLDDLDDLDDLDDL, where every third nucleoside is a LNA nucleoside.

A totalmer is an antisense oligonucleotide or a contiguous nucleotide sequence thereof which does not comprise DNA or RNA nucleosides, and may for example comprise only 2′-O-MOE nucleosides, such as a fully MOE phosphorothioate, e.g. MMMMMMMMMMMMMMMMMMMM, where M=2′-O-MOE, which are reported to be effective splice modulators for therapeutic use. Alternatively, a mixmer may comprise a mixture of modified nucleosides, such as MLMLMLMLMLMLMLMLMLML, wherein L=LNA and M=a non LNA modified nucleoside such as a 2′-O-MOE nucleosides.

Advantageously, the internucleoside nucleosides in mixmers and totalmers may be phosphorothioate, or a majority of nucleoside linkages in mixmers may be phosphorothioate. Mixmers and totalmers may comprise other internucleoside linkages, such as phosphodiester or phosphorodithioate, by way of example.

### Region D′ or D″ in an Oligonucleotide

The contiguous sequence of nucleobases of the oligonucleotide of the invention is typically complementary to multiple TDP-43 binding sites present in distinct TDP-43 RNA targets. The region of the antisense oligonucleotide which is complementary to, such as fully complementary to, the TDP-43 binding site is referred to as the contiguous nucleotide sequence. In some embodiments all of the nucleosides of the antisense oligonucleotide are within the contiguous nucleotide sequence (i.e. the antisense oligonucleotide and contiguous nucleotide sequence are of the same length of nucleotides). In some embodiments the antisense oligonucleotide comprises the contiguous nucleotide sequence and optionally a nucleotide-based linker region which may link the oligonucleotide to an optional functional group such as a conjugate, or other non-complementary terminal nucleotides (e.g. region D′ or D″).

The oligonucleotide of the invention may in some embodiments comprise or consist of the contiguous nucleotide sequence of the oligonucleotide which is complementary to the target nucleic acid, such as a mixmer or totalmer region, and further 5′ and/or 3′ nucleosides. The further 5′ and/or 3′ nucleosides may or may not be fully complementary to the target nucleic acid. Such further 5′ and/or 3′ nucleosides may be referred to as region D′ and D″ herein.

The addition of region D′ or D″ may be used for the purpose of joining the contiguous nucleotide sequence, such as the mixmer or totalmer, to a conjugate moiety or another functional group. When used for joining the contiguous nucleotide sequence with a conjugate moiety is can serve as a biocleavable linker. Alternatively, it may be used to provide exonuclease protection or for ease of synthesis or manufacture.

Region D′ or D″ may independently comprise or consist of 1, 2, 3, 4 or 5 additional nucleotides, which may be complementary or non-complementary to the target nucleic acid. The nucleotide adjacent to the F or F′ region is not a sugar-modified nucleotide, such as a DNA or RNA or base modified versions of these. The D′ or D′ region may serve as a nuclease susceptible biocleavable linker (see definition of linkers). In some embodiments the additional 5′ and/or 3′ end nucleotides are linked with phosphodiester linkages, and are DNA or RNA. Nucleotide based biocleavable linkers suitable for use as region D′ or D″ are disclosed in WO2014/076195, which include by way of example a phosphodiester linked DNA dinucleotide. The use of biocleavable linkers in poly-oligonucleotide constructs is disclosed in WO2015/113922, where they are used to link multiple antisense constructs within a single oligonucleotide.

In one embodiment the oligonucleotide of the invention comprises a region D′ and/or D″ in addition to the contiguous nucleotide sequence which constitutes a mixmer or a totalmer.

In some embodiments the internucleoside linkage positioned between region D′ or D″ and the mixmer or totalmer region is a phosphodiester linkage.

### Conjugate

The term conjugate as used herein refers to an oligonucleotide which is covalently linked to a non-nucleotide moiety (conjugate moiety or region C or third region). The conjugate moiety may be covalently linked to the antisense oligonucleotide, optionally via a linker group, such as region D′ or D″.

Oligonucleotide conjugates and their synthesis has also been reported in comprehensive reviews by Manoharan in Antisense Drug Technology, Principles, Strategies, and Applications, S. T. Crooke, ed., Ch. 16, Marcel Dekker, Inc., 2001 and Manoharan, Antisense and Nucleic Acid Drug Development, 2002, 12, 103.

In some embodiments, the non-nucleotide moiety (conjugate moiety) is selected from the group consisting of carbohydrates (e.g. GalNAc), cell surface receptor ligands, drug substances, hormones, lipophilic substances, polymers, proteins, peptides, toxins (e.g. bacterial toxins), vitamins, viral proteins (e.g. capsids) or combinations thereof.

### Linkers

A linkage or linker is a connection between two atoms that links one chemical group or segment of interest to another chemical group or segment of interest via one or more covalent bonds. Conjugate moieties can be attached to the oligonucleotide directly or through a linking moiety (e.g. linker or tether). Linkers serve to covalently connect a third region, e.g. a conjugate moiety (Region C), to a first region, e.g. an oligonucleotide or contiguous nucleotide sequence complementary to the target nucleic acid (region A).

In some embodiments of the invention the conjugate or oligonucleotide conjugate of the invention may optionally, comprise a linker region (second region or region B and/or region Y) which is positioned between the oligonucleotide or contiguous nucleotide sequence complementary to the target nucleic acid (region A or first region) and the conjugate moiety (region C or third region).

Region B refers to biocleavable linkers comprising or consisting of a physiologically labile bond that is cleavable under conditions normally encountered or analogous to those encountered within a mammalian body. Conditions under which physiologically labile linkers undergo chemical transformation (e.g., cleavage) include chemical conditions such as pH, temperature, oxidative or reductive conditions or agents, and salt concentration found in or analogous to those encountered in mammalian cells. Mammalian intracellular conditions also include the presence of enzymatic activity normally present in a mammalian cell such as from proteolytic enzymes or hydrolytic enzymes or nucleases. In one embodiment the biocleavable linker is susceptible to S1 nuclease cleavage. In some embodiments the nuclease susceptible linker comprises between 1 and 5 nucleosides, such as DNA nucleoside(s) comprising at least two consecutive phosphodiester linkages. Phosphodiester containing biocleavable linkers are described in more detail in WO 2014/076195.

Region Y refers to linkers that are not necessarily biocleavable but primarily serve to covalently connect a conjugate moiety (region C or third region), to an oligonucleotide (region A or first region). The region Y linkers may comprise a chain structure or an oligomer of repeating units such as ethylene glycol, amino acid units or amino alkyl groups The oligonucleotide conjugates of the present invention can be constructed of the following regional elements A-C, A-B-C, A-B-Y-C, A-Y-B-C or A-Y-C. In some embodiments the linker (region Y) is an amino alkyl, such as a C2-C36 amino alkyl group, including, for example C6 to C12 amino alkyl groups. In some embodiments the linker (region Y) is a C6 amino alkyl group.

### Treatment

The term ‘treatment’ as used herein refers to both treatment of an existing disease (e.g. a disease or disorder as herein referred to), or prevention of a disease, i.e. prophylaxis. It will therefore be recognized that treatment as referred to herein may, in some embodiments, be prophylactic. In some embodiments, the treatment is not prophylactic, for example, the treatment is treatment of an existing disease condition which has been diagnosed in the patient.

### DETAILED DESCRIPTION OF THE INVENTION

### The Oligonucleotides of the Invention

The oligonucleotide of the invention is an antisense oligonucleotide which is complementary to an RNA binding site on multiple independent pre-mRNA transcripts, such as to a TDP-43 RNA binding site on multiple pre-mRNA transcripts. The oligonucleotide of the invention is capable or modulating the expression of the multiple pre-mRNA transcripts, for example via (independently) modulation of pre-mRNA splicing, enhancing RNA stabilization, enhancing expression of the encoded protein, reducing expression of truncated proteins encoded by the pre-mRNA(s). As illustrated in the examples, the oligonucleotide of the invention may therefore be used to enhance the fidelity of pre-mRNA processing into mature mRNA encoding correctly expressed and functional proteins. The oligonucleotides of the invention may therefore be suitable for use in the treatment of diseases which are associated with a dysregulation of pre-mRNA maturation.

In some embodiments, the oligonucleotide of the invention may comprise one, two, three, four, five, six, seven, eight or more mismatches between the oligonucleotide and the target nucleic acid TDP-43 binding region. Despite mismatches hybridization to the target nucleic acid may still be sufficient to show a desired modulation of the TDP-43 RNA target RNA. Reduced binding affinity resulting from mismatches may advantageously be compensated by increased number of nucleotides in the oligonucleotide and/or an increased number of modified nucleosides capable of increasing the binding affinity to the target, such as 2′ sugar modified nucleosides, including LNA, present within the oligonucleotide sequence.

In some embodiments, one, two, three, four, five six, seven, eight or more universal nucleosides, such as inosine, may be used at mismatch positions.

Inosine is a nucleoside having the following structure:

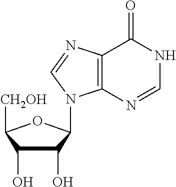

Universal nucleosides are particularly useful when oligonucleotides are targeted to different TDP-43 target RNAs which have non-identical TDP-43 binding regions.

In some embodiments the contiguous nucleotide sequence may include one or more universal nucleotides at positions representing mismatches to a TDP-43 binding site, or target sequence.

In some embodiments the contiguous nucleotide sequence may include two or more universal nucleotides at positions representing mismatches to a TDP-43 binding site, or target sequence.

In some embodiments the contiguous nucleotide sequence may include three or more universal nucleotides at positions representing mismatches to a TDP-43 binding site, or target sequence.

In some embodiments the contiguous nucleotide sequence may include four or more universal nucleotides at positions representing mismatches to a TDP-43 binding site, or target sequence.

In some embodiments the contiguous nucleotide sequence may include five or more universal nucleotides at positions representing mismatches to a TDP-43 binding site, or target sequence.

In some embodiments the contiguous nucleotide sequence may include six or more universal nucleotides at positions representing mismatches to a TDP-43 binding site, or target sequence.

In some embodiments the contiguous nucleotide sequence may include seven or more universal nucleotides at positions representing mismatches to a TDP-43 binding site, or target sequence.

In some embodiments the contiguous nucleotide sequence may include eight or more universal nucleotides at positions representing mismatches to a TDP-43 binding site, or target sequence.

In some embodiments, oligonucleotides of the present invention which contain one more, such as two or more, three or more, four or more, five or more, six or more, seven or more, or eight or more universal nucleotides at positions representing mismatches, may hybridize to a target nucleic acid with estimated ΔG° values below −10 kcal for oligonucleotides that are 10-32 nucleotides in length.

In some embodiments, oligonucleotides of the present invention which contain one more, such as two or more, three or more, four or more, five or more, six or more, seven or more, or eight or more mismatches, may hybridize to a target nucleic acid with estimated ΔG° values below −12 kcal, −15 kcal, −17 kcal, −20 kcal, −30 kcal, −40 kcal, −50 kcal or −60 kcal for oligonucleotides that are 10-32 nucleotides in length.

Calculation of ΔG° values is discussed above.

In some embodiments, the antisense oligonucleotide of the invention or the contiguous nucleotide sequence thereof comprises or consists of 10, 11, 12, 13, 14, 15, 16, 17, 18, 19, 20, 21, 22, 23, 24, 25, 26, 27, 28, 29, 30, 31, 32, 33, 34, 35, 36, 37, 38, 39 or 40 contiguous nucleotides in length.

In some embodiments, the oligonucleotide or contiguous nucleotide sequence comprises or consists of a sequence selected from the group consisting of sequences SEQ ID NO: 1-18 or a sequence selected from SEQ ID NOs 1-34 and SEQ ID NOs 50-103. It will be understood that the sequence shown in SEQ ID NO: 1-18 or a sequence selected from SEQ ID NOs 1-34 and SEQ ID NOs 50-103 may include modified nucleobases which function as the shown nucleobase in base pairing, for example 5-methyl cytosine may be used in place of methyl cytosine. Inosine may be used as a universal base.

In some embodiments, the antisense oligonucleotide or contiguous nucleotide sequence comprises or consists of 8 to 30 or 8 to 40 nucleotides in length with at least 75%, such as at least 80%, at least 85%, at least 90% identity, at least 90% or more identity to a sequence selected from the group consisting of SEQ ID NO: 1 to 18 or a sequence selected from SEQ ID NOs 1-34 and SEQ ID NOs 50-103. In some embodiments the antisense oligonucleotide or contiguous nucleotide sequence comprises or consists of 8 to 30 or 8 to 40 nucleotides in length with 100% identity to a sequence selected from the group consisting of SEQ ID NO: 1 to 18 or a sequence selected from SEQ ID NOs 1-34 and SEQ ID NOs 50-103.

It is understood that the contiguous nucleobase sequences (motif sequence) can be modified to for example increase nuclease resistance and/or binding affinity to the target nucleic acid.

The pattern in which the modified nucleosides (such as high affinity modified nucleosides) are incorporated into the oligonucleotide sequence is generally termed oligonucleotide design.

The oligonucleotides of the invention are designed with modified nucleosides and DNA nucleosides. Advantageously, high affinity modified nucleosides are used.

In an embodiment, the oligonucleotide comprises at least 1 modified nucleoside, such as at least 2, at least 3, at least 4, at least 5, at least 6, at least 7, at least 8, at least 9, at least 10, at least 11, at least 12, at least 13, at least 14, at least 15, at least 16 modified nucleosides, at least 17 modified nucleosides, at least 18 modified nucleosides, at least 19 modified nucleosides, at least 20 modified nucleosides, at least 21 modified nucleosides, at least 22 modified nucleosides, at least 23 modified nucleosides, at least 24 modified nucleosides, at least 25 modified nucleosides, at least 26 modified nucleosides, at least 27 modified nucleosides, at least 28 modified nucleosides, at least 29 modified nucleosides, at least 30 modified nucleosides, at least 31 modified nucleosides, at least 32 modified nucleosides or more. In an embodiment the oligonucleotide comprises from 1 to 10 modified nucleosides, such as from 2 to 9 modified nucleosides, such as from 3 to 8 modified nucleosides, such as from 4 to 7 modified nucleosides, such as 6 or 7 modified nucleosides. Suitable modifications are described in the “Definitions” section under “modified nucleoside”, “high affinity modified nucleosides”, “sugar modifications”, “2′ sugar modifications” and Locked nucleic acids (LNA)”.

In an embodiment, the oligonucleotide comprises one or more sugar modified nucleosides, such as 2′ sugar modified nucleosides. Preferably the oligonucleotide of the invention comprise one or more 2′ sugar modified nucleoside independently selected from the group consisting of 2′-O-alkyl-RNA, 2′-O-methyl-RNA, 2′-alkoxy-RNA, 2′-O-methoxyethyl-RNA, 2′-amino-DNA, 2′-fluoro-DNA, arabino nucleic acid (ANA), 2′-fluoro-ANA and LNA nucleosides. It is advantageous if one or more of the modified nucleoside(s) is a locked nucleic acid (LNA).

In a further embodiment the oligonucleotide comprises at least one modified internucleoside linkage. Suitable internucleoside modifications are described in the “Definitions” section under “Modified internucleoside linkage”. It is advantageous if at least 75%, such as all, the internucleoside linkages within the contiguous nucleotide sequence are phosphorothioate or boranophosphate internucleoside linkages. In some embodiments all the internucleotide linkages in the contiguous sequence of the oligonucleotide are phosphorothioate linkages.

### Pharmaceutically Acceptable Salts

The invention contemplates pharmaceutically acceptable salts of the antisense oligonucleotides of the invention. In some embodiments, the pharmaceutically acceptable salt is a sodium salt, a potassium salt or an ammonium salt.

### Method of Manufacture

In a further aspect, the invention provides methods for manufacturing the oligonucleotides of the invention comprising reacting nucleotide units and thereby forming covalently linked contiguous nucleotide units comprised in the oligonucleotide. Preferably, the method uses phophoramidite chemistry (see for example Caruthers et al, 1987, Methods in Enzymology vol. 154, pages 287-313). In a further embodiment the method further comprises reacting the contiguous nucleotide sequence with a conjugating moiety (ligand) to covalently attach the conjugate moiety to the oligonucleotide. In a further aspect a method is provided for manufacturing the composition of the invention, comprising mixing the oligonucleotide or conjugated oligonucleotide of the invention with a pharmaceutically acceptable diluent, solvent, carrier, salt and/or adjuvant.

### Pharmaceutical Composition

In a further aspect, the invention provides pharmaceutical compositions comprising any of the aforementioned oligonucleotides and/or oligonucleotide conjugates or salts thereof and a pharmaceutically acceptable diluent, carrier, salt and/or adjuvant. A pharmaceutically acceptable diluent includes phosphate-buffered saline (PBS) and pharmaceutically acceptable salts include, but are not limited to, sodium and potassium salts. In some embodiments the pharmaceutically acceptable diluent is sterile phosphate buffered saline. In some embodiments the oligonucleotide is used in the pharmaceutically acceptable diluent at a concentration of 50-300μM solution.

### Applications

The oligonucleotides of the invention may be utilized as research reagents for, for example, diagnostics, therapeutics and prophylaxis.

In research, such oligonucleotides may be used to mimic the activity of TDP-43 in cells (e.g. in vitro cell cultures, such as in neuronal cells) and experimental animals thereby facilitating functional analysis of the target or an appraisal of its usefulness as a target for therapeutic intervention.

The present invention provides a method, such as an in vivo or in vitro method, for enhancing TDP-43 functionality in a cell which is expressing aberrant or depleted levels of TDP-43, said method comprising administering an oligonucleotide, the conjugate, the salt or composition according to the invention, in an effective amount to said cell. In some embodiments, the target cell, is a mammalian cell in particular a human cell. The target cell may be an in vitro cell culture or an in vivo cell forming part of a tissue in a mammal. In preferred embodiments the target cell is a neuronal cell, such as a neuronal cell which is depleted in normal TDP-43 activity. In some embodiments, the target cell may express a disease associated variant of TDP-43, and/or express dysfunctional TDP-43.

For therapeutics, the oligonucleotides may be administered to an animal or a human, suspected of having a disease or disorder, which can be treated by mimicking TDP-43.

The invention provides methods for treating or preventing a disease, comprising administering a therapeutically or prophylactically effective amount of an oligonucleotide, an oligonucleotide conjugate or a pharmaceutical composition of the invention to a subject suffering from or susceptible to the disease.

The invention also relates to an oligonucleotide, a composition or a conjugate as defined herein for use as a medicament.

The oligonucleotide, oligonucleotide conjugate or a pharmaceutical composition according to the invention is typically administered in an effective amount.

The invention also provides for the use of the oligonucleotide or oligonucleotide conjugate of the invention as described for the manufacture of a medicament for the treatment of a disorder as referred to herein, or for a method of the treatment of as a disorder as referred to herein.

The invention further relates to use of an oligonucleotide, oligonucleotide conjugate or a pharmaceutical composition as defined herein for the manufacture of a medicament for the treatment of a neurological disorder as neurodegenerative disorders characterized by TDP-43 pathology or mis-localization of TDP-43 from the nucleus, such as ALS.

The invention also provides the oligonucleotide or antisense oligonucleotide of the invention for use in a method of treating a disorder as referred to herein.

In one embodiment, the invention relates to oligonucleotides, oligonucleotide conjugates or pharmaceutical compositions for use in the treatment of a neurological disorder as neurodegenerative disorders characterized by TDP-43 pathology or mis-localization of TDP-43 from the nucleus, such as ALS.

### Administration

The oligonucleotides or pharmaceutical compositions of the present invention may, for example be administered for example via intracerebral, intracerebroventricular or intrathecal administration.

In a preferred embodiment the oligonucleotide or pharmaceutical compositions of the present invention are administered by a parenteral route including intravenous, intraarterial, subcutaneous, intraperitoneal or intramuscular injection or infusion, intrathecal or intracranial, e.g. intracerebral or intraventricular, intravitreal administration. In one embodiment the active oligonucleotide or oligonucleotide conjugate is administered intravenously. In another embodiment the active oligonucleotide or oligonucleotide conjugate is administered subcutaneously.

### EXAMPLES

### Example 1—Evaluation of a Range of “TDP-43” Mimic ASOs in Their Ability to Correct Splicing of STMN2 Pre-mRNA in TDP-43 Depleted Neuronal Cells

### Compounds Used:

Compounds 1, 1-19,1 (compound A)

### Material and Methods

### Cell Culture Material:

96 well, black/clear, Tissue Culture Treated plate—Flat bottom with Lid, Falcon 353219
Laminin 521 Biolamina
DPBS (1×) CaCl2+/MgCl2+, Gibco 14040-091
DAPT Sigma Aldrich D5942
iCell Motor Neurons Kit, 01279 Cat. R1049 (Fujifilm Cellular Dynamics)

### RNA Isolation Material:

Rneasy Plus 96 Kit, Qiagen Cat No.: 74192
Rnase-Free Dnase Set, Qiagen Cat No.: 79254

### Library Preparation Material:

TruSeq® Stranded Total RNA Library Prep Gold, Illumina Cat No.: 20020599

### Cell Seeding, Maintenance and LNA Oligonucleotides Treatment:

Human glutamatergic neurons were plated at 2.0×105 cells/cm2 on 96-well plates coated with Poly-L-Ornithine/Laminin. To knockdown TDP-43, cells were incubated with compound A at 5 μM from day 3. From day 10, cells were treated additionally with compound 1,1 to 18,1 at 10 μM for 72 hours and then lysed in lysis buffer (supplied with the kit). Control cells were treated with the diluent alone (PBS). RNA isolation was performed according to the manufacturer's instructions including DNase treatment step.

### TDP-43 LNA Oligonucleotide—Compound A:

SEQ ID NO: 19,1=TCcacactgaacaAACC (Upper case letters are beta-D-oxy LNA, lower case letters are DNA, LNA Cs are 5-methyl cytosine, all internucleoside linkages are phosphorothioate).

Medium was removed and 100 μl/well (96 mwp) Lysis Buffer (PureLink® Pro 96 Thermo Fisher Kit) was added. RNA purification was performed as per the manufacturer's instructions, including Dnase I treatment (RNA Purification PureLink® Pro 96 Thermo Fisher Kit)

### RNA Sequencing Analysis

Sequencing libraries were generated using TruSeq stranded total RNA library preparation protocol with RiboZero to remove rRNA (Illumina). Libraries were subjected to paired end sequencing on a NovaSeq6000 sequencer (Illumina) with 150-bp read length.

Samples: The human motor neurons (hMNS) treated with and without TARDBP (encoding TDP-43) targeting oligonucleotide 19,1 and the range of TDP-43 mimic ASOs, compound 1,1-18,1. Data analysis was carried out using Partek Flow (mapping using hg38 and Cufflinks).

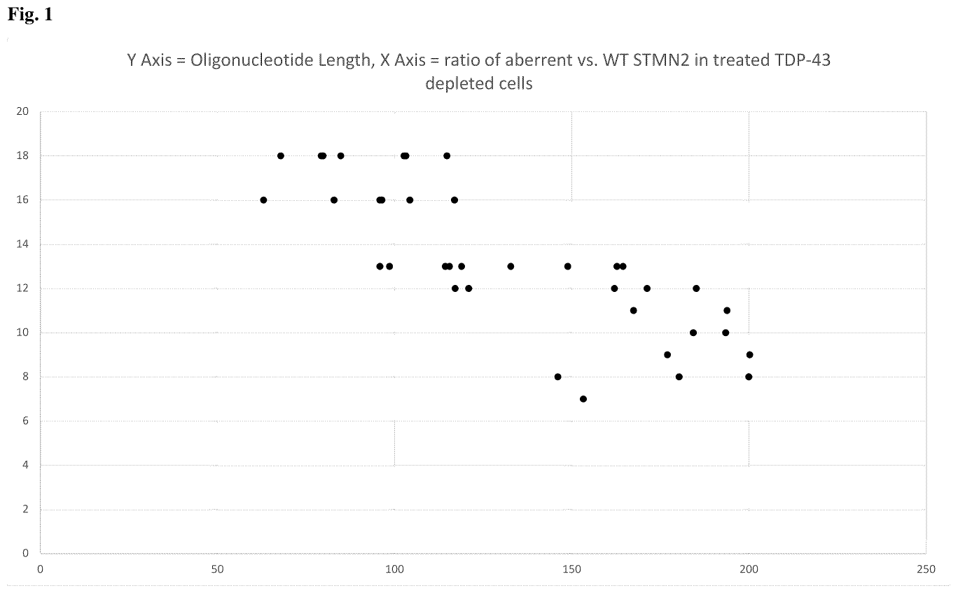

The expression level of STMN2 containing the cryptic exon, and the level of wildtype STMN2 was normalized to the expression level in the untreated control cells (100%), and are shown in Results Table A and illustrated in FIGS. 1-3.

The following compounds resulted in an enhanced expression of the WT STMN2 mRNA in TDP-43 depleted cells: 5,1, 14,1, 1,1, 15,5, 14,2, 15,1, 6,1, 6,4, 13,1, 15,4, 9,1, 10,1, 6,3, 7,1, 18,1, 8,1, 7,3, 16,3, 14,4, 15,3, 6,2, 8,2, 7,2, 18,3, 16,1, 16,2, 15,2, 18,2, and 8,3

The following compounds resulted in an enhanced expression of the WT STMN2 mRNA in TDP-43 depleted cells greater than the expression level in the control cells: 18,1, 8,1, 7,3, 16,3, 14,4, 15,3, 6,2, 8,2, 7,2, 18,3, 16,1, 16,2, 15,2, 18,2, and 8,3. These particularly effective compounds ranged in 12-18 nts in length. These particularly effective compounds ranged in Gibbs free energy of dG of about −12 to about −21.

Of note is that the most effective compounds had a length of 18 nts and a Gibbs free energy of about −20, indicating that compounds of at least 12 nucleotides in length with a Gibbs free energy of at least about −12 are advantageous.

The following compounds reduced the expression of the STMN2 aberrantly spliced mRNA in TDP-43 depleted cells: 18,4, 16,1, 14,2, 6,4, 8,3, 18,3, 8,1, 16,3, 14,3, 6,3, 16,2, 7,1, 3,1, 15,5, 7,3, 18,1, 5,1, 15,3, 18,2, 2,1, 15,4, and 12,1.

The following TDP-43 mimic oligonucleotide compounds resulted in a notable decrease in the ratio of the STMN2 cryptic exon containing STMN2 transcripts compared to the WT STMN2 transcripts (less than 150): 16,1, 8,3, 18,3, 18,4, 16,2, 18,2, 7,2, 15,2, 16,3, 15,3, 8,1, 8,2, 7,3, 6,4, 6,3, 18,1, 7,1, 14,2, 6,2, 14,4, 15,4, 10,1, 15,5.

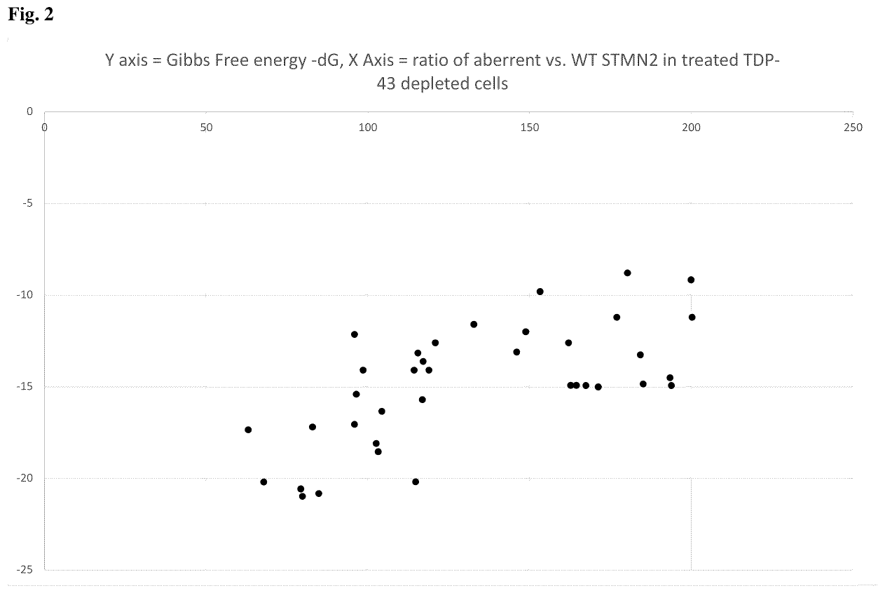

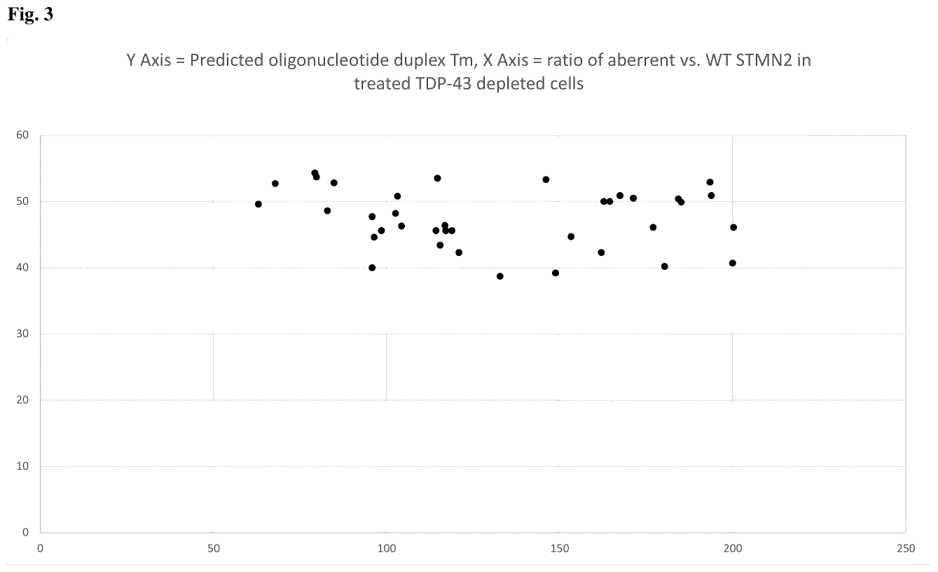

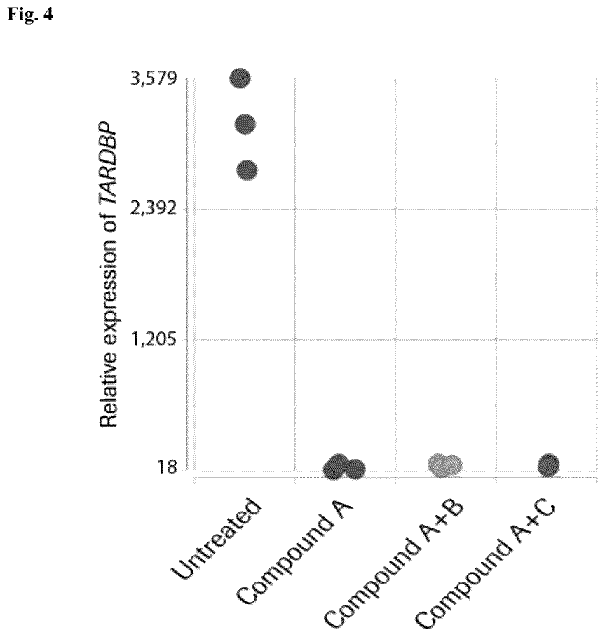

As illustrated in FIGS. 1-3, there is a clear tendency that compounds with a longer length (FIG. 1) and/or a lower Gibbs free energy (ΔG) (FIG. 2) to be more effective in correcting the TDP-43 mediated correct splicing. Notable the melting temperature of the oligonucleotide/RNA duplex was not found to correlate to a correction of TDP-43 mediated splice correction (FIG. 3).

RESULTS TABLE A

| | | | STMN2 | | | | | |
|---|---|---|---|---|---|---|---|---|
| | | | cryptic | | | | | Gibbs |
| COMP | SEQ | | exon | STMN2 | Ratio | Oligo | | Free |
| ID No | ID NO | Base sequence | 2a (A) | wt (8) | (A/B) × 100 | length | Tm | energy |
| 1.1 | 1 | 5′-ACACACAC-3′ | 140 | 78 | 180 | 8 | 64 | −8.8 |
| 2.1 | 2 | 5′-ACACACACA-3′ | 126 | 71 | 177 | 9 | 64 | −11.2 |
| 3.1 | 3 | 5′-ACACACACAC-3′ | 118 | 64 | 184 | 10 | 65 | −13.3 |
| 4.1 | 4 | 5′-ACACACACACA-3′ | 137 | 71 | 194 | 11 | 66 | −14.9 |
| 5.1 | 5 | 5′-ACACACACACAC-3′ | 124 | 72 | 171 | 12 | 63 | −15.0 |
| 6.1 | 6 | 5′-ACACACACACACA-3′ | 137 | 83 | 164 | 13 | 62 | −14.9 |
| 6.2 | 6 | 5′-ACACACACACACA-3′ | 150 | 126 | 119 | 13 | 54 | −14.1 |
| 6.3 | 6 | 5′-ACACACACACACA-3′ | 114 | 98 | 115 | 13 | 54 | −13.2 |
| 6.4 | 6 | 5′-ACACACACACACA-3′ | 98 | 86 | 114 | 13 | 53 | −14.1 |
| 7.1 | 7 | 5′-ACACACACACACACAC-3′ | 116 | 99 | 117 | 16 | 54 | −15.7 |
| 7.2 | 7 | 5′-ACACACACACACACAC-3′ | 131 | 136 | 96 | 16 | 49 | −15.4 |
| 7.3 | 7 | 5′-ACACACACACACACAC-3′ | 119 | 114 | 104 | 16 | 51 | −16.3 |
| 8.1 | 8 | 5′-ACACACACACACACACAC-3′ | 111 | 108 | 103 | 18 | 51 | −18.5 |
| 8.2 | 8 | 5′-ACACACACACACACACAC-3′ | 137 | 133 | 103 | 18 | 51 | −18.1 |
| 8.3 | 8 | 5′-ACACACACACACACACAC-3′ | 101 | 149 | 68 | 18 | 40 | −20.2 |
| 9.1 | 9 | 5′-CACACAC-3′ | 149 | 97 | 153 | 7 | 61 | −9.8 |
| 10.1 | 10 | 5′-CACACACA-3′ | 142 | 97 | 146 | 8 | 59 | −13.1 |
| 10.2 | 10 | 5′-CACACACA-3′ | 142 | 71 | 200 | 8 | 67 | −9.2 |
| 11.1 | 11 | 5′-CACACACAC-3′ | 133 | 66 | 200 | 9 | 67 | −11.2 |
| 12.1 | 12 | 5′-CACACACACA-3′ | 129 | 66 | 193 | 10 | 66 | −14.5 |
| 13.1 | 13 | 5′-CACACACACAC-3′ | 147 | 88 | 167 | 11 | 63 | −14.9 |
| 14.1 | 14 | 5′-CACACACACACA-3′ | 139 | 75 | 185 | 12 | 65 | −14.9 |
| 14.2 | 14 | 5′-CACACACACACA-3′ | 96 | 82 | 117 | 12 | 54 | −13.6 |
| 14.3 | 14 | 5′-CACACACACACA-3′ | 113 | 70 | 162 | 12 | 62 | −12.6 |
| 14.4 | 14 | 5′-CACACACACACA-3′ | 145 | 120 | 121 | 12 | 55 | −12.6 |
| 15.1 | 15 | 5′-CACACACACACAC-3′ | 133 | 82 | 163 | 13 | 62 | −14.9 |
| 15.2 | 15 | 5′-CACACACACACAC-3′ | 137 | 143 | 96 | 13 | 49 | −12.1 |
| 15.3 | 15 | 5′-CACACACACACAC-3′ | 124 | 125 | 99 | 13 | 50 | −14.1 |
| 15.4 | 15 | 5′-CACACACACACAC-3′ | 127 | 96 | 133 | 13 | 57 | −11.6 |
| 15.5 | 15 | 5′-CACACACACACAC-3′ | 118 | 79 | 149 | 13 | 60 | −12.0 |
| 16.1 | 16 | 5′-CACACACACACACACA-3′ | 87 | 138 | 63 | 16 | 39 | −17.3 |
| 16.2 | 16 | 5′-CACACACACACACACA-3′ | 115 | 139 | 83 | 16 | 45 | −17.2 |
| 16.3 | 16 | 5′-CACACACACACACACA-3′ | 111 | 116 | 96 | 16 | 49 | −17.1 |
| 18.1 | 18 | 5′-CACACACACACACACACA-3′ | 121 | 105 | 115 | 18 | 53 | −20.2 |
| 18.2 | 18 | 5′-CACACACACACACACACA-3′ | 124 | 146 | 85 | 18 | 46 | −20.8 |
| 18.3 | 18 | 5′-CACACACACACACACACA-3′ | 107 | 136 | 79 | 18 | 44 | −20.6 |
| 18.4 | 18 | 5′-CACACACACACACACACA-3′ | 48 | 60 | 80 | 18 | 44 | −21.0 |
| 19.1 | 19 | 5′-TCCACACTGAACAAACC-3′ | 130 | 71 | 183 | — | — | — |

### Example 2—Human Pluripotent Stem Cell-Derived Neuronal Culture, Oligonucleotide Treatment and RNA Isolation-Evaluation of TDP-43 Mimic ASOs to Correct Aberrant Slicing of Multiple Independent RNAs in TDP-43 Depleted Cells

### Oligonucleotides Used

### Compound A: A TDP-43 Targeting LNA Gapmer Oligonucleotide, SEQ ID NO: 19

TCcacactgaacaAACC (Upper case letters are beta-D-oxy LNA, lower case letters are DNA, LNA Cs are 5-methyl cytosine, all internucleoside linkages are phosphorothioate)—referred to herein as Compound #19,1.

### Compound B—STMN2

A LNA/DNA mixmer which targets and upregulates the expression of STMN2

Compound ID NO 36: ^{m}CₛaₛCₛAₛCₛAₛCₛGₛCₛAₛCₛAₛCₛaₛTₛG (SEQ ID NO: 36) wherein capital letters are beta-D-oxy LNA nucleotides, lower case letters are 2′deoxyribose nucleosides (DNA nucleoside), ^{m}C are 5-methyl cytosine beta-D-oxy LNA nucleosides, and subscript s is a phosphorothioate internucleoside linkage.

### Compound C—TDP43

A LNA/DNA 18 nt mixmer (compound (8,3) of formula

Aₛ^{m}CₛAₛCₛaₛCₛAₛCₛAₛCₛaₛCₛAₛCₛAₛCₛa^{m}C (SEQ ID NO: 8)

wherein capital letters are beta-D-oxy LNA nucleotides, lower case letters are 2′deoxyribose nucleosides (DNA nucleoside), ^{m}C are 5-methyl cytosine beta-D-oxy LNA nucleosides, and subscript s is a phosphorothioate internucleoside linkage.

The methodology used for performing the experiment was as described in example 1, however after the pre-treatment with compound A, the cells were either treated with compound B or compound C, or no additional compound.

Sequencing libraries were generated using TruSeq stranded total RNA library preparation protocol with RiboZero to remove rRNA (Illumina). Libraries were subjected to paired end sequencing on a NovaSeq6000 sequencer (Illumina) with 150-bp read length and an average output of 100 million paired end reads. Data analysis was performed after trimming of the reads (removal of short reads as well as removal of the last nucleotide from the 3′ end). Reads were mapped against hg38 by usage of TopHat 2, and quantification of transcripts was performed by usage of Cufflinks. Identification of alternative spliced transcripts was performed by usage of a pipeline included in Partek Flow.

Sequencing of mRNA was performed at Fasteris (Swiss)—Sequencing was performed in triplicates with an average output of 100 million paired end reads. Data analysis was performed after trimming of the reads (removal of short reads as well as removal of the last nucleotide from the 3′ end). Reads were mapped against hg38 by usage of TopHat 2, and quantification of transcripts was performed by usage of Cufflinks. Identification of alternative spliced transcripts was performed by usage of a pipeline included in Partek Flow.

### Results

Since TDP-43 is known to be important in canonical splicing of a number of pre-mRNAs (Conti et al., 2015 and Humphrey et al. 2017), we chose to sequence Glutaneurons treated with compound A, A+B, A+C as well as untreated cells. The next generation sequencing (Illumina) was performed on isolated mRNA as paired end (PE) sequencing (2×150 bp) to obtain more than 100 million PE reads. To identify alternative splicing within the transcriptome, the number of reads were subsampled to have 104 million PE reads per sample (treatments were performed in triplicates).

To make sure that TARDBP was removed after treatment with the gapmer against TARDBP, we investigated the expression level based on the mRNA-Seq data. There was observed approximate 75-fold knock down of TARDBP encoding TDP-43 protein (remaining TARDBP transcripts less than 2%) (FIG. 4). As seen from the relative expression, treatment with compound A+B or A+C did not change the expression level of TARDBP compared to treatment with TARDBP ASO (compound A) alone (FIG. 4).

### STMN2

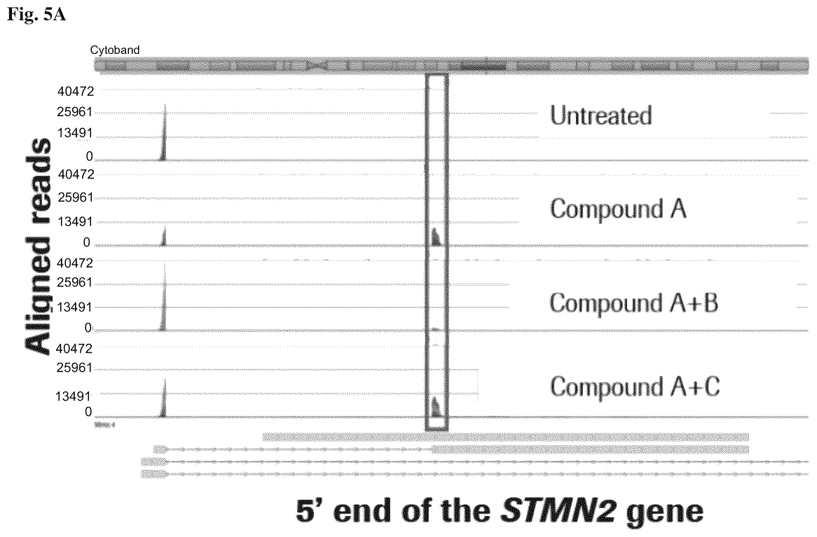

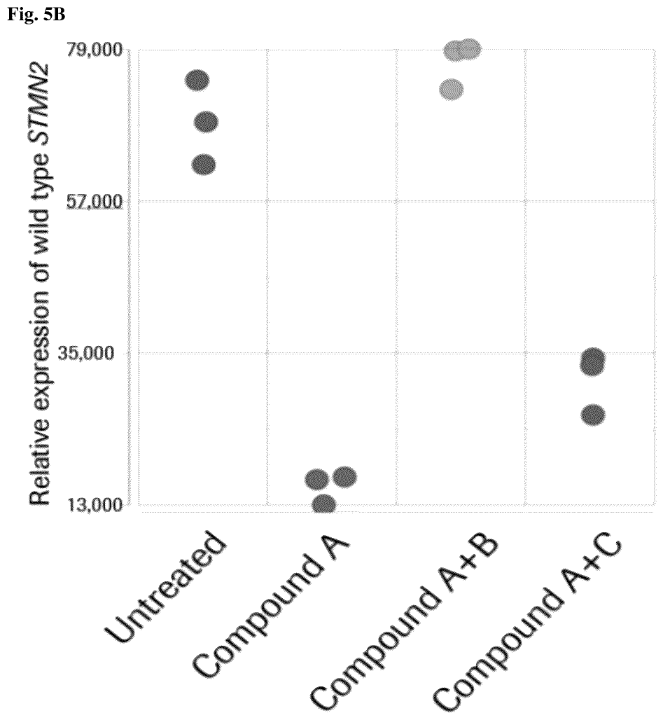

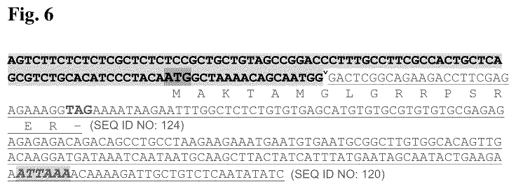

Next we investigated other transcripts that changed splicing pattern upon treatment with compound A (TDP-43 KD), and which could be restored upon treatment with our TDP-43 mimicking oligo (compound C). A TDP-43 mimicking ASO targeting the TDP-43 binding site of STMN2 was included as control (compound B). Initially we looked at the inclusion of the new splice acceptor site that previously was published was affected by our STMN2 specific ASO (compound B) or the general TDP-43 mimicking ASO (compound C). The analysis showed that the amount of wild type (wt) STMN2 was fully restored by treatment with compound A+B, and partially restored with compound A+C (FIGS. 5A and 5B). The restoration of the wild type STMN2 is due to blocking binding to the TDP-43 binding site and thereby preventing the usage of STMN2 splice acceptor site in intron 1. This new splice acceptor site that was published in 2019 (Klim et al.2019 and Melamed et al. 2019) (positioned at chr8: 79,616,822) replaces the acceptor site normally used for STMN2 exon 2. Usage of this new splice acceptor site results in a splice variant that is shortened due to usage of a polyadenylation site 203 nucleotides 3′ to the splice acceptor site (FIG. 6).

STMN2 encodes a protein necessary for normal hMN outgrowth and repair. Importantly, we established that post-translational stabilization of STMN2 can rescue deficits in motor neurite outgrowth and axon regeneration induced by TDP-43 knockdown.

Klim, J. R., Williams, L. A., Limone, F. Et al. ALS-implicated protein TDP-43 sustains levels of STMN2, a mediator of motor neuron growth and repair. Nat Neurosci 22, 167-179 (2019) (doi.org/10.1038/s41593-018-0300-4).

### ARHGAP32

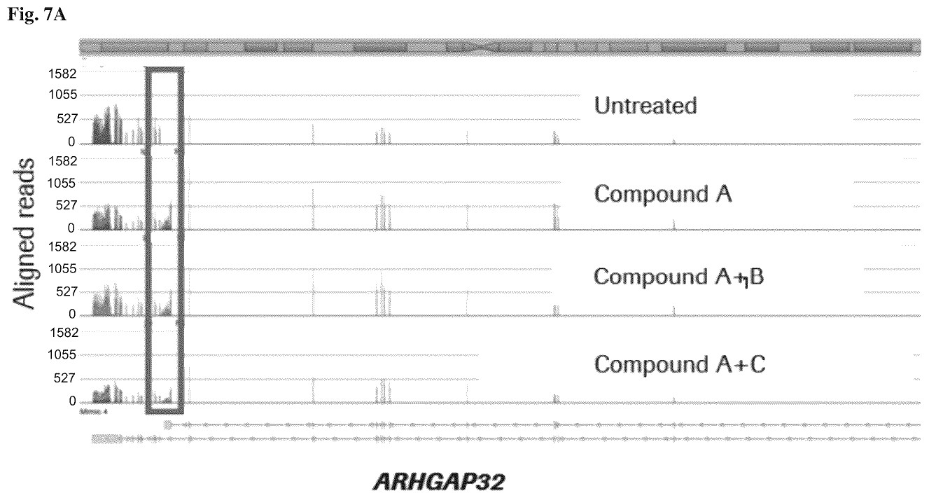

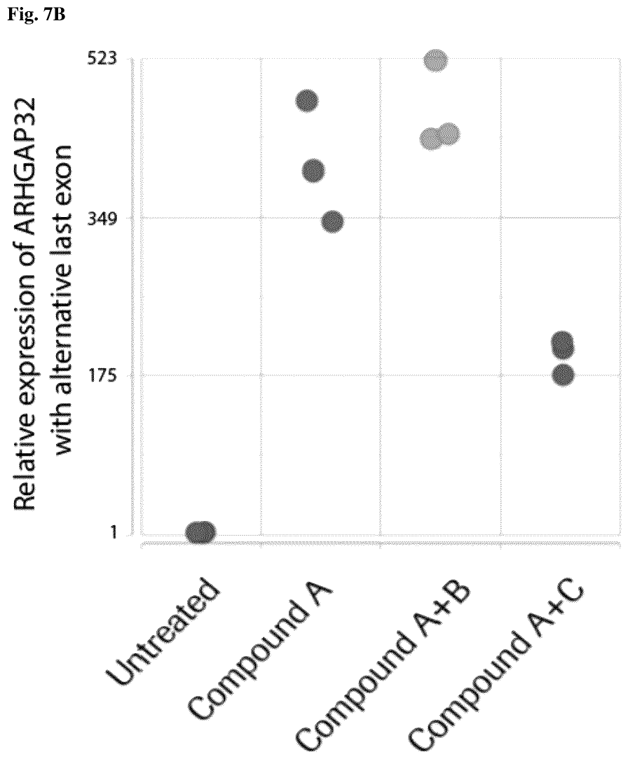

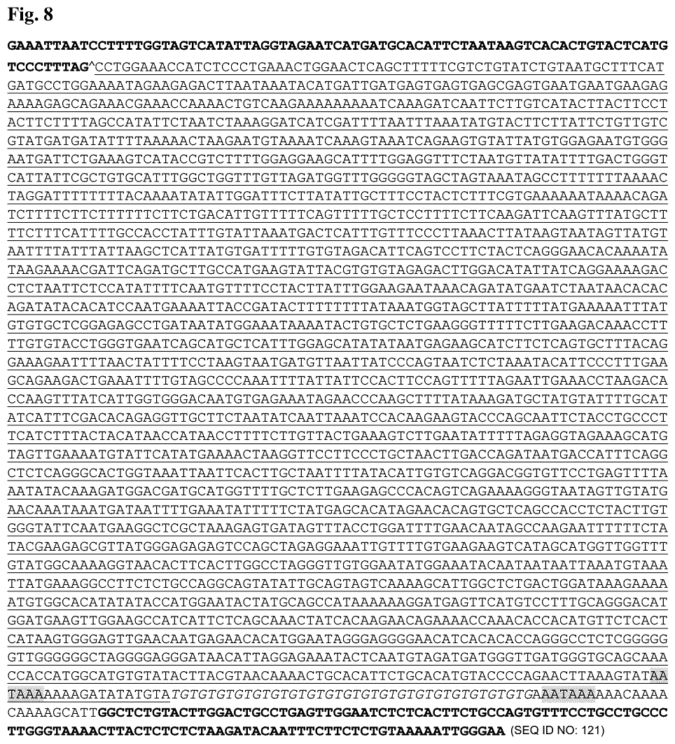

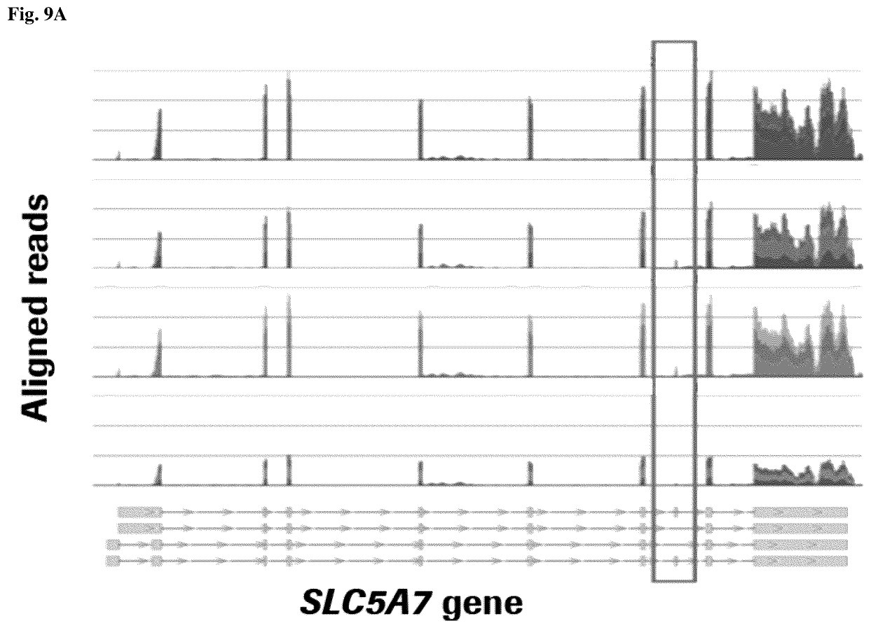

By thorough investigation of the called alternative transcripts, another gene name previously shown to be involved neuronal development and maintenance. This gene, ARHGAP32 encodes the Rho GTPase-activating protein 32, which is expressed primarily in the brain and has been shown to be involved proper neuronal development and maintenance. Knockdown of TDP-43 results in the use of an alternative splice acceptor site at pos. Chr11: 128992046 (resulting in an alternative last exon in ENSG00000134909 position 200153 in the gene, experimentally validated as shown in FIG. 7, and illustrated with reference to the ARHGAP32 pre-mRNA sequence in FIG. 8—the alternative splice site is indicated by \hat{ }, note only a fragment of the ARHGAP32 pre-mRNA is shown in FIG. 8, also disclosed herein as SEQ ID NO: 35). The new exon created by the use of the alternative splice site also contains two possible polyadenylation site 2471 and 2532 nts downstream of the splice acceptor site (See FIG. 8, shaded text), respectively. The treatment of Glutaneurons where TDP-43 have been reduced with TARDBP gapmer (i.e. TDP-43 depleted cells), shows that addition of an oligonucleotide of the invention (compound C) reduces the usage of the alternative splice acceptor site, whereas the STMN2 targeting mixmer (compound B) did not have any effect (FIG. 7). The second polyadenylation site illustrated in FIG. 8 is preceded by GU-repeat RNA binding protein sequence that is the potential TDP-43 binding site (FIG. 8). The consequence of the use of the alternative splice acceptor site, is that it generates a C-terminal truncated ARHGAP32 protein-resulting in a truncation of Rho GTPase-activating protein 32, as the open reading frame contains a stop codon in the new last exon of this variant. The new protein variant is 390 amino acids long with 6 new amino acids in the C-terminus, compared to wild type Rho GTPase-activating protein 32 which is 2,087 amino acids long. The majority of the Rho-GAP domain is lacking in this variant. The C-terminal part of wild type Rho GTPase-activating protein 32 which is also lacking has been shown to directly interact with TrkA, a high-affinity receptor for nerve growth factor (NGF), that regulates neuronal outgrowth.

### SLC5A7

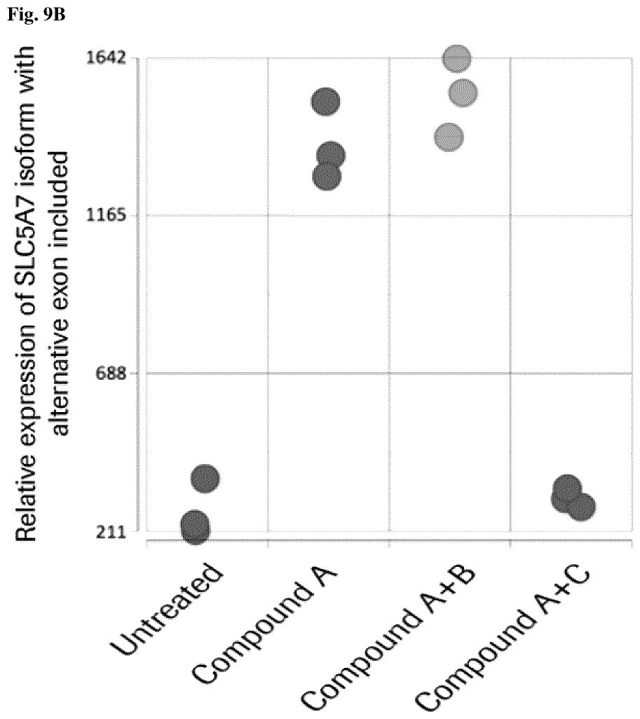

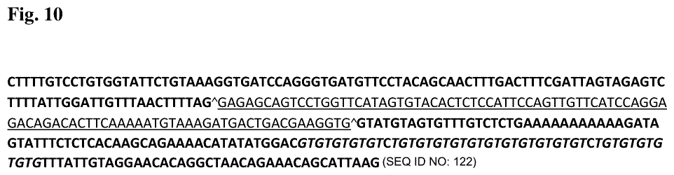

Another pre-mRNA that shows alternative splice is the SLC5A7 transcript encoding High affinity choline transporter 1. Loss of TDP-43 protein increases the use of a splice acceptor site (chr2: 108,007,307) and a splice donor site (chr2: 108,007,400) resulting in the inclusion of a novel exon with a length of 94 base pairs (chr2: 108,007,307-108,007,400) (FIGS. 9A and 9B). As seen from the relative expression, the novel isoform increases more than 6-fold upon knock down of TDP-43 (Compound A). However, subsequent treatment with the TDP-43 mimicking ASO (Compound C) completely abolished the inclusion of this alternative exon, so the expression profile for this transcript variant resembles untreated cells (FIGS. 9A and 9B). The inclusion this exon results in a frameshift during translation and a premature stop codon within the novel exon, to generate a potential truncated variant of 326 amino acids (wild type protein is 580 amino acids long (Uniprot entry Q9GZV3). C-terminal truncations of High affinity choline transporter 1 (SLC5A7) has been shown to cause motor neuron defects, and in some cases an initial diagnosis of ALS (Salter et al. 2018; Neurol Genet. 2018 April; 4 (2): e222). The protein consequence of this observed exon inclusion resembles the C-terminal truncations seen in these patients with mutations causing the C-terminal truncation.

### CERT1

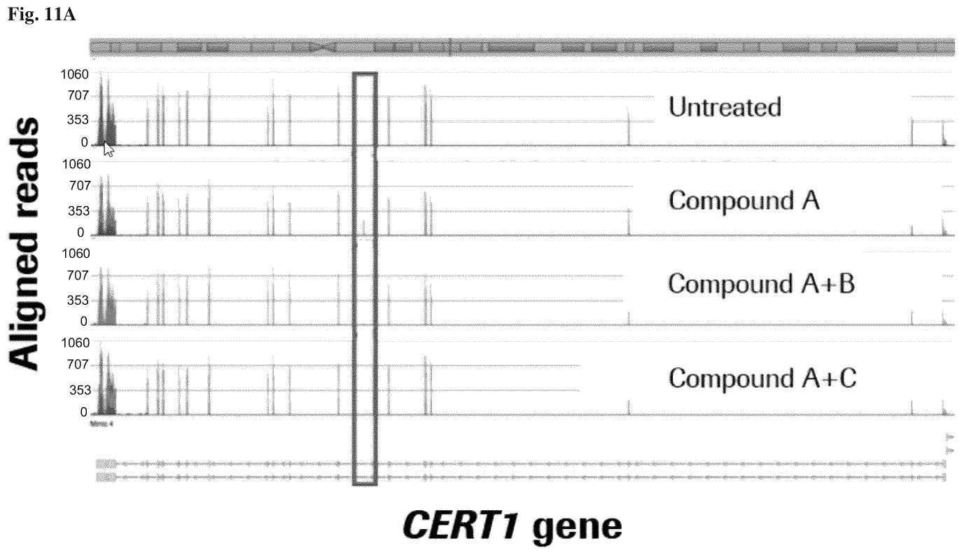

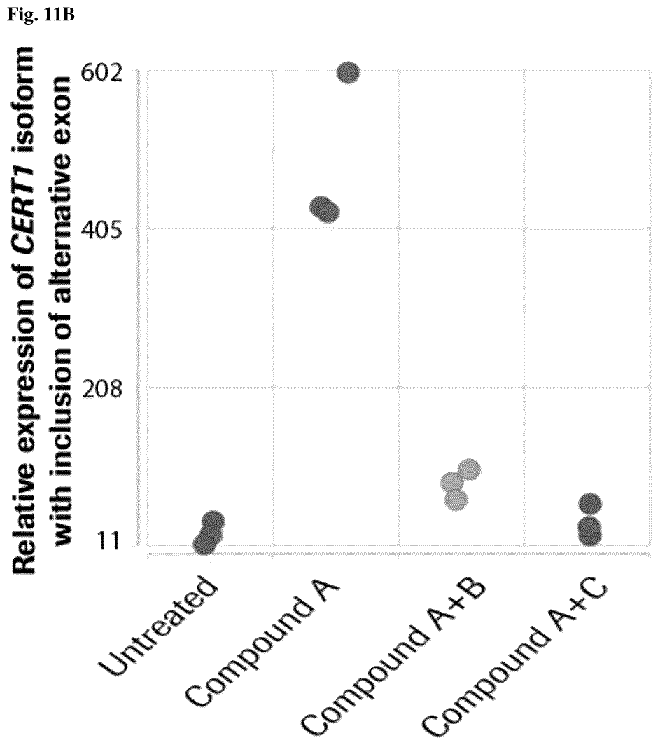

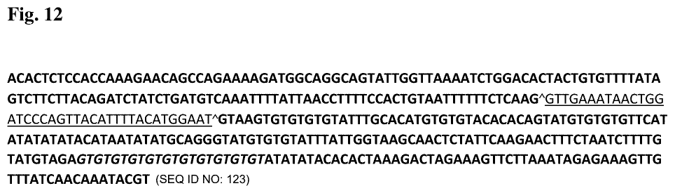

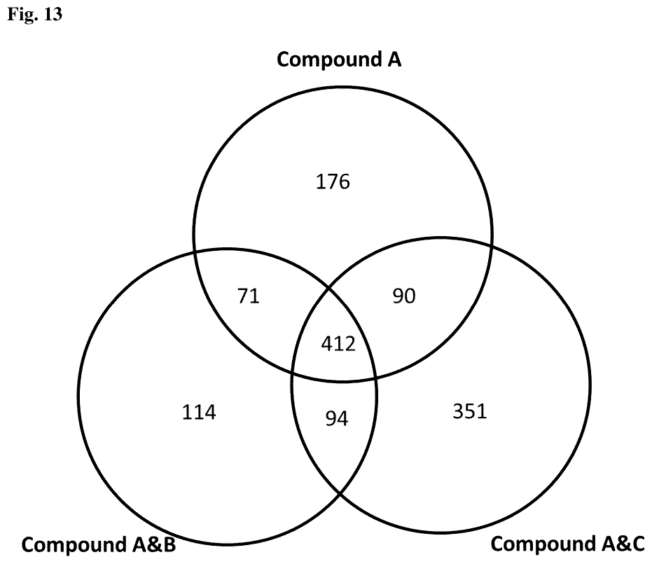

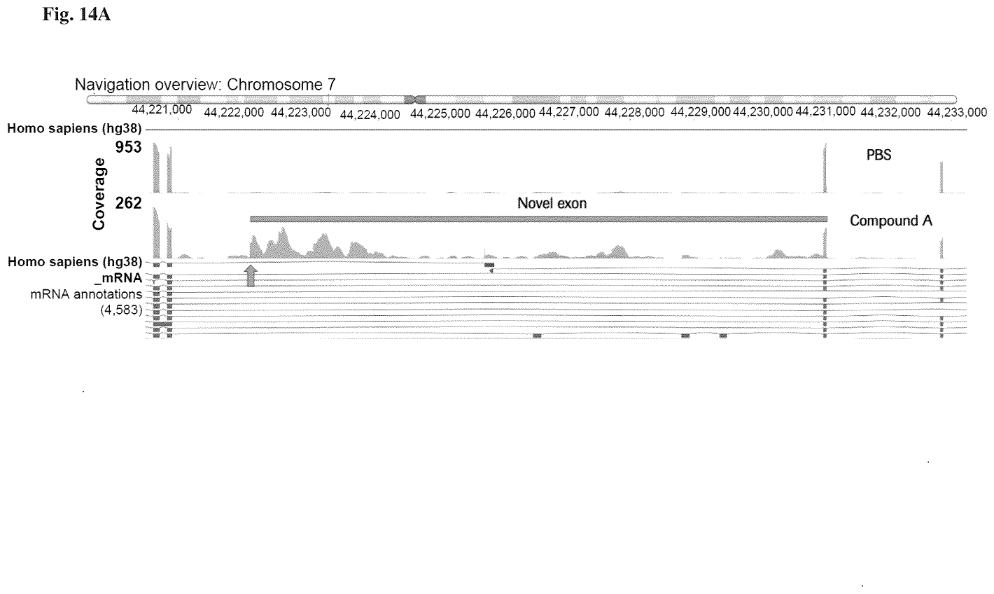

Another transcript that shows alternative splicing is the CERT1 mRNA encoding ceramide transfer protein or (CERT1). Loss of TDP-43 results in the use of a new splice donor and splice acceptor site to include a novel exon located at hg38 chr5: 75415197-75415235 (position 96395-96433 in ENSG00000113163) (FIGS. 11A-11B and 12). Loss of TDP-43 (compound A) increases novel isoform approximately 18-fold, whereas subsequent treatment with both the STMN2 ASO (Compound A+B) and the TDP-43 mimicking ASO (Compound A+C) remove the inclusion of this novel in frame exon to resemble wild type cells (FIGS. 11A and 11B). Within the pre-mRNA the cryptic exon is followed by a number of potential TDP-43 binding sites (FIG. 12). The cryptic exon is 39 bp in length and thereby does not change the ORF, but introduces 13 amino acids within the ceramide transfer protein (CERT1). The consequence of this is unknown. CERT1 is responsible for the transfer of ceramide from the endoplasmic reticulum (ER) to the Golgi apparatus. Ceramide is synthesized at the ER, then is transferred by CERT1 to Golgi where it is converted to sphingomyelin. Sphingomyelin is a type of sphingolipid found in animal cell membranes, especially in the membranous myelin sheath that surrounds some nerve cell axons. Regulation of the ceramide signaling pathways is involved in several neurodegenerative and neuroinflammatory diseases (Jana et al. J Neurol Sci. 2009 Mar. 15; 278 (1-2): 5-15).

To compare whether the TDP-43 targeting ASOs (Compound B and compound C) had similar targets, an analysis of alternative spliced transcripts from which a few selection criteria was set up. The p-value should be less than 0.01, and there should be more than a 2-fold change compared to the control analysis. The number of transcripts that shows alternative splicing upon knock down of TARDBP (Compound A versus untreated cells) is 749, whereas the number of transcripts that where alternative spliced using the STMN2 targeting ASO after initial TARDBP KD was 691 transcripts (compound A+B versus compound A). Of these 691 alternatively expressed transcripts, 483 were overlapping with the transcripts identified from TDP-43 KD. Finally, a comparison of the TDP-43 mimicking ASO after TARDBP Knock-down (KD, TDP-43 depletion) was compared to the TARDBP KD (compound A+C versus compound A). That revealed 502 alternatively spliced transcripts that were the same as seen in the initial TDP-43 KD versus untreated cells (FIG. 13). This analysis indicates that the antisense oligonucleotides of the invention have a more global effect on modulating RNA splicing as compared to compounds which are targeted specifically to STMN2, a result which is entirely consistent with the specific transcript analysis for ARHGAP32, SLC5A7 and CERT1 where the STMN2 specific compound B was not able to correct the aberrant splicing caused by TDP-43 depletion, whereas the compounds of the present invention were effective.

### Example 3: Rescue of Erroneous mRNA Splicing Caused by the Lack of TDP43 Using CA-Repeat ASO

Here we show the ability of CA-repeat antisense oligonucleotides to induce proper splicing on some TDP43 targets (STMN2, KALRN, CAMK2B, CERT1 and UNC13A).

### CAMK2B

Another pre-mRNA that shows alternative splicing is the CAMK2B transcript encoding calcium/calmodulin-dependent protein kinase type II subunit beta, and is involved in dendritic spine and synapse formation and neuronal plasticity. CAMK2B is located on chromosome 7:44217150-44334577:−1 (hg38) (Ensembl entry ENSG00000058404)

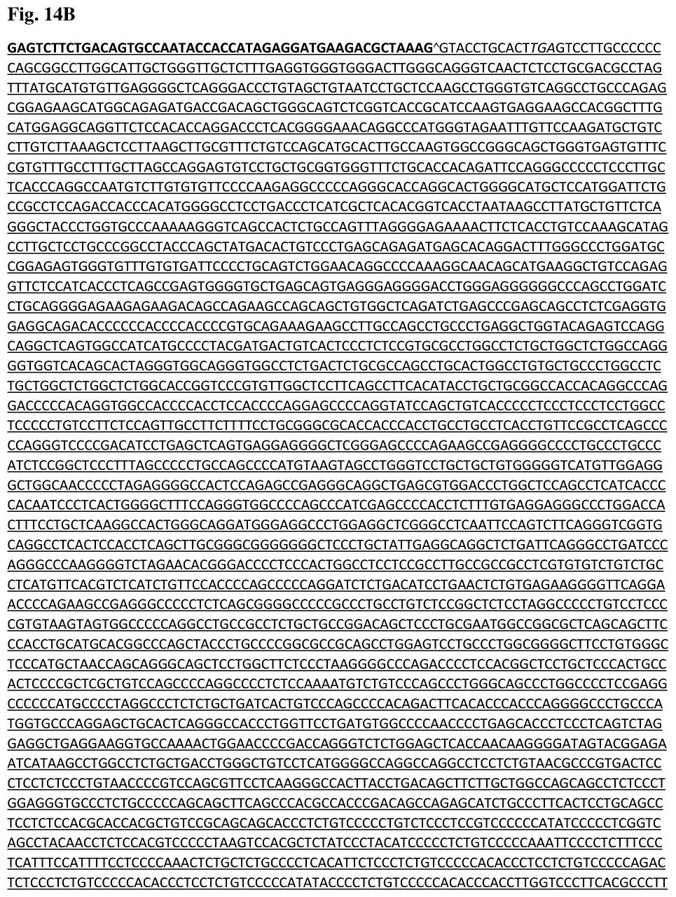

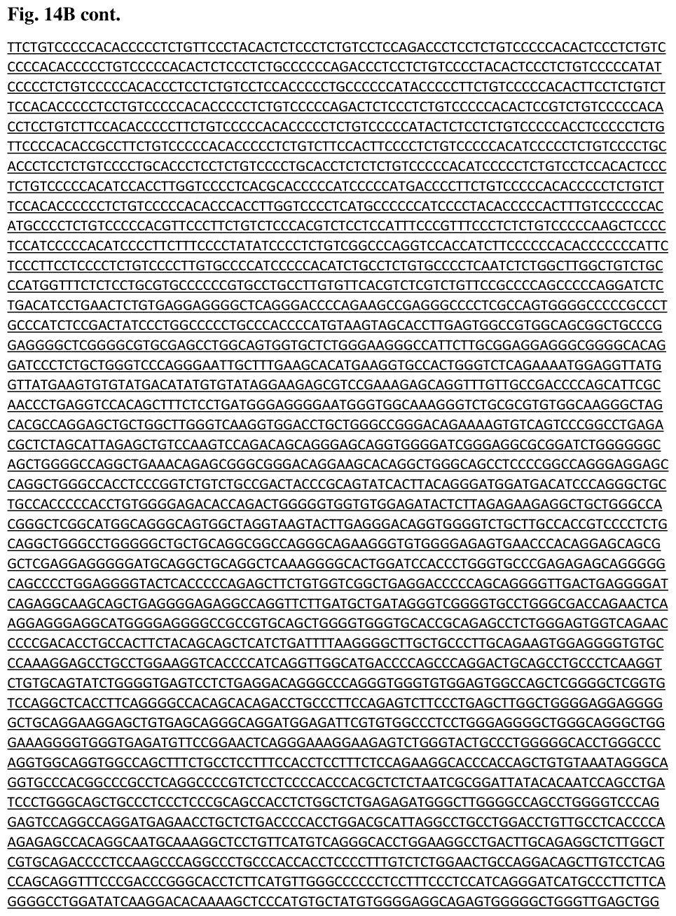

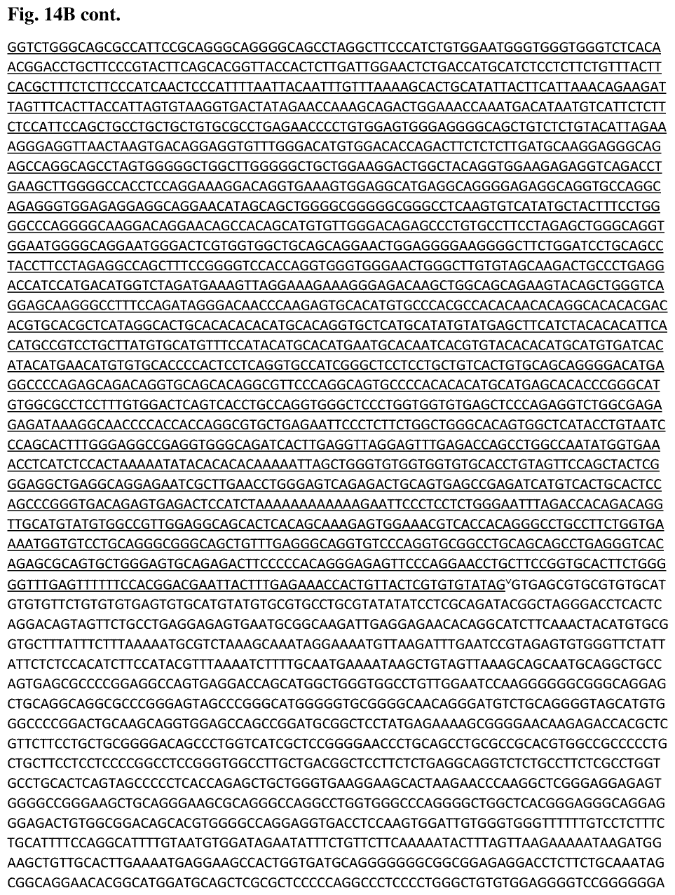

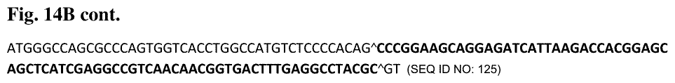

Loss of TDP43 protein decreases the use of the canonical splice donor site at position chr7: 44,231,004-44,231,005, and results in an extended exon. Within this sequence, a novel splice acceptor site is also used upon loss of TDP43 protein located at chr7: 44,222, 113-44,222,114. The novel exon is therefore located on chr7: 44,222,115-44,231,054. This exon is 8938 nucleotides long, and contains a large number of stop codons, resulting in nonsense mediated decay of the CAMK2B mRNA (FIG. 14B).

### KALRN

KALRN gene (Ensembl entry ENSG00000160145) is located on chromosome 3 (Chromosome 3:124, 033,369-124,726,325) (hg38). KALRN encodes kalirin RhoGEF kinase, also known as Huntingtin-associated protein-interacting protein, Protein Duo or Serine/threonine-protein kinase with Dbl- and pleckstrin homology domain.

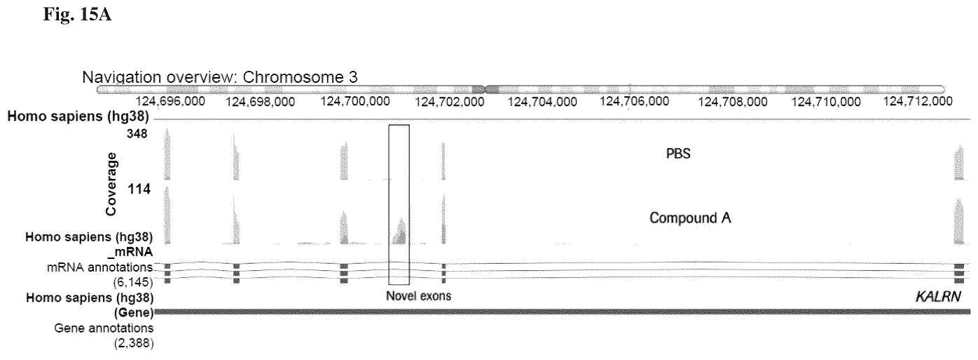

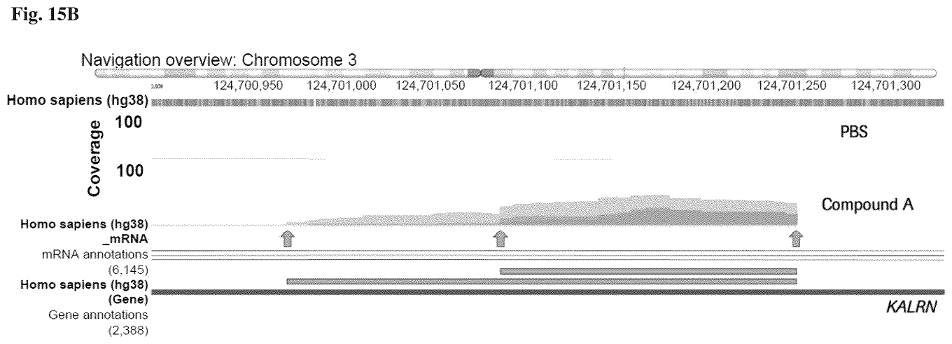

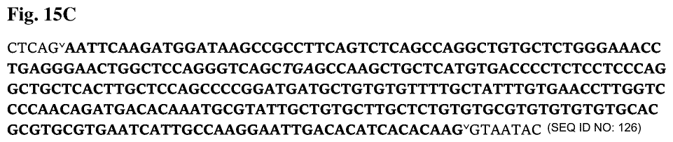

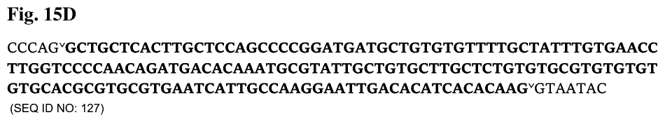

Loss of TDP43 protein increases the use of two novel alternative splice acceptor sites within KALRN pre-mRNA (chr3: 124,700,977; AG positioned at chr3: 124,700,975-124,700,976) and a splice donor site located (chr3: 124,701,255, GT positioned at chr3: 124,701,256-124,701,257) (FIGS. 15A and 15B). This results in the inclusion of a novel exon of 279 nucleotides (chr3: 124,700,977-124,701,255), which potentially encodes 93 amino acids. However, as seen from FIG. 15C, the ORF quickly enters a nonsense codon shown in italic and underlined TGA.

### UNC13A

UNC13A encodes Protein unc-13 homolog A. The UNC13A gene (Ensembl entry, ENSG00000130477) is located on chromosome 19:17601336-17688365:−1 (hg38) (minus strand). Protein unc-13 homolog A is involved in neurotransmitter release by acting in synaptic vesicle priming prior to vesicle fusion and participates in the activity-dependent refilling of readily releasable vesicle pool (RRP). Essential for synaptic vesicle maturation in most excitatory/glutamatergic but not inhibitory/GABA-mediated synapses (UniProt). Variants within the UNC13A gene have long been known to increase risk of amyotrophic lateral sclerosis (ALS) and frontotemporal dementia (FTD), and in two recent papers the inclusion of a cryptic exon upon TDP43 loss was described (Anna-Leigh Brown et al, “Common ALS/FTD risk variants in UNC13A exacerbate its cryptic splicing and loss upon TDP-43 mis-localization” bioRxiv 2021.04.02.438170; doi.org/10.1101/2021.04.02.438170; X. Rosa Ma et al., TDP-43 represses cryptic exon inclusion in FTD/ALS gene UNC13A, bioRxiv 2021.04.02.438213; doi.org/10.1101/2021.04.02.438213). Furthermore, two known polymorphisms strongly associated with ALS/FTD risk which alter TDP-43 binding within UNC13A pre-mRNA and thereby lead to inclusion of the cryptic exon Brown et al, 2021).

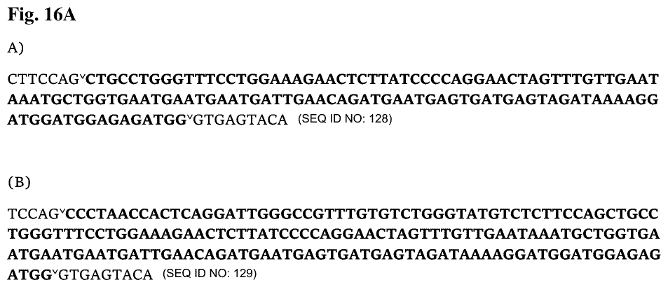

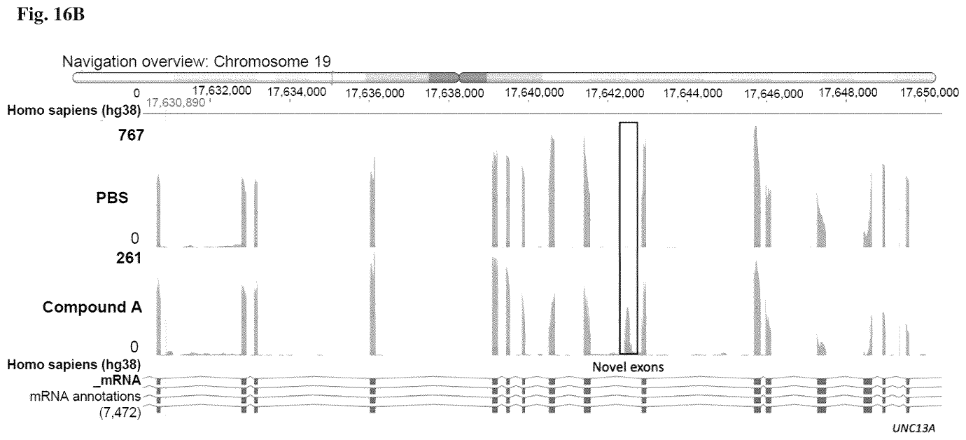

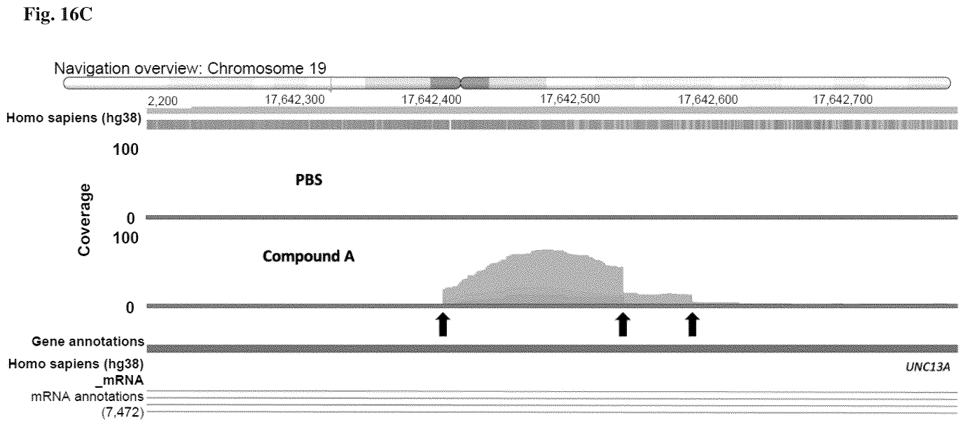

Our data shows in a similar way inclusion of the cryptic exons described by Brown et al, 2021 as described below. Upon TDP43 loss, two exons within UNC13A are observed by usage of alternative splice acceptor and splice donor sites. One exon is 128 nucleotides in length and is located on chromosome 19:17,642,414-17,642,541, and the other is 178 nucleotides in length and is located on chromosome 19:17,642,414-17,642,591 (FIG. 16A). The two exons are overlapping and use the same splice acceptor site chr19: 17,642,414, whereas the splice donor sites differ, chr19: 17,642,591 and chr19: 17,642,541, respectively (FIGS. 16B and 16C).Both exons are disrupting the open reading frame of UNC13A (FIG. 16A).

Human glutamatergic neurons (Fujifilms) were plated at 60000 cells per 96-well plates coated with Laminin and Poly (ethyleneimine) solution (Sigma Aldrich) in 200 ul Culture medium on Day 0. Half the cell culture medium was changed 3 times a week during the whole experiment (day 1, 5, 8, 11, 13, 15). To knockdown TDP-43, compound A (SEQ ID 19,1) was added to the culture medium at 5 μM on day 1 and day 8 (Except for two control wells). The CA-repeat antisense oligonucleotide was added to the culture medium on day 5 and day 13 at 10 μM. 48 different CA-repeat antisense oligonucleotides were added in total. 12 wells received only the compound A (Compound #19,1) to serve as a baseline reference.

The cells were harvested on day 18 using Magnapure lysis buffer (Roche) and RNA was isolated on MagNA pure 96 system (Roche) according to the manufacturer's instructions including DNase treatment step. The purified RNA was denatured 30 seconds at 90° C. before cDNA synthesis. cDNA was created using the iScript Advanced cDNA Synthesis Kit for RT-qPCR (Biorad) according to the manufacturer's instructions.

Measurements of the expression levels of the target genes was done by droplet digital PCR using the QX1 system (Bio-Rad). The PCR-probe assays used to measure the expressed of normally spliced target mRNA was designed to span the two exons, where in-between the new “mutant” exon would occur.

Data shown in Results Table B was normalized to the expression of the house keeping gene HPRT1, and finally normalized to the average expression value of the two control wells (PBS) that did not receive any TDP43 knock-down or CA-repeat antisense oligonucleotide.

The following PCR probe assay synthesized at (Integrated DNA technologies (IDT)) were used:

TARDBP
Primer 1:
(SEQ ID NO: 44)
CAGCTCATCCTCAGTCATGTC
Primer 2:
(SEQ ID NO: 45)
GATGGTGTGACTGCAAACTTC
Probe:
SEQ ID NO: 104)
/5Cy5/CAGCGCCCCACAAACACTTTTCT/3IAbRQSp/
STMN2
Primer 1:
(SEQ ID NO: 105)
CTGCTCAGCGTCTGC
Primer 2:
(SEQ ID NO: 106)
GTTGCGAGGTTCCGG
Probe:
(SEQ ID NO: 107)
/5HEX/CTAAAACAG/ZEN/CAATGGCCTACAAGGAAAAAATGAAG/
3IABkFQ/
CERT1
Primer 1:
(SEQ ID NO: 108)
CTAATGGTTAAACGTGAGGACAGC
Primer 2:
(SEQ ID NO: 109)
ATCTGGTCCTCCAAAGTGGG
Probe:
(SEQ ID NO: 110)
/5HEX/CAGAAGAGA/ZEN/CTGGATAAGGAAACTGAGAAGAAAAGAAGA
ACAG/3IABkFQ/
KALRN
Primer 1:
(SEQ ID NO: 111)
CGAGCCCTCGGAGTTTG
Primer 2:
(SEQ ID NO: 112)
TCCTTCCAAGAAATGGTGGC
Probe:
(SEQ ID NO: 113)
/5HEX/CGACTTCCA/ZEN/GAATATGATGCTGCTGCTGATG/
3IABkFQ/
CAMK2B
Primer 1:
(SEQ ID NO: 114)
CTGACAGTGCCAATACCACC
Primer 2:
(SEQ ID NO: 115)
GCTGCTCCGTGGTCTTAAT
Probe:
(SEQ ID NO: 116)
/5Cy5/ATGAAGACGCTAAAGCCCGGAAGCAG/3IAbRQSp/
UNC13A
Primer 1:
(SEQ ID NO: 117)
GATCAAAGGCGAGGAGAAGG
Primer 2:
(SEQ ID NO: 118)
TGGCATCTGGGATCTTCAC
Probe:
(SEQ ID NO: 119)
/56-FAM/ACCTGTCTG/ZEN/CATGAGAACCTGTTCCACTTC/
3IABkFQ/

The following CY5.5 labelled HPRT1 probe was purchased from BioRad: dHsaCPE13136107.

RESULTS TABLE B: Displays the expression value of the genes TARDBP, STMN2, CERT1, CAMK2B, KALRN and UNC13A. Expression is displayed as percentage expression compared to the average of untreated glutaneurons (PBS). As can be seen, several of the CA repeat antisense oligonucleotides are capable of elevating the expression of the target genes, compared to cells only treated with the TDP43 knockdown antisense oligonucleotide TDP43 KD (Seq ID 19,1).

Results Table B

| | | | Rescue | | | | | | |
|---|---|---|---|---|---|---|---|---|---|
| | SEQ | | Compound | | | | | | |
| Compound | ID NO | Base sequence | class | TARDBP | STMN2 | CERT1 | CAMK2B | UNC13A | KALRN |
| TDP43 KD | 19.1 | | | 2.5 | 44.0 | 75.9 | 21.7 | 50.4 | 1.2 |
| TDP43 KD | 19.1 | | | 2.0 | 35.8 | 60.6 | 21.9 | 35.4 | 2.2 |
| TDP43 KD | 19.1 | | | 2.2 | 45.9 | 63.6 | 20.7 | 42.4 | 0.0 |
| TDP43 KD | 19.1 | | | 2.2 | 46.4 | 68.2 | 16.4 | 40.9 | 1.4 |
| TDP43 KD | 19.1 | | | 1.6 | 44.9 | 60.5 | 22.1 | 48.3 | 1.9 |
| TDP43 KD | 19.1 | | | 2.1 | 42.2 | 60.9 | 21.5 | 38.6 | 2.1 |
| TDP43 KD | 19.1 | | | 2.2 | 39.6 | 56.7 | 16.8 | 37.0 | 1.3 |
| TDP43 KD | 19.1 | | | 1.7 | 46.7 | 51.4 | 18.7 | 26.7 | 1.1 |
| TDP43 KD | 19.1 | | | 2.1 | 39.4 | 59.7 | 16.9 | 43.3 | 1.8 |
| TDP43 KD | 19.1 | | | 1.4 | 39.5 | 64.8 | 19.3 | 34.4 | 2.2 |
| TDP43 KD | 19.1 | | | 3.3 | 45.3 | 69.3 | 20.3 | 41.1 | 1.6 |
| TDP43 KD | 19.1 | | | 3.8 | 46.0 | 79.9 | 25.4 | 47.2 | 0.9 |
| PBS | | | | 91.0 | 92.7 | 92.9 | 91.2 | 95.2 | 112.5 |
| PBS | | | | 109.0 | 107.3 | 107.1 | 108.8 | 104.8 | 87.5 |
| SEQ ID 20 | 20 | CACACACACACACACACACACAC | LNA mixmer | 1.7 | 152.4 | 128.6 | 89.6 | 78.6 | 53.6 |
| SEQ ID 21 | 21 | ACACACACACACACACACACACAC | LNA mixmer | 2.8 | 48.1 | 96.7 | 28.8 | 41.5 | 8.0 |
| SEQ ID 22 | 22.1 | CACACACACACAACACACACACAC | LNA mixmer | 2.7 | 74.9 | 132.0 | 37.6 | 29.2 | 19.3 |
| SEQ ID 22 | 22.2 | CACACACACACAACACACACACAC | LNA mixmer | 1.6 | 37.0 | 78.2 | 25.8 | 23.6 | 4.7 |
| SEQ ID 23 | 23.6 | CACACACACACACACACACACACAC | LNA mixmer | 2.0 | 83.2 | 113.7 | 37.3 | 60.8 | 5.8 |
| SEQ ID 23 | 23.7 | CACACACACACACACACACACACAC | LNA mixmer | 2.0 | 95.7 | 96.1 | 25.9 | 25.7 | 6.0 |
| SEQ ID 23 | 23.8 | CACACACACACACACACACACACAC | LNA mixmer | 3.4 | 71.1 | 121.0 | 42.6 | 48.0 | 21.7 |
| SEQ ID 23 | 23.9 | CACACACACACACACACACACACAC | LNA mixmer | 1.9 | 81.3 | 115.5 | 35.4 | 51.5 | 15.1 |
| SEQ ID 24 | 24 | ACACACACACACACACACACACACAC | LNA mixmer | 3.3 | 50.3 | 101.3 | 25.2 | 34.1 | 7.1 |
| SEQ ID 25 | 25.1 | CACACACACACACACACACACACACAC | LNA mixmer | 2.2 | 87.1 | 113.9 | 37.7 | 47.6 | 14.6 |
| SEQ ID 25 | 25.2 | CACACACACACACACACACACACACAC | LNA mixmer | 1.7 | 90.1 | 110.1 | 27.5 | 35.2 | 6.8 |
| SEQ ID 25 | 25.3 | CACACACACACACACACACACACACAC | LNA mixmer | 2.7 | 56.4 | 100.3 | 34.2 | 42.7 | 16.4 |
| SEQ ID 25 | 25.4 | CACACACACACACACACACACACACAC | LNA mixmer | 2.9 | 86.9 | 97.0 | 34.0 | 49.7 | 16.4 |
| SEQ ID 26 | 26 | ACACACACACACACACACACACACACAC | LNA mixmer | 2.6 | 51.6 | 98.8 | 28.7 | 46.0 | 7.7 |
| SEQ ID 27 | 27.1 | CACACACACACACACACACACACACACA | LNA mixmer | 2.3 | 88.2 | 118.9 | 36.8 | 65.3 | 14.4 |
| SEQ ID 27 | 27.2 | CACACACACACACACACACACACACACA | LNA mixmer | 1.9 | 70.7 | 115.3 | 33.1 | 52.7 | 11.9 |
| SEQ ID 27 | 27.3 | CACACACACACACACACACACACACACAC | LNA mixmer | 2.7 | 58.9 | 97.5 | 31.1 | 53.3 | 8.3 |
| SEQ ID 27 | 27.4 | CACACACACACACACACACACACACACAC | LNA mixmer | 2.4 | 90.8 | 119.9 | 30.7 | 32.7 | 3.7 |
| SEQ ID 28 | 28 | ACACACACACACACACACACACACACACAC | LNA mixmer | 1.8 | 41.6 | 94.0 | 29.1 | 43.2 | 8.5 |
| SEQ ID 29 | 29.1 | CACACACACACACACACACACACACACACAC | LNA mixmer | 3.3 | 63.6 | 87.4 | 26.7 | 35.7 | 12.8 |
| SEQ ID 29 | 29.2 | CACACACACACACACACACACACACACACAC | LNA mixmer | 3.4 | 70.9 | 123.3 | 34.3 | 46.1 | 7.9 |
| SEQ ID 29 | 29.3 | CACACACACACACACACACACACACACACAC | LNA mixmer | 2.1 | 48.3 | 90.0 | 23.8 | 45.7 | 13.7 |
| SEQ ID 29 | 29.4 | CACACACACACACACACACACACACACACA | LNA mixmer | 3.0 | 48.4 | 116.6 | 22.4 | 20.6 | 7.6 |
| | | C-3′ | | | | | | | |
| SEQ ID 30 | 30 | 5′-ACACACACACACACACACCACACACACA | LNA mixmer | 2.2 | 57.5 | 108.9 | 27.3 | 32.3 | 2.1 |
| | | CACACA | | | | | | | |
| SEQ ID 31 | 31.1 | CACACACACACACACACACACACACACACAC | LNA mixmer | 2.6 | 61.7 | 105.6 | 29.7 | 45.1 | 10.2 |
| | | ACA-3′ | | | | | | | |
| SEQ ID 31 | 31.2 | CACACACACACACACACACACACACACACAC | LNA mixmer | 3.2 | 58.7 | 110.4 | 25.3 | 31.7 | 9.6 |
| | | ACA | | | | | | | |
| SEQ ID 32 | 32 | CACACACACACACACACAACACACACACACA | LNA mixmer | 2.5 | 59.0 | 98.4 | 26.8 | 26.6 | 8.3 |
| | | CACAC | | | | | | | |
| SEQ ID 33 | 33 | ACACACACACACACACACCACACACACACAC | LNA mixmer | 1.7 | 57.7 | 79.6 | 23.7 | 38.8 | 3.0 |
| | | ACACA | | | | | | | |
| SEQ ID 22 | 22.3 | CACACACACACAACACACACACAC | MOE | 2.2 | 60.6 | 100.3 | 31.6 | 55.3 | 12.3 |
| SEQ ID 23 | 23.1 | CACACACACACACACACACACACAC | MOE | 2.7 | 89.0 | 90.4 | 29.0 | 35.7 | 24.9 |
| SEQ ID 25 | 25.5 | CACACACACACACACACACACACACAC | MOE | 1.9 | 79.5 | 103.1 | 28.8 | 45.6 | 16.4 |
| SEQ ID 27 | 27.5 | CACACACACACACACACACACACACACAC | MOE | 3.0 | 91.3 | 95.3 | 24.9 | 37.8 | 22.5 |
| SEQ ID 29 | 29.5 | CACACACACACACACACACACACACACACAC | MOE | 2.5 | 87.3 | 109.4 | 30.8 | 37.4 | 18.1 |
| SEQ ID 23 | 23.2 | CACACACACACACACACACACACAC | MOE | 4.3 | 36.1 | 75.8 | 25.9 | 37.8 | 1.5 |
| | | | mixmer | | | | | | |
| SEQ ID 25 | 25.6 | CACACACACACACACACACACACACAC | MOE | 3.4 | 37.9 | 69.1 | 20.3 | 37.2 | 1.0 |
| | | | mixmer | | | | | | |
| SEQ ID 23 | 23.3 | CACACACACACACACACACACACAC | MOE | | | | | | |
| | | | mixmer | 2.5 | 56.4 | 100.5 | 25.4 | 53.9 | 5.5 |
| SEQ ID 25 | 25.7 | CACACACACACACACACACACACACAC | MOE | 2.0 | 52.7 | 101.9 | 24.2 | 42.1 | 5.2 |
| | | | mixmer | | | | | | |
| SEQ ID 27 | 27.6 | CACACACACACACACACACACACACACAC | MOE | 3.2 | 42.2 | 93.1 | 27.2 | 51.9 | 1.2 |
| | | | mixmer | | | | | | |
| SEQ ID 27 | 27.7 | CACACACACACACACACACACACACACAC | MOE | 2.2 | 58.0 | 97.3 | 24.9 | 44.0 | 1.8 |
| | | | mixmer | | | | | | |
| SEQ ID 29 | 29.6 | CACACACACACACACACACACACACACACAC | MOE | 3.4 | 31.3 | 62.2 | 14.0 | 19.1 | 1.3 |
| | | | mixmer | | | | | | |
| SEQ ID 29 | 29.7 | CACACACACACACACACACACACACACACAC | MOE | 2.4 | 60.2 | 97.6 | 26.0 | 41.7 | 5.3 |
| | | | mixmer | | | | | | |
| SEQ ID 23 | 23.4 | CACACACACACACACACACACACAC | 2′O-Methyl | 2.9 | 42.5 | 96.4 | 26.2 | 43.0 | 3.2 |
| SEQ ID 25 | 25.8 | CACACACACACACACACACACACACAC | 2′O-Methyl | 3.3 | 49.3 | 106.6 | 28.2 | 45.9 | 3.2 |
| SEQ ID 27 | 27.8 | CACACACACACACACACACACACACACAC | 2′O-Methyl | 2.9 | 46.8 | 104.1 | 29.1 | 52.4 | 1.1 |
| SEQ ID 29 | 29.8 | CACACACACACACACACACACACACACACAC | 2′O-Methyl | 2.4 | 46.2 | 96.5 | 27.1 | 33.9 | 1.0 |
| SEQ ID 23 | 23.5 | CACACACACACACACACACACACAC | 2′O-Methyl | 2.6 | 31.7 | 72.1 | 17.6 | 25.7 | 2.0 |
| | | | Mixmer | | | | | | |
| SEQ ID 25 | 25.9 | CACACACACACACACACACACACACAC | 2′O-Methyl | 3.0 | 40.7 | 85.7 | 17.3 | 27.5 | 0.3 |
| | | | Mixmer | | | | | | |
| SEQ ID 27 | 27.9 | CACACACACACACACACACACACACACAC | 2′O-Methyl | 3.5 | 44.5 | 102.3 | 22.2 | 40.2 | 1.9 |
| | | | Mixmer | | | | | | |
| SEQ ID 29 | 29.9 | CACACACACACACACACACACACACACACAC | 2′O-Methyl | 2.4 | 42.3 | 85.0 | 15.7 | 37.7 | 1.4 |
| | | | Mixmer | | | | | | |
| SEQ ID 34 | 34 | ACACACACACACACACACACACACACACACA | 2′O-Methyl | | | | | | |
| | | C | Mixmer | 2.4 | 52.0 | 110.4 | 30.1 | 40.4 | 9.2 |

### Example 4—Rescue of Erroneous mRNA Splicing Caused by the Lack of TDP43 Using CA-Repeat Rich ASOs

In order to illustrate that CA-repeat ASO containing some sequence variations are still capable of correcting erroneous mRNA splicing caused by the lack of TDP43, we produced CA-repeat ASO with up to 5 nucleotide changes and show that they are still capable of correcting mRNA splicing. These CA-repeat rich ASOs are tested for their ability to induce proper splicing on 3 known TDP43 targets (STMN2, KALRN, CAMK2B) in absence of TDP43.

Human glutamatergic neurons (Fujifilms) were plated at 60000 cells per 96-well plates coated with Laminin and Poly (ethyleneimine) solution (Sigma Aldrich) in 200 ul Culture medium on Day 0. Half the cell culture medium was changed 3 times a week during the whole experiment (day 1, 3, 6, 8, 10, 13, 15). To knockdown TDP-43, compound A (SEQ ID 19,1) was added to the culture medium at 5 μM on day 1 and day 8 (Except for four control wells). The CA-repeat rich ASOs were added to the culture medium on day 10 and day 13 at 10 μM. 60 different CA-repeat rich ASO were added, as indicated in Table C below. 10 wells received only the compound A (SEQ ID 19,1) to serve as a baseline reference.

The cells were harvested on day 16 using Magnapure lysis buffer (Roche) and RNA was isolation on MagNA pure 96 system (Roche) according to the manufacturer's instructions including DNase treatment step. The purified RNA was denatured 30 seconds at 90° C. before cDNA synthesis. cDNA was created using the iScript Advanced cDNA Synthesis Kit for RT-qPCR (Biorad) according to the manufacturer's instructions.

Measurements of the expression levels of the target genes were done by droplet digital PCR using the QX1 system (Bio-Rad). The PCR-probe assays used to measure the expression of normally spliced target mRNA was designed to span the two exons, where in-between the new “mutant” exon would occur.

Data shown in table C was normalized to the expression of the housekeeping gene HPRT1, and finally normalized to the average expression value of the two control wells (PBS) that did not receive any TDP43 knock-down or CA-repeat ASO.

The following PCR probe assay synthesized at (Integrated DNA technologies (IDT)) were used TARDBP:

Primer 1:
(SEQ ID NO: 44)
CAGCTCATCCTCAGTCATGTC
Primer 2:
(SEQ ID NO: 45)
GATGGTGTGACTGCAAACTTC
Probe:
(SEQ ID NO: 104)
/5Cy5/CAGCGCCCCACAAACACTTTTCT/3IAbRQSp/)
STMN2:
Primer 1:
(SEQ ID NO: 105)
CTGCTCAGCGTCTGC
Primer 2:
(SEQ ID NO: 106)
GTTGCGAGGTTCCGG
Probe:
(SEQ ID NO: 107)
/5HEX/CTAAAACAG/ZEN/CAATGGCCTACAAGGAAAAAATGAAG/
3IABkFQ/)
KALRN:
Primer 1:
(SEQ ID NO: 111)
CGAGCCCTCGGAGTTTG
Primer 2:
(SEQ ID NO: 112)
TCCTTCCAAGAAATGGTGGC
Probe:
(SEQ ID NO: 113)
/5HEX/CGACTTCCA/ZEN/GAATATGATGCTGCTGCTGATG/3IABkFQ/)
CAMK2B:
Primer 1:
(SEQ ID NO: 114)
CTGACAGTGCCAATACCACC
Primer 2:
(SEQ ID NO: 115)
GCTGCTCCGTGGTCTTAAT
Probe:
(SEQ ID NO: 116)
/5Cy5/ATGAAGACGCTAAAGCCCGGAAGCAG/3IAbRQSp/)

The following CY5.5 labelled HPRT1 probe was purchased from BioRad: dHsaCPE13136107.

Results Table C—Displays the expression value of the genes: TDP43, CAMK2B, STMN2 and KALRN. Expression is displayed as percentage expression compared to the average of untreated glutaneurons (PBS).

RESULTS TABLE C

| Compound | SEQ ID NO | TDP43 | CAMK2B | STMN2 | KALRN |
|---|---|---|---|---|---|
| PBS | | 99.2 | 99.4 | 101.7 | 103.9 |
| PBS | | 94.9 | 99.8 | 91.5 | 80.6 |
| PBS | | 97.6 | 86.7 | 99.7 | 97.5 |
| PBS | | 108.2 | 114.2 | 107.1 | 118.0 |
| TDP43 KD | Seq ID 19, 1 | 2.3 | 26.7 | 34.1 | 3.1 |
| TDP43 KD | Seq ID 19, 1 | 0.9 | 28.5 | 30.2 | 2.3 |
| TDP43 KD | Seq ID 19, 1 | 1.2 | 24.9 | 35.4 | 1.9 |
| TDP43 KD | Seq ID 19, 1 | 1.0 | 22.5 | 35.6 | 1.8 |
| TDP43 KD | Seq ID 19, 1 | 1.5 | 25.4 | 40.0 | 3.2 |
| TDP43 KD | Seq ID 19, 1 | 1.5 | 25.3 | 36.1 | 1.1 |
| TDP43 KD | Seq ID 19, 1 | 2.1 | 25.1 | 34.0 | 1.6 |
| TDP43 KD | Seq ID 19, 1 | 2.1 | 26.9 | 35.4 | 1.6 |
| TDP43 KD | Seq ID 19, 1 | 2.1 | 19.8 | 30.5 | 1.7 |
| TDP43 KD | Seq ID 19, 1 | 2.2 | 19.3 | 32.6 | 2.2 |
| Seq ID 20 | Seq ID 20 | 2.2 | 32.6 | 42.5 | 6.7 |
| Seq ID 20 | Seq ID 20, 2 | 2.1 | 31.6 | 53.8 | 21.8 |
| Seq ID 50 | Seq ID 50 | 1.9 | 27.4 | 36.5 | 8.6 |
| Seq ID 51 | Seq ID 51 | 2.1 | 36.4 | 46.8 | 9.3 |
| Seq ID 52 | Seq ID 52 | 2.4 | 35.8 | 46.4 | 18.7 |
| Seq ID 53 | Seq ID 53 | 2.9 | 28.1 | 45.6 | 7.8 |
| Seq ID 54 | Seq ID 54 | 1.6 | 33.3 | 37.2 | 5.8 |
| Seq ID 55 | Seq ID 55 | 2.7 | 28.3 | 48.5 | 17.2 |
| Seq ID 56 | Seq ID 56 | 2.4 | 41.1 | 62.5 | 14.3 |
| Seq ID 57 | Seq ID 57 | 2.0 | 31.9 | 53.6 | 6.0 |
| Seq ID 58 | Seq ID 58 | 2.6 | 35.3 | 54.9 | 5.7 |
| Seq ID 59 | Seq ID 59 | 2.3 | 27.3 | 38.1 | 11.7 |
| Seq ID 60 | Seq ID 60 | 2.9 | 37.7 | 49.5 | 11.8 |
| Seq ID 61 | Seq ID 61 | 2.0 | 29.4 | 33.5 | 3.0 |
| Seq ID 62 | Seq ID 62 | 2.6 | 28.0 | 53.1 | 4.4 |
| Seq ID 63 | Seq ID 63 | 2.6 | 27.8 | 42.0 | 4.7 |
| Seq ID 64 | Seq ID 64 | 2.0 | 34.6 | 37.8 | 6.7 |
| Seq ID 65 | Seq ID 65 | 2.3 | 30.6 | 47.2 | 12.4 |
| Seq ID 66 | Seq ID 66 | 2.7 | 31.4 | 38.6 | 3.9 |
| Seq ID 67 | Seq ID 67 | 3.0 | 33.6 | 42.5 | 4.7 |
| Seq ID 68 | Seq ID 68 | 3.0 | 33.9 | 44.9 | 8.9 |
| Seq ID 69 | Seq ID 69 | 2.5 | 29.9 | 44.3 | 4.7 |
| Seq ID 27 | Seq ID 27, 1 | 2.7 | 33.5 | 49.8 | 19.0 |
| Seq ID 70 | Seq ID 70 | 2.6 | 35.9 | 52.2 | 12.1 |
| Seq ID 71 | Seq ID 71, 1 | 2.8 | 34.1 | 49.2 | 8.9 |
| Seq ID 71 | Seq ID 71, 2 | 2.5 | 30.0 | 49.5 | 4.5 |
| Seq ID 72 | Seq ID 72 | 1.3 | 22.7 | 32.7 | 3.1 |
| Seq ID 73 | Seq ID 73 | 1.8 | 26.2 | 45.7 | 6.0 |
| Seq ID 74 | Seq ID 74 | 3.1 | 23.9 | 41.4 | 5.2 |
| Seq ID 75 | Seq ID 75 | 2.4 | 26.6 | 43.0 | 4.1 |
| Seq ID 76 | Seq ID 76 | 2.0 | 30.5 | 47.3 | 4.7 |
| Seq ID 77 | Seq ID 77 | 2.0 | 31.2 | 48.2 | 5.6 |
| Seq ID 78 | Seq ID 78 | 3.0 | 39.3 | 67.3 | 8.0 |
| Seq ID 79 | Seq ID 79 | 2.2 | 40.9 | 45.5 | 4.9 |
| Seq ID 80 | Seq ID 80 | 2.4 | 27.6 | 43.8 | 6.9 |
| Seq ID 81 | Seq ID 81 | 2.8 | 40.4 | 49.4 | 13.5 |
| Seq ID 82 | Seq ID 82 | 3.4 | 29.8 | 55.1 | 9.5 |
| Seq ID 83 | Seq ID 83 | 2.0 | 35.1 | 62.6 | 8.8 |
| Seq ID 84 | Seq ID 84 | 2.4 | 31.1 | 52.6 | 8.6 |
| Seq ID 85 | Seq ID 85 | 1.8 | 30.2 | 44.4 | 3.9 |
| Seq ID 86 | Seq ID 86 | 2.0 | 35.5 | 32.8 | 2.9 |
| Seq ID 23 | Seq ID 23, 1 | 1.7 | 33.4 | 61.5 | 27.5 |
| Seq ID 87 | Seq ID 87 | 1.8 | 34.0 | 72.1 | 7.4 |
| Seq ID 88 | Seq ID 88 | 2.1 | 29.9 | 58.5 | 6.5 |
| Seq ID 23 | Seq ID 23, 10 | 2.3 | 31.6 | 36.1 | 13.3 |
| Seq ID 89 | Seq ID 89 | 2.6 | 27.4 | 63.3 | 10.1 |
| Seq ID 90 | Seq ID 90 | 1.3 | 26.0 | 41.3 | 10.8 |
| Seq ID 91 | Seq ID 91 | 1.6 | 19.6 | 44.2 | 6.8 |
| Seq ID 92 | Seq ID 92 | 2.0 | 30.9 | 90.7 | 7.3 |
| Seq ID 93 | Seq ID 93 | 1.2 | 26.6 | 57.1 | 7.1 |
| Seq ID 94 | Seq ID 94 | 1.5 | 34.7 | 51.0 | 14.3 |
| Seq ID 95 | Seq ID 95 | 1.8 | 22.6 | 44.5 | 8.3 |
| Seq ID 96 | Seq ID 96 | 2.1 | 24.4 | 72.8 | 14.3 |
| Seq ID 97 | Seq ID 97 | 1.5 | 31.7 | 82.0 | 16.9 |
| Seq ID 98 | Seq ID 98 | 1.9 | 34.5 | 43.8 | 19.4 |
| Seq ID 99 | Seq ID 99 | 2.1 | 25.1 | 45.0 | 10.6 |
| Seq ID 100 | Seq ID 100 | 1.9 | 32.4 | 59.6 | 7.1 |
| Seq ID 101 | Seq ID 101 | 1.6 | 36.9 | 73.7 | 25.9 |
| Seq ID 102 | Seq ID 102 | 1.8 | 29.5 | 74.0 | 30.5 |
| Seq ID 103 | Seq ID 103 | 1.4 | 33.6 | 68.1 | 18.5 |

### CERTAIN REFERENCES

Salter CG, Beijer D, Hardy H, et al. Truncating SLC5A7 mutations underlie a spectrum of dominant hereditary motor neuropathies. Neurol Genet. 2018;4 (2): e222.
Yukiko Nasu-Nishimura 1, Tomoatsu Hayashi, Tomohiro Ohishi, Toshio Okabe, Susumu Ohwada, Yoshimi Hasegawa, Takao Senda, Chikashi Toyoshima, Tsutomu Nakamura, Tetsu Akiyama. Role of the Rho GTPase-activating Protein RICS in Neurite Outgrowth. Genes Cells. 2006 Jun; 11 (6): 607-14.
Arundhati Jana, Edward L. Hogan, and Kalipada Pahan. Ceramide and neurodegeneration: Susceptibility of neurons and oligodendrocytes to cell damage and death. J Neurol Sci. 2009 Mar 15; 278 (1-2): 5-15.
Conti et al. TDP-43 affects splicing profiles and isoform production of genes involved in the apoptotic and mitotic cellular pathways. Nucleic Acids Res. 2015 Oct 15; 43 (18): 8990-9005.
Humphrey et al. Quantitative analysis of cryptic splicing associated with TDP-43 depletion. BMC Medical Genomics 2017; volume 10, Article number: 38 (2017).
Melamed et al. Premature polyadenylation-mediated loss of stathmin-2 is a hallmark of TDP-43-dependent neurodegeneration. Nat Neurosci. 2019 Feb; 22 (2): 180-190.
Klim et al., ALS-implicated protein TDP-43 sustains levels of STMN2, a mediator of motor neuron growth and repair. Nature Neuroscience 22, pages167-179 (2019)

### Compounds Used in Examples 1, 3 & 4

| SEQ | Base | Comp | |
|---|---|---|---|
| ID NO | sequence 5′-3′ | ID No | HELM |
| 1 | ACACACAC | 1.1 | [LR](A)[sP].[LR)([5meC])[sP].[LR](A)[sP].[dR](C)[sP].[dR](A)[sP]. |
| | | | [LR]([5meC])[sP].[LR](A)[sP].[LR]([5meC]) |
| 2 | ACACACACA | 2.1 | [LR](A)[sP].[LR]([5meC])[sP].[dR](A)[sP].[LR]([5meC])[sP].[LR](A) |
| | | | [sP].[LR]([5meC])[sP].[dR](A)[sP].[LR]([5meC])[sP].[LR](A) |
| 3 | ACACACACAC | 3.1 | [LR](A)[sP].[LR]([5meC])[sP].[dR](A)[sP].[LR]([5meC])[sP].[LR](A) |
| | | | [sP].[LR]([5meC])[sP][dR](A)[sP].[LR]([5meC])[sP].[LR](A)[sP]. |
| | | | [LR]([5meC]) |
| 4 | ACACACACACA | 4.1 | [LR](A)[sP].[LR]([5meC])[sP].[LR](A)[sP].[dR](C)[sP].[dR](A)[sP]. |
| | | | [LR]((5meC])[sP].[LR](A)[sP].[dR](C)[sP].[LR](A)[sP].[LR]([5meC]) |
| | | | [sP].[LR](A) |
| 5 | ACACACACACAC | 5.1 | [LR](A)[sP].[dR](C)[sP].[LR](A)[sP].[dR](C)[sP].[dR](A)[sP].[LR] |
| | | | ([5meC])[sP].[LR](A)[sP][LR]([5meC])[sP].[dR](A)[sP].[LR]([5meC]) |
| | | | [sP].[LR)(A)[sP].[LR]([5meC]) |
| 6 | ACACACACACACA | 6.1 | [LR](A)[sP].[dR](C)[sP].[LR](A)[sP].[dR](C)[sP].[LR](A)[sP].[LR] |
| | | | ([5meC])[sP].[dR](A)[sP][LR]([5meC])[sP].[dR](A)[sP].[LR]([5meC]) |
| | | | [sP].[dR](A)[sP].[LR]([5meC])[sP].[LR](A) |
| 6 | ACACACACACACA | 6.2 | [LR](A)[sP].[dR](C)[sP].[dR](A)[sP].[dR](C)[sP].[LR](A)[sP].[LR] |
| | | | ([5meC])[sP].[dR](A)[sP][dR](C)[sP].[dR](A)[sP].[LR]([5meC])[sP]. |
| | | | [dR](A)[sP].[LR]([5meC])[sP].[LR](A) |
| 6 | ACACACACACACA | 6.3 | [LR](A)[sP].[dR](C)[sP].[LR](A)[sP].[dR](C)[sP].[LR](A)[sP].[dR] |
| | | | (C)[sP].[dR](A)[sP].[LR]([5meC])[sP].[dR](A)[sP].[dR](C)[sP].[dR] |
| | | | (A)[sP].[LR]([5meC])[sP].[LR](A) |
| 6 | ACACACACACACA | 6.4 | [LR](A)[sP].[dR](C)[sP].[dR](A)[sP].[dR](C)[sP].[LR](A)[sP].[LR] |
| | | | ([5meC])[sP].[dR](A)[sP].[LR]([5meC])[sP].[dR](A)[sP].[dR](C)[sP]. |
| | | | [dR](A)[sP].[LR]([5meC])[sP].[LR](A) |
| 7 | ACACACACACACAC | 7.1 | [LR](A)[sP].[dR](C)[sP].[LR](A)[sP].[dR](C)[sP].[LR](A)[sP].[dR] |
| | AC | | (C)[sP].[LR](A)[sP].[dR](C)[sP].[LR](A)[sP].[dR](C)[sP].[LR](A) |
| | | | [sP].[dR](C)[sP].[LR](A)[sP].[dR](C)[sP].[LR](A)[sP].[LR]([5meC]) |
| 7 | ACACACACACACAC | 7.2 | [LR](A)[sP].[dR](C)[sP].[LR](A)[sP].[dR](C)[sP].[dR](A)[sP].[dR] |
| | AC | | (C)[sP].[LR](A)[sP].[dR](C)[sP].[dR](A)[sP].[dR](C)[sP].[LR](A) |
| | | | [sP].[dR](C)[sP].[LR](A)[sP].[dR](C)[sP].[LR](A)[sP].[LR]([5meC]) |
| 7 | ACACACACACACAC | 7.3 | [LR](A)[sP].[LR]([5meC])[sP].[dR](A)[sP].[dR](C)[sP].[LR](A)[sP]. |
| | AC | | [dR](C)[sP].[dR](A)[sP][dR](C)[sP].[LR](A)[sP].[dR](C)[sP].[dR] |
| | | | (A)[sP].(dR](C)[sP].[LR](A)[sP].[dR](C)[sP].[LR](A)[sP].[LR] |
| | | | ([5meC]) |
| 8 | ACACACACACACAC | 8.1 | [LR](A)[sP].[dR](C)[sP].[LR](A)[sP].[dR](C)[sP].[LR](A)[sP].[dR] |
| | AC | | (C)[sP].[LR](A)[sP].[dR](C)[sP].[LR](A)[sP].[dR](C)[sP].[LR](A) |
| | | | [sP].[dR](C)[sP].[LR](A)[sP].[dR](C)[sP].[LR](A)[sP].[dR](C)[sP]. |
| | | | [LR](A)[sP].[LR]([5meC]) |
| 8 | ACACACACACACAC | 8.2 | [LR](A)[sP].[dR](C)[sP].[LR](A)[sP].[dR](C)[sP].[dR](A)[sP].[dR] |
| | ACAC | | (C)[sP].[LR](A)[sP].[dR](C)[sP].[dR](A)[sP].[dR](C)[sP].[LR](A) |
| | | | [sP].[dR](C)[sP].[dR](A)[sP].[dR](C)[sP].[LR](A)[sP].[dR](C)[sP]. |
| | | | [LR](A)[sP].[LR]([5meC]) |
| 8 | ACACACACACACAC | 8.3 | [LR](A)[sP].[LR]([5meC])[sP].[LR](A)[sP].[dR](C)[sP].[dR](A)[sP]. |
| | ACAC | | [dR](C)[sP].[LR](A)[sP][dR](C)[sP].[LR](A)[sP].[dR](C)[sP].[dR](A) |
| | | | [sP].[dR](C)[sP].[LR](A)[sP].[dR](C)[sP].[LR](A)[sP].[dR](C)[sP]. |
| | | | [dR](A)[sP].[LR]([5meC]) |
| 9 | CACACAC | 9.1 | [LR]([5meC])[sP].[LR](A)[sP].[LR]([5meC])[sP].[LR](A)[sP].[LR] |
| | | | ([5meC])[sP].[LR](A)[sP][LR]([5meC]) |
| 10 | CACACACA | 10.1 | [LR]([5meC])[sP].[LR](A)[sP].[LR]([5meC])[sP].[LR](A)[sP].[LR] |
| | | | ([5meC])[sP].[LR](A)[sP].[LR]([5meC])[sP].[LR](A) |
| 10 | CACACACA | 10.2 | [LR]([5meC])[sP].[LR](A)[sP].[LR]([5meC])[sP].[dR](A)[sP].[LR] |
| | | | ([5meC])[sP].[dR](A)[sP].[LR)([5meC])[sP].[LR)(A) |
| 11 | CACACACAC | 11.1 | [LR]([5meC])[sP].[LR](A)[sP].[LR]([5meC])[sP].[dR](A)[sP].[LR] |
| | | | ([5meC])[sP].[dR](A)[sP].[LR]([5meC])[sP].[LR](A)[sP].[LR] |
| | | | ([5meC]) |
| 12 | CACACACACA | 12.1 | [LR]([5meC])[sP].[LR](A)[sP].[LR]([5meC])[sP].[dR](A)[sP].[LR] |
| | | | ([5meC])[sP].[LR](A)[sP][LR]([5meC])[sP].[dR](A)[sP].[LR] |
| | | | ([5meC])[sP].[LR)(A) |
| 13 | CACACACACAC | 13.1 | [LR]([5meC])[sP].[LR](A)[sP].[dR](C)[sP].[LR](A)[sP].[LR] |
| | | | ([5meC])[sP].[LR](A)[sP].[dR](C)[sP].[dR](A)[sP].[LR]([5meC])[sP]. |
| | | | [LR](A)[sP].[LR]([5meC]) |
| 14 | CACACACACACA | 14.1 | [LR]([5meC])[sP].[LR](A)[sP].[dR](C)[sP].[LR](A)[sP].[dR](C)[sP]. |
| | | | [LR](A)[sP].[LR]([5meC])[sP].[dR](A)[sP].[LR]([5meC])[sP].[dR](A) |
| | | | [sP].[LR]([5meC])[sP].[LR](A) |
| 14 | CACACACACACA | 14.2 | [LR]([5meC])[sP].[LR](A)[sP].[dR](C)[sP].[LR](A)[sP].[dR](C)[sP]. |
| | | | [dR](A)[sP].[LR]([5meC])[sP].[dR](A)[sP].[dR](C)[sP].[dR](A)[sP]. |
| | | | [LR]([5meC])[sP].[LR](A) |
| 14 | CACACACACACA | 14.3 | [LR]([5meC])[sP].[LR](A)[sP].[dR](C)[sP].[dR](A)[sP].[dR](C)[sP]. |
| | | | [LR](A)[sP].[LR]([5meC])[sP].[dR](A)[sP].[LR]([5meC])[sP].[dR](A) |
| | | | [sP].[dR](C)[sP].[LR](A) |
| 14 | CACACACACACA | 14.4 | [LR]([5meC])[sP].[dR](A)[sP].[dR](C)[sP].[LR](A)[sP].[dR](C)[sP]. |
| | | | [LR](A)[sP].[LR]([5meC])[sP].[dR](A)[sP].[dR](C)[sP].[dR](A)[sP]. |
| | | | [LR]([5meC])[sP].[LR](A) |
| 15 | CACACACACACAC | 15.1 | [LR]([5meC])[sP].[dR](A)[sP].[LR]([5meC])[sP].[dR](A)[sP].[LR] |
| | | | ([5meC])[sP].[dR](A)[sP].[LR]([5meC])[sP].[LR](A)[sP].[dR](C) |
| | | | [sP].[LR](A)[sP].(dR](C)[sP].[LR)(A)[sP].[LR]([5meC]) |
| 15 | CACACACACACAC | 15.2 | [LR]([5meC])[sP].[dR](A)[sP].[dR](C)[sP].[dR](A)[sP].[LR]([5meC]) |
| | | | [sP].[dR](A)[sP].[dR](C)[sP].[LR](A)[sP].[dR](C)[sP].[LR](A)[sP]. |
| | | | [dR](C)[sP].[LR](A)[sP].[LR]([5meC]) |
| 15 | CACACACACACAC | 15.3 | [LR]([5meC])[sP].[dR](A)[sP].[LR]([5meC])[sP].[dR](A)[sP].[dR](C) |
| | | | [sP].[dR](A)[sP].[LR]([5meC])[sP].[LR](A)[sP].[dR](C)[sP].[dR](A) |
| | | | [sP].[dR](C)[sP].[LR](A)[sP].[LR]([5meC]) |
| 15 | CACACACACACAC | 15.4 | [LR]([5meC])[sP].[dR](A)[sP].[dR](C)[sP].[dR](A)[sP].[LR]([5meC]) |
| | | | [sP].[dR](A)[sP].[dR](C)[sP].[LR](A)[sP].[dR](C)[sP].[LR](A)[sP]. |
| | | | [dR](C)[sP].[dR](A)[sP].[LR]([5meC]) |
| 15 | CACACACACACAC | 15.5 | [LR]([5meC])[sP].[dR](A)[sP].[dR](C)[sP].[dR](A)[sP].[LR]([5meC]) |
| | | | [sP].[dR](A)[sP].[dR](C)[sP].[LR](A)[sP].[dR](C)[sP].[dR](A)[sP]. |
| | | | [dR](C)[sP].[LR](A)[sP].[LR]([5meC]) |
| 16 | CACACACACACACA | 16.1 | [LR]([5meC])[sP].[dR](A)[sP].[dR](C)[sP].[LR](A)[sP].[dR](C)[sP]. |
| | CA | | [LR](A)[sP].[dR](C)[sP][LR](A)[sP].[dR](C)[sP].[LR](A)[sP].[dR] |
| | | | (C)[sP].[LR](A)[sP].[dR](C)[sP].[dR](A)[sP].(LR]([5meC])[sP].[LR] |
| | | | (A) |
| 16 | CACACACACACACA | 16.2 | [LR]([5meC])[sP].[dR](A)[sP].[dR](C)[sP].[LR](A)[sP].[dR](C)[sP]. |
| | CA | | [dR](A)[sP].[dR](C)[sP][LR](A)[sP].[dR](C)[sP].[LR](A)[sP].[dR] |
| | | | (C)[sP].[LR](A)[sP].[dR](C)[sP].[dR](A)[sP].[LR]([5meC])[sP].[LR] |
| | | | (A) |
| 16 | CACACACACACACA | 16.3 | [LR]([5meC])[sP].[dR](A)[sP].[dR](C)[sP].[LR](A)[sP].[dR](C)[sP]. |
| | CA | | [dR](A)[sP].[dR](C)[sP][LR](A)[sP].[dR](C)[sP].[dR](A)[sP].[dR] |
| | | | (C)[sP].[LR](A)[sP].[dR](C)[sP].[dR](A)[sP].[LR]([5meC])[sP].[LR] |
| | | | (A) |
| 18 | CACACACACACACA | 18.1 | [LR]([5meC])[sP].[dR](A)[sP].[dR](C)[sP].[LR](A)[sP].[dR](C)[sP]. |
| | CACA | | [LR](A)[sP].[dR](C)[sP][LR](A)[sP].[dR](C)[sP].[LR)(A)[sP].[dR] |
| | | | (C)[sP].[LR](A)[sP].[dR](C)[sP].[LR](A)[sP].[dR](C)[sP].[dR](A) |
| | | | [sP].[LR]([5meC])[sP].[LR](A) |
| 18 | CACACACACACACA | 18.2 | [LR]([5meC])[sP].[dR](A)[sP].[dR](C)[sP].[LR](A)[sP].[dR](C)[sP]. |
| | CACA | | [dR](A)[sP].[dR](C)[sP][LR](A)[sP].[dR](C)[sP].[dR](A)[sP].[dR] |
| | | | (C)[sP].(LR](A)[sP].[LR]([5meC])[sP].[dR](A)[sP].[dR](C)[sP].[dR] |
| | | | (A)[sP].(LR]([5meC])[sP].[LR](A) |
| 18 | CACACACACACACA | 18.3 | [LR]([5meC])[sP].[dR](A)[sP].[LR]([5meC])[sP].[dR](A)[sP].[dR](C) |
| | CACA | | [sP].[LR](A)[sP].[dR](C)[sP].[LR](A)[sP].[dR](C)[sP].[LR](A)[sP]. |
| | | | [dR](C)[sP].[dR](A)[sP].[dR](C)[sP].[LR](A)[sP].[dR](C)[sP].[dR] |
| | | | (A)[sP].[LR]([5meC])[sP].[LR](A) |
| 18 | CACACACACACACA | 18.4 | [LR]([5meC])[sP].[dR](A)[sP].[dR](C)[sP].[LR](A)[sP].[dR](C)[sP]. |
| | CACA | | [LR](A)[sP].[dR](C)[sP][dR](A)[sP].[dR](C)[sP].[LR](A)[sP].[LR] |
| | | | ([5meC])[sP].[dR](A)[sP].[dR](C)[sP].[LR](A)[sP].[dR](C)[sP].[dR] |
| | | | (A)[sP].[LR]([5meC])[sP].[LR](A) |
| 19 | TCCACACTGAACAA | 19.1 | [LR](T)[sP].[LR]([5meC])[sP].[dR](C)[sP].[dR](A)[sP].[dR](C)[sP]. |
| | ACC | | [dR](A)[sP].[dR](C)[sP][dR](T)[sP].[dR](G)[sP].[dR](A)[sP].[dR](A) |
| | | | [sP].[dR](C)[sP].[dR](A)[sP].[LR](A)[sP].[LR](A)[sP].[LR]([5meC]) |
| | | | [sP].[LR]([5meC]) |
| 20 | CACACACACACACA | 20.1 | [LR]([5meC])[sP].[LR](A)[sP].[dR](C)[sP].[dR](A)[sP].[LR]([5meC]) |
| | CACACACAC | | [sP].[dR](A)[sP].[LR]([5meC])[sP].[dR](A)[sP].[LR]([5meC])[sP]. |
| | | | [dR](A)[sP].[LR]([5meC])[sP].[dR](A)[sP].[LR]([5meC])[sP].[dR](A) |
| | | | [sP].[LR]([5meC])[sP].[dR](A)[sP].[LR]([5meC])[sP].[dR](A)[sP]. |
| | | | [LR]([5meC])[sP].[dR](A)[sP].[dR](C)[sP].[LR](A)[sP].[LR]([5meC]) |
| 21 | ACACACACACACAC | 21 | [LR](A)[sP].[LR]([5meC])[sP].[dR](A)[sP].[LR]((5meC])[sP].[dR](A) |
| | ACACACACAC | | [sP].(LR]([5meC])[sP][dR](A)[sP].[LR]([5meC])[sP].[dR](A)[sP]. |
| | | | [LR]([5meC])[sP].[dR](A)[sP].[LR]([5meC])[sP].[dR](A)[sP].[LR] |
| | | | ([5meC])[sP].[dR](A)[sP].[LR]([5meC])[sP].[dR](A)[sP].[LR] |
| | | | ([5meC])[sP].[dR](A)[sP].[LR]([5meC])[sP].[dR](A)[sP].[LR] |
| | | | ([5meC])[sP].[dR](A)[sP].[LR]([5meC]) |
| 22 | CACACACACACAAC | 22.1 | [LR]([5meC])[sP].[LR](A)[sP].[dR](C)[sP].[LR](A)[sP].[dR](C)[sP]. |
| | ACACACACAC | | [LR](A)[sP].[dR](C)[sP][LR](A)[sP].[dR](C)[sP].[dR](A)[sP].[LR] |
| | | | ([5meC])[sP].[LR](A)[sP].[LR](A)[sP].[LR]([5meC])[sP].[dR](A) |
| | | | [sP].[dR](C)[sP].[LR](A)[sP].[dR](C)[sP].[LR](A)[sP].[dR](C) |
| | | | [sP].[LR](A)[sP].[dR](C)[sP].[LR](A)[sP].[LR]([5meC]) |
| 22 | CACACACACACAAC | 22.2 | [LR]([5meC])[sP].[LR](A)[sP].[LR]([5meC])[sP].[LR](A)[sP].[LR] |
| | ACACACACAC | | ([5meC])[sP].[LR](A)[sP][LR]([5meC])[sP].[LR](A)[sP].[LR] |
| | | | ([5meC])[sP].[LR](A)[sP].[LR]([5meC])[sP].[LR](A)[sP][LR](A)[sP]. |
| | | | [LR]([5meC])[sP].[LR](A)[sP].[LR]([5meC])[sP].[LR](A)[sP].[LR] |
| | | | ([5meC])[sP].[LR](A)[sP].[LR]([5meC])[sP].[LR](A)[sP].[LR] |
| | | | ([5meC])[sP].[LR](A)[sP].[LR]([5meC]) |
| 23 | CACACACACACACA | 23.6 | [LR]([5meC])[sP].[LR](A)[sP].[dR](C)[sP].[dR](A)[sP].[LR] |
| | CACACACACAC | | ([5meC])[sP].[LR](A)[sP].[dR](C)[sP].[dR](A)[sP].[LR]([5meC]) |
| | | | [sP].[LR)(A)[sP].[dR](C)[sP].[dR](A)[sP].[LR]([5meC])[sP][LR] |
| | | | (A)[sP].[dR](C)[sP].[dR](A)[sP].[LR]([5meC])[sP].[LR](A)[sP]. |
| | | | [dR](C)[sP].[dR](A)[sP].[LR]([5meC])[sP].[LR](A)[sP].[dR](C) |
| | | | [sP].[dR](A)[sP].[LR]([5meC]) |
| 23 | CACACACACACACA | 23.7 | [LR]([5meC])[sP].[LR](A)[sP].[dR](C)[sP].[dR](A)[sP].[dR](C) |
| | CACACACACAC | | [sP].[LR](A)[sP].[LR]([5meC])[sP].[dR](A)[sP].[dR](C)[sP].[LR] |
| | | | (A)[sP].[LR]([5meC])[sP].[dR](A)[sP].[dR](C)[sP].[dR](A)[sP]. |
| | | | [LR]([5meC])[sP].[LR)(A)[sP].[dR](C)[sP].[dR](A)[sP].[dR](C) |
| | | | [sP].[LR](A)[sP][dR](C)[sP].[dR](A)[sP].[dR](C)[sP].[LR](A) |
| | | | [sP].[LR]([5meC]) |
| 23 | CACACACACACACA | 23.8 | [LR]([5meC])[sP].[LR](A)[sP].[dR](C)[sP].[dR](A)[sP].[LR] |
| | CACACACACAC | | ([5meC])[sP].[dR](A)[sP].[LR]([5meC])[sP].[dR](A)[sP].[LR] |
| | | | ([5meC])[sP].[dR](A)[sP].[LR]([5meC])[sP].[dR](A)[sP].[LR] |
| | | | ([5meC])[sP].[dR](A)[sP].[LR]([5meC])[sP].[dR](A)[sP].[LR] |
| | | | ([5meC])[sP].[dR](A)[sP].[LR]([5meC])[sP].[dR](A)[sP].[LR] |
| | | | ([5meC])[SP].[dR](A)[sP].[dR](C)[sP].[LR](A)[sP][LR]([5meC]) |
| 23 | CACACACACACACA | 23.9 | [LR]([5meC])[sP].[LR](A)[sP].[dR](C)[sP].[LR](A)[sP].[dR](C)[sP]. |
| | CACACACACAC | | [LR](A)[sP].[dR](C)[sP].[LR](A)[sP].[dR](C)[sP].[LR](A)[sP].[dR] |
| | | | (C)[sP].[LR](A)[sP].[dR](C)[sP].[LR](A)[sP].[dR](C)[sP].[LR](A) |
| | | | [sP].[dR](C)[sP].[LR](A)[sP].[dR](C)[sP].[LR](A)[sP].[dR](C)[sP]. |
| | | | [LR](A)[sP].[dR](C)[sP].[LR](A)[sP].[LR]([5meC]) |
| 24 | ACACACACACACAC | 24 | [LR](A)[sP].[LR]([5meC])[sP].[dR](A)[sP].[LR]([5meC])[sP].[dR](A) |
| | ACACACACACAC | | [sP].[LR]([5meC])[sP].[dR](A)[sP].[LR]([5meC])[sP].[dR](A)[sP]. |
| | | | [LR]([5meC])[sP].[dR](A)[sP].[LR]([5meC])[sP].[dR](A)[sP].[LR] |
| | | | ([5meC])[sP].[dR](A)[sP].[LR]([5meC])[sP].[dR](A)[sP].[LR] |
| | | | ([5meC])[sP].[dR](A)[sP].[LR]([5meC])[sP].[dR](A)[sP].[LR] |
| | | | ([5meC])[sP].[dR](A)[sP].[LR]([5meC])[sP].[dR](A)[sP].[LR] |
| | | | ([5meC]) |
| 25 | CACACACACACACA | 25.1 | [LR]([5meC])[sP].[LR](A)[sP].[dR](C)[sP].[dR](A)[sP].[LR]([5meC]) |
| | CACACACACACAC | | [sP].[LR](A)[sP].[dR](C)[sP].[dR](A)[sP].[LR]([5meC])[sP].[LR] |
| | | | (A)[sP].[dR](C)[sP].[dR](A)[sP].[LR]([5meC])[sP].[LR](A)[sP].[dR] |
| | | | (C)[sP].[dR](A)[sP].[LR]([5meC])[sP].[LR](A)[sP].[dR](C)[sP].[dR] |
| | | | (A)[sP].[LR]([5meC])[sP].[LR](A)[sP].[dR](C)[sP].[dR](A)[sP].[LR] |
| | | | ([5meC])[sP].[LR](A)[sP].[LR]([5meC]) |
| 25 | CACACACACACACA | 25.2 | [LR]([5meC])[sP].[LR](A)[sP].[dR](C)[sP].[dR](A)[sP].[dR](C)[sP]. |
| | CACACACACACAC | | [LR](A)[sP].[LR]([5meC])[sP].[dR](A)[sP].[dR](C)[sP].[LR](A)[sP]. |
| | | | [LR]([5meC])[sP].[dR](A)[sP].[dR](C)[sP].[dR](A)[sP].[LR]([5meC]) |
| | | | [sP].[LR](A)[sP].[dR](C)[sP].[dR](A)[sP].[LR]([5meC])[sP].[dR](A) |
| | | | [sP].[dR](C)[sP].[LR](A)[sP].[dR](C)[sP].[dR](A)[sP].[dR](C)[sP]. |
| | | | [LR](A)[sP].[LR]([5meC]) |
| 25 | CACACACACACACA | 25.3 | [LR]([5meC])[sP].[LR](A)[sP].[dR](C)[sP].[dR](A)[sP].[LR]([5meC]) |
| | CACACACACACAC | | [sP].[dR](A)[sP].[LR]([5meC])[sP].[dR](A)[sP].[LR]([5meC])[sP]. |
| | | | [dR](A)[sP].[LR]([5meC])[sP].[dR](A)[sP].[LR]([5meC])[sP].[dR](A) |
| | | | [sP].[LR]([5meC])[sP].[dR](A)[sP].[LR]([5meC])[sP].[dR](A)[sP]. |
| | | | [LR]([5meC])[sP].[dR](A)[sP].[LR]([5meC])[sP].[dR](A)[sP].[LR] |
| | | | ([5meC])[sP].[dR](A][sP].[dR](C)[sP].[LR](A)[sP].[LR]([5meC]) |
| 25 | CACACACACACACA | 25.4 | [LR]([5meC])[sP].[LR](A)[sP].[dR](C)[sP].[LR](A)[sP].[dR](C)[sP]. |
| | CACACACACACAC | | [LR](A)[sP].[dR](C)[sP].[LR](A)[sP].[dR](C)[sP].[LR](A)[sP].[dR] |
| | | | (C)[sP].[LR](A)[sP].[dR](C)[sP].[LR](A)[sP].[dR](C)[sP].[LR](A) |
| | | | [sP].[dR](C)[sP].[LR](A)[sP].[dR](C)[sP].[LR](A)[sP].[dR](C)[sP]. |
| | | | [LR](A)[sP].[dR](C)[sP].[LR](A)[sP].[dR](C)[sP].[LR](A)[sP].[LR] |
| | | | ([5meC]) |
| 26 | ACACACACACACAC | 26 | [LR](A)[sP].[LR]([5meC])[sP].[dR](A)[sP].[LR]([5meC])[sP].[dR](A) |
| | ACACACACACACAC | | [sP].[LR]([5meC])[sP].[dR](A)[sP].[LR]([5meC])[sP].[dR](A)[sP]. |
| | | | [LR]([5meC])[sP].[dR](A)[sP].[LR]([5meC])[sP].[dR](A)[sP].[LR] |
| | | | ([5meC])[sP].[dR](A)[sP].[LR]([5meC])[sP].[dR](A)[sP].[LR] |
| | | | ([5meC])[sP].[dR](A)[sP].[LR]([5meC])[sP].[dR](A)[sP].[LR]([5meC]) |
| | | | [sP].[dR](A)[sP].[LR]([5meC])[sP].[dR](A)[sP].[LR]([5meC])[sP]. |
| | | | [dR](A)[sP].[LR]([5meC]) |
| 27 | CACACACACACACA | 27.1 | [LR]([5meC])[sP].[LR](A)[sP].[dR](C)[sP].[LR](A)[sP].[dR](C)[sP]. |
| | CACACACACACACA | | [LR](A)[sP].[dR](C)[sP].[LR](A)[sP].[dR](C)[sP].[LR](A)[sP].[dR] |
| | C | | (C)[sP].[LR](A)[sP].[dR](C)[sP].[LR](A)[sP].[dR](C)[sP].[LR](A) |
| | | | [sP].[dR](C)[sP].[LR](A)[sP].[dR](C)[sP].[LR](A)[sP].[dR](C)[sP]. |
| | | | [LR](A)[sP].[dR](C)[sP].[LR](A)[sP].[dR](C)[sP].[LR](A)[sP].[dR] |
| | | | (C)[sP].[LR](A)[sP].[LR]([5meC]) |
| 27 | CACACACACACACA | 27.2 | [LR]([5meC])[sP].[LR](A)[sP].[dR](C)[sP].[dR](A)[sP].[LR]([5meC]) |
| | CACACACACACACA | | [sP].[LR](A)[sP].[dR](C)[sP].[dR](A)[sP].[LR]([5meC])[sP].[LR](A) |
| | C | | [sP].[dR](C)[sP].[dR](A)[sP].[LR]([5meC])[sP].[LR](A)[sP].[dR](C) |
| | | | [sP].[dR](A)[sP].[LR]([5meC])[sP].[LR](A)[sP].[dR](C)[sP].[dR](A) |
| | | | [sP].[LR]([5meC])[sP].[LR](A)[sP].[dR](C)[sP].[dR](A)[sP].[LR] |
| | | | ([5meC])[sP].[LR](A)[sP].[dR](C)[sP].[dR](A)[sP].[LR]([5meC]) |
| 27 | CACACACACACACA | 27.3 | [LR]([5meC])[sP].[LR](A)[sP].[dR](C)[sP].[dR](A)[sP].[LR]([5meC]) |
| | CACACACACACACA | | [sP].[dR](A)[sP].[LR]([5meC])[sP].[dR](A)[sP].[LR]([5meC])[sP]. |
| | C | | [dR](A)[sP].[LR]([5meC])[sP].[dR](A)[sP].[LR]([5meC])[sP].[dR](A) |
| | | | [sP].[LR]([5meC])[sP].[dR](A)[sP].[LR]([5meC])[sP].[dR](A)[sP]. |
| | | | [LR]([5meC])[sP].[dR](A)[sP].[LR]([5meC])[sP].[dR](A)[sP].[LR] |
| | | | ([5meC])[sP].[dR](A][sP].[LR]([5meC])[sP].[dR](A)[sP].[dR](C)[sP]. |
| | | | [LR](A)[sP].[LR]([5meC]) |
| 27 | CACACACACACACA | 27.4 | [LR]([5meC])[sP].[LR](A)[sP].[dR](C)[sP].[dR](A)[sP].[dR](C)[sP]. |
| | CACACACACACACA | | [LR](A)[sP].[LR]([5meC])[sP].[dR](A)[sP].[dR](C)[sP].[LR](A)[sP]. |
| | C | | [LR]([5meC])[sP].[dR](A)[sP].[dR](C)[sP].[dR](A)[sP].[LR]([5meC]) |
| | | | [sP].[LR](A)[sP].[dR](C)[sP].[dR](A)[sP].[LR]([5meC])[sP].[LR](A) |
| | | | [sP].[dR](C)[sP].[dR](A)[sP].[dR](C)[sP].[LR](A)[sP].[dR](C)[sP]. |
| | | | [dR](A)[sP].[dR](C)[sP].[LR](A)[sP].[LR]([5meC]) |
| 28 | ACACACACACACAC | 28 | [LR](A)[sP].[LR]([5meC])[sP].[dR](A)[sP].[LR]([5meC])[sP].[dR](A) |
| | ACACACACACACAC | | [sP].[LR]([5meC])[sP].[dR](A)[sP].[LR]([5meC])[sP].[dR](A)[sP]. |
| | AC | | [LR]([5meC])[sP].[dR](A)[sP].[LR]([5meC])[sP].[dR](A)[sP].[LR] |
| | | | ([5meC])[sP].[dR](A)[sP].[LR]([5meC])[sP].[dR](A)[sP].[LR]([5meC]) |
| | | | [sP].[dR](A)[sP].[LR]([5meC])[sP].[dR](A)[sP].[LR]([5meC])[sP]. |
| | | | [dR](A)[sP].[LR]([5meC])[sP].[dR](A)[sP].[LR]([5meC])[sP].[dR](A) |
| | | | [sP].[LR]([5meC])[sP].[dR](A)[sP].[LR]([5meC]) |
| 29 | CACACACACACACA | 29.1 | [LR]([5meC])[sP].[LR](A)[sP].[dR](C)[sP].[LR](A)[sP].[dR](C)[sP]. |
| | CACACACACACACA | | [LR](A)[sP].[dR](C)[sP].[LR](A)[sP].[dR](C)[sP].[LR](A)[sP].[dR] |
| | CAC | | (C)[sP].[LR](A)[sP].[dR](C)[sP].[LR](A)[sP].[dR](C)[sP].[LR](A) |
| | | | [sP].[dR](C)[sP].[LR](A)[sP].[dR](C)[sP].[LR](A)[sP].[dR](C)[sP]. |
| | | | [LR](A)[sP].[dR](C)[sP].[LR](A)[sP].[dR](C)[sP].[LR](A)[sP].[dR] |
| | | | (C)[sP].[LR](A)[sP].[dR](C)[sP].[LR](A)[sP].[LR]([5meC]) |
| 29 | CACACACACACACA | 29.2 | [LR]([5meC])[sP].[LR](A)[sP].[dR](C)[sP].[dR](A)[sP].[LR]([5meC]) |
| | CACACACACACACA | | [sP].[LR](A)[sP].[dR](C)[sP].[dR](A)[sP].[LR]([5meC])[sP].[LR](A) |
| | CAC | | [sP].[dR](C)[sP].[dR](A)[sP].[LR]([5meC])[sP].[LR](A)[sP].[dR](C) |
| | | | [sP].[dR](A)[sP].[LR]([5meC])[sP].[LR](A)[sP].[dR](C)[sP].[dR](A) |
| | | | [sP].[LR]([5meC])[sP].[LR](A)[sP].[dR](C)[sP].[dR](A)[sP].[LR] |
| | | | ([5meC])[sP].[LR](A)[sP].[dR](C)[sP].[dR](A)[sP].[LR]([5meC])[sP]. |
| | | | [LR](A)[sP].[LR]([5meC]) |
| 29 | CACACACACACACA | 29.3 | [LR]([5meC])[sP].[LR](A)[sP].[dR](C)[sP].[dR](A)[sP].[LR]([5meC]) |
| | CACACACACACACA | | [sP].[dR](A)[sP].[LR]([5meC])[sP].[dR](A)[sP].[LR]([5meC])[sP]. |
| | CAC | | [dR](A)[sP].[LR]([5meC])[sP].[dR](A)[sP].[LR]([5meC])[sP].[dR](A) |
| | | | [sP].[LR]([5meC])[sP].[dR](A)[sP].[LR]([5meC])[sP].[dR](A)[sP]. |
| | | | [LR]([5meC])[sP].[dR](A)[sP].[LR]([5meC])[sP].[dR](A)[sP].[LR] |
| | | | ([5meC])[sP].[dR](A][sP].[LR]([5meC])[sP].[dR](A)[sP].[LR] |
| | | | ([5meC])[sP].[dR](A)[sP].[dR](C)[sP].[LR](A)[sP].[LR]([5meC]) |
| 29 | CACACACACACACA | 29.4 | [LR]([5meC])[sP].[LR](A)[sP].[dR](C)[sP].[dR](A)[sP].[dR](C)[sP]. |
| | CACACACACACACA | | [LR](A)[sP].[LR]([5meC])[sP].[dR](A)[sP].[dR](C)[sP].[LR](A)[sP]. |
| | CAC | | [LR]([5meC])[sP].[dR](A)[sP].[dR](C)[sP].[dR](A)[sP].[LR]([5meC]) |
| | | | [sP].[LR](A)[sP].[dR](C)[sP].[dR](A)[sP].[LR]([5meC])[sP].[LR](A) |
| | | | [sP].[dR](C)[sP].[dR](A)[sP].[dR](C)[sP].[LR](A)[sP].[LR]([5meC]) |
| | | | [sP].[LR](A)[sP].[dR](C)[sP].[dR](A)[sP].[dR](C)[sP].[LR](A)[sP]. |
| | | | [LR]([5meC]) |
| 29 | ACACACACACACAC | 30 | [LR](A)[sP].[LR]([5meC])[sP].[LR](A)[sP].[dR](C)[sP].[dR](A)[sP]. |
| | ACACCACACACACA | | [dR](C)[sP].[LR](A)[sP].[dR](C)[sP].[LR](A)[sP].[dR](C)[sP].[dR] |
| | CACACA | | (A)[sP].[dR](C)[sP].[LR](A)[sP].[dR](C)[sP].[LR](A)[sP].[dR](C) |
| | | | [sP].[dR](A)[sP].[LR]([5meC])[sP].[LR]([5meC])[sP].[dR](A)[sP]. |
| | | | [dR](C)[sP].[LR](A)[sP].[dR](C)[sP].[LR](A)[sP].[dR](C)[sP].[LR] |
| | | | (A)[sP].[dR](C)[sP].[LR](A)[sP].[dR](C)[sP].[LR](A)[sP].[dR](C) |
| | | | [sP].[dR](A)[sP].[LR]([5meC])[sP].[LR](A) |
| 31 | CACACACACACACA | 31.1 | [LR]([5meC])[sP].[dR](A)[sP].[dR](C)[sP].[LR](A)[sP].[dR](C)[sP]. |
| | CACACACACACACA | | [LR](A)[sP].[dR](C)[sP].[LR](A)[sP].[dR](C)[sP].[LR](A)[sP].[dR] |
| | CACACA | | (C)[sP].[LR](A)[sP].[dR](C)[sP].[dR](A)[sP].[LR]([5meC])[sP].[LR] |
| | | | (A)[sP].[LR]([5meC])[sP].[dR](A)[sP].[dR](C)[sP].[LR](A)[sP].[dR] |
| | | | (C)[sP].[LR](A)[sP].[dR](C)[sP].[LR](A)[sP].[dR](C)[sP].[LR](A) |
| | | | [sP].[dR](C)[sP].[LR](A)[sP].[dR](C)[sP].[LR](A)[sP].[dR](C)[sP]. |
| | | | [dR](A)[sP].[LR]([5meC])[sP].[LR](A) |
| 31 | CACACACACACACA | 31.2 | [LR]([5meC])[sP].[dR](A)[sP].[dR](C)[sP].[LR](A)[sP].[dR](C)[sP]. |
| | CACACACACACACA | | [LR](A)[sP].[dR](C)[sP].[LR](A)[sP].[dR](C)[sP].[LR](A)[sP].[dR] |
| | CACACA | | (C)[sP].[LR](A)[sP].[dR](C)[sP].[LR](A)[sP].[dR](C)[sP].[dR](A) |
| | | | [sP].[LR]([5meC])[sP].[LR](A)[sP].[LR]([5meC])[sP].[dR](A)[sP]. |
| | | | [dR](C)[sP].[LR](A)[sP].[dR](C)[sP].[LR](A)[sP].[dR](C)[sP].[LR] |
| | | | (A)[sP].[dR](C)[sP].[LR](A)[sP].[dR](C)[sP].[LR](A)[sP].[dR](C) |
| | | | [sP].[dR](A)[sP].[LR]([5meC])[sP].[LR](A) |
| 31 | CACACACACACACA | 32 | [LR]([5meC])[sP].[dR](A)[sP].[dR](C)[sP].[LR](A)[sP].[dR](C)[sP]. |
| | CACAACACACACAC | | [LR](A)[sP].[dR](C)[sP].[LR](A)[sP].[dR](C)[sP].[LR](A)[sP].[dR] |
| | ACACACAC | | (C)[sP].[LR](A)[sP].[dR](C)[sP].[LR](A)[sP].[dR](C)[sP].[dR](A) |
| | | | [sP].[LR]([5meC])[sP].[LR](A)[sP].[LR](A)[sP].[LR]([5meC])[sP]. |
| | | | [LR](A)[sP].[dR](C)[sP].[dR](A)[sP].[dR](C)[sP].[LR](A)[sP].[dR] |
| | | | (C)[sP].[LR](A)[sP].[dR](C)[sP].[dR](A)[sP].[dR](C)[sP].[LR](A) |
| | | | [sP].[dR](C)[sP].[LR](A)[sP].[dR](C)[sP].[dR](A)[sP].[LR]([5meC]) |
| 33 | ACACACACACACAC | 33 | [LR](A)[sP].[LR]([5meC])[sP].[LR](A)[sP].[dR](C)[sP].[dR](A)[sP]. |
| | ACACCACACACACA | | [dR](C)[sP].[LR](A)[sP].[dR](C)[sP].[LR](A)[sP].[dR](C)[sP].[dR] |
| | CACACACA | | (A)[sP].[dR](C)[sP].[LR](A)[sP].[dR](C)[sP].[LR](A)[sP].[dR](C) |
| | | | [sP].[dR](A)[sP].[LR]([5meC])[sP].[LR]([5meC])[sP].[dR](A)[sP]. |
| | | | [dR](C)[sP].[LR](A)[sP].[dR](C)[sP].[LR](A)[sP].[dR](C)[sP].[LR] |
| | | | (A)[sP].[dR](C)[sP].[LR](A)[sP].[dR](C)[sP].[LR](A)[sP].[dR](C) |
| | | | [sP].[LR](A)[sP].[dR](C)[sP].[dR](A)[sP].[LR]([5meC])[sP].[LR](A) |
| 22 | CACACACACACAAC | 22.3 | [MOE]([5meC])[sP].[MOE](A)[sP].[MOE]([5meC])[sP].[MOE](A)[sP]. |
| | ACACACACAC | | [MOE]([5meC])[sP].[MOE](A)[sP].[MOE]([5meC])[sP].[MOE](A)[sP]. |
| | | | [MOE]([5meC])[sP].[MOE](A)[sP].[MOE]([5meC])[sP].[MOE](A)[sP]. |
| | | | [MOE](A)[sP].[MOE]([5meC])[sP].[MOE](A)[sP].[MOE]([5meC])[sP]. |
| | | | [MOE](A)[sP].[MOE]([5meC])[sP].[MOE](A)[sP].[MOE]([5meC])[sP]. |
| | | | [MOE](A)[sP].[MOE]([5meC])[sP].[MOE](A)[sP].[MOE]([5meC]) |
| 23 | CACACACACACACA | 23.1 | [MOE]([5meC])[sP].[MOE](A)[sP].[MOE]([5meC])[sP].[MOE](A)[sP]. |
| | CACACACACAC | | [MOE]([5meC])[sP].[MOE](A)[sP].[MOE]([5meC])[sP].[MOE](A)[sP]. |
| | | | [MOE]([5meC])[sP].[MOE](A)[sP].[MOE]([5meC])[sP].[MOE](A)[sP]. |
| | | | [MOE]([5meC])[sP].[MOE](A)[sP].[MOE]([5meC])[sP].[MOE](A)[sP]. |
| | | | [MOE]([5meC])[sP].[MOE](A)[sP].[MOE]([5meC])[sP].[MOE](A)[sP]. |
| | | | [MOE]([5meC])[sP].[MOE](A)[sP].[MOE]([5meC])[sP].[MOE](A)[sP]. |
| | | | [MOE]([5meC]) |
| 25 | CACACACACACACA | 25.5 | [MOE]([5meC])[sP].[MOE](A)[sP].[MOE]([5meC])[sP].[MOE](A)[sP]. |
| | CACACACACACAC | | [MOE]([5meC])[sP].[MOE](A)[sP].[MOE]([5meC])[sP].[MOE](A)[sP]. |
| | | | [MOE]([5meC])[sP].[MOE](A)[sP].[MOE]([5meC])[sP].[MOE](A)[sP]. |
| | | | [MOE]([5meC])[sP].[MOE](A)[sP].[MOE]([5meC])[sP].[MOE](A)[sP]. |
| | | | [MOE]([5meC])[sP].[MOE](A)[sP].[MOE]([5meC])[sP].[MOE](A)[sP]. |
| | | | [MOE]([5meC])[sP].[MOE](A)[sP].[MOE]([5meC])[sP].[MOE](A)[sP]. |
| | | | [MOE]([5meC])[sP].[MOE](A)[sP].[MOE]([5meC]) |
| 27 | CACACACACACACA | 27.5 | [MOE]([5meC])[sP].[MOE](A)[sP].[MOE]([5meC])[sP].[MOE](A)[sP]. |
| | CACACACACACACA | | [MOE]([5meC])[sP].[MOE](A)[sP].[MOE]([5meC])[sP].[MOE](A)[sP]. |
| | C | | [MOE]([5meC])[sP].[MOE](A)[sP].[MOE]([5meC])[sP].[MOE](A)[sP]. |
| | | | [MOE]([5meC])[sP].[MOE](A)[sP].[MOE]([5meC])[sP].[MOE](A)[sP]. |
| | | | [MOE]([5meC])[sP].[MOE](A)[sP].[MOE]([5meC])[sP].[MOE](A)[sP]. |
| | | | [MOE]([5meC])[sP].[MOE](A)[sP].[MOE]([5meC])[sP].[MOE](A)[sP]. |
| | | | [MOE]([5meC])[sP].[MOE](A)[sP].[MOE]([5meC])[sP].[MOE](A)[sP]. |
| | | | [MOE]([5meC]) |
| 29 | CACACACACACACA | 29.5 | [MOE]([5meC])[sP].[MOE](A)[sP].[MOE]([5meC])[sP].[MOE](A)[sP]. |
| | CACACACACACACA | | [MOE]([5meC])[sP].[MOE](A)[sP].[MOE]([5meC])[sP].[MOE](A)[sP]. |
| | CAC | | [MOE]([5meC])[sP].[MOE](A)[sP].[MOE]([5meC])[sP].[MOE](A)[sP]. |
| | | | [MOE]([5meC])[sP].[MOE](A)[sP].[MOE]([5meC])[sP].[MOE](A)[sP]. |
| | | | [MOE]([5meC])[sP].[MOE](A)[sP].[MOE]([5meC])[sP].[MOE](A)[sP]. |
| | | | [MOE]([5meC])[sP].[MOE](A)[sP].[MOE]([5meC])[sP].[MOE](A)[sP]. |
| | | | [MOE]([5meC])[sP].[MOE](A)[sP].[MOE]([5meC])[sP].[MOE](A)[sP]. |
| | | | [MOE]([5meC])[sP].[MOE](A)[sP].[MOE]([5meC]) |
| 23 | CACACACACACACA | 23.2 | [MOE]([5meC])[sP].[MOE](A)[sP].[dR](C)[sP].[MOE](A)[sP].[dR](C) |
| | CACACACACAC | | [sP].[MOE](A)[sP].[dR](C)[sP].[MOE](A)[sP].[dR](C)[sP].[MOE](A) |
| | | | [sP].[dR](C)[sP].[MOE](A)[sP].[dR](C)[sP].[MOE](A)[sP].[dR](C) |
| | | | [sP].[MOE](A)[sP].[dR](C)[sP].[MOE](A)[sP].[dR](C)[sP].[MOE](A) |
| | | | [sP].[dR](C)[sP].[MOE](A)[sP].[dR](C)[sP].[MOE](A)[sP].[MOE] |
| | | | ([5meC]) |
| 25 | CACACACACACACA | 25.6 | [MOE]([5meC])[sP].[MOE](A)[sP].[dR](C)[sP].[MOE](A)[sP].[dR](C) |
| | CACACACACACAC | | [sP].[MOE](A)[sP].[dR](C)[sP].[MOE](A)[sP].[dR](C)[sP].[MOE](A) |
| | | | [sP].[dR](C)[sP].[MOE](A)[sP].[dR](C)[sP].[MOE](A)[sP].[dR](C) |
| | | | [sP].[MOE](A)[sP].[dR](C)[sP].[MOE](A)[sP].[dR](C)[sP].[MOE](A) |
| | | | [sP].[dR](C)[sP].[MOE](A)[sP].[dR](C)[sP].[MOE](A)[sP].[dR](C) |
| | | | [sP].[MOE](A)[sP].[MOE]([5meC]) |
| 23 | CACACACACACACA | 23.3 | [MOE]([5meC])[sP].[MOE](A)[sP].[dR](C)[sP].[dR](A)[sP].[MOE] |
| | CACACACACAC | | ([5meC])[sP].[dR](A)[sP].[MOE]([5meC])[sP].[dR](A)[sP].[MOE] |
| | | | ([5meC])[sP].[dR](A)[sP].[MOE]([5meC])[sP].[dR](A)[sP].[MOE] |
| | | | ([5meC])[sP].[dR](A)[sP].[MOE]([5meC])[sP].[dR](A)[sP].[MOE] |
| | | | ([5meC])[sP].[dR](A)[sP].[MOE]([5meC])[sP].[dR](A)[sP].[MOE] |
| | | | ([5meC])[sP].[dR](A)[sP].[dR](C)[sP].[MOE](A)[sP].[MOE]([5meC]) |
| 25 | CACACACACACACA | 25.7 | [MOE]([5meC])[sP].[MOE](A)[sP].[dR](C)[sP].[dR](A)[sP].[MOE] |
| | CACACACACACAC | | ([5meC])[sP].[dR](A)[sP].[MOE]([5meC])[sP].[dR](A)[sP].[MOE] |
| | | | ([5meC])[sP].[dR](A)[sP].[MOE]([5meC])[sP].[dR](A)[sP].[MOE] |
| | | | ([5meC])[sP].[dR](A)[sP].[MOE]([5meC])[sP].[dR](A)[sP].[MOE] |
| | | | ([5meC])[sP].[dR](A)[sP].[MOE]([5meC])[sP].[dR](A)[sP].[MOE] |
| | | | ([5meC])[sP].[dR](A)[sP].[MOE]([5meC])[sP].[dR](A)[sP].[dR] |
| | | | (C)[sP].[MOE](A)[sP].[MOE]([5meC]) |
| 27 | CACACACACACACA | 27.6 | [MOE]([5meC])[sP].[MOE](A)[sP].[dR](C)[sP].[MOE](A)[sP].[dR] |
| | CACACACACACACA | | (C)[sP].[MOE](A)[sP].[dR](C)[sP].[MOE](A)[sP].[dR](C)[sP]. |
| | C | | [MOE](A)[sP].[dR](C)[sP].[MOE](A)[sP].[dR](C)[sP].[MOE](A)[sP]. |
| | | | [dR](C)[sP].[MOE](A)[sP].[dR](C)[sP].[MOE](A)[sP].[dR](C)[sP]. |
| | | | [MOE](A)[sP].[dR](C)[sP].[MOE](A)[sP].[dR](C)[sP].[MOE](A)[sP]. |
| | | | [dR](C)[sP].[MOE](A)[sP].[dR](C)[sP].[MOE](A)[sP].[MOE]([5meC]) |
| 27 | CACACACACACACA | 27.7 | [MOE]([5meC])[sP].[MOE](A)[sP].[dR](C)[sP].[dR](A)[sP].[MOE] |
| | CACACACACACACA | | ([5meC])[sP].[dR](A)[sP].[MOE]([5meC])[sP].[dR](A)[sP].[MOE] |
| | C | | ([5meC])[sP].[dR](A)[sP].[MOE]([5meC])[sP].[dR](A)[sP].[MOE] |
| | | | ([5meC])[sP].[dR](A)[sP].[MOE]([5meC])[sP].[dR](A)[sP].[MOE] |
| | | | ([5meC])[sP].[dR](A)[sP].[MOE]([5meC])[sP].[dR](A)[sP].[MOE] |
| | | | ([5meC])[sP].[dR](A)[sP].[MOE]([5meC])[sP].[dR](A)[sP].[MOE] |
| | | | ([5meC])[sP].[dR](A)[sP].[dR](C)[sP].[MOE](A)[sP].[MOE]([5meC]) |
| 29 | CACACACACACACA | 29.6 | [MOE]([5meC])[sP].[MOE](A)[sP].[dR](C)[sP].[MOE](A)[sP].[dR](C) |
| | CACACACACACACA | | [sP].[MOE](A)[sP].[dR](C)[sP].[MOE](A)[sP].[dR](C)[sP].[MOE] |
| | CAC | | (A)[sP].[dR](C)[sP].[MOE](A)[sP].[dR](C)[sP].[MOE](A)[sP].[dR] |
| | | | (C)[sP].[MOE](A)[sP].[dR](C)[sP].[MOE](A)[sP].[dR](C)[sP].[MOE] |
| | | | (A)[sP].[dR](C)[sP].[MOE](A)[sP].[dR](C)[sP].[MOE](A)[sP].[dR] |
| | | | (C)[sP].[MOE](A)[sP].[dR](C)[sP].[MOE](A)[sP].[dR](C)[sP].[MOE] |
| | | | (A)[sP].[MOE]([5meC]) |
| 29 | CACACACACACACA | 29.7 | [MOE]([5meC])[sP].[MOE](A)[sP].[dR](C)[sP].[dR](A)[sP].[MOE] |
| | CACACACACACACA | | ([5meC])[sP].[dR](A)[sP].[MOE]([5meC])[sP].[dR](A)[sP].[MOE] |
| | CAC | | ([5meC])[sP].[dR](A)[sP].[MOE]([5meC])[sP].[dR](A)[sP].[MOE] |
| | | | ([5meC])[sP].[dR](A)[sP].[MOE]([5meC])[sP].[dR](A)[sP].[MOE] |
| | | | ([5meC])[sP].[dR](A)[sP].[MOE]([5meC])[sP].[dR](A)[sP].[MOE] |
| | | | ([5meC])[sP].[dR](A)[sP].[MOE]([5meC])[sP].[dR](A)[sP].[MOE] |
| | | | ([5meC])[sP].[dR](A)[sP].[MOE]([5meC])[sP].[dR](A)[sP].[dR] |
| | | | (C)[sP].[MOE](A)[sP].[MOE]([5meC]) |
| 23 | CACACACACACACA | 23.4 | [mR](C)[sP].[mR](A)[sP].[mR](C)[sP].[mR](A)[sP].[mR](C)[sP].[mR] |
| | CACACACACAC | | (A)[sP].[mR](C)[sP].[mR](A)[sP].[mR](C)[sP].[mR](A)[sP].[mR](C) |
| | | | [sP].[mR](A)[sP].[mR](C)[sP].[mR](A)[sP].[mR](C)[sP].[mR](A)[sP]. |
| | | | [mR](C)[sP].[mR](A)[sP].[mR](C)[sP].[mR](A)[sP].[mR](C)[sP].[mR] |
| | | | (A)[sP].[mR](C)[sP].[mR](A)[sP].[mR](C) |
| 25 | CACACACACACACA | 25.8 | [mR](C)[sP].[mR](A)[sP].[mR](C)[sP].[mR](A)[sP].[mR](C)[sP].[mR] |
| | CACACACACACAC | | (A)[sP].[mR](C)[sP].[mR](A)[sP].[mR](C)[sP].[mR](A)[sP].[mR](C) |
| | | | [sP].[mR](A)[sP].[mR](C)[sP].[mR](A)[sP].[mR](C)[sP].[mR](A)[sP]. |
| | | | [mR](C)[sP].[mR](A)[sP].[mR](C)[sP].[mR](A)[sP].[mR](C)[sP].[mR] |
| | | | (A)[sP].[mR](C)[sP].[mR](A)[sP].[mR](C)[sP].[mR](A)[sP].[mR](C) |
| 27 | CACACACACACACA | 27.8 | [mR](C)[sP].[mR](A)[sP].[mR](C)[sP].[mR](A)[sP].[mR](C)[sP].[mR] |
| | CACACACACACACA | | (A)[sP].[mR](C)[sP].[mR](A)[sP].[mR](C)[sP].[mR](A)[sP].[mR](C) |
| | C | | [sP].[mR](A)[sP].[mR](C)[sP].[mR](A)[sP].[mR](C)[sP].[mR](A)[sP]. |
| | | | [mR](C)[sP].[mR](A)[sP].[mR](C)[sP].[mR](A)[sP].[mR](C)[sP].[mR](A) |
| | | | [sP].[mR](C)[sP].[mR](A)[sP].[mR](C)[sP].[mR](A)[sP].[mR](C)[sP]. |
| | | | [mR](A)[sP].[mR](C) |
| 29 | CACACACACACACA | 29.8 | [mR](C)[sP].[mR](A)[sP].[mR](C)[sP].[mR](A)[sP].[mR](C)[sP].[mR](A) |
| | CACACACACACACA | | [sP].[mR](C)[sP].[mR](A)[sP].[mR](C)[sP].[mR](A)[sP].[mR](C)[sP]. |
| | CAC | | [mR](A)[sP].[mR](C)[sP].[mR](A)[sP].[mR](C)[sP].[mR](A)[sP].[mR](C) |
| | | | [sP].[mR](A)[sP].[mR](C)[sP].[mR](A)[sP].[mR](C)[sP].[mR](A)[sP]. |
| | | | [mR](C)[sP].[mR](A)[sP].[mR](C)[sP].[mR](A)[sP].[mR](C)[sP].[mR] |
| | | | (A)[sP].[mR](C)[sP].[mR](A)[sP].[mR](C) |
| 23 | CACACACACACACA | 23.5 | [mR](C)[sP].[mR](A)[sP].[dR](C)[sP].[dR](A)[sP].[mR](C)[sP].[dR](A) |
| | CACACACACAC | | [sP].[mR](C)[sP].[dR](A)[sP].[mR](C)[sP].[dR](A)[sP].[mR](C)[sP]. |
| | | | [dR](A)[sP].[mR](C)[sP].[dR](A)[sP].[mR](C)[sP].[dR](A)[sP].[mR](C) |
| | | | [sP].[dR](A)[sP].[mR](C)[sP].[dR](A)[sP].[mR](C)[sP].[dR](A)[sP]. |
| | | | [dR](C)[sP].[mR](A)[sP].[mR](C) |
| 25 | CACACACACACACA | 25.9 | [mR](C)[sP].[mR](A)[sP].[dR](C)[sP].[dR](A)[sP].[mR](C)[sP].[dR](A) |
| | CACACACACACAC | | [sP].[mR](C)[sP].[dR](A)[sP].[mR](C)[sP].[dR](A)[sP].[mR](C)[sP]. |
| | | | [dR](A)[sP].[mR](C)[sP].[dR](A)[sP].[mR](C)[sP].[dR](A)[sP].[mR](C) |
| | | | [sP].[dR](A)[sP].[mR](C)[sP].[dR](A)[sP].[mR](C)[sP].[dR](A)[sP]. |
| | | | [mR](C)[sP].[dR](A)[sP].[dR](C)[sP].[mR](A)[sP].[mR](C) |
| 27 | CACACACACACACA | 27.9 | [mR](C)[sP].[mR](A)[sP].[dR](C)[sP].[dR](A)[sP].[mR](C)[sP].[dR](A) |
| | CACACACACACACA | | [sP].[mR](C)[sP].[dR](A)[sP].[mR](C)[sP].[dR](A)[sP].[mR](C)[sP]. |
| | C | | [dR](A)[sP].[mR](C)[sP].[dR](A)[sP].[mR](C)[sP].[dR](A)[sP].[mR](C) |
| | | | [sP].[dR](A)[sP].[mR](C)[sP].[dR](A)[sP].[mR](C)[sP].[dR](A)[sP].[mR] |
| | | | (C)[sP].[dR](A)[sP].[mR](C)[sP].[dR](A)[sP].[dR](C)[sP].[mR](A)[sP]. |
| | | | [mR](C) |
| 29 | CACACACACACACA | 29.9 | [mR](C)[sP].[mR](A)[sP].[dR](C)[sP].[dR](A)[sP].[mR](C)[sP].[dR](A) |
| | CACACACACACACA | | [sP].[mR](C)[sP].[dR](A)[sP].[mR](C)[sP].[dR](A)[sP].[mR](C)[sP]. |
| | CAC | | [dR](A)[sP].[mR](C)[sP].[dR](A)[sP].[mR](C)[sP].[dR](A)[sP].[mR](C) |
| | | | [sP].[dR](A)[sP].[mR](C)[sP].[dR](A)[sP].[mR](C)[sP].[dR](A)[sP]. |
| | | | [mR](C)[sP].[dR](A)[sP].[mR](C)[sP].[dR](A)[sP].[mR](C)[sP].[dR](A) |
| | | | [sP].[dR](C)[sP].[mR](A)[sP].[mR](C) |
| 34 | ACACACACACACAC | 34 | [LR](A)[sP].[LR]([5meC])[sP].[dR](A)[sP].[LR]([5meC])[sP].[dR](A) |
| | ACACACACACACAC | | [sP].[LR]([5meC])[sP].[dR](A)[sP].[LR]([5meC])[sP].[dR](A)[sP]. |
| | ACAC | | [LR]([5meC])[sP].[dR](A)[sP].[LR]([5meC])[sP].[dR](A)[sP].[LR] |
| | | | ([5meC])[sP].[dR](A)[sP].[LR]([5meC])[sP].[dR](A)[sP].[LR]([5meC]) |
| | | | [sP].[dR](A)[sP].[LR]([5meC])[sP].[dR](A)[sP].[LR]([5meC])[sP]. |
| | | | [dR](A)[sP].[LR]([5meC])[sP].[dR](A)[sP].[LR]([5meC])[sP].[dR](A) |
| | | | [sP].[LR]([5meC])[sP].[dR](A)[sP].[LR]([5meC])[sP].[dR](A)[sP].[LR] |
| | | | ([5meC]) |
| 20 | CACACACACACACA | 20.2 | [LR]([5meC])[sP].[LR](A)[sP].[dR](C)[sP].[MOE](A)[sP].[LR]([5meC]) |
| | CACACACAC | | [sP].[MOE](A)[sP].[LR]([5meC])[sP].[dR](A)[sP].[LR]([5meC])[sP]. |
| | | | [MOE](A)[sP].[LR]([5meC])[sP].[dR](A)[sP].[LR]([5meC])[sP].[dR] |
| | | | (A)[sP].[LR]([5meC])[sP].[dR](A)[sP].[LR]([5meC])[sP].[dR](A)[sP]. |
| | | | [LR]([5meC])[sP].[dR](A)[sP].[dR](C)[sP].[LR](A)[sP].[LR]([5meC]) |
| 50 | CACACACCCACICA | 50 | [LR]([5meC])[sP].[LR](A)[sP].[dR](C)[sP].[dR](A)[sP].[LR]([5meC]) |
| | CACACGCAC | | [sP].[dR](A)[sP].[LR]([5meC])[sP].[dR](C)[sP].[LR]([5meC])[sP]. |
| | | | [dR](A)[sP].[LR]([5meC])[sP].[dR](A)[sP].[LR]([5meC])[sP].[dR](A) |
| | | | [sP].[LR]([5meC])[sP].[dR](A)[sP].[LR]([5meC])[sP].[dR](A)[sP]. |
| | | | [LR]([5meC])[sP].[dR](G)[sP].[dR](C)[sP].[LR](A)[sP].[LR]([5meC]) |
| 51 | CACACICACACACA | 51 | [LR]([5meC])[sP].[LR](A)[sP].[dR](C)[sP].[dR](A)[sP].[LR]([5meC]) |
| | CACACACAC | | [sP].[dR]([In])[sP].[LR]([5meC])[sP].[dR](A)[sP].[LR]([5meC])[sP]. |
| | | | [dR](A)[sP].[LR]([5meC])[sP].[dR](A)[sP].[LR]([5meC])[sP].[dR](A) |
| | | | [sP].[LR]([5meC])[sP].[dR](A)[sP].[LR]([5meC])[sP].[dR](A)[sP]. |
| | | | [LR]([5meC])[sP].[dR](A)[sP].[dR](C)[sP].[LR](A)[sP].[LR]([5meC]) |
| 52 | CACACACACACICA | 52 | [LR]([5meC])[sP].[LR](A)[sP].[dR](C)[sP].[dR](A)[sP].[LR]([5meC]) |
| | CACACACAC | | [sP].[dR](A)[sP].[LR]([5meC])[sP].[dR](A)[sP].[LR]([5meC])[sP]. |
| | | | [dR](A)[sP].[LR]([5meC])[sP].[dR]([In])[sP].[LR]([5meC])[sP].[dR] |
| | | | (A)[sP].[LR]([5meC])[sP].[dR](A)[sP].[LR]([5meC])[sP].[dR](A)[sP]. |
| | | | [LR]([5meC])[sP].[dR](A)[sP].[dR](C)[sP].[LR](A)[sP].[LR]([5meC]) |
| 53 | CACACICACACACA | 53 | [LR]([5meC])[sP].[LR](A)[sP].[dR](C)[sP].[dR](A)[sP].[LR]([5meC]) |
| | CACACACTC | | [sP].[dR](In])[sP].[LR]([5meC])[sP].[dR](A)[sP].[LR]([5meC])[sP]. |
| | | | [dR](A)[sP].[LR]([5meC])[sP].[dR](A)[sP].[LR]([5meC])[sP].[dR](A) |
| | | | [sP].[LR]([5meC])[sP].[dR](A)[sP].[LR]([5meC])[sP].[dR](A)[sP]. |
| | | | [LR]([5meC])[sP].[dR](A)[sP].[dR](C)[sP].[LR](T)[sP].[LR]([5meC]) |
| 54 | CACATACACACCCA | 54 | [LR]([5meC])[sP].[LR](A)[sP].[dR](C)[sP].[dR](A)[sP].[LR](T)[sP]. |
| | CACACACAC | | [LR](A)[sP].[LR]([5meC])[sP].[dR](A)[sP].[LR]([5meC])[sP].[dR] |
| | | | (A)[sP].[LR]([5meC])[sP].[dR](C)[sP].[LR]([5meC])[sP].[dR](A) |
| | | | [sP].[LR]([5meC])[sP].[dR](A)[sP].[LR]([5meC])[sP].[dR](A)[sP]. |
| | | | [LR]([5meC])[sP].[dR](A)[sP].[dR](C)[sP].[LR](A)[sP].[LR]([5meC]) |
| 55 | CACACACACACACA | 55 | [LR]([5meC])[sP].[LR](A)[sP].[dR](C)[sP].[dR](A)[sP].[LR]([5meC]) |
| | CACGCACAC | | [sP].[dR](A)[sP].[LR]([5meC])[sP].[dR](A)[sP].[LR]([5meC])[sP]. |
| | | | [dR](A)[sP].[LR]([5meC])[sP].[dR](A)[sP].[LR]([5meC])[sP].[dR](A) |
| | | | [sP].[LR]([5meC])[sP].[dR](A)[sP].[LR]([5meC])[sP].[dR](G)[sP]. |
| | | | [LR]([5meC])[sP].[dR](A)[sP].[dR](C)[sP].[LR](A)[sP].[LR]([5meC]) |
| 56 | CACICACICACACA | 56 | [LR]([5meC])[sP].[LR](A)[sP].[dR](C)[sP].[dR]([In])[sP].[LR] |
| | CICACICAC | | ([5meC])[sP].[dR](A)[sP].[LR]([5meC])[sP].[dR]([In])[sP].[LR] |
| | | | ([5meC])[sP].[dR](A)[sP].[LR]([5meC])[sP].[dR](A)[sP].[LR] |
| | | | ([5meC])[sP].[dR](A)[sP].[LR]([5meC])[sP].[dR]([In])[sP].[LR] |
| | | | ([5meC])[sP].[dR](A)[sP].[LR]([5meC])[sP].[dR]([In])[sP].[dR](C) |
| | | | [sP].[LR](A)[sP].[LR]([5meC]) |
| 57 | CACACACACACACA | 57 | [LR]([5meC])[sP].[LR](A)[sP].[dR](C)[sP].[dR](A)[sP].[LR]([5meC]) |
| | CTCTCACAC | | [sP].[dR](A)[sP].[LR]([5meC])[sP].[dR](A)[sP].[LR]([5meC])[sP]. |
| | | | [dR](A)[sP].[LR]([5meC])[sP].[dR](A)[sP].[LR]([5meC])[sP].[dR](A) |
| | | | [sP].[LR]([5meC])[sP].[dR](T)[sP].[LR]([5meC])[sP].[dR](T)[sP]. |
| | | | [LR]([5meC])[sP].[dR](A)[sP].[dR](C)[sP].[LR](A)[sP].[LR]([5meC]) |
| 58 | CTCACACACACACA | 58 | [LR]([5meC])[sP].[LR](T)[sP].[dR](C)[sP].[dR](A)[sP].[LR]([5meC]) |
| | CACACACAC | | [sP].[dR](A)[sP].[LR]([5meC])[sP].[dR](A)[sP].[LR]([5meC])[sP]. |
| | | | [dR](A)[sP].[LR]([5meC])[sP].[dR](A)[sP].[LR]([5meC])[sP].[dR](A) |
| | | | [sP].[LR]([5meC])[sP].[dR](A)[sP].[LR]([5meC])[sP].[dR](A)[sP]. |
| | | | [LR]([5meC])[sP].[dR](A)[sP].[dR](C)[sP].[LR](A)[sP].[LR]([5meC]) |
| 59 | CACACACACACAAA | 59 | [LR]([5meC])[sP].[LR](A)[sP].[dR](C)[sP].[dR](A)[sP].[LR]([5meC]) |
| | CACACACAC | | [sP].[dR](A)[sP].[LR]([5meC])[sP].[dR](A)[sP].[LR]([5meC])[sP]. |
| | | | [dR](A)[sP].[LR]([5meC])[sP].[dR](A)[sP].[LR](A)[sP].[dR](A)[sP]. |
| | | | [LR]([5meC])[sP].[dR](A)[sP].[LR]([5meC])[sP].[dR](A)[sP].[LR] |
| | | | ([5meC])[sP].[dR](A)[sP].[dR](C)[sP].[LR](A)[sP].[LR]([5meC]) |
| 60 | CACACGCACGCACA | 60 | [LR]([5meC])[sP].[LR](A)[sP].[dR](C)[sP].[dR](A)[sP].[LR]([5meC]) |
| | CACACACAC | | [sP].[dR](G)[sP].[LR]([5meC])[sP].[dR](A)[sP].[LR]([5meC])[sP]. |
| | | | [dR](G)[sP].[LR]([5meC])[sP].[dR](A)[sP].[LR]([5meC])[sP].[dR](A) |
| | | | [sP].[LR]([5meC])[sP].[dR](A)[sP].[LR]([5meC])[sP].[dR](A)[sP]. |
| | | | [LR]([5meC])[sP].[dR](A)[sP].[dR](C)[sP].[LR](A)[sP].[LR]([5meC]) |
| 61 | CACGCACACACCCA | 61 | [LR]([5meC])[sP].[LR](A)[sP].[dR](C)[sP].[dR](G)[sP].[LR]([5meC]) |
| | CACACTCAC | | [sP].[dR](A)[sP].[LR]([5meC])[sP].[dR](A)[sP].[LR]([5meC])[sP]. |
| | | | [dR](A)[sP].[LR]([5meC])[sP].[dR](C)[sP].[LR]([5meC])[sP].[dR](A) |
| | | | [sP].[LR]([5meC])[sP].[dR](A)[sP].[LR]([5meC])[sP].[dR](A)[sP]. |
| | | | [LR]([5meC])[sP].[dR](T)[sP].[dR](C)[sP].[LR](A)[sP].[LR]([5meC]) |
| 62 | CACACACACACACT | 62 | [LR]([5meC])[sP].[LR](A)[sP].[dR](C)[sP].[dR](A)[sP].[LR]([5meC]) |
| | CTCTCACAC | | [sP].[dR](A)[sP].[LR]([5meC])[sP].[dR](A)[sP].[LR]([5meC])[sP]. |
| | | | [dR](A)[sP].[LR]([5meC])[sP].[dR](A)[sP].[LR]([5meC])[sP].[dR](T) |
| | | | [sP].[LR]([5meC])[sP].[dR](T)[sP].[LR]([5meC])[sP].[dR](T)[sP]. |
| | | | [LR]([5meC])[sP].[dR](A)[sP].[dR](C)[sP].[LR](A)[sP].[LR]([5meC]) |
| 63 | CACACACACACACI | 63 | [LR]([5meC])[sP].[LR](A)[sP].[dR](C)[sP].[dR](A)[sP].[LR]([5meC]) |
| | CICICICAC | | [sP].[dR](A)[sP].[LR]([5meC])[sP].[dR](A)[sP].[LR]([5meC])[sP]. |
| | | | [dR](A)[sP].[LR]([5meC])[sP].[dR](A)[sP].[LR]([5meC])[sP].[dR] |
| | | | ([In])[sP].[LR]([5meC])[sP].[dR]([In])[sP].[LR]([5meC])[sP].[dR] |
| | | | ([In])[sP].[LR]([5meC])[sP].[dR]([In])[sP].[dR](C)[sP].[LR](A)[sP]. |
| | | | [LR]([5meC]) |
| 64 | CACTCACACACTCA | 64 | [LR]([5meC])[sP].[LR](A)[sP].[dR](C)[sP].[dR](T)[sP].[LR]([5meC]) |
| | CACACACAC | | [sP].[dR](A)[sP].[LR]([5meC])[sP].[dR](A)[sP].[LR]([5meC])[sP]. |
| | | | [dR](A)[sP].[LR]([5meC])[sP].[dR](T)[sP].[LR]([5meC])[sP].[dR](A) |
| | | | [sP].[LR]([5meC])[sP].[dR](A)[sP].[LR]([5meC])[sP].[dR](A)[sP]. |
| | | | [LR]([5meC])[sP].[dR](A)[sP].[dR](C)[sP].[LR](A)[sP].[LR]([5meC]) |
| 65 | CACGCACACACGCA | 65 | [LR]([5meC])[sP].[LR](A)[sP].[dR](C)[sP].[dR](G)[sP].[LR]([5meC]) |
| | CACACACAC | | [sP].[dR](A)[sP].[LR]([5meC])[sP].[dR](A)[sP].[LR]([5meC])[sP]. |
| | | | [dR](A)[sP].[LR]([5meC])[sP].[dR](G)[sP].[LR]([5meC])[sP].[dR] |
| | | | (A)[sP].[LR]([5meC])[sP].[dR](A)[sP].[LR]([5meC])[sP].[dR](A)[sP]. |
| | | | [LR]([5meC])[sP].[dR](A)[sP].[dR](C)[sP].[LR](A)[sP].[LR]([5meC]) |
| 66 | CACCCACACACCCA | 66 | [LR]([5meC])[sP].[LR](A)[sP].[dR](C)[sP].[dR](C)[sP].[LR]([5meC]) |
| | CACACACAC | | [sP].[dR](A)[sP].[LR]([5meC])[sP].[dR](A)[sP].[LR]([5meC])[sP]. |
| | | | [dR](A)[sP].[LR]([5meC])[sP].[dR](C)[sP].[LR]([5meC])[sP].[dR](A) |
| | | | [sP].[LR]([5meC])[sP].[dR](A)[sP].[LR]([5meC])[sP].[dR](A)[sP]. |
| | | | [LR]([5meC])[sP].[dR](A)[sP].[dR](C)[sP].[LR](A)[sP].[LR]([5meC]) |
| 67 | CACACGCACATACA | 67 | [LR]([5meC])[sP].[LR](A)[sP].[dR](C)[sP].[dR](A)[sP].[LR]([5meC]) |
| | CACACCCAC | | [sP].[dR]([In])[sP].[LR]([5meC])[sP].[dR](A)[sP].[LR]([5meC])[sP]. |
| | | | [dR](A)[sP].[LR](T)[sP].[dR](A)[sP].[LR]([5meC])[sP].[dR](A)[sP]. |
| | | | [LR]([5meC])[sP].[dR](A)[sP].[LR]([5meC])[sP].[dR](A)[sP].[LR] |
| | | | ([5meC])[sP].[dR](C)[sP].[dR](C)[sP].[LR](A)[sP].[LR]([5meC]) |
| 68 | TTCACACACACACA | 68 | [LR](T)[sP].[LR](T)[sP].[dR](C)[sP].[dR](A)[sP].[LR]([5meC])[sP]. |
| | CACACACAC | | [dR](A)[sP].[LR]([5meC])[sP].[dR](A)[sP].[LR]([5meC])[sP].[dR](A) |
| | | | [sP].[LR]([5meC])[sP].[dR](A)[sP].[LR]([5meC])[sP].[dR](A)[sP].[LR] |
| | | | ([5meC])[sP].[dR](A)[sP].[LR]([5meC])[sP].[dR](A)[sP].[LR] |
| | | | ([5meC])[sP].[dR](A)[sP].[dR](C)[sP].[LR](A)[sP].[LR]([5meC]) |
| 69 | CACACACACACACA | 69 | [LR]([5meC])[sP].[LR](A)[sP].[dR](C)[sP].[dR](A)[sP].[LR]([5meC]) |
| | CACACACAA | | [sP].[dR](A)[sP].[LR]([5meC])[sP].[dR](A)[sP].[LR]([5meC])[sP]. |
| | | | [dR](A)[sP].[LR]([5meC])[sP].[dR](A)[sP].[LR]([5meC])[sP].[dR](A) |
| | | | [sP].[LR]([5meC])[sP].[dR](A)[sP].[LR]([5meC])[sP].[dR](A)[sP].[LR] |
| | | | ([5meC])[sP].[dR](A)[sP].[dR](C)[sP].[LR](A)[sP].[LR](A) |
| 70 | CACACACACACACA | 70 | [LR]([5meC])[sP].[LR](A)[sP].[dR](C)[sP].[LR](A)[sP].[dR](C)[sP]. |
| | CACACAIACACACA | | [LR](A)[sP].[dR](C)[sP].[LR](A)[sP].[dR](C)[sP].[LR](A)[sP].[dR] |
| | C | | (C)[sP].[LR](A)[sP].[dR](C)[sP].[LR](A)[sP].[dR](C)[sP].[LR](A) |
| | | | [sP].[dR](C)[sP].[LR](A)[sP].[dR](C)[sP].[LR](A)[sP].[dR]([In]) |
| | | | [sP].[LR](A)[sP].[dR](C)[sP].[LR](A)[sP].[dR](C)[sP].[LR](A)[sP]. |
| | | | [dR](C)[sP].[LR](A)[sP].[LR]([5meC]) |
| 71 | CACAIACACACACA | 71.1 | [LR]([5meC])[sP].[LR](A)[sP].[dR](C)[sP].[LR](A)[sP].[dR]([In]) |
| | CACACACACACACA | | [sP].[LR](A)[sP].[dR](C)[sP].[LR](A)[sP].[dR](C)[sP].[LR](A)[sP]. |
| | C | | [dR](C)[sP].[LR](A)[sP].[dR](C)[sP].[LR](A)[sP].[dR](C)[sP].[LR] |
| | | | (A)[sP].[dR](C)[sP].[LR](A)[sP].[dR](C)[sP].[LR](A)[sP].[dR](C) |
| | | | [sP].[LR](A)[sP].[dR](C)[sP].[LR](A)[sP].[dR](C)[sP].[LR](A)[sP]. |
| | | | [dR](C)[sP].[LR](A)[sP].[LR]([5meC]) |
| 72 | CATACACACACACA | 72 | [LR]([5meC])[sP].[LR](A)[sP].[dR](T)[sP].[LR](A)[sP].[dR](C)[sP]. |
| | CACACATACACACA | | [LR](A)[sP].[dR](C)[sP].[LR](A)[sP].[dR](C)[sP].[LR](A)[sP].[dR] |
| | C | | (C)[sP].[LR](A)[sP].[dR](C)[sP].[LR](A)[sP].[dR](C)[sP].[LR](A) |
| | | | [sP].[dR](C)[sP].[LR](A)[sP].[dR](C)[sP].[LR](A)[sP].[dR](T)[sP]. |
| | | | [LR](A)[sP].[dR](C)[sP].[LR](A)[sP].[dR](C)[sP].[LR](A)[sP].[dR](C) |
| | | | [sP].[LR](A)[sP].[LR]([5meC]) |
| 73 | CAAACACACACACA | 73 | [LR]([5meC])[sP].[LR](A)[sP].[dR](A)[sP].[LR](A)[sP].[dR](C)[sP]. |
| | CACACACACACACA | | [LR](A)[sP].[dR](C)[sP].[LR](A)[sP].[dR](C)[sP].[LR](A)[sP].[dR] |
| | C | | (C)[sP].[LR](A)[sP].[dR](C)[sP].[LR](A)[sP].[dR](C)[sP].[LR](A) |
| | | | [sP].[dR](C)[sP].[LR](A)[sP].[dR](C)[sP].[LR](A)[sP].[dR](C)[sP]. |
| | | | [LR](A)[sP].[dR](C)[sP].[LR](A)[sP].[dR](C)[sP].[LR](A)[sP].[dR] |
| | | | (C)[sP].[LR](A)[sP].[LR]([5meC]) |
| 74 | CACACACATCACAC | 74 | [LR]([5meC])[sP].[LR](A)[sP].[dR](C)[sP].[LR](A)[sP].[dR](C)[sP]. |
| | ACACACACACACAC | | [LR](A)[sP].[dR](C)[sP].[LR](A)[sP].[LR]([5meC])[sP].[LR](T)[sP]. |
| | | | [dR](C)[sP].[LR](A)[sP].[dR](C)[sP].[LR](A)[sP].[dR](C)[sP].[LR] |
| | | | (A)[sP].[dR](C)[sP].[LR](A)[sP].[dR](C)[sP].[LR](A)[sP].[dR](C) |
| | | | [sP].[LR](A)[sP].[dR](C)[sP].[LR](A)[sP].[dR](C)[sP].[LR](A)[sP]. |
| | | | [dR](C)[sP].[LR](A)[sP].[LR]([5meC]) |
| 75 | CACAGACACACACA | 75 | [LR]([5meC])[sP].[LR](A)[sP].[dR](C)[sP].[LR](A)[sP].[dR](G)[sP]. |
| | CACACACACACACA | | [LR](A)[sP].[dR](C)[sP].[LR](A)[sP].[dR](C)[sP].[LR](A)[sP].[dR] |
| | C | | (C)[sP].[LR](A)[sP].[dR](C)[sP].[LR](A)[sP].[dR](C)[sP].[LR](A) |
| | | | [sP].[dR](C)[sP].[LR](A)[sP].[dR](C)[sP].[LR](A)[sP].[dR](C)[sP]. |
| | | | [LR](A)[sP].[dR](C)[sP].[LR](A)[sP].[dR](C)[sP].[LR](A)[sP].[dR] |
| | | | (C)[sP].[LR](A)[sP].[LR]([5meC]) |
| 71 | CACAIACACACACA | 71.2 | [LR]([5meC])[sP].[LR](A)[sP].[dR](C)[sP].[LR](A)[sP].[dR]([In]) |
| | CACACACACACACA | | [sP].[LR](A)[sP].[dR](C)[sP].[LR](A)[sP].[dR](C)[sP].[LR](A)[sP]. |
| | C | | [dR](C)[sP].[LR](A)[sP].[dR](C)[sP].[LR](A)[sP].[dR](C)[sP].[LR] |
| | | | (A)[sP].[dR](C)[sP].[LR](A)[sP].[dR](C)[sP].[LR](A)[sP].[dR](C) |
| | | | [sP].[LR](A)[sP].[dR](C)[sP].[LR](A)[sP].[dR](C)[sP].[LR](A)[sP]. |
| | | | [dR](C)[sP].[LR](A)[sP].[LR]([5meC]) |
| 76 | CACAAACACACACA | 76 | [LR]([5meC])[sP].[LR](A)[sP].[dR](C)[sP].[LR](A)[sP].[dR](A)[sP]. |
| | CACACACACACACA | | [LR](A)[sP].[dR](C)[sP].[LR](A)[sP].[dR](C)[sP].[LR](A)[sP].[dR] |
| | C | | (C)[sP].[LR](A)[sP].[dR](C)[sP].[LR](A)[sP].[dR](C)[sP].[LR](A) |
| | | | [sP].[dR](C)[sP].[LR](A)[sP].[dR](C)[sP].[LR](A)[sP].[dR](C)[sP]. |
| | | | [LR](A)[sP].[dR](C)[sP].[LR](A)[sP].[dR](C)[sP].[LR](A)[sP].[dR] |
| | | | (C)[sP].[LR](A)[sP].[LR]([5meC]) |
| 77 | CACATACACACACA | 77 | [LR]([5meC])[sP].[LR](A)[sP].[dR](C)[sP].[LR](A)[sP].[dR](T)[sP]. |
| | CACACACACACACA | | [LR](A)[sP].[dR](C)[sP].[LR](A)[sP].[dR](C)[sP].[LR](A)[sP].[dR] |
| | C | | (C)[sP].[LR](A)[sP].[dR](C)[sP].[LR](A)[sP].[dR](C)[sP].[LR](A) |
| | | | [sP].[dR](C)[sP].[LR](A)[sP].[dR](C)[sP].[LR](A)[sP].[dR](C)[sP]. |
| | | | [LR](A)[sP].[dR](C)[sP].[LR](A)[sP].[dR](C)[sP].[LR](A)[sP].[dR] |
| | | | (C)[sP].[LR](A)[sP].[LR]([5meC]) |
| 78 | CATACGCACATACA | 78 | [LR]([5meC])[sP].[LR](A)[sP].[dR](T)[sP].[LR](A)[sP].[dR](C)[sP]. |
| | CGCACACACAAACA | | [LR](G)[sP].[dR](C)[sP].[LR](A)[sP].[dR](C)[sP].[LR](A)[sP].[dR] |
| | C | | (T)[sP].[LR](A)[sP].[dR](C)[sP].[LR](A)[sP].[dR](C)[sP].[LR](G) |
| | | | [sP].[dR](C)[sP].[LR](A)[sP].[dR](C)[sP].[LR](A)[sP].[dR](C)[sP]. |
| | | | [LR](A)[sP].[dR](C)[sP].[LR](A)[sP].[dR](A)[sP].[LR](A)[sP].[dR] |
| | | | (C)[sP].[LR](A)[sP].[LR]([5meC]) |
| 79 | CACATATACACATA | 79 | [LR]([5meC])[sP].[LR](A)[sP].[dR](C)[sP].[LR](A)[sP].[dR](T)[sP]. |
| | CACACACACACACA | | [LR](A)[sP].[dR](T)[sP].[LR](A)[sP].[dR](C)[sP].[LR](A)[sP].[dR] |
| | C | | (C)[sP].[LR](A)[sP].[dR](T)[sP].[LR](A)[sP].[dR](C)[sP].[LR](A) |
| | | | [sP].[dR](C)[sP].[LR](A)[sP].[dR](C)[sP].[LR](A)[sP].[dR](C)[sP]. |
| | | | [LR](A)[sP].[dR](C)[sP].[LR](A)[sP].[dR](C)[sP].[LR](A)[sP].[dR] |
| | | | (C)[sP].[LR](A)[sP].[LR]([5meC]) |
| 80 | CACACACACACACA | 80 | [LR]([5meC])[sP].[LR](A)[sP].[dR](C)[sP].[LR](A)[sP].[dR](C)[sP]. |
| | CACACTCTCTCTCT | | [LR](A)[sP].[dR](C)[sP].[LR](A)[sP].[dR](C)[sP].[LR](A)[sP].[dR] |
| | C | | (C)[sP].[LR](A)[sP].[dR](C)[sP].[LR](A)[sP].[dR](C)[sP].[LR](A) |
| | | | [sP].[dR](C)[sP].[LR](A)[sP].[dR](C)[sP].[LR](T)[sP].[dR](C)[sP]. |
| | | | [LR](T)[sP].[dR](C)[sP].[LR](T)[sP].[dR](C)[sP].[LR](T)[sP].[dR] |
| | | | (C)[sP].[LR](T)[sP].[LR]([5meC]) |
| 81 | CACGCACACACACA | 81 | [LR]([5meC])[sP].[LR](A)[sP].[dR](C)[sP].[LR](G)[sP].[dR](C)[sP]. |
| | CACACACACACACA | | [LR](A)[sP].[dR](C)[sP].[LR](A)[sP].[dR](C)[sP].[LR](A)[sP].[dR] |
| | C | | (C)[sP].[LR](A)[sP].[dR](C)[sP].[LR](A)[sP].[dR](C)[sP].[LR](A) |
| | | | [sP].[dR](C)[sP].[LR](A)[sP].[dR](C)[sP].[LR](A)[sP].[dR](C)[sP]. |
| | | | [LR](A)[sP].[dR](C)[sP].[LR](A)[sP].[dR](C)[sP].[LR](A)[sP].[dR] |
| | | | (C)[sP].[LR](A)[sP].[LR]([5meC]) |
| 82 | CACACACACACACA | 82 | [LR]([5meC])[sP].[LR](A)[sP].[dR](C)[sP].[LR](A)[sP].[dR](C)[sP]. |
| | CAITITITCACACA | | [LR](A)[sP].[dR](C)[sP].[LR](A)[sP].[dR](C)[sP].[LR](A)[sP].[dR] |
| | C | | (C)[sP].[LR](A)[sP].[dR](C)[sP].[LR](A)[sP].[dR](C)[sP].[LR](A) |
| | | | [sP].[dR]([In])[sP].[LR](T)[sP].[dR]([In])[sP].[LR](T)[sP].[dR] |
| | | | P].[LR](T)[sP].[dR](C)[sP].[LR](A)[sP].[dR](C)[sP].[LR](A)[sP]. |
| | | | [dR](C)[sP].[LR](A)[sP].[LR]([5meC]) |
| 83 | CGCACACACACACA | 83 | [LR]([5meC])[sP].[LR](G)[sP].[dR](C)[sP].[LR](A)[sP].[dR](C)[sP]. |
| | CACACACACACACA | | [LR](A)[sP].[dR](C)[sP].[LR](A)[sP].[dR](C)[sP].[LR](A)[sP].[dR] |
| | C | | (C)[sP].[LR](A)[sP].[dR](C)[sP].[LR](A)[sP].[dR](C)[sP].[LR](A) |
| | | | [sP].[dR](C)[sP].[LR](A)[sP].[dR](C)[sP].[LR](A)[sP].[dR](C)[sP]. |
| | | | [LR](A)[sP].[dR](C)[sP].[LR](A)[sP].[dR](C)[sP].[LR](A)[sP].[dR] |
| | | | (C)[sP].[LR](A)[sP].[LR]([5meC]) |
| 84 | CACACACACACACA | 84 | [LR]([5meC])[sP].[LR](A)[sP].[dR](C)[sP].[LR](A)[sP].[dR](C)[sP]. |
| | CAGACAGACAGACA | | [LR](A)[sP].[dR](C)[sP].[LR](A)[sP].[dR](C)[sP].[LR](A)[sP].[dR] |
| | C | | (C)[sP].[LR](A)[sP].[dR](C)[sP].[LR](A)[sP].[dR](C)[sP].[LR](A) |
| | | | [sP].[dR](G)[sP].[LR](A)[sP].[dR](C)[sP].[LR](A)[sP].[dR](G)[sP]. |
| | | | [LR](A)[sP].[dR](C)[sP].[LR](A)[sP].[dR](G)[sP].[LR](A)[sP].[dR] |
| | | | (C)[sP].[LR](A)[sP].[LR]([5meC]) |
| 85 | CACACACACAAAAA | 85 | [LR]([5meC])[sP].[LR](A)[sP].[dR](C)[sP].[LR](A)[sP].[dR](C)[sP]. |
| | AACACACACACACA | | [LR](A)[sP].[dR](C)[sP].[LR](A)[sP].[dR](C)[sP].[LR](A)[sP].[dR] |
| | C | | (A)[sP].[LR](A)[sP].[dR](A)[sP].[LR](A)[sP].[dR](A)[sP].[LR](A) |
| | | | [sP].[dR](C)[sP].[LR](A)[sP].[dR](C)[sP].[LR](A)[sP].[dR](C)[sP]. |
| | | | [LR](A)[sP].[dR](C)[sP].[LR](A)[sP].[dR](C)[sP].[LR](A)[sP].[dR] |
| | | | (C)[sP].[LR](A)[sP].[LR]([5meC]) |
| 86 | CACACACACACCCC | 86 | [LR]([5meC])[sP].[LR](A)[sP].[dR](C)[sP].[LR](A)[sP].[dR](C)[sP]. |
| | CCCACACACACACA | | [LR](A)[sP].[dR](C)[sP].[LR](A)[sP].[dR](C)[sP].[LR](A)[sP].[dR] |
| | C | | (C)[sP].[LR]([5meC])[sP].[dR](C)[sP].[LR]([5meC])[sP].[dR](C)[sP]. |
| | | | [sP].[LR]([5meC])[sP].[dR](C)[sP].[LR](A)[sP].[dR](C)[sP].[LR](A) |
| | | | [sP].[dR](C)[sP].[LR](A)[sP].[dR](C)[sP].[LR](A)[sP].[dR](C)[sP]. |
| | | | [LR](A)[sP].[dR](C)[sP].[LR](A)[sP].[LR]([5meC]) |
| 87 | CACACACACTCTCT | 87 | [MOE]([5meC])[sP].[MOE](A)[sP].[MOE]([5meC])[sP].[MOE](A)[sP]. |
| | CACACACACAC | | [MOE]([5meC])[sP].[MOE](A)[sP].[MOE]([5meC])[sP].[MOE](A)[sP]. |
| | | | [MOE]([5meC])[sP].[MOE](T)[sP].[MOE]([5meC])[sP].[MOE](T)[sSP]. |
| | | | [MOE]([5meC])[sP].[MOE](T)[sP].[MOE]([5meC])[sP].[MOE](A)[sP]. |
| | | | [MOE]([5meC])[sP].[MOE](A)[sP].[MOE]([5meC])[sP].[MOE](A)[sP]. |
| | | | [MOE]([5meC])[sP].[MOE](A)[sP].[MOE]([5meC])[sP].[MOE](A)[sP]. |
| | | | [MOE]([5meC]) |
| 88 | CACAAACACACACA | 88 | [MOE]([5meC])[sP].[MOE](A)[sP].[MOE]([5meC])[sP].[MOE](A)[sP]. |
| | CACACACACAC | | [MOE](A)[sP].[MOE](A)[sP].[MOE]([5meC])[sP].[MOE](A)[sP].[MOE] |
| | | | ([5meC])[sP].[MOE](A)[sP].[MOE]([5meC])[sP].[MOE](A)[sP].[MOE] |
| | | | ([5meC])[sP].[MOE](A)[sP].[MOE]([5meC])[sP].[MOE](A)[sP].[MOE] |
| | | | ([5meC])[sP].[MOE](A)[sP].[MOE]([5meC])[sP].[MOE](A)[sP].[MOE] |
| | | | ([5meC])[sP].[MOE](A)[sP].[MOE]([5meC])[sP].[MOE](A)[sP].[MOE] |
| | | | ([5meC]) |
| 23 | CACACACACACACA | 23.10 | [MOE]([5meC])[sP].[MOE](A)[sP].[LR]([5meC])[sP].[MOE](A)[sP].[LR] |
| | CACACACACAC | | ([5meC])[sP].[MOE](A)[sP].[LR]([5meC])[sP].[MOE](A)[sP].[LR] |
| | | | ([5meC])[sP].[MOE](A)[sP].[LR]([5meC])[sP].[MOE](A)[sP].[LR] |
| | | | ([5meC])[sP].[MOE](A)[sP].[LR]([5meC])[sP].[MOE](A)[sP].[LR] |
| | | | ([5meC])[sP].[MOE](A)[sP].[LR]([5meC])[sP].[MOE](A)[sP].[LR] |
| | | | ([5meC])[sP].[MOE](A)[sP][LR]([5meC])[sP].[MOE](A)[sP].[MOE] |
| | | | ([5meC]) |
| 89 | CACTCACACACACA | 89 | [MOE]([5meC])[sP].[MOE](A)[sP].[MOE]([5meC])[sP].[MOE](T)[sP]. |
| | CACACACACAC | | [MOE]([5meC])[sP].[MOE](A)[sP].[MOE]([5meC])[sP].[MOE](A)[sP]. |
| | | | [MOE]([5meC])[sP].[MOE](A)[sP].[MOE]([5meC])[sP].[MOE](A)[sP]. |
| | | | [MOE]([5meC])[sP].[MOE](A)[sP].[MOE]([5meC])[sP].[MOE](A)[sP]. |
| | | | [MOE]([5meC])[sP].[MOE](A)[sP].[MOE]([5meC])[sP].[MOE](A)[sP]. |
| | | | [MOE]([5meC])[sP].[MOE](A)[sP].[MOE]([5meC])[sP].[MOE](A)[sP]. |
| | | | [MOE]([5meC]) |
| 90 | CACACACACAAACA | 90 | [MOE]([5meC])[sP].[MOE](A)[sP].[MOE]([5meC])[sP].[MOE](A)[sP]. |
| | CACACACACAC | | [MOE]([5meC])[sP].[MOE](A)[sP].[MOE]([5meC])[sP].[MOE](A)[sP]. |
| | | | [MOE]([5meC])[sP].[MOE](A)[sP].[MOE](A)[sP].[MOE](A)[sP].[MOE] |
| | | | ([5meC])[sP].[MOE](A)[sP].[MOE]([5meC])[sP].[MOE](A)[sP].[MOE] |
| | | | ([5meC])[sP].[MOE](A)[sP].[MOE]([5meC])[sP].[MOE](A)[sP].[MOE] |
| | | | ([5meC])[sP].[MOE](A)[sP].[MOE]([5meC])[sP].[MOE](A)[sP].[MOE] |
| | | | ([5meC]) |
| 91 | CACCCACACACACA | 91 | [MOE]([5meC])[sP].[MOE](A)[sP].[MOE]([5meC])[sP].[MOE]([5meC]) |
| | CACACACACAC | | [sP].[MOE]([5meC])[sP].[MOE](A)[sP].[MOE]([5meC])[sP].[MOE](A) |
| | | | [sP].[MOE]([5meC])[sP].[MOE](A)[sP].[MOE]([5meC])[sP].[MOE](A) |
| | | | [sP].[MOE]([5meC])[sP].[MOE](A)[sP].[MOE]([5meC])[sP].[MOE](A) |
| | | | [sP].[MOE]([5meC])[sP].[MOE](A)[sP].[MOE]([5meC])[sP].[MOE](A) |
| | | | [sP].[MOE]([5meC])[sP].[MOE](A)[sP].[MOE]([5meC])[sP].[MOE](A) |
| | | | [sP].[MOE]([5meC]) |
| 92 | CTCTCTCACACACA | 92 | [MOE]([5meC])[sP].[MOE](T)[sP].[MOE]([5meC])[sP].[MOE](T)[sP]. |
| | CACACACACAC | | [MOE]([5meC])[sP].[MOE](T)[sP].[MOE]([5meC])[sP].[MOE](A)[sP]. |
| | | | [MOE]([5meC])[sP].[MOE](A)[sP].[MOE]([5meC])[sP].[MOE](A)[sP]. |
| | | | [MOE]([5meC])[sP].[MOE](A)[sP].[MOE]([5meC])[sP].[MOE](A)[sP]. |
| | | | [MOE]([5meC])[sP].[MOE](A)[sP].[MOE]([5meC])[sP].[MOE](A)[sP]. |
| | | | [MOE]([5meC])[sP].[MOE](A)[sP].[MOE]([5meC])[sP].[MOE](A)[sP]. |
| | | | [MOE]([5meC]) |
| 93 | CAAACACACACACA | 93 | [MOE]([5meC])[sP].[MOE](A)[sP].[MOE](A)[sP].[MOE](A)[sP].[MOE] |
| | CACACACACAC | | ([5meC])[sP].[MOE](A)[sP].[MOE]([5meC])[sP].[MOE](A)[sP].[MOE] |
| | | | ([5meC])[sP].[MOE](A)[sP].[MOE]([5meC])[sP].[MOE](A)[sP].[MOE] |
| | | | ([5meC])[sP].[MOE](A)[sP].[MOE]([5meC])[sP].[MOE](A)[sP].[MOE] |
| | | | ([5meC])[sP].[MOE](A)[sP].[MOE]([5meC])[sP].[MOE](A)[sP].[MOE] |
| | | | ([5meC])[sP].[MOE](A)[sP].[MOE]([5meC])[sP].[MOE](A)[sP].[MOE] |
| | | | ([5meC]) |
| 94 | CACACACACCCACA | 94 | [MOE]([5meC])[sP].[MOE](A)[sP].[MOE]([5meC])[sP].[MOE](A)[sP]. |
| | CACACACACAC | | [MOE]([5meC])[sP].[MOE](A)[sP].[MOE]([5meC])[sP].[MOE](A)[sP]. |
| | | | [MOE]([5meC])[sP].[MOE]([5meC])[sP].[MOE]([5meC])[sP].[MOE](A) |
| | | | [sP].[MOE]([5meC])[sP].[MOE](A)[sP].[MOE]([5meC])[sP].[MOE](A) |
| | | | [sP].[MOE]([5meC])[sP].[MOE](A)[sP].[MOE]([5meC])[sP].[MOE](A) |
| | | | [sP].[MOE]([5meC])[sP].[MOE](A)[sP].[MOE]([5meC])[sP].[MOE](A) |
| | | | [sP].[MOE]([5meC]) |
| 95 | CACACACACACACA | 95 | [MOE]([5meC])[sP].[MOE](A)[sP].[MOE]([5meC])[sP].[MOE](A)[sP]. |
| | CCCACACACAC | | [MOE]([5meC])[sP].[MOE](A)[sP].[MOE]([5meC])[sP].[MOE](A)[sP]. |
| | | | [MOE]([5meC])[sP].[MOE](A)[sP].[MOE]([5meC])[sP].[MOE](A)[sP]. |
| | | | [MOE]([5meC])[sP].[MOE](A)[sP].[MOE]([5meC])[sP].[MOE]([5meC]) |
| | | | [sP].[MOE]([5meC])[sP].[MOE](A)[sP].[MOE]([5meC])[sP].[MOE](A) |
| | | | [sP].[MOE]([5meC])[sP].[MOE](A)[sP].[MOE]([5meC])[sP].[MOE](A) |
| | | | [sP].[MOE]([5meC]) |
| 96 | CACACACACACACA | 96 | [MOE]([5meC])[sP].[MOE](A)[sP].[MOE]([5meC])[sP].[MOE](A)[sP]. |
| | CGCACACACAC | | [MOE]([5meC])[sP].[MOE](A)[sP].[MOE]([5meC])[sP].[MOE](A)[sP]. |
| | | | [MOE]([5meC])[sP].[MOE](A)[sP].[MOE]([5meC])[sP].[MOE](A)[sP]. |
| | | | [MOE]([5meC])[sP].[MOE](A)[sP].[MOE]([5meC])[sP].[MOE](G)[sP]. |
| | | | [MOE]([5meC])[sP].[MOE](A)[sP].[MOE]([5meC])[sP].[MOE](A)[sP]. |
| | | | [MOE]([5meC])[sP].[MOE](A)[sP].[MOE]([5meC])[sP].[MOE](A)[sP]. |
| | | | [MOE]([5meC]) |
| 97 | CACACACACACACA | 97 | [MOE]([5meC])[sP].[MOE](A)[sP].[MOE]([5meC])[sP].[MOE](A)[sP]. |
| | CTCACACACAC | | [MOE]([5meC])[sP].[MOE](A)[sP].[MOE]([5meC])[sP].[MOE](A)[sP]. |
| | | | [MOE]([5meC])[sP].[MOE](A)[sP].[MOE]([5meC])[sP].[MOE](A)[sP]. |
| | | | [MOE]([5meC])[sP].[MOE](A)[sP].[MOE]([5meC])[sP].[MOE](T)[sP]. |
| | | | [MOE]([5meC])[sP].[MOE](A)[sP].[MOE]([5meC])[sP].[MOE](A)[sP]. |
| | | | [MOE]([5meC])[sP].[MOE](A)[sP].[MOE]([5meC])[sP].[MOE](A)[sP]. |
| | | | [MOE]([5meC]) |
| 98 | CACACACACATACA | 98 | [MOE]([5meC])[sP].[MOE](A)[sP].[MOE]([5meC])[sP].[MOE](A)[sP]. |
| | CACACACACAC | | [MOE]([5meC])[sP].[MOE](A)[sP].[MOE]([5meC])[sP].[MOE](A)[sP]. |
| | | | [MOE]([5meC])[sP].[MOE](A)[sP].[MOE](TilsP].[MOE](A)[sP].[MOE] |
| | | | ([5meC])[sP].[MOE](A)[sP].[MOE]([5meC])[sP].[MOE](A)[sP].[MOE] |
| | | | ([5meC])[sP].[MOE](A)[sP].[MOE]([5meC])[sP].[MOE](A)[sP].[MOE] |
| | | | ([5meC])[sP].[MOE](A)[sP].[MOE]([5meC])[sP].[MOE](A)[sP].[MOE] |
| | | | ([5meC]) |
| 99 | CACACACACAGACA | 99 | [MOE]([5meC])[sP].[MOE](A)[sP].[MOE]([5meC])[sP].[MOE](A)[sP]. |
| | CACACACACAC | | [MOE]([5meC])[sP].[MOE](A)[sP].[MOE]([5meC])[sP].[MOE](A)[sP]. |
| | | | [MOE]([5meC])[sP].[MOE](A)[sP].[MOE](G)[sP].[MOE](A)[sP].[MOE] |
| | | | ([5meC])[sP].[MOE](A)[sP].[MOE]([5meC])[sP].[MOE](A)[sP].[MOE] |
| | | | ([5meC])[sP].[MOE](A)[sP].[MOE]([5meC])[sP].[MOE](A)[sP].[MOE] |
| | | | ([5meC])[sP].[MOE](A)[sP].[MOE]([5meC])[sP].[MOE](A)[sP].[MOE] |
| | | | ([5meC]) |
| 100 | TTTACACACACACA | 100 | [MOE](T)[sP].[MOE](T)[sP].[MOE](T)[sP].[MOE](A)[sP].[MOE]([5meC]) |
| | CACACACACAC | | [sP].[MOE](A)[sP].[MOE]([5meC])[sP].[MOE](A)[sP].[MOE]([5meC]) |
| | | | [sP].[MOE](A)[sP].[MOE]([5meC])[sP].[MOE](A)[sP].[MOE]([5meC]) |
| | | | [sP].[MOE](A)[sP].[MOE]([5meC])[sP].[MOE](A)[sP].[MOE]([5meC]) |
| | | | [sP].[MOE](A)[sP].[MOE]([5meC])[sP].[MOE](A)[sP].[MOE]([5meC]) |
| | | | [sP].[MOE](A)[sP].[MOE]([5meC])[sP].[MOE](A)[sP].[MOE]([5meC]) |
| 101 | CACGCACACGCACA | 101 | [MOE]([5meC])[sP].[MOE](A)[sP].[MOE]([5meC])[sP].[MOE](G)[sP]. |
| | CACACACACAC | | [MOE]([5meC])[sP].[MOE](A)[sP].[MOE]([5meC])[sP].[MOE](A)[sP]. |
| | | | [MOE]([5meC])[sP].[MOE](G)[sP].[MOE]([5meC])[sP].[MOE](A)[sP]. |
| | | | [MOE]([5meC])[sP].[MOE](A)[sP].[MOE]([5meC])[sP].[MOE](A)[sP]. |
| | | | [MOE]([5meC])[sP].[MOE](A)[sP].[MOE]([5meC])[sP].[MOE](A)[sP]. |
| | | | [MOE]([5meC])[sP].[MOE](A)[sP].[MOE]([5meC])[sP].[MOE](A)[sP]. |
| | | | [MOE]([5meC]) |
| 102 | CACGCACACACGCA | 102 | [MOE]([5meC])[sP].[MOE](A)[sP].[MOE]([5meC])[sP].[MOE](G)[sP]. |
| | CACACACGCAC | | [MOE]([5meC])[sP].[MOE](A)[sP].[MOE]([5meC])[sP].[MOE](A)[sP]. |
| | | | [MOE]([5meC])[sP].[MOE](A)[sP].[MOE]([5meC])[sP].[MOE](G)[sP]. |
| | | | [MOE]([5meC])[sP].[MOE](A)[sP].[MOE]([5meC])[sP].[MOE](A)[sP]. |
| | | | [MOE]([5meC])[sP].[MOE](A)[sP].[MOE]([5meC])[sP].[MOE](A)[sP]. |
| | | | [MOE]([5meC])[sP].[MOE](GilsP].[MOE]([5meC])[sP].[MOE](A)[sP]. |
| | | | [MOE]([5meC]) |
| 103 | CACACGCACACACA | 103 | [MOE]([5meC])[sP].[MOE](A)[sP].[MOE]([5meC])[sP].[MOE](A)[sP]. |
| | CACACACACAC | | [MOE]([5meC])[sP].[MOE](G)[sP].[MOE]([5meC])[sP].[MOE](A)[sP]. |
| | | | [MOE]([5meC])[sP].[MOE](A)[sP].[MOE]([5meC])[sP].[MOE](A)[sP]. |
| | | | [MOE]([5meC])[sP].[MOE](A)[sP].[MOE]([5meC])[sP].[MOE](A)[sP]. |
| | | | [MOE]([5meC])[sP].[MOE](A)[sP].[MOE]([5meC])[sP].[MOE](A)[sP]. |
| | | | [MOE]([5meC])[sP].[MOE](A)[sP].[MOE]([5meC])[sP].[MOE](A)[sP]. |
| | | | [MOE]([5meC]) |

Helm Annotation Key:
[LR](G) is a beta-D-oxy-LNA guanine nucleoside,
[LR](T) is a beta-D-oxy-LNA thymine nucleoside,
[LR](A) is a beta-D-oxy-LNA adenine nucleoside,
[LR]([5meC] is a beta-D-oxy-LNA 5 methyl cytosine nucleoside,
[dR](G) is a DNA guanine nucleoside,
[dR](T) is a DNA thymine nucleoside,
[dR](A) is a DNA adenine nucleoside,
[dR]([C] is a DNA cytosine nucleoside,
[sP] is a phosphorothioate internucleoside linkage.
[mR]([C] is a 2′O-Methyl cytidine nucleoside
[mR]([A] is a 2′O-Methyl adenine nucleoside
[MOE]([5meC]) is a 2′O-MOE [2′O-(2-methoxyethyl)] 5-methyl cytidine nucleoside
[MOE](A) is a 2′O-MOE [2′O-(2-methoxyethyl)] adenine nucleoside
[dR]([In] is a DNA inosine nucleoside

1. A method of treating a disease characterized by TDP-43 pathology in a subject in need thereof, the method comprising administering to the subject an antisense oligonucleotide, of 8 to 40 nucleotides in length, which comprises a contiguous nucleotide sequence of at least 8 nucleotides in length with at least 90% complementarity to a sequence selected from the group consisting of (5′-3′) (UG)n, (GU)n, wherein n is 4-20, UGUGUGUG, UGUGUGUGU, UGUGUGUGUG, UGUGUGUGUGU, UGUGUGUGUGUG, UGUGUGUGUGUGU, GUGUGUGU, GUGUGUGUG, GUGUGUGUGU, GUGUGUGUGUGU, GUGUGUGUGUGUG, and GUGAAUGA, wherein the TDP-43 pathology is a neurological disorder.

2. The method according to claim 1, wherein the contiguous nucleotide sequence comprises one or more modified nucleosides.

3. The method according to claim 1, wherein the contiguous nucleotide sequence comprises SEQ ID NO: 27.

4. The method according to claim 1, wherein the contiguous nucleotide sequence is at least 12 nucleotides in length.

5. The method according to claim 1, wherein the antisense oligonucleotide does not comprise a region of more than 3, or more than 4, contiguous DNA nucleosides.

6. The method according to claim 2, wherein the one or more modified nucleosides, is a 2′ sugar modified nucleoside, such as a 2′ sugar modified nucleoside independently selected from the group consisting of 2′-O-alkyl-RNA; 2′-O-methyl RNA (2′-OMe); 2′-alkoxy-RNA; 2′-O-methoxyethyl-RNA (2′-MOE); 2′-amino-DNA; 2′-fluro-RNA; 2′-fluoro-DNA; arabino nucleic acid (ANA); 2′-fluoro-ANA; locked nucleic acid (LNA), and any combination thereof.

7. The method according to claim 6, wherein the 2′ sugar modified nucleoside is an affinity enhancing 2′ sugar modified nucleoside.

8. The method according to claim 2, wherein the contiguous nucleotide sequence of the antisense oligonucleotide comprises 2′-O-methoxyethyl-RNA (2′-MOE) nucleosides.

9. The method according to claim 8, wherein all the nucleosides of the contiguous nucleotide sequence are 2′-O-methoxyethyl-RNA (2′-MOE) nucleosides.

10. The method according to claim 9, wherein all the contiguous nucleotide sequence of the antisense oligonucleotide are linked by phosphorothioate internucleoside linkages.

11. The method according to claim 2, wherein one or more of the modified nucleosides is a locked nucleic acid nucleoside (LNA).

12. The method according to claim 11, wherein the LNA nucleoside is selected from the group consisting of constrained ethyl nucleoside (cEt), and β-Doxy-LNA.

13. The method according to claim 2, wherein the contiguous nucleotide sequence of the antisense oligonucleotide comprises or consists of LNA nucleosides and DNA nucleosides.

14. The method according to claim 2, wherein the antisense oligonucleotide or contiguous nucleotide sequence thereof is a mixmer or a totalmer.

15. The method according to claim 2, wherein the cytosine bases present in the antisense oligonucleotide or contiguous nucleotide sequence thereof are independently selected from the group consisting of cytosine and 5-methyl cytosine.

16. The method according to claim 2, wherein one or more of the internucleoside linkages positioned between the nucleosides on the contiguous nucleotide sequence are modified.

17. The method according to claim 16, wherein one or more, or all of the modified internucleoside linkages comprise a phosphorothioate linkage.

18. The method according to claim 2, wherein all the internucleoside linkages present in the antisense oligonucleotide are phosphorothioate internucleoside linkages.

19. The method according to claim 1, wherein the length of the contiguous nucleotide sequence is 12, 13, 14, 15, 16, 17, 18, 19, 20, 21, 22, 23, 24, 25, 26, 27, 28, 29, or 30 nucleotides.

20. The method according to claim 1, wherein the antisense oligonucleotide consists of the contiguous nucleotide sequence.

21. The method according to claim 1, wherein the antisense oligonucleotide is formulated as a pharmaceutically acceptable salt.

22. The method according to claim 21, wherein the pharmaceutically acceptable salt is a sodium salt or a potassium salt.

23. The method according to claim 1, wherein the antisense oligonucleotide is formulated as a pharmaceutical composition, wherein the pharmaceutical composition comprises the antisense oligonucleotide and a pharmaceutically acceptable diluent, solvent, carrier, salt, and/or adjuvant.

24. The method according to claim 1, wherein the neurological disorder is amyotrophic lateral sclerosis (ALS), frontotemporal lobar degeneration (FTLD), Progressive supranuclear palsy (PSP), Primary lateral sclerosis, Progressive muscular atrophy, Alzheimer's disease, Parkinsons disease, Autism, Hippocampal sclerosis dementia, Down syndrome, Huntington's disease, a polyglutamine disease, spinocerebellar ataxia 3, a myopathy, or Chronic Traumatic Encephalopathy.

25. A method of treating a disease characterized by TDP-43 pathology in a subject in need thereof, the method comprising administering to the subject a conjugate comprising:
(i) an antisense oligonucleotide, of 8 to 40 nucleotides in length, which comprises a contiguous nucleotide sequence of at least 8 nucleotides in length with at least 90% complementarity to a sequence selected from the group consisting of (5′-3′) (UG)n, (GU)n, wherein n is 4-20, UGUGUGUG, UGUGUGUGU, UGUGUGUGUG, UGUGUGUGUGU, UGUGUGUGUGUG, UGUGUGUGUGUGU, GUGUGUGU, GUGUGUGUG, GUGUGUGUGU, GUGUGUGUGUGU, GUGUGUGUGUGUG, and GUGAAUGA; and
(ii) at least one conjugate moiety covalently attached to said oligonucleotide, and wherein the TDP-43 pathology is a neurological disorder.

26. The method according to claim 25, wherein the antisense oligonucleotide is formulated as a pharmaceutically acceptable salt.

27. The method according to claim 26, wherein the pharmaceutically acceptable salt is a sodium salt or a potassium salt.

28. The method according to claim 25, wherein the antisense oligonucleotide is formulated as a pharmaceutical composition, wherein the pharmaceutical composition comprises the antisense oligonucleotide and a pharmaceutically acceptable diluent, solvent, carrier, salt, and/or adjuvant.

29. The method according to claim 25, wherein the neurological disorder is amyotrophic lateral sclerosis (ALS), frontotemporal lobar degeneration (FTLD), Progressive supranuclear palsy (PSP), Primary lateral sclerosis, Progressive muscular atrophy, Alzheimer's disease, Parkinsons disease, Autism, Hippocampal sclerosis dementia, Down syndrome, Huntington's disease, a polyglutamine disease, spinocerebellar ataxia 3, a myopathy, or Chronic Traumatic Encephalopathy.
